(19) United States
(12) Patent Application Publication
(10) Pub. No.: US 2026/0144857 A1
(43) Pub. Date: May 28, 2026

(54) SHIGELLA MULTIPLE ANTIGEN PRESENTING IMMUNOGENIC COMPOSITION AND FUSION PROTEINS THEREOF

(71) Applicant: THE CHILDREN'S MEDICAL CENTER CORPORATION, Boston, MA (US)

(72) Inventors: Richard MALLEY, Beverly, MA (US); Fan ZHANG, West Roxbury, MA (US); Yingjie LU, West Roxbury, MA (US)

(73) Assignee: THE CHILDREN'S MEDICAL CENTER CORPORATION, Boston, MA (US)

(21) Appl. No.: 19/120,008

(22) PCT Filed: Oct. 13, 2023
(86) PCT No.: PCT/US2023/076822

§ 371 Date: Apr. 10, 2025

Related U.S. Application Data
(60) Provisional application No. 63/416,485, filed on Oct. 14, 2022.
(60) Provisional application No. 63/428,633, filed on Nov. 29, 2022.

(51) Int. Cl. A61K 39/112; A61K 39/00; A61K 39/05; A61P 37/04; C07K 14/25; C07K 14/255

(52) U.S. Cl. CPC A61K 39/0283; A61K 39/0275; A61K 39/05; A61P 37/04; C07K 14/25; C07K 14/255; A61K 2039/70; C07K 2319/00

(57) ABSTRACT

Technologies for the prevention and/or treatment of Shigella infections.

### CROSS REFERENCE TO RELATED APPLICATIONS

This application claims benefit under 35 U.S.C. § 119(e) of U.S. Provisional Application No. 63/416,485 filed Oct. 14, 2022 and U.S. Provisional Application No. 63/428,633 filed Nov. 29, 2022, the contents of each of which are incorporated herein by reference in their entireties.

### SEQUENCE LISTING

The instant application contains a Sequence Listing which has been submitted electronically in XML format, and is hereby incorporated by reference in its entirety. Said XML copy, created on Oct. 2, 2023 is named “701039-000101WOPT_SL.xml” and is 73,744 bytes in size.

### FIELD OF THE INVENTION

The present invention relates to technologies, compositions, and methods for the prevention and/or treatment of Shigella infections.

### BACKGROUND OF INVENTION

Shigella species are the major pathogenic bacteria causing bacterial diarrhea in developing countries, which infect 164.7 million people and lead to 0.11 million deaths per year, most of which are children under 5 years old (Kotloff, et al. 1999. Global burden of Shigella infections: implications for vaccine development and implementation of control strategies. Bull World Health Organ 77:651-66). Among the four serogroups of Shigella, Shigella flexneri is the predominant serogroup that affects the low-income population.

Shigellosis is a severe diarrheal disease caused by infection by bacteria of the genus Shigella. Shigellosis is associated with high morbidity and mortality rates, particularly in the developing world. It is also responsible for long-term effects on cognitive and physical development in children. The global burden has been estimated at more than 160 million cases per year; the most affected being children under 5 years of age living in endemic areas. The causative agent can be transmitted from person to person through fecal-oral contact or through contaminated fomites; ingestion of as few as 10 organisms can cause illness in adult volunteers. In industrialized countries, Shigella is known to be responsible for cases of pediatric diarrhea and to cause occasional foodborne outbreaks. Other susceptible groups include travelers, military personnel and refugees. Additionally, the Centers for Disease Control and Prevention lists Shigella as a food safety threat.

Worldwide, Shigella is estimated to cause 80-165 million cases of disease and 600,000 deaths annually. Shigella spp. are endemic in temperate and tropical climates. Transmission of Shigella spp. is most likely when hygiene and sanitation are insufficient. Shigellosis is predominantly caused by S. sonnei in industrialized countries, whereas S. flexneri prevails in the developing world. Infections caused by S. boydii and S. dysenteriae are less common globally but can make up a substantial proportion of Shigella spp. isolated in sub-Saharan Africa and South Asia. Shigella spp. are detected in the stools of 5%—18% of patients with travelers' diarrhea. In a study of travel-associated enteric infections diagnosed after return to the United States, Shigella was the third most common bacterial pathogen isolated by clinical laboratories (of note, these laboratories did not test for enterotoxigenic Escherichia coli, a common cause of travelers' diarrhea). Many infections caused by S. dysenteriae (56%) and S. boydii (44%) were travel-associated, but infections caused by S. flexneri and S. sonnei were less often associated with travel (24% and 12%, respectively). Outbreaks of infections caused by multidrug-resistant Shigella, including isolates resistant to azithromycin or ciprofloxacin, have been reported in Australia, Europe, and North America.

Shigella is a genus of bacteria that is Gram negative, facultative anaerobic, non-spore-forming, nonmotile, rod shape and genetically related to E. coli. It is the causative agent of human shigellosis, which results in dysentery. The Shigella genus comprises four different species, classified by four different serogroups. Serogroup A comprises of 15 different serotypes; an exemplary example is S. dysenteriae. Serogroup B comprises of 9 serotypes; an exemplary example is S. flexneri. Serogroup C comprises of 19 serotypes; an exemplary example is S. boydii. Serogroup D comprises of one serotype, S. sonnei. Serogroups A-C are physiologically similar whereas Serogroup D is different based on its metabolism.

These species of Shigella are further divided into multiple serotypes, based on the structure of the O-polysaccharide portion of their outer membrane lipopolysaccharide (LPS), thereby increasing their antigenic variability. One serotype in particular, S. dysenteriae serotype 1, which produces Shiga toxin, is responsible for the most severe infections, including hemolytic uremic syndrome and it is the cause of epidemic dysentery. Additionally, serotypes can drift during outbreaks, further limiting the efficacy of vaccines that are restricted to particular serotypes. The emergence of strains resistant to antimicrobial drugs, including ciprofloxacin, currently the first-line antibiotic treatment against Shigella infections, heightens the difficulty of controlling this pathogen and makes the development of an effective vaccine even more urgent.

Shigella flexneri is further classified into various serotypes according to the different 0-antigen structures. To date, at least 15 serotypes have been reported, i.e. 1a, 1b, 1c, 2a, 2b, 3a, 3b, 4a, 4b, 5a, 5b, X, Xv, Y, and F6 (Simmons, D. A., and E. Romanowska. 1987. Structure and biology of Shigella flexneri O antigens. J. Med. Microbiol. 23:289-302; Stagg, R. M., S. S. Tang, N. I. Carlin, K. A. Talukder, P. D. Cam, and N. K. Verma. 2009. A novel glucosyltransferase involved in O-antigen modification of Shigella flexneri serotype 1c. J. Bacteriol. 191:6612-7; Ye, C., R. Lan, S. Xia, J. Zhang, Q. Sun, S. Zhang, H. Jing, L. Wang, Z. Li, Z. Zhou, A. Zhao, Z. Cui, J. Cao, D. Jin, L. Huang, Y. Wang, X. Luo, X. Bai, P. Wang, Q. Xu, and J. Xu. 2010. Emergence of a new multidrug-resistant serotype X variant in an epidemic clone of Shigella flexneri. J. Clin. Microbiol. 48:419-26). Serotyping has long been used to characterize isolates for epidemiological purposes.

The LPSs of all Shigella flexneri serotypes except F6 have the same polysaccharide backbone consisting of repeating tetrasaccharide units, and serotype Y has the basic tetrasaccharide backbone (Simmons, D. A., and E. Romanowska. 1987. Structure and biology of Shigella flexneri O antigens. J. Med. Microbiol. 23:289-302). Modification by glycosylation and/or acetylation of different sugars on the backbone gives rise to various type-specific antigenic determinants (e.g., I, II, III, IV, V, and VI), group-specific antigenic determinants (e.g., 3,4; 6; and 7,8), and antigenic determinant 1c (Stagg, R. M., S. S. Tang, N. I. Carlin, K. A. Talukder, P. D. Cam, and N. K. Verma. 2009. A novel glucosyltransferase involved in 0-antigen modification of Shigella flexneri serotype 1c. J. Bacteriol. 191:6612-7).

Three genes (gtrA, gtrB, and gtr[type]) are responsible for glycosylation modifications on Shigella flexneri. The first two genes are highly homologous and interchangeable, whereas the third gene gtr[type] is unique, and encodes serotype-specific glycosyltransferases (Allison, G. E., and N. K. Verma. 2000. Serotype-converting bacteriophages and O-antigen modification in Shigella flexneri. Trends. Microbiol. 8:17-23; Stagg, R. M., S. S. Tang, N. I. Carlin, K. A. Talukder, P. D. Cam, and N. K. Verma. 2009. A novel glucosyltransferase involved in O-antigen modification of Shigella flexneri serotype 1c. J. Bacteriol. 191:6612-7). Gtr genes specific to type-antigens I, II, IV and V, group antigen 7,8 and antigen 1c are gtrI, gtrII, gtrIV, gtrV, gtrX and gtrIC, respectively (Adams, M. M., G. E. Allison, and N. K. Verma. 2001. Type IV O antigen modification genes in the genome of Shigella flexneri NCTC 8296. Microbiology 147:851-60; Adhikari, P., G. Allison, B. Whittle, and N. K. Verma. 1999. Serotype 1a 0-antigen modification: molecular characterization of the genes involved and their novel organization in the Shigella flexneri chromosome. J Bacteriol 181:4711-8; Guan, S., D. A. Bastin, and N. K. Verma. 1999. Functional analysis of the O antigen glucosylation gene cluster of Shigella flexneri bacteriophage SfX. Microbiology 145: 1263-73; Huan, P. T., D. A. Bastin, B. L. Whittle, A. A. Lindberg, and N. K. Verma. 1997. Molecular characterization of the genes involved in O-antigen modification, attachment, integration and excision in Shigella flexneri bacteriophage SfV. Gene 195:217-27; Mavris, M., P. A. Manning, and R. Morona. 1997. Mechanism of bacteriophage SflI-mediated serotype conversion in Shigella flexneri. Mol Microbiol 26:939-50; Stagg, R. M., S. S. Tang, N. I. Carlin, K. A. Talukder, P. D. Cam, and N. K. Verma. 2009. A novel glucosyltransferase involved in O-antigen modification of Shigella flexneri serotype 1c. J Bacteriol 191:6612-7). The gtr genes are carried by prophages integrated in the genome of host bacteria. O-acetylation, which confers group-antigen 6 and/or type-antigen III on strains of serotypes 1b, 3a, 3b and 4b, is mediated by the oac gene carried in bacteriophage Sf6 (Clark, C. A., J. Beltrame, and P. A. Manning. 1991. The oac gene encoding a lipopolysaccharide 0-antigen acetylase maps adjacent to the integrase-encoding gene on the genome of Shigella flexneri bacteriophage Sf6. Gene 107:43-52; Verma, N. K., J. M. Brandt, D. J. Verma, and A. A. Lindberg. 1991. Molecular characterization of the O-acetyl transferase gene of converting bacteriophage Sf6 that adds group antigen 6 to Shigella flexneri. Mol. Microbiol. 5:71-5). Strains of different serotypes carry one or more serotype-specific prophages that encode different specific 0-antigen modifications genes (see FIG. 1).

Despite being a longstanding priority for the World Health Organization, and the progress made in recent years, no licensed vaccine for Shigella spp. currently exists. Efforts to develop a vaccine against this pathogen have included the use of killed bacteria, live attenuated and recombinant carrier organisms, polysaccharide conjugates and LPS/protein mixtures. When tested in humans, these vaccines were either too reactogenic or poorly immunogenic. A major disadvantage of these candidates is the O antigen restriction, which limits the scope of protection they can offer and requires the development of a multi-serotype vaccine to provide adequate protective coverage in Shigella endemic areas.

There remains a need in the art for vaccines and agents for preventing Shigella infections, as well as controlling Shigella in several critical areas, such as clinical applications, food safety-related uses, and environmental decontamination. In particular, there is a medical need for a vaccine that provides T-cell dependent immunity against a broad range of serotypes of Shigella. There remains a medical need for a vaccine that provides immunity against a broad range of serotypes of Shigella.

### SUMMARY OF INVENTION

The present disclosure addresses the lack of suitable technologies for the prevention and/or treatment of Shigella infections (i.e., Shigellosis). Among other things, the present disclosure addresses challenges in providing vaccines with sufficient immunogenicity to protect against Shigella. Technologies described herein can induce a T-and B-cell response and/or provide immunity against a broad range of Shigella serotypes.

In some embodiments, a vaccine comprises an immunogenic complex, wherein the immunogenic complex comprises: (a) a biotinylated polysaccharide antigen; and (b) a fusion protein comprising: (i) a biotin-binding moiety; and (ii) at least one polypeptide antigen; wherein the biotinylated polysaccharide antigen comprises an O-specific polysaccharide (OSP) from Shigella, and further wherein the biotinylated polysaccharide antigen is non-covalently associated with the biotin-binding moiety of the fusion protein to form an immunogenic complex.

In some embodiments, the biotinylated polysaccharide antigen comprises a polysaccharide of Shigella selected from Shigella flexneri or Shigella sonnei, e.g., Shigella flexneri serotype selected from: 2a, 3a or 6, or Shigella sonnei.

In some embodiments, the at least one polypeptide antigen of the fusion protein is or comprises a polypeptide antigen from Salmonella, Shigella, or Streptococcus pneumoniae.

In some embodiments, the at least one polypeptide antigen of the fusion protein comprises a Salmonella SseB polypeptide or antigenic fragment thereof.

In some embodiments, the at least one polypeptide antigen of the fusion protein comprises a Shigella IpaB polypeptide or antigenic fragment thereof.

In some embodiments, the at least one polypeptide antigen of the fusion protein comprises a Streptococcus pneumoniae SP1500 polypeptide or antigenic fragment thereof, a Streptococcus pneumoniae SP0785 polypeptide or antigenic fragment thereof, or both.

In some embodiments, the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 (SseB)

In some embodiments, the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5 (IpaB)

In some embodiments, the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8 (SP0785) or SEQ ID NO: 9 (SP1500), or a combination of SEQ ID NO: 8 and SEQ ID NO: 9.

In some embodiments, the biotin-binding moiety is a polypeptide comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 3 (Rhavi) or a biotin-binding fragment thereof.

Herein, one aspect of the present invention relates to a multi-valent Shigella vaccine using the Multiple Antigen Presenting System (MAPS). The disclosed multi-valent MAPS-Shigella vaccine is an advancement on the MAPS system previously disclosed in U.S. Pat. No. 10,766,932, which is incorporated herein in its entirety by reference.

In particular, one aspect of the present invention relates to a MAPS-Shigella immunogenic complex comprising a biotinylated O-specific polysaccharide (OSP) antigen from Shigella spp. serogroup, and (b) a fusion protein comprising: (i) a biotin-binding moiety (BBM); and (ii) at least one polypeptide antigen comprises a IpaB polypeptide, wherein the fusion protein comprises (i) an amino acid sequence that is at least 80% identical to SEQ ID NO: 3 (Rhavi) and (ii) an amino acid sequence that is at least 80% identical to SEQ ID NO: 5 (IpaB), and wherein in each species of the immunogenic complexes, the biotinylated OSP antigen is non-covalently associated with the biotin-binding moiety of the fusion protein.

In another aspect of the present invention relates to a MAPS-Shigella immunogenic complex comprising a biotinylated O-specific polysaccharide (OSP) antigen from Shigella spp serogroup, and (b) a fusion protein comprising: (i) a biotin-binding moiety (BBM); and (ii) at least one polypeptide antigen comprises a SseB polypeptide, wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB), and wherein in each species of the immunogenic complexes, the biotinylated OSP antigen is non-covalently associated with the biotin-binding moiety of the fusion protein.

In another aspect of the present invention relates to a MAPS-Shigella immunogenic complex comprising a biotinylated O-specific polysaccharide (OSP) antigen from Shigella spp serogroup, and (b) a fusion protein comprising: (i) a biotin-binding moiety (BBM); and (ii) at least one polypeptide antigen comprises a SP1500 polypeptide or SP0785 polypeptide, wherein the fusion protein comprises (i) an amino acid sequence that is at least 80% identical to SEQ ID NO: 3 (Rhavi) and (ii) an amino acid sequence that is at least 80% identical to any one of: SEQ ID NO: 8 (SP785), SEQ ID NO: 9 (SP1500) or SEQ ID NO: 6 (CP1), and wherein in each species of the immunogenic complexes, the biotinylated OSP antigen is non-covalently associated with the biotin-binding moiety of the fusion protein.

In some embodiments, an immunogenic composition (e.g., a vaccine) comprises a plurality of different species of immunogenic complexes, wherein the different species comprise: a plurality of biotinylated polysaccharide antigens comprising polysaccharide antigens of one or more Shigella serotypes; and a plurality of fusion proteins, each fusion protein comprising: a biotin-binding moiety; and a polypeptide antigen, wherein each of the plurality of biotinylated polysaccharide antigens is non-covalently associated with the biotin-binding moiety of one or more of the plurality of fusion proteins to form an immunogenic complex.

In some embodiments, an immunogenic composition (e.g., a vaccine) as disclosed herein further comprises a plurality of different species of immunogenic complexes, wherein the different species comprise: a plurality of biotinylated polysaccharide antigens comprising polysaccharide antigens of one or more Shigella serotypes, and a plurality of biotinylated polysaccharide antigens comprising polysaccharide antigens of one or more Salmonella serotypes and a plurality of fusion proteins, each fusion protein comprising: a biotin-binding moiety; and a polypeptide antigen, wherein each of the plurality of biotinylated polysaccharide antigens is non-covalently associated with the biotin-binding moiety of one or more of the plurality of fusion proteins to form an immunogenic complex.

In some embodiments, the different species each comprise a distinct polysaccharide antigen of one or more Shigella serotypes and/or a distinct polypeptide antigen.

In some embodiments, the one or more Shigella serotypes is Shigella flexneri or Shigella sonnei, e.g., Shigella flexneri serotype selected from: 2a, 3a or 6, or Shigella sonnei, or combinations thereof.

In some embodiments, the polypeptide antigen is or comprises a polypeptide antigen from any one or more of: Salmonella, Shigella, and/or Streptococcus pneumoniae.

In some embodiments, the polypeptide antigen is or comprises: an SseB polypeptide antigen of Salmonella, an IpaB polypeptide antigen from Shigella, and/or a polypeptide antigen comprising an SP1500 polypeptide and/or an SP0785 polypeptide, of S. pneumoniae.

In some embodiments, the immunogenic composition comprises at least two different species of immunogenic complexes, wherein the different species comprise: a first biotinylated polysaccharide antigen comprising a polysaccharide of Shigella flexneri non-covalently complexed with a first fusion protein; and a second biotinylated polysaccharide antigen comprising a polysaccharide of Shigella sonnei, non-covalently complexed with a second fusion protein, wherein the first fusion protein and the second fusion protein each independently comprise: a biotin-binding moiety; and a polypeptide antigen, wherein each of the first and second biotinylated polysaccharide antigens is non-covalently associated with the biotin-binding moiety of the respective fusion protein to form an immunogenic complex.

In some embodiments, the polypeptide antigen of the first fusion protein and of the second fusion protein is or comprises an SseB polypeptide antigen of Salmonella.

In some embodiments, the immunogenic composition comprises at least two different species of immunogenic complexes, wherein the different species comprise: a first biotinylated polysaccharide antigen comprising a polysaccharide of Shigella flexneri serotype 2a non-covalently complexed with a first fusion protein; and a second biotinylated polysaccharide antigen comprising a polysaccharide of any of: Shigella flexneri serotype 3a, 6 or Shigella sonnei non-covalently complexed with a second fusion protein, wherein the first fusion protein and the second fusion protein each independently comprise: a biotin-binding moiety; and a polypeptide antigen, wherein each of the first and second biotinylated polysaccharide antigens is non-covalently associated with the biotin-binding moiety of the respective fusion protein to form an immunogenic complex.

In some embodiments, the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof.

In some embodiments, the polypeptide antigen of the first fusion protein and of the second fusion protein is or comprises an IpaB polypeptide antigen of Shigella.

In some embodiments, the IpaB polypeptide antigen of Shigella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5 or an immunogenic fragment thereof.

In some embodiments, the polypeptide antigen of the first fusion protein and of the second fusion protein is or comprises an SP1500 polypeptide, an SP0785 polypeptide, or both, of S. pneumoniae, and/or the fusion protein is CP1 having an amino acid sequence that is at least 85%, or least 90%, at least 91%, at least 92%, at least 93%, at least 94%, at least 95%, at least 96%, at least 97%, at least 98%, at least 99%, or at least 99.5% identical to SEQ ID NO: 6, or an immunogenic fragment thereof.

In some embodiments, the SP1500 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 9, or an immunogenic fragment thereof, the SP0785 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, and the CP1 fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 6, or an immunogenic fragment thereof.

In some embodiments, the immunogenic composition as disclosed herein, comprises at least four different species of immunogenic complexes, wherein the different species comprise: a first biotinylated polysaccharide antigen comprising a polysaccharide of Shigella flexneri serotype 2a non-covalently complexed with a first fusion protein; and a second biotinylated polysaccharide antigen comprising a polysaccharide of Shigella flexneri serotype 3a non-covalently complexed with a second fusion protein, a third biotinylated polysaccharide antigen comprising a polysaccharide of Shigella flexneri serotype 6 non-covalently complexed with a third fusion protein; and a fourth biotinylated polysaccharide antigen comprising a polysaccharide of Shigella sonnei non-covalently complexed with a fourth fusion protein, wherein the first fusion protein, the second fusion protein, the third fusion protein, and the fourth fusion protein each independently comprise: a biotin-binding moiety; and a polypeptide antigen, wherein each of the first, second, third, and fourth biotinylated polysaccharide antigens is non-covalently associated with the biotin-binding moiety of the respective fusion protein to form an immunogenic complex.

In some embodiments, the polypeptide antigen of the first fusion protein and of the second fusion protein is or comprises an IpaB polypeptide antigen as disclosed herein, and the polypeptide antigen of the third fusion protein and of the fourth fusion protein is or comprises an SP1500 polypeptide, an SP0785 polypeptide, or both, of S. pneumoniae, and/or the fusion protein is CP1.

In some embodiments, the polypeptide antigen of the first fusion protein and of the second fusion protein is or comprises an SseB polypeptide antigen, and the polypeptide antigen of the third fusion protein and of the fourth fusion protein is or comprises an SP1500 polypeptide, an SP0785 polypeptide, or both, of S. pneumoniae, and/or the fusion protein is CP1.

In some embodiments, the SseB polypeptide antigen is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof, the SP1500 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, the SP0785 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 9, or an immunogenic fragment thereof, and the CP1 fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 6, or an immunogenic fragment thereof.

In some embodiments, the immunogenic composition comprises at least four different species of immunogenic complexes, wherein the different species comprise: a first biotinylated polysaccharide antigen comprising a polysaccharide of Shigella flexneri serotype 2a non-covalently complexed with a first fusion protein; and a second biotinylated polysaccharide antigen comprising a polysaccharide of Shigella flexneri serotype 3a non-covalently complexed with a second fusion protein, a third biotinylated polysaccharide antigen comprising a polysaccharide of Shigella flexneri serotype 6 non-covalently complexed with a third fusion protein; and a fourth biotinylated polysaccharide antigen comprising a polysaccharide of Shigella sonnei non-covalently complexed with a fourth fusion protein, wherein the first fusion protein, the second fusion protein, the third fusion protein, and the fourth fusion protein each independently comprise: a biotin-binding moiety; and a polypeptide antigen, wherein each of the first, second, third, and fourth biotinylated polysaccharide antigens is non-covalently associated with the biotin-binding moiety of the respective fusion protein to form an immunogenic complex.

In some embodiments, the polypeptide antigen of the first fusion protein, the second fusion protein, the third fusion protein, and the fourth fusion protein is or comprises an SseB polypeptide antigen of Salmonella.

In some embodiments, the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof.

In some embodiments, the polypeptide antigen of the first fusion protein, the second fusion protein, the third fusion protein, and the fourth fusion protein is or comprises an IpaB polypeptide.

In some embodiments, the IpaB polypeptide antigen of Shigella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5 or an immunogenic fragment thereof.

In some embodiments, a vaccine comprises a plurality of different species of immunogenic complexes, wherein the different species comprise: a biotinylated polysaccharide antigen of Shigella flexneri serotype 2a non-covalently complexed with a fusion protein; a biotinylated polysaccharide antigen of Shigella flexneri serotype 3a non-covalently complexed with a fusion protein; wherein each fusion protein comprises (a) a biotin-binding moiety; and (b) a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and wherein each of the biotinylated polysaccharide antigens is non-covalently associated with the biotin-binding moiety of at least one fusion protein to form an immunogenic complex.

In some embodiments, a vaccine comprises a plurality of different species of immunogenic complexes, wherein the different species comprise: a biotinylated polysaccharide antigen of Shigella flexneri serotype 2a non-covalently complexed with a fusion protein; a biotinylated polysaccharide antigen of Shigella flexneri serotype 6 non-covalently complexed with a fusion protein; wherein each fusion protein comprises (a) a biotin-binding moiety; and (b) a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and wherein each of the biotinylated polysaccharide antigens is non-covalently associated with the biotin-binding moiety of at least one fusion protein to form an immunogenic complex.

In some embodiments, a vaccine comprises a plurality of different species of immunogenic complexes, wherein the different species comprise: a biotinylated polysaccharide antigen of Shigella flexneri serotype 2a non-covalently complexed with a fusion protein; a biotinylated polysaccharide antigen of Shigella sonnei non-covalently complexed with a fusion protein; wherein each fusion protein comprises (a) a biotin-binding moiety; and (b) a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and wherein each of the biotinylated polysaccharide antigens is non-covalently associated with the biotin-binding moiety of at least one fusion protein to form an immunogenic complex.

In some embodiments, a vaccine comprises a plurality of different species of immunogenic complexes, wherein the different species comprise: a biotinylated polysaccharide antigen of Shigella flexneri serotype 3a non-covalently complexed with a fusion protein; a biotinylated polysaccharide antigen of Shigella flexneri serotype 6 non-covalently complexed with a fusion protein; wherein each fusion protein comprises (a) a biotin-binding moiety; and (b) a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and wherein each of the biotinylated polysaccharide antigens is non-covalently associated with the biotin-binding moiety of at least one fusion protein to form an immunogenic complex.

In some embodiments, a vaccine comprises a plurality of different species of immunogenic complexes, wherein the different species comprise: a biotinylated polysaccharide antigen of Shigella flexneri serotype 3a non-covalently complexed with a fusion protein; a biotinylated polysaccharide antigen of Shigella sonnei non-covalently complexed with a fusion protein; wherein each fusion protein comprises (a) a biotin-binding moiety; and (b) a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof; and wherein each of the biotinylated polysaccharide antigens is non-covalently associated with the biotin-binding moiety of at least one fusion protein to form an immunogenic complex.

In some embodiments, a vaccine comprises a plurality of different species of immunogenic complexes, wherein the different species comprise: a biotinylated polysaccharide antigen of Shigella flexneri serotype 6 non-covalently complexed with a fusion protein; a biotinylated polysaccharide antigen of Shigella sonnei non-covalently complexed with a fusion protein; wherein each fusion protein comprises (a) a biotin-binding moiety; and (b) a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof; and wherein each of the biotinylated polysaccharide antigens is non-covalently associated with the biotin-binding moiety of at least one fusion protein to form an immunogenic complex.

In some embodiments, the first polypeptide antigen comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

In some embodiments, the first polypeptide antigen comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

In some embodiments, the first polypeptide antigen comprises an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8, SEQ ID NO: 9, or both SEQ ID NO: 8 and SEQ ID NO:9.

In some embodiments, the vaccine comprises a stoichiometrically equal ratio, by weight, of each of the polysaccharide antigens of the different species.

In some embodiments, the vaccine comprises at least one of the polysaccharide antigens of the different species at a stoichiometrically different ratio, by weight.

In some embodiments, the vaccine comprises a stoichiometrically different ratio, by weight, of each of the polysaccharide antigens of the different species.

In some embodiments, the biotin-binding moiety is a polypeptide comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 3.

In some embodiments, an immunogenic complex comprises a biotinylated polysaccharide antigen of Shigella non-covalently associated with a fusion protein, wherein the fusion protein comprises a biotin-binding moiety and at least one polypeptide antigen.

In some embodiments, the biotinylated polysaccharide antigen comprises a polysaccharide of Shigella having a serotype selected from Shigella flexneri serotype selected from: 2a, 3a or 6, or Shigella sonnei.

In some embodiments, the fusion protein comprises at least one polypeptide selected from: SseB, IpaB, SP1500 polypeptide, or SP0785, or a combination of at least two polypeptides selected from any of: SseB, IpaB, SP1500 or SP0785. In some embodiments, exemplary fusion proteins envisioned for use in the immunogenic compositions or vaccine compositions are disclosed in Table 1.

In some embodiments, the fusion protein comprises: (a) a polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an antigenic fragment thereof, (b) a polypeptide antigen comprising an amino acid sequence at least 90%, at least 910%, at least 92%, at least 93%, at least 94%, at least 95%, at least 96%, at least 97%, at least 98%, at least 99%, or at least 99.5% identical to SEQ ID NO: 5 or an antigenic fragment thereof, or (c) a polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof.

In some embodiments, the immunogenic complex comprises a ratio of fusion protein to polysaccharide antigen of about 1:1, about 2:1, about 3:1, about 4:1, about 5:1, about 6:1, about 7:1, about 8:1, about 9:1, or about 10:1, by weight.

In some embodiments, a vaccine comprises one or more of the above immunogenic complexes.

In some embodiments, a pharmaceutical composition comprises a vaccine and a pharmaceutically acceptable carrier.

In some embodiments, a pharmaceutical composition comprises an immunogenic complex, and a pharmaceutically acceptable carrier.

In some embodiments, the pharmaceutical composition further comprises one or more adjuvants.

In some embodiments, the one or more adjuvants is or comprises a co-stimulation factor.

In some embodiments, the one or more adjuvants are selected from the group consisting of aluminum phosphate, aluminum hydroxide, and phosphated aluminum hydroxide.

In some embodiments, the one or more adjuvants is or comprises aluminum phosphate.

In some embodiments, the pharmaceutical composition is formulated for injection.

In some embodiments, upon administration to a subject, the pharmaceutical composition induces an immune response.

In some embodiments, the immune response is to (i) at least one polysaccharide antigen of the vaccine or immunogenic complex, and/or (ii) at least one polypeptide antigen of the vaccine or immunogenic complex.

In some embodiments, a method of making a vaccine, comprises non-covalently complexing a plurality of biotinylated polysaccharide antigens with a plurality of fusion proteins, wherein each fusion protein comprises at least one polypeptide antigen selected SseB, IpaB, SP0785 or SP1500; wherein the plurality of biotinylated polysaccharide antigens comprises polysaccharides of one or more Shigella selected from Shigella flexneri or Shigella sonnei, e.g., Shigella flexneri serotype selected from: 2a, 3a or 6, or Shigella sonnei.

In some embodiments, a method of immunizing a subject against Shigella infection and/or colonization comprises administering to the subject an immunologically effective amount of the vaccine.

In some embodiments, a method of immunizing a subject against Shigella infection and/or colonization comprises administering to the subject an immunologically effective amount of the immunogenic complex.

In some embodiments, a method of immunizing a subject against Shigella infection and/or colonization comprises administering to the subject an immunologically effective amount of the pharmaceutical composition.

In some embodiments, the vaccine, immunogenic composition, or pharmaceutical composition induces an immune response. In some embodiments, the immune response is to at least one polysaccharide antigen or at least one polypeptide of a fusion protein.

In some embodiments, the subject is immunized against Shigella infection and/or colonization with one dose of a vaccine. In some embodiments, the subject is immunized against Shigella infection and/or colonization with two doses of a vaccine. In some embodiments, the subject is immunized against Shigella infection and/or colonization with three doses of a vaccine.

In some embodiments, a fusion protein comprising a rhizavidin protein and at least one peptide or polypeptide antigen, wherein the rhizavidin protein comprises amino acids of SEQ ID NO: 3, or 85% sequence identity to amino acids of SEQ ID NO: 3, and Salmonella peptide or polypeptide comprises a fragment of at least 20 amino acids of the SseB protein, or the Shigella peptide or polypeptide comprises a fragment of at least 20 amino acids of the IpaB protein.

In some embodiments, the SseB protein comprises at least SEQ ID NO: 4 or a protein of at least 20 amino acids that has at least 85% sequence identity to SEQ ID NO: 4.

In some embodiments, the IpaB protein comprises at least SEQ ID NO: 5 or a protein of at least 20 amino acids that has at least 85% sequence identity to SEQ ID NO: 5.

In some embodiments, the fusion protein comprises at least SEQ ID NO: 1, or comprises a fusion protein comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 1. In some embodiments, a fusion protein of SEQ ID NO: 1 (Rhavi-SseB) can comprise at least one or more additional polypeptides, for example, one or more additional polypeptides selected from any of: SEQ ID NO: 5 (IpaB), SEQ ID NO: 8 (SP0785) or SEQ ID NO: 9 (SP1500), or a polypeptide that has a sequence at least 90%, at least 91%, at least 92%, at least 93%, at least 94%, at least 95%, at least 96%, at least 97%, at least 98%, at least 99%, or at least 99.5% identical to any of SEQ ID NO: 5, 8 or 9, respectively.

In some embodiments, the fusion protein comprises at least SEQ ID NO: 2, or comprises a fusion protein comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 2. In some embodiments, a fusion protein of SEQ ID NO: 2 (Rhavi-IpaB) can comprise at least one or more additional polypeptides, for example, one or more additional polypeptides selected from any of: SEQ ID NO: 4 (SseB), SEQ ID NO: 8 (SP0785) or SEQ ID NO: 9 (SP1500), or a polypeptide that has a sequence at least 90%, at least 91%, at least 92%, at least 93%, at least 94%, at least 95%, at least 96%, at least 97%, at least 98%, at least 99%, or at least 99.5% identical to any of SEQ ID NO: 4, 8 or 9, respectively.

In some embodiments, an immunogenic composition is or comprises a vaccine.

One aspect of the present invention relates to an immune composition comprising at least one species of the immunogenic complex as disclosed herein, wherein each species comprises an immunogenic complex comprising OSP from a different Shigella serotype (e.g., S. flexneri serotypes 2a, 3a or 6, or Shigella sonnei). In some embodiments, the immune composition or vaccine as disclosed herein further comprises at least one species of the immunogenic complex comprising OSP from a Salmonella serovers, such as Salmonella enterica, including but not limited to Salmonella enterica serotypes S. typhimurium, S. enteritidis, S. typhi vi, S. paratyphi A, or combinations thereof.

Another aspect of the present invention relates to a fusion protein comprising, in any order: a biotin-binding moiety (BBM) and a IpaB polypeptide, wherein the fusion protein comprises (i) an amino acid sequence that is at least 80% identical to SEQ ID NO: 3 (Rhavi) and (ii) an amino acid sequence that is at least 80% identical to SEQ ID NO: 5 (IpaB). Another aspect of the present invention relates to a fusion protein comprising, in any order: a biotin-binding moiety (BBM) and a SseB polypeptide, wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB). In some embodiments, the fusion protein can comprise one or more additional polypeptides selected from: SP1500 (SEQ ID NO: 8), SP1500 (SEQ ID NO: 9), CP1 (SEQ ID NO: 6), SEQ ID NO: 4 (SseB), or SEQ ID NO: 5 (IpaB), or a polypeptide that has a sequence at least 90%, at least 91%, at least 92%, at least 93%, at least 94%, at least 95%, at least 96%, at least 97%, at least 98%, at least 99%, or at least 99.5% identical to any of SEQ ID NO: 4, 5, 6, 8 or 9, respectively. Exemplary fusion proteins are disclosed in Table 1 herein. Other aspects relate to nucleic acids encoding the fusion proteins as disclosed herein, and cells, expression vectors comprising the nucleic acids.

Other aspects of the present invention relate to pharmaceutical compositions, vaccine compositions comprising the immune compositions, or fusion proteins, as disclosed herein. Other aspects of the present invention relate to method of making or producing a multivalent vaccine comprising two or more immunogenic complexes as disclosed herein.

Other aspects of the present invention relate to methods to induce an immune response to a IpaB protein, by administering an immune composition as disclosed herein, or an IpaB fusion protein as disclosed herein, wherein the immune response is an antibody and/or B cell response.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

FIG. 1 shows the Shigella OSP structures for S. flexneri 2a, S. flexneri 3a, S. flexneri 6, and S. sonnei.

FIGS. 2A-2B examines carrier and detergent selection using Shigella flexneri 3a MAPs through analysis of the antibody titer (FIG. 2A) or the bactericidal titer (FIG. 2B). SDOC denotes sodium doxycholate and LDAO denotes dodecyldimethylaminoxid. For each MAPS injection, it was 5 μg per dose per rabbit.

FIGS. 3A-3B shows the formulation test for OSP antibody and function through the examination of IpaB-S. flexneri 3a OSP ELISA (FIG. 3A) or IpaB-S. flexneri 3a killing (FIG. 3B). Buffer conditions include 20 mM Tris-HCl, 150 mM NaCl, and 0.02% Tween-80, pH 8 and 20 mM Histidine, 150 mM NaCl, and 0.02% Tween-80, pH 6.

FIGS. 4A-4D examines the bactericidal titer for all 4 Shigella strains (S. flexneri 3a (FIG. 4A), S. flexneri 2a (FIG. 4B), S. flexneri 6 (FIG. 4C), and S. sonnei (FIG. 4D) in monovalent MAPS.

FIGS. 5A-5D shows MAPS antibodies are specific to each serotype including S. flexneri 2a (Sf2a) (FIG. 5A), S. flexneri 3a (Sf3a) (FIG. 5B), S. flexneri 6 (Sf6) (FIG. 5C), and S. sonnei (53G) (FIG. 5D).

FIG. 6 depicts how there is little to no cross-serotype killing when targeting S. flexneri 2a, S. flexneri 6, S. flexneri 3a, and S. sonnei.

FIGS. 7A-7D examines the killing of other clinical strains with S. flexneri 2a (FIG. 7A), S. flexneri 3a (FIG. 7B), S. flexneri 6 (FIG. 7C), and S. sonnei 53G (FIG. 7D).

FIG. 8A-8B shows the components of quadrivalent Shigella MAPS vaccine for S. flexneri 2a (2a), S. flexneri 3a (3a), S. flexneri 6 (6), and S. sonnei (sonnei), which examine the antibody titer (FIG. 8A) or the bactericidal titer (FIG. 8B). Immunizations were three weeks apart and 5 μg each polysaccharide per dose, 20 μg total per dose.

FIGS. 9A and 9B show that quadrivalent (4V) Salmonella-MAPS generates robust polysaccharide antibody titers. FIG. 9A shows results of ELISA for a quadrivalent Salmonella-MAPS comprising: Rhavi-SseB MAPS made with Typhimurium and Enteritidis OSP, CP1 MAPS with Vi and Paratyphi A OSP MAPS. FIG. 9B shows results of ELISA of a quadrivalent MAPS containing Rhavi-SseB MAPS made with Typhimurium and Enteritidis OSP, CP1 MAPS made with Vi and Paratyphi A OSP MAPS. The carrier protein is shown on top for each MAPS. Sera were tested prior to first immunization (Pre), and two weeks after the first immunization (P1).

FIGS. 10A and 10B shows immunogenicity of multivalent (2V), and Quadrivent (4V) Salmonella-MAPS in rabbits. FIG. 10A shows ELISA results of protein antibodies multivalent (2V) Salmonella-MAPS comprising polysaccharides from both S. typhi (comprising the Vi polysaccharide) and S. Paratyphi A (comprising the OSP polysaccharide) and comprising the carrier protein Rhavi-SseB or CP1, as compared to the quadrivalent MAPS containing Rhavi-SseB and polysaccharides from: S. typhimurium (S12), S. enteritidis (173), S. typhi and S. Paratyphi A. FIG. 10B shows ELISA results of OSP and Vi antibodies from the quadrivalent Salmonella-MAPS containing Rhavi-SseB and polysaccharides from: S. typhimurium (S12), S. enteritidis (173), S. typhi and S. Paratyphi A. Sera were tested prior to first immunization (Pre), and two weeks after the first immunization (P1), and two weeks after the second immunization (P2).

FIG. 11A-11C shows BCA and OPA titers for bivalent (2V) or quadrivalent (4V) Salmonella-MAPS. BCA was done by the incubation of sera with bacteria and complement while OPA was done by the incubation of sera with bacteria, complement and differentiated HL-60 cells. FIG. 11A shows the killing titer of sera from rabbits received Salmonella-MAPS comprising the Rhavi-SseB carrier protein and polysaccharides from both S. typhimurium (S12), S. enteritidis (J73). FIG. 11B shows the results from the killing assay of S. typhi and S. paratyphi A by Salmonella-MAPS comprising the CP1 carrier protein and polysaccharides from Vi and ParaOSP polysaccharides. FIG. 11C shows the results from the killing assay by the quadrivalent Salmonella-MAPS comprising the Rhavi-SseB carrier protein and polysaccharides from typhimurium (S12), S. enteritidis (J73), S. typhi and S. paratyphi A.

FIG. 12A-12B shows the OPA titer by SseB antisera. FIG. 12A shows killing of S. typhimurium (S12) by SseB antisera. FIG. 12B shows killing of S. enteritidis by SseB antisera. Bacteria (either S. typhimurium or S. enteritidis) were incubated with heat-inactivated antisera for 20 minutes at room temperature. Differentiated HL-60 and baby rabbit complement were added, and the killing was carried out at 37° C. for 1 hour. Cells were lysed with Saponin and plated for CFU count. Opsonophagocytic killing (OPK) titer was defined as the reciprocal serum dilution required to mediate 50% bacterial killing.

FIG. 13A-13B shows the addition of SseB antisera enhanced killing by the OSP antisera. FIG. 13A shows OPK assay results of S. typhimurium by OSP sera, or OSP sera plus SseB sera. FIG. 13B shows OPK assay results of S. enteritidis by OSP sera, or OSP sera plus SseB sera. OSP antisera were either mixed with pre-sera or SseB antisera and then used in the OPK assay. OPK titer was defined as the reciprocal serum dilution required to mediate 50% bacterial killing.

FIG. 14A-14B shows the bactericidal activity for Monovalent (1V), Bivalent (2V) and quadrivalent (4V) Salmonella-MAPS comprising Rhavi-SseB and OSPs against S. typhimurium and S. paratyphi A. FIG. 14A shows bactericidal titers of P2 sera against S. Typhimurium. FIG. 14B shows bactericidal titers of P2 sera against S. Paratyphi A.

FIG. 15A-15B shows OPA titers of sera against S. enteritidis (J73) or S. typhi with quadrivalent (4V) and bivalent (2V) Salmonella MAPS. FIG. 15A shows OPA titer of sera from the immunization of bivalent (2V) or quadrivalent (4V) Salmonella-MAPS against Vi containing bacteria. FIG. 15B shows OPA titers of sera from the immunization of the bivalent (2V) or quadrivalent (4V) Salmonella-MAPS against S. enteritidis (J73). No killing was observed for pre-sera.

FIG. 16A-16B examines the pre sera and the IpaB alone sera using an opsonophagocytic killing assay (OPK) for Shigella flexneri 3a (FIG. 16A) and Shigella flexneri 6 (FIG. 16B). Rabbits were immunized with IpaB two times. The OPK activity of pre-immunization and post 2 immune sera were compared.

FIG. 17A-17B analyzes S. flexneri 6 O-specific polysaccharide (OSP) and S. Flexneri OSP and IpaB antibody titers with different adjuvants through the use of ELISAs.

FIG. 18 examines the killing activity of the adjuvant test, where killing activity is correlated to an OSP antibody.

FIG. 19 shows the ratio of bactericidal titer to OSP antibody titer for adjuvant test. The quality of the OSP antibody is similar regardless of the adjuvant.

FIG. 20A-20C examines OSP ELISAs for 8-valent and quadrivalent MAPS vaccines. The 8-valent Shigella-MAPS and Salmonella MAPS induced a robust immune response after P1, which was enhanced after by a second immunization P2 (FIG. 20A). Similar increase in antibody titer (AU) was seen with the quadrivalent Shigella-MAPS vaccine with the IpaB carrier protein comprising polysaccharides from S. flexneri 2a, S. flexneri 3a, S. flexneri 6, and S. sonnei (FIG. 20B) and quadrivalent salmonella-MAPS vaccine (FIG. 20C) with the SseB carrier protein comprising polysaccharides from: S. Typhimurium (S12), S. Enteritidis (J13), S. Typhi and S. Paratyphi A (FIG. 20C).

FIG. 21A-21C shows the protein antibody titer for 8-valent MAPS is comparable to quadrivalent MAPS. FIG. 21A examines the antibody titer using 8-valent MAPS in both Shigella and Salmonella. FIG. 21B-21C examines the antibody titer using quadrivalent MAPS in Shigella (FIG. 21B) and Salmonella (FIG. 21C).

FIG. 22A-22B examines the killing activity of 8-valent MAPS serum. High, medium, and low antibody titer sera was analyzed for killing activity. FIG. 22A examined the bactericidal titer using the 8-valent MAPS serum. FIG. 22B examined the OPA titer using the 8-valent MAPS serum.

The present teachings described herein will be more fully understood from the following description of various illustrative embodiments, when read together with the accompanying drawings. It should be understood that the drawings described below are for illustration purposes only and are not intended to limit the scope of the present teachings in any way.

### DETAILED DESCRIPTION OF INVENTION

The present disclosure relates, generally, to compositions, systems, and methods that include novel complexed proteins and polysaccharides, e.g., vaccines of complexed proteins and polysaccharides. Such complexes can be used, e.g., to induce and/or increase an immunoprotective response in subjects at risk of or suffering from Shigella infection.

### Immunogenic Complexes

The present disclosure encompasses immunogenic complexes that include one or more polysaccharides and/or polypeptides of Shigella.

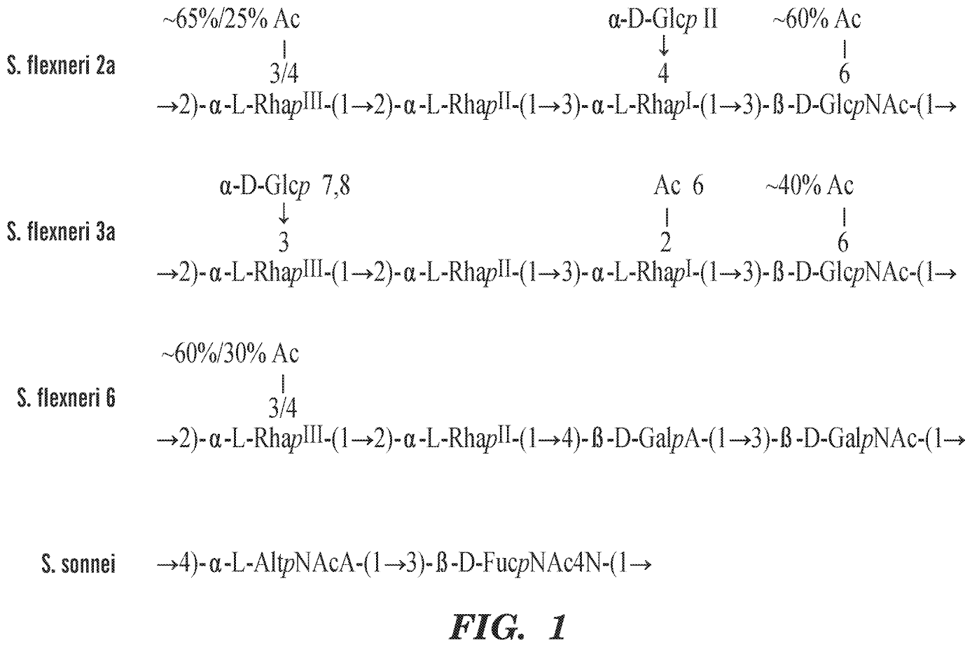

In some embodiments, immunogenic complexes are, or are based on, Multiple Antigen Presenting System (MAPS) complexes. Aspects of the MAPS platform have been previously described in WO2012/155007, the contents of which are herein incorporated by reference in their entirety, and are shown schematically in FIG. 1. See also Zhang et al, 2013.

As described herein, immunogenic complexes of the disclosure include one or more antigenic polypeptides non-covalently complexed with one or more antigenic polysaccharides. In some embodiments, one or more antigenic polypeptides are complexed via affinity interaction with one or more antigenic polysaccharides. In some embodiments, immunogenic complexes of the disclosure include one or more antigenic polypeptides non-covalently complexed with one or more antigenic polysaccharides using one or more affinity molecule/complementary affinity molecule pairs. In some embodiments, an immunogenic complex includes (i) a first affinity molecule described herein conjugated to one or more antigenic polysaccharides, and (ii) a fusion protein that is or comprises a complementary affinity molecule described herein and a polypeptide. In some embodiments, an immunogenic complex includes (i) a plurality of a first affinity molecule described herein conjugated to one or more antigenic polysaccharides, and (ii) a fusion protein that is or comprises a complementary affinity molecule described herein and a polypeptide. Upon association of the first affinity molecule and the complementary affinity molecule, the one or more antigenic polypeptides are non-covalently complexed to the one or more antigenic polysaccharides.

In some embodiments, one or more antigenic polypeptides are complexed via affinity interaction with one antigenic polysaccharide. In some embodiments, immunogenic complexes of the disclosure include one or more antigenic polypeptides non-covalently complexed with one antigenic polysaccharide using one affinity molecule/complementary affinity molecule pair. In some embodiments, immunogenic complexes of the disclosure include one or more antigenic polypeptides non-covalently complexed with one antigenic polysaccharide using one or more affinity molecule/complementary affinity molecule pairs. In some embodiments, each of the affinity molecule/complementary affinity molecule pairs is the same, e.g., biotin/biotin-binding moiety pairs. In some embodiments, an immunogenic complex includes (i) a first affinity molecule described herein conjugated to one antigenic polysaccharide, and (ii) a fusion protein that is or comprises a complementary affinity molecule described herein and at least one immunogenic polypeptide. In some embodiments, an immunogenic complex includes (i) a plurality of a first affinity molecule described herein conjugated to one antigenic polysaccharide, and (ii) a fusion protein that is or comprises a complementary affinity molecule described herein and a polypeptide. Upon association of the first affinity molecule and the complementary affinity molecule, the one or more antigenic polypeptides are non-covalently complexed to the one antigenic polysaccharide.

In some embodiments, the affinity molecule/complementary affinity molecule pair is selected from one or more of biotin/biotin-binding moiety, antibody/antigen, enzyme/substrate, receptor/ligand, metal/metal-binding protein, carbohydrate/carbohydrate binding protein, lipid/lipid-binding protein, and His tag/His tag-binding molecule.

In some embodiments, the first affinity molecule is biotin (or a derivative or fragment thereof), and the complementary affinity molecule is a moiety, e.g., a biotin-binding protein, or a biotin-binding domain or biotin-binding fragment thereof. In some embodiments, the biotin-binding moiety is rhizavidin, avidin, streptavidin, bradavidin, tamavidin, lentiavidin, zebavidin, NeutrAvidin, CaptAvidin™, or a biotin-binding domain or biotin-binding fragment thereof, or a combination thereof. In some embodiments, the biotin-binding moiety is rhizavidin, or a biotin-binding domain or biotin-binding fragment thereof. In some embodiments, the biotin binding moiety is or comprises a polypeptide of SEQ ID NO: 3, or is or comprises a polypeptide that has at least 80%, or at least 85% or at least 90% or more than 90% sequence identity to SEQ ID NO: 3, or a biotin-binding domain or biotin-binding fragment thereof.

In some embodiments, the one or more antigenic polysaccharides are, or are derived from Gram-negative bacteria and/or Gram-positive bacteria. In some embodiments, one or more bacterial antigenic polysaccharides are, or are derived from Shigella. In some embodiments, one or more antigenic polysaccharides are, or are derived from one or more pathogens. In some embodiments, one or more antigenic polysaccharides are, or are derived from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 serotypes or strains of a pathogen. In some embodiments, one or more antigenic polysaccharides are, or are derived from more than 25 serotypes or strains of a pathogen, e.g., 26, 27, 28, 29, 30, 35, 40, 45, or 50 serotypes or strains. In some embodiments, one or more antigenic polysaccharides are, or are derived from more than 60, 70, 80, 90, or 100 serotypes or strains of a pathogen.

In some embodiments, the one or more antigenic polysaccharides comprise one or more affinity molecules conjugated to the antigenic polysaccharides. In some embodiments, the one or more affinity molecules comprise biotin or biotin derivatives.

In some embodiments, the antigenic polysaccharides comprise a plurality of affinity molecules conjugated to the antigenic polysaccharides. In some embodiments, the affinity molecules comprise biotin or biotin derivatives.

In some embodiments, one or more antigenic polypeptides are covalently linked (e.g., fused) to a complementary affinity molecule described herein. In some embodiments a fusion protein comprises one or more antigenic polypeptides and a complementary affinity molecule disclosed herein. In some embodiments, the complementary affinity molecule is or comprises a biotin-binding moiety. In some embodiments, the biotin-binding moiety comprises rhizavidin or a biotin-binding portion thereof. In some embodiments, antigenic polysaccharides and/or antigenic polypeptides that may be included in immunogenic complexes are recombinantly or synthetically produced.

In some embodiments, antigenic polysaccharides and/or antigenic polypeptides that may be included in immunogenic complexes are isolated and/or derived from natural sources. In some embodiments antigenic polysaccharides and/or antigenic polypeptides that may be included in immunogenic complexes are isolated from bacterial cells. Exemplary polysaccharides and/or polypeptides are described below.

### Antigenic Polypeptides

In some embodiments, an immunogenic complex described herein comprises one or more polypeptide antigens. In some embodiments, a polypeptide antigen is a bacterial polypeptide, a fungal polypeptide, and/or a viral polypeptide. In some embodiments, a polypeptide antigen is a polypeptide of, or derived from S. enterica, S. pneumoniae, or Shigella flexneri. In some embodiments, the one or more polypeptide antigen is a polypeptide of, or derived from, a pathogen other than S. enterica, S. pneumoniae, or S. flexneri. In some embodiments, an immunogenic complex includes one or more of the following S. enterica, S. pneumoniae, or S. flexneri antigenic polypeptides, or portions thereof.

### SseB Polypeptides

The SseB polypeptide is a Salmonella type 3 secretion system protein conserved across Salmonella strains. In some embodiments, an SseB polypeptide is or comprises a full-length SseB polypeptide. For example, in some embodiments, a full-length SseB polypeptide is represented by the amino acid sequence as set forth in SEQ ID NO: 4. In some embodiments, an SseB polypeptide includes a portion of an SseB polypeptide (e.g., a portion of the SseB polypeptide of SEQ ID NO: 4, which portion includes at least 7, 8, 9, 10, 11, 12, 13, 14, 15, 16, 17, 18, 19, 20, 25,30,35,40, 45, 50,55, 60, 65, 70, 75, 80, 85, 90, 95, 100, 150 or more contiguous amino acids of SEQ ID NO: 4). In some embodiments, an SseB polypeptide contains one or more amino acid alterations (e.g., deletion, substitution, and/or insertion) from a naturally-occurring wild-type SseB polypeptide sequence. For example, an SseB polypeptide may contain an amino acid sequence that is at least 60% or more (e.g., at least 65%, 70%, 75%, 80%, 85%, 90%, 95%, or 98%) identical to SEQ ID NO: 4 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or more consecutive amino acids of the sequence shown in SEQ ID NO: 4). Alternatively, an SseB polypeptide may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or more consecutive amino acids) of a sequence that is at least 60% or more (e.g., at least 65%, 70%, 75%, 80%, 85%, 90%, 95%, or 98%) identical to SEQ ID NO: 4.

Purification of SseB from E. coli (either on its own or as a fusion protein as disclosed herein) needs co-expression of chaperone SseA. Accordingly, SseB can be co-expressed from E. coli with SseA. The SseB polypeptide can be separated from its SseA chaperone using detergent wash (Dodecyldimethylaminoxid (LDAO) or sodium deoxycholate (SDOC)). In some embodiments, SseB is co-expressed with His-tagged SseA in E. co/i, the SseB and SseA complex is isolated by using the His-tagged SseA protein, the complex is then washed with LDAO or SDOC to elute the SseB polypeptide.

### IpaB Polypeptides

The IpaB polypeptide is a S. flexneri type 3 secretion system protein conserved across Shigella strains. In some embodiments, an IpaB polypeptide is or comprises a full-length IpaB polypeptide. For example, in some embodiments, a full-length IpaB polypeptide is represented by the amino acid sequence as set forth in SEQ ID NO: 5. In some embodiments, an IpaB polypeptide includes a portion of an IpaB polypeptide (e.g., a portion of the IpaB polypeptide of SEQ ID NO: 5, which portion includes at least 7, 8, 9, 10, 11, 12, 13, 14, 15, 16, 17, 18, 19, 20, 25,30,35,40, 45, 50,55, 60, 65, 70, 75, 80, 85, 90, 95, 100, 150, 200, 250, 300, 350, or more contiguous amino acids of SEQ ID NO: 5). In some embodiments, an IpaB polypeptide contains one or more amino acid alterations (e.g., deletion, substitution, and/or insertion) from a naturally-occurring wild-type IpaB polypeptide sequence. For example, an IpaB polypeptide may contain an amino acid sequence that is at least 60% or more (e.g., at least 65%, 70%, 75%, 80%, 85%, 90%, 95%, or 98%) identical to SEQ ID NO: 5 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or more consecutive amino acids of the sequence shown in SEQ ID NO: 5). Alternatively, an IpaB polypeptide may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or more consecutive amino acids) of a sequence that is at least 60% or more (e.g., at least 65%, 70%, 75%, 80%, 85%, 90%, 95%, or 98%) identical to SEQ ID NO: 5. An exemplary IpaB polypeptide is provided herein as the amino acid of SEQ ID NO: 5.

SseB and IpaB are components of the type III secretion system and their purification from E. Coli needs co-expression of chaperones SseA and IpgC, respectively.

Purification of IpaB from E. co/i (either on its own or as a fusion protein as disclosed herein) needs co-expression of chaperone IpgC. Accordingly, IpaB can be co-expressed from E. co/i with IpgC. The IpaB polypeptide can be separated from its IpgC chaperone using detergent wash (Dodecyldimethylaminoxid (LDAO) or sodium deoxycholate (SDOC)). In some embodiments, IpaB is co-expressed with His-tagged IpgC in E. coli, the IpaB and IpgC complex is isolated by using the His-tagged IpgC protein, the complex is then washed with LDAO or SDOC to elute the IpaB polypeptide.

### Type III Secretion System

The type three (III) secretion system (abbreviated T3SS) is a protein appendage found in several Gram negative bacteria. This secretion system is distinguished from at least five other secretion systems found in Gram-negative bacteria. Bacteria that contain T3SS include, but are not limited to Shigella, Salmonella, Escherichia coli, Vibrio, Burkholderia, Yersinia, Chlamydia, Pseudomonas, Erwinia, Ralstonia, Xanthomonas, and Rhizobium.

The T3SS is composed of approximately 30 different proteins and is used both for secreting infection-related proteins and flagellar components. T3SSs are essential for the pathogenicity of many pathogenic bacteria. The needle-like structure (also known as the Needle Complex or T3SS apparatus (T3SA)) is used as a sensory probe to detect the presence of eukaryotic organisms and secrete proteins that help the bacteria infect them. The secreted effector proteins are secreted directly from the bacterial cell into the eukaryotic (host) cell, where they exert a number of effects that help the pathogen to survive and to escape an immune response.

The needle complex comprises of an inner membrane ring(s), a connector, an outer membrane ring(s), a needle, and a tip. The inner membrane ring(s), connector, and outer membrane ring(s) are embedded in the inner membrane, periplasm, and outer membrane of the bacterium. The needle is found in the extracellular space or the cytoplasm of a eukaryotic host cell. A single bacterium can have several hundred needle complexes spread across its membrane. The needle complex is a universal feature of all T3SSs of pathogenic bacteria and shares similarities with bacterial flagella. The needle is comprised of needle monomer protein piled upon each other, which each monomer protein is about 9 kDa. The needle measures around 60-80 nm in length, 8 nm in external width and a 3 nm diameter. This structure prevents interference during secretion by the adhesins and the lipopolysaccharide layer, but only allows for unfolded proteins to be passed through into the eukaryotic host cell.

T3SS proteins can be groups into three categories: structural proteins, effector proteins, and chaperones. Structural proteins build the base and needle. Effector proteins get secreted into the host cell and promote infection/suppress host cell defenses. Chaperones bind effectors in the bacterial cytoplasm and protect them from aggregation and degradation and direct them to the needle complex.

Effector proteins contain a secretion signal, which is a short sequence of amino acids located at the beginning of the protein (usually within the first 20 amino acids at the N-terminus), that the needle complex is able to recognize. The secretion signal of T3SS proteins is never cleaved off of the proteins.

T3SS effectors manipulate host cells in several ways, including, but not limited to promoting of uptake of the bacterium by the host cell, producing a pore or a channel in the host cell membrane through which other effectors may enter, utilization of the host cell's own machinery, tamper with the host's cell cycle, and induce apoptosis.

An effector protein from Shigella includes the invasion plasmid antigen (Ipa). Examples of this protein include, but are not limited to, IpaA, IpaB, IpaC, IpaD, and IpaJ. Another effector protein from Shigella includes the invasion plasmid gene. Examples of this gene include, but are not limited to IpgC and IpgG.

These effector proteins and genes can have homologs in different species. For example, the IpgC genes in Shigella has a homolog in Salmonella (SicA) and in Yersinia (SycD).

Residing atop the TTSS needle tip is a tip complex composed of IpaB and IpaD sequentially assembled, which is required for pathogenesis. Several studies have reported the presence of antibodies against Ipa proteins in serum from infected individuals, in subjects immunized with live attenuated organisms during clinical trials and following vaccination in animal models. The conserved nature of the IpaB and IpaD and their critical role in pathogenesis make them ideal targets for vaccine development. An Ipa-based vaccine would provide broader coverage across multiple serotypes. It would also simplify vaccine production and formulation.

The proteins, polypeptides or peptides which may be used in the practice of the invention include IpaB, IpaD and IpgC, and variants or derivatives thereof which have about at least about 50, 55, 60, 65, 70, 75, 80, 85, or 90% identity to the sequences presented herein, and preferably the level of identity is at least about 92, 93, 94, 95, 96, 97, 98 or 99%. Those of skill in the art are familiar with methods and software programs for sequence comparison to determine identity.

The invention also encompasses the use of nucleic acids encoding the proteins/polypeptides/peptides described herein, i.e. nucleic acid vaccines are also contemplated. Those of skill in the art are aware of the many protocols for preparing and administering nucleic acid vaccines, such as those described in issued U.S. Pat. Nos. 7,927,870 and 7,094,410, the complete contents of which is hereby incorporated by referenced in entirety.

### IpaB Effector Protein

IpaB is a 580 amino acid protein that contains two chaperone binding domains (between residues 11 and 76 near the IpaB N terminus), a coiled-coil domain, a hydrophobic region, and a protein-protein contacts region at the IpaB C terminus. Within the hydrophobic regions is an α-helical hairpin region, which has been observed in some membrane-interacting colicins. The region involved in IpaB's ability to associated with and activate caspase 1 is reported to also lie within this region (between residue 316 and 401).

IpaB in Shigella has homologs in Salmonella (SipB), enteropathogenic E. coli (EspD), and Yersina spp. (YopB). Homologs refer to similarity due to shared ancestry between a pair of structures or genes in different taxa.

The IpaB effector protein has many roles during the pathogenesis of Shigella. It is the first hydrophobic translocator protein present at the maturing needle complex. IpaB then senses contact with a host cell membrane, forming the translocon pore thorugh which effectors are delivered to the host cytoplasm. Within the bacterium, IpaB exists as a heterodimer with its chaperone, IpgC.

IpaB has dual roles as an effector protein: it serves by both creating a pore in the host cell membrane and as an effector, exerting multiple detrimental effects on the host cell. IpaB induces apoptosis in macrophages by interacting with caspase 1. This interaction results in a release of IL-10 as a precursor to the inflammation responsible for the pathology of shigellosis.

### SP0785 Polypeptides

SP0785 is a conserved hypothetical S. pneumoniae protein described in WO2014/124228, which is incorporated herein in its entirety by reference. In some embodiments, an SP0785 polypeptide is an efflux transporter protein conserved across S. pneumoniae strains. In some embodiments, an SP0785 polypeptide is or comprises a full-length SP0785 polypeptide. For example, in some embodiments, a full-length SP0785 polypeptide has 399 amino acids (38 kDa). In some embodiments, an SP0785 polypeptide includes a portion of an SP0785 polypeptide (e.g., a portion which portion includes at least 7, 8, 9, 10, 11, 12, 13, 14, 15, 16, 17, 18, 19, 20, 25,30,35,40, 45, 50,55, 60, 65, 70, 75, 80, 85, 90, 95, 100, 150, 200, 250, 300, 350, or more contiguous amino acids of SEQ ID NO: 8). In some embodiments, an SP0785 polypeptide contains one or more amino acid alterations (e.g., deletion, substitution, and/or insertion) from a naturally-occurring wild-type SP0785 polypeptide sequence. For example, an SP0785 polypeptide may contain an amino acid sequence that is at least 60% or more (e.g., at least 65%, 70%, 75%, 80%, 85%, 90%, 95%, or 98%) identical to SEQ ID NO: 8 or a portion thereof (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or more consecutive amino acids of the sequence shown in SEQ ID NO: 8). Alternatively, an SP0785 polypeptide may contain a portion (e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids) of a sequence that is at least 60% or more (e.g., at least 65%, 70%, 75%, 80%, 85%, 90%, 95%, or 98%) identical to SEQ ID NO: 8.

In some embodiments, a fusion protein comprises a SP0785 polypeptide of S. pneumoniae. In some embodiments, a fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 367, or 399 consecutive amino acids of a SP0785 polypeptide.

In some embodiments, a SP0785 polypeptide of the fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 367, or 399 consecutive amino acids of the sequence shown in SEQ ID NO: 8. In some embodiments, a SP0785 polypeptide of the fusion protein comprises an amino acid sequence that is at least 60% or more (including, e.g., at least 65%, 70%, 75%, 80%, 85%, 90%, 95%, 96%, 97%, 98%, 99%, or 100%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 367, or 399 consecutive amino acids of the sequence shown in SEQ ID NO: 8.

In some embodiments, a SP0785 polypeptide of the fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 367 consecutive amino acids of the sequence shown in SEQ ID NO:8. In some embodiments, a SP0785 polypeptide of the fusion protein comprises an amino acid sequence that is at least 60% or more (including, e.g., at least 65%, 70%, 75%, 80%, 85%, 90%, 95%, 96%, 97%, 98%, 99%, or 100%) identical to at least 7, 8, 9, 10, 11, 12, 13, 14, 15, 16, 17, 18, 19,20, 25, 30, 35, 45, 50, 60, 65, 70, 75, 80, 85, 90, 95, 100, 150, 200, 250, 300, 350, or 367 consecutive amino acids of the sequence shown in SEQ ID NO: 8.

An exemplary nucleotide sequence encoding a SP0785 polypeptide is provided herein as SEQ ID NO: 62 or a nucleic acid sequence having at least 85% or 90% sequence identity to SEQ ID NO: 62.

### SP1500 Polypeptides

SP1500 is a S. pneumoniae protein described in WO 2014/124228, which is incorporated herein in its entirety by reference. In some embodiments, an SP1500 polypeptide is an Amino Acid ABC Transporter, amino acid-binding polypeptide conserved across S. pneumoniae strains. In some embodiments, an SP1500 polypeptide is or comprises a full-length SP1500 polypeptide. For example, in some embodiments, a full-length SP1500 polypeptide has 278 amino acids (28 kDa). In some embodiments, an SP1500 polypeptide includes a portion of an SP1500 polypeptide (e.g., a portion of the SP1500 polypeptide of SEQ ID NO: 9, which portion includes at least 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, or more contiguous amino acids of SEQ ID NO: 9). In some embodiments, an SP1500 polypeptide contains one or more amino acid alterations (e.g., deletion, substitution, and/or insertion) from a naturally-occurring wild-type SP1500 polypeptide sequence. For example, an SP1500 polypeptide may contain an amino acid sequence that is at least 60% or more (e.g., at least 65%, 70%, 75%, 80%, 85%, 90%, 95%, or 98%) identical to SEQ ID NO: 9.

An exemplary nucleotide sequence encoding a SP1500 polypeptide is provided herein as SEQ ID NO: 63, or a nucleic acid sequence having at least 85% or 90% sequence identity to SEQ ID NO: 62.

### Complementary Affinity Molecules

In some embodiments, a complementary affinity molecule comprises a biotin-binding moiety. In some embodiments, a fusion protein of the immunogenic complex comprises a biotin-binding moiety, and one or more polypeptide antigens. In some embodiments, a fusion protein comprises a biotin-binding moiety and two or more polypeptide antigens. As used herein, a “biotin-binding moiety” refers to a biotin-binding protein, a biotin-binding fragment thereof, or a biotin-binding domain thereof.

In some embodiments, MAPS complexes disclosed herein utilize the high affinity (dissociation constant [KD]≈10⁻¹⁵ M) non-covalent binding between biotin and rhizavidin, a biotin-binding protein that has no significant predicted homology with human proteins. Rhizavidin, a naturally occurring dimeric protein in the avidin protein family, was first discovered in Rhizobium etli, a symbiotic bacterium of the common bean. Rhizavidin has only a 22% amino acid identity with chicken avidin, a protein commonly found in eggs, but with high conservation of amino acid residues involved in biotin binding. No cross-reactivity to rhizavidin is observed in human serum samples obtained from subjects exposed to avidin [Helppolainen et al, 2007], suggesting that rhizavidin antibodies may not cross-react with chicken avidin. Biotin conjugates have been used in several clinical applications without any reported adverse events [Buller et al, 2014; Paty et al, 2010; Lazzeri et al, 2004],

In some embodiments, the biotin-binding moiety of the fusion protein comprises rhizavidin or a biotin-binding domain or biotin-binding fragment thereof, as further described in WO 2012/155053, the contents of which are herein incorporated by reference in their entirety. In some embodiments, a biotin-binding moiety is or comprises a polypeptide having at least 80%, 85%, 90%, 95%, 96%, 97%, 98%, or 99% identity to rhizavidin, or a biotin-binding domain or biotin-binding fragment thereof. In some embodiments, the biotin-binding moiety comprises a polypeptide of SEQ ID NO: 3 or a biotin-binding domain or biotin-binding fragment thereof. In some embodiments, the biotin-binding moiety is or comprises a polypeptide having at least 80%, 85%, 90%, 95%, 96%, 97%, 98%, or 99% identity to the sequence of SEQ ID NO: 3, or biotin-binding domain or biotin-binding fragment thereof.

In some embodiments, the biotin-binding moiety comprises an amino acid sequence of at least 80%, or 90%, or 95% sequence identity to SEQ ID NO: 3 that has any one or more of the amino acid modifications: N80, T108, N118, S119A, N138A. For example, biotin-binding moiety for use in the fusion proteins as disclosed herein comprises an amino acid sequence of at least 80%, or 90%, or 95%, 98%, 99% or 100% sequence identity to SEQ ID NO: 55, wherein the biotin binding moiety has at least one or more of the amino acid modifications: N80, T108, N118, S119A, N138A.

In some embodiments, a fusion protein as disclosed herein comprises a lipidated biotin-binding protein or lipidated rhizavidin polypeptide. As used herein, the term “lipidated biotin-binding protein” refers to a biotin-binding protein that is covalently conjugated with a lipid. The lipid moieties could be a diacyl or triacyl lipid. The lipidated biotin-binding protein can be made using a lipidation sequence. As used herein, the term “lipidation sequence” refers to an amino acid sequence that facilitates lipidation in a bacteria, e.g., E. coli, of a polypeptide carrying the lipidating sequence. The lipidation sequence can be present at the N-terminus or the C-terminus of the rhizavidin protein. The lipidation sequence can be linked to the recombinant biotin-binding protein to form a fusion protein, which is in lipidated form when expressed in E. coli by conventional recombinant technology. In some embodiments, a lipidation sequence is located at the N-terminus of the biotin-binding protein, e.g, rhizavidin polypeptide as disclosed herein. Exemplary lipidated rhizavidin proteins are disclosed in U.S. Pat. No. 9,499,593, which is incorporated herein in its entirety by reference.

Fusion Proteins that Include Antigenic Polypeptides

Antigenic polypeptides described herein can be part of a fusion protein. For example, in some embodiments, an immunogenic complex described herein comprises a fusion protein that is or comprises a complementary affinity molecule and one or more antigenic polypeptides described herein. In some embodiments, a fusion protein of the immunogenic complex has carrier properties. In some embodiments, a fusion protein of the immunogenic complex has antigenic properties. In some embodiments, a fusion protein of the immunogenic complex has carrier properties and antigenic properties.

In some embodiments, the fusion protein is or comprises a complementary affinity molecule described herein (e.g., a biotin-binding moiety described herein), and one or more polypeptides of or derived from S. enterica, S. pneumoniae, and/or S. flexneri.

In some embodiments, the fusion protein of the immunogenic complex comprises a biotin-binding moiety that is or comprises a polypeptide having at least 80%, 85%, 90%, 95%, 96%, 97%, 98%, or 99% identity to the sequence of SEQ ID NO: 3 (rhizavidin), or biotin-binding fragment thereof.

In some embodiments, the fusion protein of the immunogenic complex comprises a SseB polypeptide that is a Salmonella type 3 secretion system protein that is conserved across Salmonella strains. In some embodiments, the fusion protein comprises a complementary affinity molecule described herein (e.g., a biotin-binding moiety described herein) and a polypeptide comprising an amino acid sequence having at least 80%, 85%, 90%, 95%, 96%, 97%, 98%, 99%, or 100% identity to the sequence of SEQ ID NO: 4 or an antigenic fragment thereof.

In some embodiments, the fusion protein of the immunogenic complex comprises IpaB. In some embodiments, the fusion protein comprises a complementary affinity molecule described herein (e.g., a biotin-binding moiety described herein) and a polypeptide comprising an amino acid sequence having at least 80%, 85%, 90%, 95%, 96%, 97%, 98%, 99%, or 100% identity to the sequence of SEQ ID NO: 5 or an antigenic fragment thereof.

In some embodiments, the fusion protein of the immunogenic complex is CP1, further described in PCT Application WO2021/17016516 entitled “Pneumococcal Fusion Protein Vaccines” and filed Sep. 12, 2019, the contents of each of which are incorporated herein by reference in their entirety. Aspects of the CP1 have also been previously described in WO2020/056127, the contents of which are herein incorporated by reference in their entirety, In some embodiments, the fusion protein comprises a complementary affinity molecule described herein (e.g., a biotin-binding moiety described herein) and a polypeptide comprising an amino acid sequence having at least 80%, 85%, 90%, 95%, 96%, 97%, 98%, 99%, or 100% identity to the sequence of SEQ ID NO: 6 or an antigenic fragment thereof.

### SseB-Fusion Proteins

Another aspect of the present invention relates to a fusion protein comprising, in any order: (a) a biotin-binding moiety and (b) a SseB polypeptide, wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB). An exemplary SseB polypeptide is provided herein as the amino acid of SEQ ID NO: 4.

One aspect of the present invention relates to a fusion protein comprising, in the following order: (a) a biotin-binding moiety and (b) a SseB polypeptide, wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB).

In some embodiments, the present invention provides a fusion protein comprising, in any order: (a) a biotin-binding moiety and (b) at least two SseB polypeptides, wherein the fusion protein comprises, in any order (i) an amino acid sequence that is at least 80% identical to SEQ ID NO: 3 (Rhavi) and (ii) a polypeptide comprising an amino acid sequence that is at least 80% identical to SEQ ID NO: 4 (SseB) and (iii) a polypeptide comprising an amino acid sequence that is at least 80% identical to SEQ ID NO: 4 (SseB).

In all aspects of the fusion protein as disclosed herein, the biotin-binding moiety comprises an amino acid sequence that is at least 80%, 85%, 90% identical to SEQ ID NO:3 or a biotin binding portion thereof. In all aspects of the fusion protein as disclosed herein, SseB polypeptide comprises an amino acid sequence that is at least 80%, 85%, 90% identical to SEQ ID NO:4.

In some embodiments, the fusion protein comprises, in order of N- to C-terminal: a biotin-binding moiety comprises an amino acid sequence of SEQ ID NO: 3, or an amino acid sequence that is at least 80%, 85%, 90% identical to SEQ ID NO:3 or a biotin binding portion thereof, and a Salmonella SseB polypeptide comprising an amino acid sequence of SEQ ID NO: 4 or an amino acid sequence an amino acid sequence that is at least 80%, 85%, 90% identical to SEQ ID NO: 4.

In some embodiments, the fusion protein comprises, in order of N- to C-terminal: a Salmonella SseB polypeptide comprising an amino acid sequence of SEQ ID NO: 4 (SseB), or an amino acid sequence that is at least 80%, 85%, 90% identical to SEQ ID NO: 4, and a biotin-binding moiety comprises an amino acid sequence of SEQ ID NO: 3, or an amino acid sequence that is at least 80%, 85%, 90% identical to SEQ ID NO:3 or a biotin binding portion thereof

In some embodiments, fusion protein comprises an amino acid sequence that is at least 80%, 85%, or 90% identical to SEQ ID NO: 1. In some embodiments, fusion protein comprises an amino acid sequence that consists of SEQ ID NO: 1 (Rhavi-SseB). In some embodiments, fusion protein comprises an amino acid sequence that is at least 80%, 85%, or 90% identical to SEQ ID NO: 11. In some embodiments, fusion protein comprises, or consists of, an amino acid sequence that comprises the amino acid sequence of: SEQ ID NO: 11 (SseB-Rhavi), or an amino acid sequence that is at least 80%, 85%, or 90% identical to SEQ ID NO: 11.

### IpaB Fusion Proteins

One aspect of the present invention relates to a fusion protein comprising, in any order: (a) a biotin-binding moiety and (b) a IpaB polypeptide, wherein the fusion protein comprises (i) an amino acid sequence that is at least 80% identical to SEQ ID NO: 3 (Rhavi) and (ii) an amino acid sequence that is at least 80% identical to SEQ ID NO: 5 (IpaB).

In all aspects of the fusion protein as disclosed herein, the biotin-binding moiety comprises an amino acid sequence that is at least 80%, 85%, 90% identical to SEQ ID NO:3 or a biotin binding portion thereof. In all aspects of the fusion protein as disclosed herein, IpaB polypeptide comprises an amino acid sequence that is at least 80%, 85%, 90% identical to SEQ ID NO:5.

In some embodiments, the fusion protein comprises, in order of N- to C-terminal: a biotin-binding moiety comprises an amino acid sequence of SEQ ID NO: 3, or an amino acid sequence that is at least 80%, 85%, 90% identical to SEQ ID NO:3 or a biotin binding portion thereof, and a IpaB polypeptide comprising an amino acid sequence of SEQ ID NO: 5, or an amino acid sequence an amino acid sequence that is at least 80%, 85%, 90% identical to SEQ ID NO:5.

In some embodiments, the fusion protein comprises, in order of N- to C-terminal: a IpaB polypeptide comprising an amino acid sequence of SEQ ID NO: 5, or an amino acid sequence that is at least 80%, 85%, 90% identical to SEQ ID NO:5, and a biotin-binding moiety comprises an amino acid sequence of SEQ ID NO: 3, or an amino acid sequence that is at least 80%, 85%, 90% identical to SEQ ID NO:3 or a biotin binding portion thereof.

In some embodiments, fusion protein comprises an amino acid sequence that is at least 80%, 85%, or 90% identical to SEQ ID NO: 2. In some embodiments, fusion protein comprises an amino acid sequence that consists of SEQ ID NO: 2 (Rhavi-IpaB). In some embodiments, fusion protein comprises an amino acid sequence that is at least 80%, 85%, or 90% identical to SEQ ID NO: 10. In some embodiments, fusion protein comprises an amino acid sequence that consists of SEQ ID NO: 10 (IpaB-Rhavi).

In some embodiments, one aspect of the present invention relates to a fusion protein comprising (i) a IpaB polypeptide having an amino acid sequence having at least 80%, 85%, 90%, 95%, 96%, 97%, 98%, 99%, or 100% identity to the sequence of SEQ ID NO: 5 or an antigenic fragment thereof, and a biotin-binding moiety that is or comprises a polypeptide having at least 80%, 85%, 90%, 95%, 96%, 97%, 98%, or 99% identity to the sequence of SEQ ID NO: 3 (rhizavidin), or biotin-binding fragment thereof.

In all aspects as disclosed herein, a fusion protein comprises an amino acid sequence that is at least 80%, 85%, 90% identical to SEQ ID NO:3 or a biotin binding portion thereof. In all aspects of the fusion protein as disclosed herein, IpaB polypeptide comprises an amino acid sequence that is at least 80%, 85%, 90% identical to SEQ ID NO:3.

In some embodiments, the fusion protein comprises, in order of N- to C-terminal: a biotin-binding moiety comprises an amino acid sequence of SEQ ID NO: 3, or an amino acid sequence that is at least 80%, 85%, 90% identical to SEQ ID NO: 3 or a biotin binding portion thereof, and a IpaB polypeptide comprising an amino acid sequence of SEQ ID NO: 5, or an amino acid sequence an amino acid sequence that is at least 80%, 85%, 90% identical to SEQ ID NO:5.

In some embodiments, the fusion protein comprises, in order of N- to C-terminal: a IpaB polypeptide comprising an amino acid sequence of SEQ ID NO: 5, or an amino acid sequence that is at least 80%, 85%, 90% identical to SEQ ID NO: 5, and a biotin-binding moiety comprises an amino acid sequence of SEQ ID NO: 3, or an amino acid sequence that is at least 80%, 85%, 90% identical to SEQ ID NO: 3 or a biotin binding portion thereof.

In some embodiments, fusion protein comprises an amino acid sequence that is at least 80%, 85%, or 90% identical to SEQ ID NO: 2.

### CP1

In some embodiments, the complex comprises a fusion protein comprising Rhazavidin (Rhavi), SP1500 and SP785 as disclosed herein, which has been previously described in WO2020056127, which is incorporated herein in its entirety by reference. In some embodiments, the fusion protein useful in the MAPS complex comprises a CP1 fusion protein having an amino acid sequence of SEQ ID NO: 6, which is Rhavi-SP1500-SP785 fusion protein and corresponds to acids of SEQ ID NO: 23 as disclosed in WO2020/056127. In some embodiments, the fusion protein useful in the MAPS complex comprises a CP1 fusion protein having an amino acid sequence having at least 80%, 85%, 90%, 95%, 96%, 97%, 98%, or 99% identity to the sequence of SEQ ID NO: 6. In some embodiments, the fusion protein useful in the MAPS complex comprises a CP1 fusion protein having an amino acid sequence selected from any of SEQ ID NO: 16, 17, 21, 22, 23, 24, 25 or 26 as disclosed in WO2020/056127, which is incorporated herein in its entirety. In some embodiments, the fusion protein useful in the MAPS complex comprises a CP1 fusion protein having an amino acid sequence having at least 80%, 85%, 90%, 95%, 96%, 97%, 98%, or 99% identity to a sequence selected from any of SEQ ID NO: 17, 16, 21, 22, 23, 24, 25 or 26 as disclosed in WO2020/056127, which is incorporated herein in its entirety.

In some embodiments, the fusion protein useful in the MAPS complex comprises a CP1 fusion protein having an amino acid sequence having at least 80%, 85%, 90%, 95%, 96%, 97%, 98%, or 99% identity to any of the sequences selected from: SEQ ID NO: 6, 12, 13, 14, 15, 16, or 17 as disclosed herein. Double and triple fusion proteins comprising Rhizavidin and comprising more than two antigenic polypeptides.

As disclosed herein, in some embodiments, a MAPS-Shigella immunogenic complex comprises a double fusion polypeptide comprising: Rhizavidin (SEQ ID NO: 3), SP1500 (SEQ ID NO: 9) and SP0785 (SEQ ID NO: 8). The term “CP1” is used refer to a polypeptide comprising Rhizavidin (SEQ ID NO: 3), SP1500 (SEQ ID NO: 9) and SP0785 (SEQ ID NO: 8), or polypeptides having at least 85% sequence identity thereto. An exemplary “CP1” double fusion polypeptide for use in the MAPS-Shigella immunogenic complex comprises the amino acid sequence of SEQ ID NO: 6 or a polypeptide having an amino acid sequence at least 80% identity thereto, however, other double fusion polypeptides comprising rhizavidin (SEQ ID NO: 3), SP1500 (SEQ ID NO: 9) and SP0785 (SEQ ID NO: 8) are encompassed for use, and are disclosed in Table 2 of WO2020/056127. Accordingly, in some embodiments, a CP1 fusion protein useful in the MAPS complex comprises a CP1 fusion protein having an amino acid sequence having at least 80%, 85%, 90%, 95%, 96%, 97%, 98%, or 99% identity to a sequence selected from any of SEQ ID NO: 16, 17, 21, 22, 23, 24, 25 or 26 as disclosed in WO2020/056127, which is incorporated herein in its entirety. Nucleic acid sequence encoding CP1 fusion proteins having amino acid sequence of SEQ ID NO: 16, 17, 21, 22, 23, 24, 25 or 26 as disclosed in WO2020/056127 are encoded by the nucleic acid sequences of SEQ ID NO: 27-36 as disclosed in WO2020/056127, respectively.

In some embodiments, a MAPS-Shigella immunogenic complex comprise a fusion polypeptide that comprise Rhizavidin (SEQ ID NO: 3) and at least two other polypeptides. Exemplary fusion proteins for use in the MAPS-Shigella immunogenic complexes are disclosed in Table 1, which discloses fusion proteins comprising Rhizavidin (SEQ ID NO: 3) and at least two or at least three antigenic polypeptides. In some embodiments, the fusion protein can comprise a Rhizavidin polypeptide of SEQ ID NO: 3 or a biotin-binding portion having at least 85% sequence identity thereto, and two polypeptides selected from any two from the group of: IpaB (SEQ ID NO: 5), SseB (SEQ ID NO: 4), SP1500 (SEQ ID NO: 9), SP0785 (SEQ ID NO: 8), or polypeptides having at least 80% sequence identity to any of SEQ ID NO: 5, 4, 8, 9. Any order of the polypeptides selected from SEQ ID NO: 5, 4, 8, 9 attached to the Rhavi polypeptide is envisioned.

In some embodiments, the fusion protein can comprise a Rhizavidin polypeptide of SEQ ID NO: 3 or a biotin-binding portion having at least 85% sequence identity thereto, and three polypeptides selected from any two from the group of: IpaB (SEQ ID NO: 5), SseB (SEQ ID NO: 4), SP1500 (SEQ ID NO: 9), SP0785 (SEQ ID NO: 8), or polypeptides having at least 80% sequence identity to any of SEQ ID NO: 5, 4, 8, 9, where order of the polypeptides selected from SEQ ID NO: 4, 5, 8, 9 attached to the Rhavi polypeptide is envisioned.

Table 1: Compositions of exemplary double and triple fusion proteins with Rhizavidin useful in the MAPS-Shigella immunogenic complexes, which can be non-covalently attached to any of the antigenic Shigella polysaccharides from Shigella flexneri or Shigella sonnei, e.g., Shigella flexneri serotype selected from: 2a, 3a or 6, or Shigella sonnei as disclosed herein.

| Exemplary double fusion proteins | Exemplary triple fusion proteins |
|---|---|
| with a Rhizavidin (Rhavi) biotin | with a Rhizavidin (Rhavi) biotin |
| binding protein | binding protein |
| Rhavi-SseB-IpaB | Rhavi-SP1500-SseB-IpaB |
| Rhavi-SseB-SP1500 | Rhavi-SP0785-SseB-IpaB |
| Rhavi-SseB-SP0785 | Rhavi-SP0785-SseB-SP1500 |
| Rhavi-IpaB-SP1500 | Rhavi-SP1500-SseB-SP0785 |
| Rhavi-IpaB-SP0785 | Rhavi-SseB-IpaB-SP1500 |
| Rhavi-IpaB-SseB | Rhavi-SseB-IpaB-SP0785 |
| Rhavi-IpaB-IpaB | Rhavi-SseB-SP1500-SP0785 |
| Rhavi-SseB-SseB | Rhavi-IpaB-SP1500-SP0785 |
| Rhavi-SP1500-SP0785 (“CP1”) | |

Table 1 shows exemplary order of antigenic polypeptides in double fusion proteins and is for purposes of illustration only. One of ordinary skill in the art can envision alternative orders or arrangements of the polypeptides in each of the fusion proteins. For purposes of illustration only and without being limited to theory, using Rhavi-SseB-IpaB as an illustrative example, the fusion protein can be arranged or selected from any of: Rhavi-SseB-IpaB, Rhavi-IpaB-SseB, SseB-IpaB-Rhavi, IpaB-SseB-Rhavi, or SseB-Rhavi-IpaB, IpaB-Rhavi-SseB. Such a variation in the order of polypeptides can be applied to any of the exemplary fusion proteins disclosed in Table 1. As disclosed herein, there may be 1 or more linkers as disclosed herein between the rhizavidin polypeptide and an antigenic polypeptide, and/or between each of the antigenic polypeptides. In some embodiments, there is no linker between rhizavidin and an antigenic polypeptide, and/or between each of the antigenic polypeptides.

One aspect of the present invention relates to an immunogenic composition comprising a fusion protein comprising, in any order: (a) a biotin-binding moiety and (b) at least one SseB polypeptide, wherein the fusion protein comprises (i) an amino acid sequence that is at least 80% or 85% identical to SEQ ID NO: 3 (Rhavi) and (ii) at least one of: polypeptide comprising an amino acid sequence that is at least 80% identical to SEQ ID NO: 4 (SseB) or at least one polypeptide comprising an amino acid sequence that is at least 80% identical to SEQ ID NO: 5 (IpaB).

In some embodiments, the present invention provides a fusion protein comprising, in any order: (a) a biotin-binding moiety and (b) at least one SseB polypeptide, wherein the fusion protein comprises, in any order (i) an amino acid sequence that is at least 80% identical to SEQ ID NO: 3 (Rhavi) and (ii) a polypeptide comprising an amino acid sequence that is at least 80% or 85% identical to SEQ ID NO: 5 (IpaB) and (iii) a polypeptide comprising an amino acid sequence that is at least 80% identical to SEQ ID NO: 4 (SseB). In some embodiment, the fusion protein comprises, in the following order: Rhavi-[SseB]-[IpaB], or Rhavi-[IpaB]-[SseB], [SseB]-[IpaB]-Rhavi, or [IpaB]-[SseB]-Rhavi, [SseB]-Rhavi-[IpaB], or [IpaB]-Rhavi-[SseB], where there can be no linkers, or one or more linkers as disclosed herein between each of the polypeptides Rhavi, SseB or IpaB, where there can be no linkers, or one or more linkers as disclosed herein between each of the polypeptides Rhavi, SseB or IpaB. In some embodiments, the fusion protein comprises SEQ ID NO: 56 (Rhavi-IpaB-SseB) or SEQ ID NO: 57 (Rhavi-SseB-IpaB) or an amino acid sequence that is at least 80%, 85%, 90% identical to SEQ ID NO: 56 or 57.

In some embodiments, a fusion protein comprising Rhavi and IpaB can further comprise at least one additional antigenic polypeptide, selected from any of: SP1500, SP0785, or SseB, as disclosed herein, for example, the SP1500 polypeptide comprises an amino acid sequence of SEQ ID NO: 9, or an amino acid sequence that is at least 80%, 85%, 90% identical to SEQ ID NO:9, and where the SP785 polypeptide comprises an amino acid sequence of SEQ ID NO: 8, or an amino acid sequence that is at least 80%, 85%, 90% identical to SEQ ID NO:8, and where the SseB polypeptide comprises an amino acid sequence of SEQ ID NO: 4, or an amino acid sequence that is at least 80%, 85%, 90% identical to SEQ ID NO:4.

In some embodiments, the fusion protein can further comprise a linker or spacer positioned between the biotin-binding moiety and the IpaB polypeptide, wherein the linker comprises the amino acid sequence selected from: GGGGSSS (SEQ ID NO: 21), TDPNSSS, SSS, AAA (SEQ ID NO: 51), or any of SEQ ID NO: 37-52 as disclosed in U.S. application Ser. No. 16/568,646.

In some embodiments, such fusion proteins disclosed in Table 1 can be used in Salmonella-MAPS immunogenic complexes, where the polysaccharide is a salmonella polysaccharide for use in, for example, Shigella-Salmonella MAPS vaccines or immunogenic compositions as disclosed herein.

In some embodiments, the biotin-binding moiety, e.g., rhizavidin in a double or triple fusion protein as disclosed herein comprises an amino acid sequence of at least 80%, or 90%, or 95% sequence identity to SEQ ID NO: 3 that has any one or more of the amino acid modifications: N80, T108, N118, S119A, N138A, for example, Rhavi for use in the fusion proteins as disclosed herein comprises an amino acid sequence of at least 80%, or 90%, or 95%, 98%, 99% or 100% sequence identity to SEQ ID NO: 55, wherein the biotin binding moiety has at least one or more of the amino acid modifications: N80, T108, N118, S119A, N138A.

### Linkers or Spacers

In some embodiments, the fusion protein of the immunogenic complex comprises one or more linkers and/or tags, e.g., a histidine tag. In some embodiments, the linker comprises a polypeptide comprising an amino acid sequence of SEQ ID NO: 44 (GGGSS). In some embodiments, the linker comprises a polypeptide comprising an amino acid sequence having at least 80%, 85%, 90%, 95%, 96%, 97%, 98%, or 99% identity to the sequence of SEQ ID NO: 44. In some embodiments, the linker comprises the amino acid sequence AAA (SEQ ID NO: 51). In some embodiments, the fusion protein of the immunogenic complex comprises a first linker comprising a polypeptide comprising the amino acid sequence of SEQ ID NO:44 (GGGSS), and a second linker comprising the amino acid sequence AAA (SEQ ID NO: 51). In some embodiments, such a linker may be synthesized, or derived from amino acid residues from a restriction site (e.g., a Not I restriction site).

In some embodiments, a fusion protein comprises one or more linkers. In some embodiments, a linker is or comprises one or more amino acids. In some embodiments, a fusion protein comprises an antigenic polypeptide joined to a biotin-binding moiety by a linker. In some embodiments, a fusion protein comprises at least a first antigenic polypeptide and a biotin-binding moiety, and at least one linker.

In some embodiments, a linker interposes a structure between two protein moieties. In some embodiments, the structure is or comprises an α-helix. In some embodiments the structure is or comprises a β-strand. In some embodiments, the structure is or comprises a coil/bend. In some embodiments, the structure is or comprises a turn. In some embodiments, a linker decreases steric hindrance between two protein moieties joined by the linker. In some embodiments, a linker decreases unfavorable interactions between two protein moieties joined by the linker. In some embodiments, a linker comprises a mixture of glycine and serine residues. In some embodiments, the linker may additionally comprise threonine, proline, and/or alanine residues. In some embodiment a linker is hydrophilic. In some embodiments a linker is hydrophobic. In some embodiments a linker increases the stability of the fusion protein containing the linker.

In some embodiments, a linker does not interfere with the folding of an antigenic polypeptide to which it is joined. In some embodiments, a linker does not interfere with the antigenicity of an antigenic polypeptide to which it is joined. In some embodiments, a linker does not reduce the antigenicity of an antigenic polypeptide to which it is joined. In some embodiments, a linker does not eliminate the antigenicity of an antigenic polypeptide to which it is joined. In some embodiments the effect of the linker is determined by comparing the polypeptide with the polypeptide joined to the linker.

In some embodiments, a linker does not interfere with the folding of a biotin-binding moiety to which it is joined. In some embodiments, a linker does not interfere with the biotin-binding ability of a biotin-binding moiety to which it is joined. In some embodiments, a linker does not reduce the biotin-binding ability of a biotin-binding moiety to which it is joined. In some embodiments, a linker does not eliminate the biotin-binding ability of a biotin-binding moiety to which it is joined. In some embodiments the effect of the linker is determined by comparing the biotin-binding moiety with the biotin-binding moiety joined to the linker.

In some embodiments, a linker is not antigenic. In some embodiments, a linker does not elicit a T cell response. In some embodiments, a linker does not elicit a B cell response. In some embodiments, a linker does not induce a T cell or a B cell response.

In some embodiments, a linker comprises two or more amino acids. In some embodiments, a linker may be 3-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20, 10-15, 3-10, 3-9, 3-8, 3-7, 3-6, 3-5, 3-4, or 2-3 amino acids in length. In some embodiments, a linker comprises between 10-100, 10-90, 10-80, 10-70, 10-60, 10-50, 10-40, 10-30, 10-20, 10-15 amino acids. In some embodiments, the linker comprises at least 10, 15, 20, 25, 30, 35, 40, 45, 50, 55, 60, 65, 70, 75, 80, 85, 90, or 95 amino acids. In some embodiments, a linker is or comprises 2, 3, 4, 5, 6, 7, 8, 9, 10, 15, 20, 25, 30, 35, 40, 45, 50, 55, 60, 65, 70, 75, 80, 85, 90, 95, or 100 amino acids, or more than 100 amino acids in length.

In some embodiments, a linker is a flexible linker. Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties. In some embodiments a linker comprises small non-polar (e.g. Gly) or polar (e.g. Ser or Thr) amino acids. In some embodiments, a linker is a Gly-Ser linker (SEQ ID NO: 52).

In some embodiments, a linker is or comprises an amino acid sequence of GGGGSSS (SEQ ID NO:21). In some embodiments, a linker is or comprises a sequence of (GGGGS)n (SEQ ID NO:22), where n represents the number of repeating GGGGS (SEQ ID NO: 22) units and is 1, 2, 3, 4, 5, 6, 7, 8, 9, 10, 11, 12, 13, 14, 15, 16, 17, 18, 19, 20, 25, 30 or more. In some embodiments, a polypeptide linker may have an amino acid sequence that is or comprises GGGGSGGGGSGGGGS (SEQ ID NO:24) (i.e., (GGGGS)3 (SEQ ID NO: 24)) or GGGGSGGGGSGGGGSGGGGSGGGGSGGGGS (SEQ ID NO:25) (i.e., (GGGGS)6 (SEQ ID NO: 25)). In some embodiments, a linker comprises one or more of Gly, Ser, Thr, Ala, Lys, and Glu. In some embodiments, a linker is or comprises KESGSVSSEQLAQFRSLD (SEQ ID NO: 26). In some embodiments, a linker is or comprises EGKSSGSGSESKST (SEQ ID NO: 27). In some embodiments, a linker is or comprises (Gly)n (SEQ ID NO:28) where n represents the number of repeating Gly residues and is 1, 2, 3, 4, 5, 6, 7, 8, 9, 10, 11, 12, 13, 14, 15, 16, 17, 18, 19, 20, 25, 30 or more. In some embodiments a linker is or comprises GGG (SEQ ID NO: 53). In some embodiments, a linker is or comprises (Gly)6 (SEQ ID NO:47). In some embodiments, a linker is or comprises (Gly)8 (SEQ ID NO:48). In some embodiments, a linker is or comprises GSAGSAAGSGEF (SEQ ID NO:30). In some embodiments, a linker is or comprises an amino acid sequence of AAA (SEQ ID NO: 51).

In some embodiments, a linker is a rigid linker. Rigid linkers are useful to keep a fixed distance between domains and to maintain their independent functions. Rigid linkers may also be useful when a spatial separation of the domains is critical to preserve the stability or bioactivity of one or more components in the fusion. In some embodiments, a linker is or comprises (EAAAK)n (SEQ ID NO:31) where n represents the number of repeating EAAAK (SEQ ID NO: 49) units and is 1, 2, 3, 4, 5, 6, 7, 8, 9, 10, 11, 12, 13, 14, 15, 16, 17, 18, 19, 20, 25, 30 or more. In some embodiments, a linker is or comprises A(EAAAK)nA, (SEQ ID NO:32) where n represents the number of repeating EAAAK (SEQ ID NO: 31) units and is 1, 2, 3, 4, 5, 6, 7, 8, 9, 10, 11, 12, 13, 14, 15, 16, 17, 18, 19, 20, 25, 30 or more. In some embodiments, a linker is or comprises A(EAAAK)nA (SEQ ID NO: 32), where n represents the number of repeating EAAAK (SEQ ID NO: 31) units and is 2, 3, 4, or 5. In some embodiments, a linker is or comprises A(EAAAK)4ALEA(EAAAK)4A (SEQ ID NO:33). In some embodiments, a linker is or comprises [A(EAAAK)nA]m, (SEQ ID NO:34) wherein n is 2, 3, or 4 and m is 1 or 2. In some embodiments, a linker is or comprises AEAAAKEAAAKA (SEQ ID NO:35).

In some embodiments a linker is or comprises (X-Pro)n (SEQ ID NO:36), with X designating any amino acid, where n represents the number of repeating X-Pro units and is 1, 2, 3, 4, 5, 6, 7, 8, 9, 10, 11, 12, 13, 14, 15, 16, 17, 18, 19, 20, 25, 30 or more. In some embodiments a linker is or comprises (Ala-Pro)n (SEQ ID NO: 37), where n represents the number of repeating Ala-Pro units and is 1, 2, 3, 4, 5, 6, 7, 8, 9, 10, 11, 12, 13, 14, 15, 16, 17, 18, 19, 20, 25, 30 or more. In some embodiments a linker is or comprises (Ala-Pro)n (SEQ ID NO: 37), where n represents the number of repeating Ala-Pro units and is 5, 6, 7, 8, 9, 10, 11, 12, 13, 14, 15, 16, or 17.

In some embodiments a linker is or comprises (Lys-Pro)n (SEQ ID NO:38), where n represents the number of repeating Lys-Pro units and is 1, 2, 3, 4, 5, 6, 7, 8, 9, 10, 11, 12, 13, 14, 15, 16, 17, 18, 19, 20, 25, 30 or more. In some embodiments a linker is or comprises (Gln-Pro)n (SEQ ID NO:54), where n represents the number of repeating Gln-Pro units and is 1, 2, 3, 4, 5, 6, 7, 8, 9, 10, 11, 12, 13, 14, 15, 16, 17, 18, 19, 20, 25, 30 or more. In some embodiments, a linker is or comprises (Ala-Pro)7 (SEQ ID NO:39).

In some embodiments a linker is or comprises GAPGGGGGAAAAAGGGGGGAP (GAG linker, SEQ ID NO:41). In some embodiments a linker is or comprises GAPGGGGGAAAAAGGGGGGAPGGGGGAAAAAGGGGGGAP (GAG2 linker, SEQ ID NO:42). In some embodiments a linker is or comprises

(GAG3 linker, SEQ ID NO: 43)
GAPGGGGGAAAAAGGGGGGAPGGGGGAAAAAGGGGGGAPGGGGGA
AAAAGGGGGGAP.

Suitable linkers or spacers also include those having an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to the above exemplary linkers.

Additional linkers suitable for use with some embodiments may be found in U.S. Patent Publication No. 2012/0232021, filed on Mar. 2, 2012, and [Chen, 2013] the disclosures of which is hereby incorporated by reference in their entireties. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

### Nucleic Acids Encoding Fusion Proteins and Expression of the Fusion Proteins

In some embodiments, the present disclosure provides nucleic acids, e.g., DNA, RNA, or analogs thereof, encoding one or more of the polypeptides and/or fusion proteins described herein. An underlying DNA sequence for the polypeptides described herein may be modified in ways that do not affect the sequence of the protein product, and such sequences are included in the invention. In some embodiments, a DNA sequence may be codon-optimized to improve expression in a host such as a bacterial cell line, e.g., E. coli, an insect cell line (e.g., using the baculovirus expression system), or a mammalian (e.g., human or Chinese Hamster Ovary) cell line.

Another aspect of the present invention relates to heterologous nucleic acid sequence encoding a fusion protein comprising, in any order: (a) a biotin-binding moiety and (b) a IpaB polypeptide, wherein the fusion protein comprises (i) an amino acid sequence that is at least 80% identical to SEQ ID NO: 3 or SEQ ID NO: 56 (Rhavi or Rhavi-A5T) and (ii) an amino acid sequence that is at least 80% identical to SEQ ID NO: 5 (IpaB). In some embodiments, the heterologous nucleic acid sequence comprises a nucleic acid sequence of SEQ ID NO: 46 (IpaB) or a nucleic acid sequence at least 85% sequence identity to SEQ ID NO: 46, and a nucleic acid sequence of SEQ ID NO: 60 (Rhavi) or a nucleic acid sequence at least 85% sequence identity to SEQ ID NO: 60. In some embodiments, the heterologous nucleic acid sequence comprises SEQ ID NO: 45, or a nucleic acid sequence having at least 85% sequence identity to SEQ ID NO: 45 (Rhavi-IpaB).

Another aspect of the present invention relates to heterologous nucleic acid sequence encoding a fusion protein comprising, in any order: (a) a biotin-binding moiety and (b) a SseB polypeptide, wherein the fusion protein comprises (i) an amino acid sequence that is at least 80% identical to SEQ ID NO: 3 or SEQ ID NO: 56 (Rhavi or Rhavi-A5T) and (ii) an amino acid sequence that is at least 80% identical to SEQ ID NO: 4 (SseB).

In some embodiments, the heterologous nucleic acid sequence comprises a nucleic acid sequence of SEQ ID NO: 61 (SseB) or a nucleic acid sequence at least 85% sequence identity to SEQ ID NO: 61, and a nucleic acid sequence of SEQ ID NO: 60 (Rhavi) or a nucleic acid sequence at least 85% sequence identity to SEQ ID NO: 60. In some embodiments, the heterologous nucleic acid sequence comprises SEQ ID NO: 66, or a nucleic acid sequence having at least 85% sequence identity to SEQ ID NO: 66 (Rhavi-SseB).

In some embodiments, the heterologous nucleic acid that encodes a fusion protein as disclosed herein comprising a biotin-binding moiety, comprises a nucleic acid sequence that encodes a Rhavi polypeptide having an amino acid sequence of at least 80%, or 90%, or 95% sequence identity to SEQ ID NO: 3 that has any one or more of the amino acid modifications: N80, T108, N118, S119A, N138A. In some embodiments, the heterologous nucleic acid encoding a fusion protein as disclosed herein comprises a nucleic acid that encodes a modified Rhizavidin polypeptide, e.g., Rhavi-5AT, wherein the nucleic acid sequence encodes a Rhizavidin polypeptide comprises an amino acid sequence of at least 80%, or 90%, or 95%, 98%, 99% or 100% sequence identity to SEQ ID NO: 56, wherein the biotin binding moiety has at least one or more of the amino acid modifications: N80, T108, N118, S119A, N138A.

In some embodiments, the heterologous nucleic acid further comprises a nucleic acid that encodes at least one linker, or a nucleic acid that encodes an expression tag, or both. In some embodiments, a nucleic acid encoding an expression tag is absent (i.e., not attached to a nucleic acid encoding the fusion protein), rather a nucleic acid encoding an expression tag can be present with a nucleic acid encoding a chaperone for SseB or IpgC. In some embodiments, the nucleic acid encoding at least one linker can be located between SEQ ID NO: 60 (Rhavi) and SEQ ID NO: 61 (SseB), or sequences at having least 85% to SEQ ID NO: 60 or 61, thereof. In some embodiments, the nucleic acid encoding at least one linker can be located between SEQ ID NO: 60 (Rhavi) and SEQ ID NO: 46 (IpaB), or sequences at having least 85% to SEQ ID NO: 60 or 46, thereof.

In some embodiments, the nucleic acid sequence encoding at least one linker encodes a linker having an amino acid sequence selected from any of GGGSS (SEQ ID NO: 44), GGGGSSS (SEQ ID NO: 21) or AAA (SEQ ID NO: 51) and any of: SEQ ID NOS: 39-60 and SEQ ID NO: 62, 63, 108, 109 as disclosed in U.S. application Ser. No. 16/568,646. In some embodiments, the heterologous nucleic acid sequence is codon-optimized to improve expression in the host cell. In some embodiments, the nucleic acid sequence further comprises a nucleic acid sequence encoding one or more of: polyadenylation sequence or termination sequence, a signal sequence.

In some embodiments, the heterologous nucleic acid sequence encodes Rhavi-IpaB fusion protein and is selected from one of the following, in the following order: (a) a nucleic acid sequence of SEQ ID NO: 60 (Rhavi) or a nucleic acid sequence at least 85% sequence identity to SEQ ID NO: 60, and a nucleic acid sequence of SEQ ID NO: 46 (IpaB) or a nucleic acid sequence at least 85% sequence identity to SEQ ID NO: 46, or (b)a nucleic acid sequence of SEQ ID NO: 46 or a nucleic acid sequence at least 85% sequence identity to SEQ ID NO: 46, and a nucleic acid sequence of SEQ ID NO: 60 or a nucleic acid sequence at least 85% sequence identity to SEQ ID NO: 60. In some embodiments, a heterologous nucleic acid sequence encoding a Rhavi-IpaB fusion protein further comprises at least one nucleic acid selected from: SEQ ID NO: 61 (SseB), SEQ ID NO: 62 (SP0785), SEQ ID NO: 63 (SP1500) or SEQ ID NO: 64 (CP1), or a nucleic acid sequence having at least 80%, or at least 85%, or at least 90%, or at least 95% sequence identity to SEQ ID NOS 46, 62, 63, or 64 respectively. In some embodiments, the nucleic acid encoding a Rhavi-SseB fusion protein is codon optimized for expression in an expression system as disclosed herein.

In some embodiments, the heterologous nucleic acid sequence encodes a Rhavi-SseB fusion protein is selected from one of the following, in the following order: a) a nucleic acid sequence of SEQ ID NO: 60 (Rhavi) or a nucleic acid sequence at least 85% sequence identity to SEQ ID NO: 60, and a nucleic acid sequence of SEQ ID NO: 61 (SseB) or a nucleic acid sequence at least 85% sequence identity to SEQ ID NO: 61 or b) a nucleic acid sequence of SEQ ID NO: 61 or a nucleic acid sequence at least 85% sequence identity to SEQ ID NO: 61, and a nucleic acid sequence of SEQ ID NO: 60 or a nucleic acid sequence at least 85% sequence identity to SEQ ID NO: 60. In some embodiments, a heterologous nucleic acid sequence encoding a Rhavi-SseB fusion protein further comprises at least one nucleic acid selected from: SEQ ID NO: 46 (IpaB), SEQ ID NO: 62 (SP0785), SEQ ID NO: 63 (SP1500) or SEQ ID NO: 64 (CP1), or a nucleic acid sequence having at least 80%, or at least 85%, or at least 90%, or at least 95% sequence identity to SEQ ID NOS 46, 62, 63, or 64 respectively. In some embodiments, the nucleic acid encoding a Rhavi-SseB fusion protein is codon optimized for expression in an expression system as disclosed herein.

In some embodiments, the heterologous nucleic acid sequence encodes any double or triple fusion protein as disclosed in Table 1 herein.

Another aspect relates to a cell transfected with the nucleic acid sequences encoding the Rhavi-IpaB or Rhavi-SseB fusion proteins as disclosed herein. In some embodiments, the cell, e.g., an expression host cell, is transfected with a nucleic acid sequence encoding the Rhavi-IpaB and/or Rhavi-SseB fusion proteins as disclosed herein.

In some embodiments, the expression host cell is selected from the group consisting of: E. coli, an insect cell line (e.g., baculovirus expression system) or a mammalian cell line (e.g., CHO cell line).

Another aspect of the invention relates to an expression vector comprising the nucleic acid sequence encoding the Rhavi-IpaB and/or Rhavi-SseB fusion proteins as disclosed herein.

In some embodiments, the present disclosure provides nucleic acids, e.g., DNA, RNA, or analogs thereof, encoding one or more of the polypeptides and/or fusion proteins described herein. An underlying DNA sequence for the polypeptides described herein may be modified in ways that do not affect the sequence of the protein product, and such sequences are included in the invention. In some embodiments, a DNA sequence may be codon-optimized to improve expression in a host such as a bacterial cell line, e.g., E. coli, an insect cell line (e.g., using the baculovirus expression system), or a mammalian (e.g., human or Chinese Hamster Ovary) cell line.

In some embodiments, the present disclosure provides nucleic acids, e.g., DNA, RNA, or analogs thereof, that are at least 70%, 80%, 85%, 90%, 95%, 97%, 98%, 99%, 99.5%, or 100% identical to a nucleic acid sequence provide in Table lor a variant or portion thereof. In some embodiments, the nucleic acid is 600-2000, 800-1800, 1000-1600, 1200-1400 nucleotides in length. In some embodiments, the nucleic acid is 600-1600, 800-1800, 1000-2000, 2000-3000, or 3000-4000 nucleotides in length. In some embodiments, a nucleic acid may be used as a vaccine.

Nucleic acids encoding polypeptides or fusion proteins as listed, or fragments thereof, can be cloned into any of a variety of expression vectors, under the control of a variety of regulatory elements, and fusions can be created with other sequences of interest. Methods of cloning nucleic acids are routine and conventional in the art. For general references describing methods of molecular biology which are mentioned in this application, e.g., isolating, cloning, modifying, labeling, manipulating, sequencing and otherwise treating or analyzing nucleic acids and/or proteins, see, e.g., Sambrook et al,1989; Ausubel et al,1995; Davis et al, 1986; Hames et al, 1985; Dracopoli et al, 2018; and Coligan et al, 2018.

### Production and Expression of the Fusion Proteins

Purification of SseB from E. coli (either on its own or as a fusion protein as disclosed herein) needs co-expression of chaperone SseA. Accordingly, SseB can be co-expressed from E. coli with SseA. The SseB polypeptide can be separated from its SseA chaperone using detergent wash (Dodecyldimethylaminoxid (LDAO) or sodium Deoxycholate (SDOC)). In some embodiments, SseB is co-expressed with His-tagged SseA in E. coli, the SseB and SseA complex is isolated by using the His-tagged SseA protein, the complex is then washed with LDAO or SDOC to elute the SseB polypeptide.

In some embodiments, a fusion protein as described herein, or its chaperone, may comprise a tag. A tag may be N-terminal or C-terminal. For instance, tags may be added to a polypeptide (via additions or modifications on the encoding DNA sequence) to facilitate purification, detection, solubility, or confer other desirable characteristics on the protein. In some embodiments a tag may be a peptide, oligopeptide, or polypeptide that may be used in affinity purification. In some embodiments, a tag is, comprises, or is derived from one or more of polyhistidine (His), Glutathione S-transferase (GST), tandem affinity purification (TAP), FLAG, myc, human influenza hemagglutinin (HA), maltose binding protein (MBP), vesicular Stomatitis viral glycoprotein (VSV-G), thioredoxin, V5, avidin, streptavidin, biotin carboxyl carrier protein (BCCP), Calmodulin, Nus, S tags, lipoprotein D, and galactosidase. In some embodiments, a His tag is or comprises an amino acid sequence of H., wherein n is an integer between 2 and 10 (SEQ ID NO: 49). Exemplary His tags include HHHHHH (SEQ ID NO:58) and MSYYHHHHH (SEQ ID NO: 59). In other embodiments, the fusion protein is free of tags such as protein purification tags, and is purified by a method not relying on affinity for a purification tag on the fusion protein. In some embodiments, a chaperone comprises a purification tag, e.g., a His-tag, and the fusion protein can be purified by the affinity purification of its chaperone protein (e.g., His-tagged SseA chaperone for purification of a fusion protein comprising SseB, and His-tagged IpgC chaperone for purification of a fusion protein comprising IpaB), where the tagged-chaperone non-covalently associates with the fusion protein and can be removed (i.e., dissociated from the fusion protein) with a detergent was as disclosed herein. In some embodiments, a fusion protein or chaperone described herein may contain a membrane translocating sequence (MTS), to facilitate introduction of the fusion protein into a mammalian cell and subsequent stimulation of the cell-mediated immune response. Exemplary membrane translocating sequences include the hydrophobic region in the signal sequence of Kaposi fibroblast growth factor, the MTS of a synuclein, the third helix of the Antennapedia homeodomain, SN50, integrin 3 h-region, HIV Tat, pAntp, PR-39, abaecin, apidaecin, Bac5, Bac7, P. berghei CS protein, and those MTSs described in U.S. Pat. Nos. 6,248,558, 6,432,680 and 6,248,558.

### Antigenic Polysaccharides

In some embodiments, an immunogenic complex described herein includes one or more Shigella 0-specific polysaccharides (OSP). In some embodiments, an immunogenic complex described herein comprises a polysaccharide from the Shigella subspecies Shigella flexneri or Shigella sonnei. In some embodiments, an immunogenic complex includes one or more Shigella O specific polysaccharides or 0-specific polysaccharides (OSP) from, or derived from, one or more Shigella serovars selected from Shigella flexneri or Shigella sonnei.

In some embodiments, an immunogenic complex described herein comprises a Shigella polysaccharide that is >60 kDa, or >70 kDa, or >80 kDa, or >90 kDa, or >100 kDa, or >110 kDa, or >120 kDa. In some embodiments, an immunogenic complex described herein comprises an OSP polysaccharide from Shigella that is between 90-110 kDa.. Shigella species are classified by three serogroups and one serotype. There are three serogroups, A, B and C. Serogroup A: S. dysenteriae (15 serotypes), Serogroup B: S. flexneri (9 serotypes), and Serogroup C: S. boydii (19 serotypes), and Serogroup D: S. sonnei (one serotype). Groups A-C are physiologically similar; S. sonnei (group D) can be differentiated on the basis of biochemical metabolism assays. Three Shigella groups are the major disease-causing species: S. flexneri is the most frequently isolated species worldwide, and accounts for 60% of cases in the developing world; S. sonnei causes 77% of cases in the developed world, compared to only 15% of cases in the developing world; and S. dysenteriae is usually the cause of epidemics of dysentery, particularly in confined populations such as refugee camps.

In some embodiments, an immunogenic complex described herein includes one Shigella polysaccharide, selected from either S. flexneri or S. Sonnei. In some embodiments, a MAPS immunogenic complex described can comprise a polysaccharide from other Shigella serogroups, including, but not limited to Shigella serogroups that cause shigellosis, such as, but not limited to Shigella boydii or Shigella dysenteriae, or an unclassified Shigella species (Shigella sp.) In some embodiments, a MAPS immunogenic complex described can comprise a polysaccharide from other Shigella serogroups, selected from the group of Shigella dysenteriae.

In some embodiments, an immunogenic complex described herein comprises a OSP polysaccharide from S. flexneri. In some embodiments, an immunogenic complex described herein comprises an O-specific polysaccharide (OSP) from any one or more of: S. sonnei or Shigella dysenteriae.

In some embodiments, an immunogenic complex described herein comprises a polysaccharide from a strain of Shigella selected from any of: S. flexneri 2a, 3a, 6 or S. sonnei.

Shigella flexneri

In some embodiments, an immunogenic complex described herein comprises at least one O-specific polysaccharide selected from Shigella subgroup S. flexneri, and can be selected from any one or more of S. flexneri strains: 2, 3a or 6 or selected from any subtypes of: 1a, 1b, 1c, 2a, 2b, 3a, 3b, 4a, 4b, 5a, 5b, X, Xv, Y, and F6. In some embodiments, an immunogenic complex described herein comprises at least one polysaccharide selected from S. flexneri, and can be selected from any one or more of S. flexneri strains: flexneri strains selected from: Shigella flexneri 1235-66, Shigella flexneri 1485-80, Shigella flexneri 1676-NY, Shigella flexneri 1997005, Shigella flexneri 1a, Shigella flexneri 1b, Shigella flexneri 1c, Shigella flexneri 2000019, Shigella flexneri 2001004, Shigella flexneri 2001020, Shigella flexneri 2001025, Shigella flexneri 2001027, Shigella flexneri 2001042, Shigella flexneri 2001044, Shigella flexneri 2001048, Shigella flexneri 2002007, Shigella flexneri 2002017, Shigella flexneri 2002021, Shigella flexneri 2002028, Shigella flexneri 2002035, Shigella flexneri 2002069, Shigella flexneri 2002091, Shigella flexneri 2002103, Shigella flexneri 2002106, Shigella flexneri 2002110, Shigella flexneri 2002127, Shigella flexneri 2002140, Shigella flexneri 2002141, Shigella flexneri 2002142 Shigella flexneri 2003035, Shigella flexneri 2003036, Shigella flexneri 2003055, Shigella flexneri 2005002 Shigella flexneri 2005025, Shigella flexneri 2005051, Shigella flexneri 2005184, Shigella flexneri 2005AH264, Shigella flexneri 2005GS061, Shigella flexneri 2747-71, Shigella flexneri 2850-71, Shigella flexneri 2930-71, Shigella flexneri 2a, Shigella flexneri 2b, Shigella flexneri 3a, Shigella flexneri 3b, Shigella flexneri 4343-70, Shigella flexneri 4a, Shigella flexneri 4c, Shigella flexneri 5, Shigella flexneri 51575, Shigella flexneri 51576, Shigella flexneri 51577, Shigella flexneri 51581, Shigella flexneri 5a, Shigella flexneri 5b, Shigella flexneri 6, Shigella flexneri 6603-63, Shigella flexneri 7a, Shigella flexneri 7b, Shigella flexneri CCH060, Shigella flexneri CDC 796-83, Shigella flexneri G1663, Shigella flexneri II;3)4,7(8), Shigella flexneri K-1770, Shigella flexneri K-218, Shigella flexneri K-227, Shigella flexneri K-272, Shigella flexneri K-304, Shigella flexneri K-315, Shigella flexneri K-404, Shigella flexneri K-671, Shigella flexneri MT1457, Shigella flexneri S5644, Shigella flexneri S5717, Shigella flexneri S6162, Shigella flexneri S6585, Shigella flexneri S6678, Shigella flexneri S6764, Shigella flexneri S7737, Shigella flexneri SFJ17B, Shigella flexneri Shi05SX04, Shigella flexneri Shi06AH028, Shigella flexneri Shi06AH091, Shigella flexneri Shi06AH116, Shigellafexneri Shi06AH130, Shigellafexneri Shi06AH135, Shigella flexneri Shi06AH66, Shigella flexneri Shi06GS02, Shigella flexneri Shi06GS07, Shigellafexneri Shi06GS37, Shigella flexneri Shi06GS43, Shigella flexneri Shi06GS48, Shigella flexneri Shi06GS55, Shigella flexneri Shi06HN006, Shigella fexneri Shi06HN016, Shigella flexneri Shi06HN023, Shigella fexneri Shi06HN091, Shigella flexneri Shi06HN118, Shigella flexneri Shi06HN159, Shigella flexneri Shi06HN244, Shigella flexneri Shi06HN250, Shigellafexneri Shi06HN344, Shigellafexneri Shi06HN347, Shigella flexneri Shi06HN378, Shigella fexneri Shi06SX36, Shigella flexneri Shi06SX53, Shigella flexneri VA-6, Shigella flexneri X variant, Shigella flexneri Y. In some embodiments, an immunogenic complex described herein comprises at least one 0-specific polysaccharide selected from Shigella flexneri, from any Shigella flexneri strain disclosed herein or known to a person of ordinary skill in the art.

In some embodiments, an immunogenic complex described herein comprises at least one lipopolysaccharide (LPS) selected from Shigella flexneri, including from serotypes S. flexneri 2a, 3a, 6.

Shigella sonnei

In some embodiments, an immunogenic complex described herein comprises at least one polysaccharide selected from Shigella sonnei.

In some embodiments, an immunogenic complex described herein comprises at least one 0-specific polysaccharide selected from Shigella sonnei, and can be selected from any Shigella sonnei strains, for example, any one or more of Shigella sonnei strains selected from the group of, but not limited to strain: Shigella sonnei 08-7761, Shigella sonnei 08-7765, Shigella sonnei 09-1032, Shigella sonnei 09-2245, Shigella sonnei 09-4962, Shigella sonnei 1DT-1, Shigella sonnei 3226-85, Shigella sonnei 3233-85, Shigella sonnei 4822-66, Shigella sonnei 53G, Shigella sonnei S6513, Shigella sonnei Ss046, Shigella sonnei str. Moseley. In some embodiments, an immunogenic complex described herein comprises at least one lipopolysaccharide (LPS) selected from Shigella sonnei, from any Shigella sonnei strain disclosed herein or known to a person of ordinary skill in the art. In some embodiments, an immunogenic complex described herein comprises at least one 0-specific polysaccharide (OPS) selected from any Shigella sonnei, from any Shigella sonnei strain disclosed herein or known to a person of ordinary skill in the art.

### Other Subgroups of Shigella

In some embodiments, an immunogenic complex described herein comprises a polysaccharide from a subgroup of Shigella selected from any of the subgroup, including, but not limited to: Shigella boydii or Shigella dysenteriae, or an unclassified Shigella species (Shigella sp.).

In some embodiments, an immunogenic complex described herein comprises a polysaccharide from a subgroup of Shigella boydii, for example, selected from any strain, including but not limited to: Shigella boydii 08-0009, Shigella boydii 08-0280, Shigella boydii 08-2671, Shigella boydii 08-2675, Shigella boydii 08-6341, Shigella boydii 09-0344, Shigella boydii 248-1B, Shigella boydii 3594-74, Shigella boydii 4444-74, Shigella boydii 965-58, Shigella boydii CDC 3083-94, Shigella boydii S6614, Shigella boydii S7334, Shigella boydii Sb227.

In some embodiments, an immunogenic complex described herein comprises a polysaccharide from a subgroup of Shigella dysenteriae, selected from any strain, including but not limited to: Shigella dysenteriae 1, Shigella dysenteriae 155-74, Shigella dysenteriae 1617, Shigella dysenteriae 225-75, Shigella dysenteriae 2a, Shigella dysenteriae 3, Shigella dysenteriae 4, Shigella dysenteriae 5514-56, Shigella dysenteriae CDC 74-1112, Shigella dysenteriae M13164, Shigella dysenteriae S6554, Shigella dysenteriae Sd197, Shigella dysenteriae SD1D, Shigella dysenteriae WRSd3, Shigella dysenteriae WRSd5.

In some embodiments, an immunogenic complex described herein comprises a polysaccharide from a subgroup of Shigella, from an unclassified Shigella species, unclassified Shigella species, selected from any strain, including but not limited to: Shigella genomosp. SF-2015,, Strains: 0310ARD15N_4, 08XMYD-9, 09-A2, 09-M2, 1.3-01, 1.5-11, 1.9-15, 102C.4, 11246, 11471, 117, 12, 121402, 121403, 121404, 121405, 121406, 127555, 15, 16, 168500, 170304, 171147, 175547, 176296, 183737, 184399, 188144, 188334, 200615, 217069, 254601, 255519, 255528, 262643, 273561, 278204, 279345, 279353, 279355, 283097, 29(2010), 291764, 296637, 298909, 312103, 312160, 4091, 4092, 4093, 4095, 4096, 4104, 4109, 44, 5, 55, 62, 63, 724B5_12EMac, 80A.2, 80A.3, 80A.4, 80A.5, 80A.6, 80A.7, 80B.3, 80B.5, 86.2, 86.3, 86.4, 86.5, 8CR, Ala-41, Ala-44, Ala-60, AB012-1, AB024, AB5121, AB5221, AB5223, AB524-2, AB5242, AR-21793, AS8, ASBCFS38, ASG33, ASS-19, BAB-3751, BAB-3752, BAB-3755, BAB-3756, BAB-3765, BAB-3787, BAB-5454, BAB-5849, BAB-625, BARD11, BBDP15, BBDP80, BBDP81, BCCO 40_FK53, BCCO 40_FK78, BCCO 40_FK81, BV23, BV29, BV30, C14, CD2, CERAR 10, CFSAN082790, CH-12, CH-24, CH-25, CH-28, CH-3, CH-30, CH-31, CH-32, CH-33, CH-35, CH-38, CH-40, CH-41, CH-42, CH-43, co-culture, CPO 6.001, CTB486, D10_76, D8, DBC-1, df-3-u8f, E10_77, ECOLX, ECOLY, EE2, ER.1.23, F-00, FC1056, FC1139, FC1172, FC1180, FC130, FC1544, FC1567, FC1655, FC1661, FC1708, FC1737, FC1764, FC1882, FC1967, FC2045, FC2117, FC2125, FC2175, FC2383, FC2531, FC2541, FC2710, FC2833, FC2928, FC3192, FC3196, FC569, FC6481, FC6482, FC6484, FC6485, FC6487, FC6488, FC6490, FC6491, FC6492, FC6493, FC6494, FC6495, FC6496, FC6498, FC6499, FC6500, FC6502, FC6505, FC6506, FC6507, FC6508, FC6509, FC6510, FC6511, FC6512, FC6513, FC6514, FC6515, FC6516, FC6517, FC6518, FC6519, FC6520, FC6521, FC6522, FC6523, FC6524, FC6525, FC6527, FC6528, FC6529, FC6532, FC6533, FC6534, FC6535, FC6536, FC6537, FC6538, FC6539, FC6540, FC6541, FC6542, FC6543, FC6544, FC6545, FC6546, FC6547, FC6548, FC6549, FC6550, FC6553, FC6554, FC6555, FC6556, FC6557, FCX13, GCP5, GDR08, H7, HED07, I_B16, IHU 1004, IS1105, ISW_13, ISW_22, JL-2/Guangxi/2014, JN-4, JN-8, K-319, K-380, k19, Kaveri_River_8, kcps77, LDB2, LN126, M2T9B4, MAC17169, MARA4, MGB7, Mm32, M017, NAIP8, NCCP-1830, NCCP-2314, NCCP-2315, p-11, P1D10, PAMC 28760, pck237, PIB, PNUSAE006842, PNUSAE075412, PNUSAE079139, PNUSAE082311, PNUSAE095383, PNUSAE095678, PNUSAE096028, PNUSAE096029, PNUSAE096030, PNUSAE096031, PNUSAE096032, PNUSAE096033, PNUSAE096034, PNUSAE096035, PNUSAE096036, PNUSAE096037, PNUSAE096038, PNUSAE096546, PNUSAE096547, PNUSAE097114, PNUSAE097115, PNUSAE109243, PNUSAE109244, PNUSAS141996, PP005, PYM-3, R-52920, R-52921, RE9, REC22, R131, R132, R144, RSI091219, S120, S2, S_CGKV243_2014, S_CGKV331_2014, Sam8-TMC1, SARITHAS-1, SFI16, SG121, SG122, SH165, SH202, SH219, SH223, SH257, SH284, SH361, Sh_2011a, Sh_2011b, Sh_2011c, Sh_2011d, Sh_20Ile, Sh_2011f, Sh_2011g, Sh_2011h, Sh_2011i, Sh_2011j, Sh_2011k, Sh_2011L, Sh_2011M, Sh_2011N, Sh_20110, Sh_2011P, Sh_2011Q, SHS-1, SHS-10, SHS-2, SHS-3 SHS-4, SHS-5, SHS-6, SHS-7, SHS-8, SHS-9, SHV-71, T47090, T47091, T47355, TYN 130607, US8, UST050418-695, UW10, UW24, VDL-SS337, XJ149-N3G1, XJDY-N2-1, YAAJ-10, environmental samples: enrichment culture clone 10(2012), enrichment culture clone E007, enrichment culture clone E031 uncultured Shigella clone MT19 uncultured.

Polysaccharides for use in the immunogenic complexes as disclosed herein can comprise a polysaccharide from any Shigella subspecies, or servar thereof that is listed in the Taxonomy data based found at world wide website: “ncbi.nlm.nih.gov/Taxonomy/Browser/wwwtax.cgi?id=623”, which is incorporated herein in its entirety by reference.

### Methods of Isolating and Purifying Polysaccharides

In some embodiments, the disclosure provides methods of purifying one or more polysaccharides described herein from one or more of S. flexneri or S. sonnei from cellular components of bacteria. In some embodiments, methods comprise purifying O specific polysaccharides from one or more cellular components of bacteria.

In some embodiments, the bacteria are Gram-negative. In some embodiments, the bacteria are Gram-positive. In some embodiments, the bacteria are Shigella. In some embodiments, the Shigella bacterial serotypes selected from Flexneri or Sonnei. Other polysaccharides from other Shigella serogroups are envisioned for use in in the MAPS immunogenic complexes as disclosed herein, including, but not limited to shigella serotypes that cause shigellosis, such as, but not limited to Shigella boydii or Shigella dysenteriae, or an unclassified Shigella species (Shigella sp.).

In some embodiments, O-specific polysaccharides purified from Shigella are small (around 40-50 kDa) which limits the cross-linking that can occur with the carrier proteins (e.g., rhizavidin binding to biotin on the polysaccharide). Accordingly, O-specific polysaccharides of between about 90-120 kDa can be purified from modified S. flexneri or S. sonnei, which have been modified for at least one of: (i) deletion of the short chain OSP enzyme WZZ, and (ii) overexpression of long chain OSP enzyme Fepe from S. paratyphi A or WZZ₂ from Pseudomonas aeruginosa.

In some embodiments, the cellular components include protein. In some embodiments, the cellular proteins include nucleic acid. In some embodiments, the cellular components include lipids. In some embodiments, the cellular components include polysaccharides. In some embodiments, the cellular components are part of a lysate.

In some embodiments, the polysaccharide purification processes incorporate a series of ethanol precipitations, washes of crude polysaccharide preparations with ethanol, diethyl ether, and/or acetone, and drying under vacuum to furnish purified products. In some embodiments, a phenol extraction step is incorporated for polysaccharide purifications. In some embodiments the purification process employs a CTAB (cetyltrimethyl ammonium bromide) precipitation step in addition to using ethanol and phenol precipitation steps.

### Methods of Biotinylating Polysaccharides

In some embodiments, the disclosure provides methods of biotinylating one or more polysaccharides described herein. In some embodiments, the method comprises reacting purified polysaccharides with 1-cyano-4-dimethylaminopyridinium tetrafluoroborate (CDAP) for activation of hydroxyl groups in the polysaccharides followed by the addition of amine PEG biotin under conditions that result in covalent linkage of biotin to the polysaccharides. In some embodiments, the desired level of biotinylation is achieved by varying the ratio of CDAP to polysaccharide. In some embodiments, the method comprises reacting purified polysaccharides with 1-Ethyl-3-[3-dimethylaminopropyl]carbodiimide Hydrochloride (EDC) and N-hydroxysulfosuccinimide (NHS). In some embodiments, the biotinylated polysaccharides are purified by filtration to remove process residuals such as unreacted biotin, dimethylaminopyridine, acetonitrile, cyanide and unreacted glycine. In some embodiments, the level of polysaccharide biotinylation described herein is optimized to reduce the amount of accessible biotin following MAPS complexation.

### Manufacture of Immunogenic Complexes

The present disclosure includes methods for manufacturing immunogenic complexes described herein. In some embodiments, a method of manufacturing immunogenic complexes comprises complexing at least one biotinylated polysaccharide with at least one biotin-binding fusion protein. In some embodiments, the fusion protein comprises an amino acid sequence having at least 80%, 85%, 90%, 95%, 96%, 97%, 98%, 99%, or 100% identity to SEQ ID NO: 1. In some embodiments, the fusion protein comprises an amino acid sequence having at least 80%, 85%, 90%, 95%, 96%, 97%, 98%, 99%, or 100% identity to SEQ ID NO: 2. In some embodiments, the fusion protein comprises an amino acid sequence having at least 80%, 85%, 90%, 95%, 96%, 97%, 98%, 99%, or 100% identity to SEQ ID NO: 6.

In some embodiments, the average (e.g., the mean) protein (e.g., antigenic protein) to polysaccharide ratio of a plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average protein to polysaccharide ratio of a plurality of immunogenic complexes is approximately 1:1 (w/w). In some embodiments, the average protein to polysaccharide ratio of a plurality of immunogenic complexes is approximately 2:1 (w/w). In some embodiments, the average protein to polysaccharide ratio of a plurality of immunogenic complexes is approximately 3:1 (w/w). In some embodiments, the average protein to polysaccharide ratio of a plurality of immunogenic complexes is approximately 4:1 (w/w). In some embodiments, the average protein to polysaccharide ratio of a plurality of immunogenic complexes is approximately 5:1 (w/w). In some embodiments, the average protein to polysaccharide ratio of a plurality of immunogenic complexes is approximately 6:1 (w/w). In some embodiments, the average protein to polysaccharide ratio of a plurality of immunogenic complexes is approximately 7:1 (w/w). In some embodiments, the average protein to polysaccharide ratio of a plurality of immunogenic complexes is approximately 8:1 (w/w). In some embodiments, the average protein to polysaccharide ratio of a plurality of immunogenic complexes is approximately 9:1 (w/w). In some embodiments, the average protein to polysaccharide ratio of a plurality of immunogenic complexes is approximately 10:1 (w/w). In some embodiments, the average protein:PS ratios are chosen to enhance the polysaccharide immunogenicity potential (carrier function) and/or to elicit protection against, or to inhibit, pneumococcal colonization by any pneumococcus (independent of polysaccharide serotype) through a protein-specific immune response. Immunogenic compositions and vaccines of the invention may comprise mixtures of immunogenic complexes with different average protein to polysaccharide ratios.

In some embodiments, a vaccine or immunogenic composition comprises a plurality of immunogenic complexes comprising any one or more of: a Rhavi-SseB, a Rhavi-IpaB, or a CP1 protein and a O specific polysaccharide, from or derived from Shigella serotype flexneri. In some embodiments, the average ratio of either Rhavi-SseB, a Rhavi-IpaB, or CP1 protein to O specific polysaccharide from or derived from Shigella serotype flexneri in the plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average ratio of either Rhavi-SseB, a Rhavi-IpaB, or CP1 protein to O specific polysaccharide from or derived from Shigella serotype flexneri in the plurality of immunogenic complexes is approximately 1:1 (w/w). In some embodiments, the average ratio of either Rhavi-SseB, a Rhavi-IpaB, or CP1 protein to O specific polysaccharide from or derived from Shigella serotype flexneri in the plurality of immunogenic complexes is approximately 2:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 3:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 4:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 5:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 6:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 7:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 8:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 9:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is approximately 10:1 (w/w). In some embodiments, the average ratio of Rhavi-SseB, a Rhavi-IpaB, or CP1 protein to O specific polysaccharide from or derived from Shigella serotype flexneri in the plurality of immunogenic complexes is chosen to enhance the polysaccharide immunogenicity potential (carrier function) and/or to elicit protection against, or to inhibit, shigella colonization by any shigella bacterium (independent of polysaccharide serotype) through a protein specific immune response. Immunogenic compositions and vaccines of the invention may comprise mixtures of immunogenic complexes with different average protein to polysaccharide ratios.

In some embodiments, a vaccine or immunogenic composition comprises a plurality of immunogenic complexes comprising any one of: an Rhavi-SseB, a Rhavi-IpaB, or a CP1 protein and a O specific polysaccharide, from or derived from Shigella serotype sonnei. In some embodiments, the average ratio of either Rhavi-SseB, a Rhavi-IpaB, or CP1 protein to O specific polysaccharide from or derived from Shigella serotype sonnei in the plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average ratio of either Rhavi-SseB, a Rhavi-IpaB, or CP1 protein to O specific polysaccharide from or derived from Shigella serotype sonnei in the plurality of immunogenic complexes is approximately 1:1 (w/w). In some embodiments, the average ratio of either Rhavi-SseB, a Rhavi-IpaB, or CP1 protein to O specific polysaccharide from or derived from Shigella serotype sonnei in the plurality of immunogenic complexes is approximately 2:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 3:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 4:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 5:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 6:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 7:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 8:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 9:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is approximately 10:1 (w/w). In some embodiments, the average ratio of Rhavi-SseB, a Rhavi-IpaB, or CP1 protein to O specific polysaccharide from or derived from Shigella serotype sonnei in the plurality of immunogenic complexes is chosen to enhance the polysaccharide immunogenicity potential (carrier function) and/or to elicit protection against, or to inhibit, shigella colonization by any shigella bacterium (independent of polysaccharide serotype) through a protein specific immune response. Immunogenic compositions and vaccines of the invention may comprise mixtures of immunogenic complexes with different average protein to polysaccharide ratios.

### Immunogenic and Vaccine Compositions

Another aspect of the disclosure provides compositions that include one or more immunogenic complexes described herein. For example, an immunogenic composition, e.g., vaccine composition, can include one or more immunogenic complexes described herein. In some embodiments, such compositions can include a plurality of one type of immunogenic complex described herein. For example, a composition can include a population of one type of immunogenic complex, where all of the immunogenic complexes include the same antigenic polypeptide and the same antigenic polysaccharide. Additionally or alternatively, such compositions can include a plurality of more than one type of immunogenic complex described herein. For example, a composition can include populations of different types of immunogenic complexes. In some embodiments, a composition can include a population of a first type of immunogenic complex and a population of a second type of immunogenic complex, where the first type and the second type of the immunogenic complex have different antigenic polypeptides and/or different antigenic polysaccharides. In some embodiments, a composition can include a population of a first type of immunogenic complex and a population of a second type of immunogenic complex, where the first type and the second type of the immunogenic complex include the same antigenic polypeptide and different antigenic polysaccharides (e.g., polysaccharides of different serotypes). In some embodiments, immunogenic complexes described herein are formulated into a pharmaceutical composition. In some embodiments a pharmaceutical composition may be a vaccine. In some embodiments a pharmaceutical composition comprises a pharmaceutically acceptable carrier. In some embodiments a pharmaceutical composition comprises an adjuvant.

### Vaccine Compositions

In some embodiments, a vaccine composition is a polyvalent or multivalent vaccine. In some embodiments, the valency of a vaccine composition refers to the number of species of immunogenic complexes present in the vaccine composition. The valency of a vaccine described herein is not limiting with respect to the total antigens present in said pharmaceutical composition, immunogenic complex, or vaccine, or to the number of pathogen strains for which administration of said pharmaceutical composition, immunogenic complex, immunogenic composition, or vaccine composition may induce an immune-protective response. In a non-limiting example, a 24-valent vaccine composition may comprise more than 24 antigenic components (e.g., peptide and/or polysaccharide components) and may induce an immunoprotective response against more than 24 pathogens, or pathogenic serotypes or strains.

In some embodiments, a vaccine composition comprises between 1-50 species of immunogenic complexes. In some embodiments, a vaccine composition comprises between 1-40 species of immunogenic complexes. In some embodiments, a vaccine composition comprises between 1-35 species of immunogenic complexes. In some embodiments, a vaccine composition comprises between 1-30 species of immunogenic complexes. In some embodiments, a vaccine composition comprises between 1-30 species of immunogenic complexes. In some embodiments, a vaccine composition comprises between 1-24 species of immunogenic complexes. In some embodiments, a vaccine composition comprises between 1-15 species of immunogenic complexes. In some embodiments, a vaccine composition comprises between 1-9 species of immunogenic complexes. In some embodiments, a vaccine composition comprises between 1-5 species of immunogenic complexes. In some embodiments, a vaccine is a polyvalent vaccine.

### Exemplary Shigella-MAPS Vaccine Composition

In some embodiments, a vaccine composition disclosed herein comprises at least 2, or at least 3, or at least 4 species of immunogenic composition, e.g., at least 2, or 3, or 4 Shigella-MAPS immunogenic composition.

In some embodiments, a vaccine composition as described herein comprises at least two immunogenic complexes, wherein the two immunogenic complexes are selected from any of: S. flexneri-2a-SseB, S. flexneri-3a-SseB, S. flexneri-6-SseB, S. sonnei-SseB, S. flexneri-2a-IpaB, S. flexneri-3a-IpaB, S. flexneri-6-IpaB, S. sonnei-IpaB, S. flexneri-2a-CP1, S. flexneri-3a-CP1, S. flexneri-6-CP1, S. sonnei-CP1. Exemplary combinations of immunogenic complexes for a bivalent (2V) shigella-MAPS immunogenic composition are shown in Table 2A.

Table 2A. Exemplary combinations of immunogenic complexes for a bivalent (2V) vaccine composition comprising two Shigella-MAPS immunogenic complexes, where two immunogenic compositions are selected from any of: 2a-SseB, 3a-SseB, 6-SseB, sonnei-SseB, 2a-IpaB, 3a-IpaB, 6-IpaB, sonnei-IpaB, 2a-CP1, 3a-CP1, 6-CP1 or sonnei-CP1, and where the SseB can comprises an amino acid sequence of SEQ ID NO: 4 or a polypeptide having at least 80% sequence identity thereto, and IpaB comprises an amino acid sequence of SEQ ID NO: 5, or a polypeptide having at least 80% sequence identity thereto, and CP1 comprises an amino acid sequence of SEQ ID NO: 6, or a polypeptide having at least 80% sequence identity thereto.

TABLE 2A
2V Shigella-MAPS immunogenic compositions

| | |
|---|---|
| 2a-SseB, 3a-SseB | 3a-SseB, 6-CP1 |
| 2a-SseB, 6-SseB | 3a-SseB, Sonnei-CP1 |
| 2a-SseB, Sonnei-SseB | 6-SseB, Sonnei-SseB |
| 2a-SseB, 2a-IpaB | 6-SseB, 2a-IpaB |
| 2a-SseB, 3a-IpaB | 6-SseB, 3a-IpaB |
| 2a-SseB, 6-IpaB | 6-SseB, 6-IpaB |
| 2a-SseB, Sonnei-IpaB | 6-SseB, Sonnei-IpaB |
| 2a-SseB, 2a-CP1 | 6-SseB, 2a-CP1 |
| 2a-SseB, 3a-CP1 | 6-SseB, 3a-CP1 |
| 2a-SseB, 6-CP1 | 6-SseB, 6-CP1 |
| 2a-SseB, Sonnei-CP1 | 6-SseB, Sonnei-CP1 |
| 3a-SseB, 6-SseB | Sonnei-SseB, 2a-IpaB |
| 3a-SseB, Sonnei-SseB | Sonnei-SseB, 3a-IpaB |
| 3a-SseB, 2a-IpaB | Sonnei-SseB, 6-IpaB |
| 3a-SseB, 3a-IpaB | Sonnei-SseB, Sonnei-IpaB |
| 3a-SseB, 6-IpaB | Sonnei-SseB, 2a-CP1 |
| 3a-SseB, Sonnei-IpaB | Sonnei-SseB, 3a-CP1 |
| 3a-SseB, 2a-CP1 | Sonnei-SseB, 6-CP1 |
| 3a-SseB, 3a-CP1 | Sonnei-SseB, Sonnei-CP1 |
| 2a-IpaB, Sonnei-CP1 | 2a-IpaB, 3a-IpaB |
| 3a-IpaB, 6-IpaB | 2a-IpaB, 6-IpaB |
| 3a-IpaB, Sonnei-IpaB | 2a-IpaB, Sonnei-IpaB |
| 3a-IpaB, 2a-CP1 | 2a-IpaB, 2a-CP1 |
| 3a-IpaB, 3a-CP1 | 2a-IpaB, 3a-CP1 |
| 3a-IpaB, 6-CP1 | 2a-IpaB, 6-CP1 |
| 3a-IpaB, Sonnei-CP1 | Sonnei-IpaB, 2a-CP1 |
| 6-IpaB, Sonnei-IpaB | Sonnei-IpaB, 3a-CP1 |
| 6-IpaB, 2a-CP1 | Sonnei-IpaB, 6-CP1 |
| 6-IpaB, 3a-CP1 | Sonnei-IpaB, Sonnei-CP1 |
| 6-IpaB, 6-CP1 | 2a-CP1, 3a-CP1 |
| 6-IpaB, Sonnei-CP1 | 2a-CP1, 6-CP1 |
| | 2a-CP1, Sonnei-CP1 |
| | 3a-CP1, 6-CP1 |
| | 3a-CP1, Sonnei-CP1 |
| | 6-CP1, Sonnei-CP1 |

In some embodiments, an immunogenic vaccine comprises at least three immunogenic complexes as described herein, wherein the three immunogenic compositions are selected from any of: 2a-SseB, 3a-SseB, 6-SseB, Sonnei-SseB, 2a-IpaB, 3a-IpaB, 6-IpaB, Sonnei-IpaB, 2a-CP1, 3a-CP1, 6-CP1, Sonnei-CP1, and where the SseB can comprises an amino acid sequence of SEQ ID NO: 4 or a polypeptide having at least 80% sequence identity thereto, and IpaB comprises an amino acid sequence of SEQ ID NO: 5, or a polypeptide having at least 80% sequence identity thereto, and CP1 comprises an amino acid sequence of SEQ ID NO: 6, or a polypeptide having at least 80% sequence identity thereto. Exemplary combinations of 3 immunogenic complexes as disclosed herein for a multivalent Shigella-MAPS vaccine are shown in Table 2B.

Table 2B. Exemplary combinations of shigella-MAPS immunogenic complexes for a multivalent (3V) vaccine composition comprising three Shigella-MAPS immunogenic complexes.

TABLE 2B
3V Shigella MAPS immunogenic compositions

| | |
|---|---|
| 2a-SseB, 3a-SseB, 6-SseB | 3a-SseB, 6-SseB, Sonnei-SseB |
| 2a-SseB, 3a-SseB, Sonnei-SseB | 3a-SseB, 6-SseB, 2a-IpaB |
| 2a-SseB, 3a-SseB, 2a-IpaB | 3a-SseB, 6-SseB, 3a-IpaB |
| 2a-SseB, 3a-SseB, 3a-IpaB | 3a-SseB, 6-SseB, 6-IpaB |
| 2a-SseB, 3a-SseB, 6-IpaB | 3a-SseB, 6-SseB, Sonnei-IpaB |
| 2a-SseB, 3a-SseB, Sonnei-IpaB | 3a-SseB, 6-SseB, 2a-CP1 |
| 2a-SseB, 3a-SseB, 2a-CP1 | 3a-SseB, 6-SseB, 3a-CP1 |
| 2a-SseB, 3a-SseB, 3a-CP1 | 3a-SseB, 6-SseB, 6-CP1 |
| 2a-SseB, 3a-SseB, 6-CP1 | 3a-SseB, 6-SseB, Sonnei-CP1 |
| 2a-SseB, 3a-SseB, Sonnei-CP1 | 3a-SseB, Sonnei-SseB, 2a-IpaB |
| 2a-SseB, 6-SseB, Sonnei-SseB | 3a-SseB, Sonnei-SseB, 3a-IpaB |
| 2a-SseB, 6-SseB, 2a-IpaB | 3a-SseB, Sonnei-SseB, 6-IpaB |
| 2a-SseB, 6-SseB, 3a-IpaB | 3a-SseB, Sonnei-SseB, Sonnei-IpaB |
| 2a-SseB, 6-SseB, 6-IpaB | 3a-SseB, Sonnei-SseB, 2a-CP1 |
| 2a-SseB, 6-SseB, Sonnei-IpaB | 3a-SseB, Sonnei-SseB, 3a-CP1 |
| 2a-SseB, 6-SseB, 2a-CP1 | 3a-SseB, Sonnei-SseB, 6-CP1 |
| 2a-SseB, 6-SseB, 3a-CP1 | 3a-SseB, Sonnei-SseB, Sonnei-CP1 |
| 2a-SseB, 6-SseB, 6-CP1 | 3a-SseB, 2a-IpaB, 3a-IpaB |
| 2a-SseB, 6-SseB, Sonnei-CP1 | 3a-SseB, 2a-IpaB, 6-IpaB |
| 2a-SseB, Sonnei-SseB, 2a-IpaB | 3a-SseB, 2a-IpaB, Sonnei-IpaB |
| 2a-SseB, Sonnei-SseB, 3a-IpaB | 3a-SseB, 2a-IpaB, 2a-CP1 |
| 2a-SseB, Sonnei-SseB, 6-IpaB | 3a-SseB, 2a-IpaB, 3a-CP1 |
| 2a-SseB, Sonnei-SseB, Sonnei-IpaB | 3a-SseB, 2a-IpaB, 6-CP1 |
| 2a-SseB, Sonnei-SseB, 2a-CP1 | 3a-SseB, 2a-IpaB, Sonnei-CP1 |
| 2a-SseB, Sonnei-SseB, 3a-CP1 | 3a-SseB, 3a-IpaB, 6-IpaB |
| 2a-SseB, Sonnei-SseB, 6-CP1 | 3a-SseB, 3a-IpaB, Sonnei-IpaB |
| 2a-SseB, Sonnei-SseB, Sonnei-CP1 | 3a-SseB, 3a-IpaB, 2a-CP1 |
| 2a-SseB, 2a-IpaB, 3a-IpaB | 3a-SseB, 3a-IpaB, 3a-CP1 |
| 2a-SseB, 2a-IpaB, 6-IpaB | 3a-SseB, 3a-IpaB, 6-CP1 |
| 2a-SseB, 2a-IpaB, Sonnei-IpaB | 3a-SseB, 3a-IpaB, Sonnei-CP1 |
| 2a-SseB, 2a-IpaB, 2a-CP1 | 3a-SseB, 6-IpaB, Sonnei-IpaB |
| 2a-SseB, 2a-IpaB, 3a-CP1 | 3a-SseB, 6-IpaB, 2a-CP1 |
| 2a-SseB, 2a-IpaB, 6-CP1 | 3a-SseB, 6-IpaB, 3a-CP1 |
| 2a-SseB, 2a-IpaB, Sonnei-CP1 | 3a-SseB, 6-IpaB, 6-CP1 |
| 2a-SseB, 3a-IpaB, 6-IpaB | 3a-SseB, 6-IpaB, Sonnei-CP1 |
| 2a-SseB, 3a-IpaB, Sonnei-IpaB | 3a-SseB, Sonnei-IpaB, 2a-CP1 |
| 2a-SseB, 3a-IpaB, 2a-CP1 | 3a-SseB, Sonnei-IpaB, 3a-CP1 |
| 2a-SseB, 3a-IpaB, 3a-CP1 | 3a-SseB, Sonnei-IpaB, 6-CP1 |
| 2a-SseB, 3a-IpaB, 6-CP1 | 3a-SseB, Sonnei-IpaB, Sonnei-CP1 |
| 2a-SseB, 3a-IpaB, Sonnei-CP1 | 3a-SseB, 2a-CP1, 3a-CP1 |
| 2a-SseB, 6-IpaB, Sonnei-IpaB | 3a-SseB, 2a-CP1, 6-CP1 |
| 2a-SseB, 6-IpaB, 2a-CP1 | 3a-SseB, 2a-CP1, Sonnei-CP1 |
| 2a-SseB, 6-IpaB, 3a-CP1 | 3a-SseB, 3a-CP1, 6-CP1 |
| 2a-SseB, 6-IpaB, 6-CP1 | 3a-SseB, 3a-CP1, Sonnei-CP1 |
| 2a-SseB, 6-IpaB, Sonnei-CP1 | 3a-SseB, 6-CP1, Sonnei-CP1 |
| 2a-SseB, Sonnei-IpaB, 2a-CP1 | Sonnei-SseB, 2a-IpaB, 3a-IpaB |
| 2a-SseB, Sonnei-IpaB, 3a-CP1 | Sonnei-SseB, 2a-IpaB, 6-IpaB |
| 2a-SseB, Sonnei-IpaB, 6-CP1 | Sonnei-SseB, 2a-IpaB, Sonnei-IpaB |
| 2a-SseB, Sonnei-IpaB, Sonnei-CP1 | Sonnei-SseB, 2a-IpaB, 2a-CP1 |
| 2a-SseB, 2a-CP1, 3a-CP1 | Sonnei-SseB, 2a-IpaB, 3a-CP1 |
| 2a-SseB, 2a-CP1, 6-CP1 | Sonnei-SseB, 2a-IpaB, 6-CP1 |
| 2a-SseB, 2a-CP1, Sonnei-CP1 | Sonnei-SseB, 2a-IpaB, Sonnei-CP1 |
| 2a-SseB, 3a-CP1, 6-CP1 | Sonnei-SseB, 3a-IpaB, 6-IpaB |
| 2a-SseB, 3a-CP1, Sonnei-CP1 | Sonnei-SseB, 3a-IpaB, Sonnei-IpaB |
| 2a-SseB, 6-CP1, Sonnei-CP1 | Sonnei-SseB, 3a-IpaB, 2a-CP1 |
| 6-SseB, Sonnei-SseB, 2a-IpaB | Sonnei-SseB, 3a-IpaB, 3a-CP1 |
| 6-SseB, Sonnei-SseB, 3a-IpaB | Sonnei-SseB, 3a-IpaB, 6-CP1 |
| 6-SseB, Sonnei-SseB, 6-IpaB | Sonnei-SseB, 3a-IpaB, Sonnei-CP1 |
| 6-SseB, Sonnei-SseB, Sonnei-IpaB | Sonnei-SseB, 6-IpaB, Sonnei-IpaB |
| 6-SseB, Sonnei-SseB, 2a-CP1 | Sonnei-SseB, 6-IpaB, 2a-CP1 |
| 6-SseB, Sonnei-SseB, 3a-CP1 | Sonnei-SseB, 6-IpaB, 3a-CP1 |
| 6-SseB, Sonnei-SseB, 6-CP1 | Sonnei-SseB, 6-IpaB, 6-CP1 |
| 6-SseB, Sonnei-SseB, Sonnei-CP1 | Sonnei-SseB, 6-IpaB, Sonnei-CP1 |
| 6-SseB, 2a-IpaB, 3a-IpaB | Sonnei-SseB, Sonnei-IpaB, 2a-CP1 |
| 6-SseB, 2a-IpaB, 6-IpaB | Sonnei-SseB, Sonnei-IpaB, 3a-CP1 |
| 6-SseB, 2a-IpaB, Sonnei-IpaB | Sonnei-SseB, Sonnei-IpaB, 6-CP1 |
| 6-SseB, 2a-IpaB, 2a-CP1 | Sonnei-SseB, Sonnei-IpaB, Sonnei-CP1 |
| 6-SseB, 2a-IpaB, 3a-CP1 | Sonnei-SseB, 2a-CP1, 3a-CP1 |
| 6-SseB, 2a-IpaB, 6-CP1 | Sonnei-SseB, 2a-CP1, 6-CP1 |
| 6-SseB, 2a-IpaB, Sonnei-CP1 | Sonnei-SseB, 2a-CP1, Sonnei-CP1 |
| 6-SseB, 3a-IpaB, 6-IpaB | Sonnei-SseB, 3a-CP1, 6-CP1 |
| 6-SseB, 3a-IpaB, Sonnei-IpaB | Sonnei-SseB, 3a-CP1, Sonnei-CP1 |
| 6-SseB, 3a-IpaB, 2a-CP1 | 2a-IpaB, 3a-IpaB, 6-IpaB |
| 6-SseB, 3a-IpaB, 3a-CP1 | 2a-IpaB, 3a-IpaB, Sonnei-IpaB |
| 6-SseB, 3a-IpaB, 6-CP1 | 2a-IpaB, 3a-IpaB, 2a-CP1 |
| 6-SseB, 3a-IpaB, Sonnei-CP1 | 2a-IpaB, 3a-IpaB, 3a-CP1 |
| 6-SseB, 6-IpaB, Sonnei-IpaB | 2a-IpaB, 3a-IpaB, 6-CP1 |
| 6-SseB, 6-IpaB, 2a-CP1 | 2a-IpaB, 3a-IpaB, Sonnei-CP1 |
| 6-SseB, 6-IpaB, 3a-CP1 | 2a-IpaB, 6-IpaB, Sonnei-IpaB |
| 6-SseB, 6-IpaB, 6-CP1 | 2a-IpaB, 6-IpaB, 2a-CP1 |
| 6-SseB, 6-IpaB, Sonnei-CP1 | 2a-IpaB, 6-IpaB, 3a-CP1 |
| 6-SseB, Sonnei-IpaB, 2a-CP1 | 2a-IpaB, 6-IpaB, 6-CP1 |
| 6-SseB, Sonnei-IpaB, 3a-CP1 | 2a-IpaB, 6-IpaB, Sonnei-CP1 |
| 6-SseB, Sonnei-IpaB, 6-CP1 | 2a-IpaB, Sonnei-IpaB, 2a-CP1 |
| 6-SseB, Sonnei-IpaB, Sonnei-CP1 | 2a-IpaB, Sonnei-IpaB, 3a-CP1 |
| 6-SseB, 2a-CP1, 3a-CP1 | 2a-IpaB, Sonnei-IpaB, 6-CP1 |
| 6-SseB, 2a-CP1, 6-CP1 | 2a-IpaB, Sonnei-IpaB, Sonnei-CP1 |
| 6-SseB, 2a-CP1, Sonnei-CP1 | 2a-IpaB, 2a-CP1, 3a-CP1 |
| 6-SseB, 3a-CP1, 6-CP1 | 2a-IpaB, 2a-CP1, 6-CP1 |
| 6-SseB, 3a-CP1, Sonnei-CP1 | 2a-IpaB, 2a-CP1, Sonnei-CP1 |
| 6-SseB, 6-CP1, Sonnei-CP1 | 2a-IpaB, 3a-CP1, 6-CP1 |
| Sonnei-SseB, 6-CP1, Sonnei-CP1 | 2a-IpaB, 3a-CP1, Sonnei-CP1 |
| 6-IpaB, Sonnei-IpaB, 2a-CP1 | 2a-IpaB, 6-CP1, Sonnei-CP1 |
| 6-IpaB, Sonnei-IpaB, 3a-CP1 | Sonnei-IpaB, 2a-CP1, 3a-CP1 |
| 6-IpaB, Sonnei-IpaB, 6-CP1 | Sonnei-IpaB, 2a-CP1, 6-CP1 |
| 6-IpaB, Sonnei-IpaB, Sonnei-CP1 | Sonnei-IpaB, 2a-CP1, Sonnei-CP1 |
| 6-IpaB, 2a-CP1, 3a-CP1 | Sonnei-IpaB, 3a-CP1, 6-CP1 |
| 6-IpaB, 2a-CP1, 6-CP1 | Sonnei-IpaB, 3a-CP1, Sonnei-CP1 |
| 6-IpaB, 2a-CP1, Sonnei-CP1 | Sonnei-IpaB, 6-CP1, Sonnei-CP1 |
| 6-IpaB, 3a-CP1, 6-CP1 | 2a-CP1, 3a-CP1, 6-CP1 |
| 6-IpaB, 3a-CP1, Sonnei-CP1 | 2a-CP1, 3a-CP1, Sonnei-CP1 |
| 6-IpaB, 6-CP1, Sonnei-CP1 | 2a-CP1, 6-CP1, Sonnei-CP1 |
| 3a-IpaB, 6-IpaB, Sonnei-IpaB | 3a-CP1, 6-CP1, Sonnei-CP1 |
| 3a-IpaB, 6-IpaB, 2a-CP1 | |
| 3a-IpaB, 6-IpaB, 3a-CP1 | |
| 3a-IpaB, 6-IpaB, 6-CP1 | |
| 3a-IpaB, 6-IpaB, Sonnei-CP1 | |
| 3a-IpaB, Sonnei-IpaB, 2a-CP1 | |
| 3a-IpaB, Sonnei-IpaB, 3a-CP1 | |
| 3a-IpaB, Sonnei-IpaB, 6-CP1 | |
| 3a-IpaB, Sonnei-IpaB, Sonnei-CP1 | |
| 3a-IpaB, 2a-CP1, 3a-CP1 | |
| 3a-IpaB, 2a-CP1, 6-CP1 | |
| 3a-IpaB, 2a-CP1, Sonnei-CP1 | |
| 3a-IpaB, 3a-CP1, 6-CP1 | |
| 3a-IpaB, 3a-CP1, Sonnei-CP1 | |
| 3a-IpaB, 6-CP1, Sonnei-CP1 | |

In some embodiments, an immunogenic vaccine comprises at least four immunogenic complexes as described herein, wherein the four immunogenic compositions are selected from any of 2a-SseB, 3a-SseB, 6-SseB, Sonnei-SseB, 2a-IpaB, 3a-IpaB, 6-IpaB, Sonnei-IpaB, 2a-CP1, 3a-CP1, 6-CP1, Sonnei-CP1, and where the SseB can comprises an amino acid sequence of SEQ ID NO: 4 or a polypeptide having at least 80% sequence identity thereto, and IpaB comprises an amino acid sequence of SEQ ID NO: 5, or a polypeptide having at least 80 sequence identity thereto, and CP1 comprises an amino acid sequence of SEQ ID NO: 6, or a polypeptide having at least 80% sequence identity thereto. Exemplary combinations of 4 immunogenic complexes as disclosed herein for a quadrivalent (4V) Shigella-MAPS vaccine are shown in Table 2C.

In one embodiment, a quadrivalent MAPS-Shigella vaccine comprises the following four immunogenic complexes: 2a-SseB, 3a-SseB, 6-CP and Sonnei-CP1 (referred to herein in the Examples as “Shigella-MAPS 1”). In one embodiment, a quadrivalent Shigella-MAPS vaccine comprises the following four immunogenic complexes: 2a-SseB, 3a-SseB, 6-SseB and Sonnei-SseB (referred to herein in the Examples as “Shigella-MAPS 2”).

Table 2C. Exemplary combination of Shigella-MAPS immunogenic complexes for a quadrivalent (4V) MAPS-Shigella vaccine composition comprising four Shigella-MAPS immunogenic complexes.

TABLE 2C
4V Shigella-MAPS vaccines

| | |
|---|---|
| 2a-SseB, 3a-SseB, 6-SseB, Sonnei-SseB | 3a-IpaB, 6-IpaB, Sonnei-IpaB, 2a-CP1 |
| 2a-SseB, 3a-SseB, 6-SseB, 2a-IpaB | 3a-IpaB, 6-IpaB, Sonnei-IpaB, 3a-CP1 |
| 2a-SseB, 3a-SseB, 6-SseB, 3a-IpaB | 3a-IpaB, 6-IpaB, Sonnei-IpaB, 6-CP1 |
| 2a-SseB, 3a-SseB, 6-SseB, 6-IpaB | 3a-IpaB, 6-IpaB, Sonnei-IpaB, Sonnei-CP1 |
| 2a-SseB, 3a-SseB, 6-SseB, Sonnei-IpaB | 3a-IpaB, 6-IpaB, 2a-CP1, 3a-CP1 |
| 2a-SseB, 3a-SseB, 6-SseB, 2a-CP1 | 3a-IpaB, 6-IpaB, 2a-CP1, 6-CP1 |
| 2a-SseB, 3a-SseB, 6-SseB, 3a-CP1 | 3a-IpaB, 6-IpaB, 2a-CP1, Sonnei-CP1 |
| 2a-SseB, 3a-SseB, 6-SseB, 6-CP1 | 3a-IpaB, 6-IpaB, 3a-CP1, 6-CP1 |
| 2a-SseB, 3a-SseB, 6-SseB, Sonnei-CP1 | 3a-IpaB, 6-IpaB, 3a-CP1, Sonnei-CP1 |
| 2a-SseB, 3a-SseB, Sonnei-SseB, 2a-IpaB | 3a-IpaB, 6-IpaB, 6-CP1, Sonnei-CP1 |
| 2a-SseB, 3a-SseB, Sonnei-SseB, 3a-IpaB | 3a-IpaB, Sonnei-IpaB, 2a-CP1, 3a-CP1 |
| 2a-SseB, 3a-SseB, Sonnei-SseB, 6-IpaB | 3a-IpaB, Sonnei-IpaB, 2a-CP1, 6-CP1 |
| 2a-SseB, 3a-SseB, Sonnei-SseB, Sonnei-IpaB | 3a-IpaB, Sonnei-IpaB, 2a-CP1, Sonnei-CP1 |
| 2a-SseB, 3a-SseB, Sonnei-SseB, 2a-CP1 | 3a-IpaB, Sonnei-IpaB, 3a-CP1, 6-CP1 |
| 2a-SseB, 3a-SseB, Sonnei-SseB, 3a-CP1 | 3a-IpaB, Sonnei-IpaB, 3a-CP1, Sonnei-CP1 |
| 2a-SseB, 3a-SseB, Sonnei-SseB, 6-CP1 | 3a-IpaB, Sonnei-IpaB, 6-CP1, Sonnei-CP1 |
| 2a-SseB, 3a-SseB, Sonnei-SseB, Sonnei-CP1 | 3a-IpaB, 2a-CP1, 3a-CP1, 6-CP1 |
| 2a-SseB, 3a-SseB, 2a-IpaB, 3a-IpaB | 3a-IpaB, 2a-CP1, 3a-CP1, Sonnei-CP1 |
| 2a-SseB, 3a-SseB, 2a-IpaB, 6-IpaB | 3a-IpaB, 2a-CP1, 6-CP1, Sonnei-CP1 |
| 2a-SseB, 3a-SseB, 2a-IpaB, Sonnei-IpaB | 3a-IpaB, 3a-CP1, 6-CP1, Sonnei-CP1 |
| 2a-SseB, 3a-SseB, 2a-IpaB, 2a-CP1 | 6-IpaB, Sonnei-IpaB, 2a-CP1, 3a-CP1 |
| 2a-SseB, 3a-SseB, 2a-IpaB, 3a-CP1 | 6-IpaB, Sonnei-IpaB, 2a-CP1, 6-CP1 |
| 2a-SseB, 3a-SseB, 2a-IpaB, 6-CP1 | 6-IpaB, Sonnei-IpaB, 2a-CP1, Sonnei-CP1 |
| 2a-SseB, 3a-SseB, 2a-IpaB, Sonnei-CP1 | 6-IpaB, Sonnei-IpaB, 3a-CP1, 6-CP1 |
| 2a-SseB, 3a-SseB, 3a-IpaB, 6-IpaB | 6-IpaB, Sonnei-IpaB, 3a-CP1, Sonnei-CP1 |
| 2a-SseB, 3a-SseB, 3a-IpaB, Sonnei-IpaB | 6-IpaB, Sonnei-IpaB, 6-CP1, Sonnei-CP1 |
| 2a-SseB, 3a-SseB, 3a-IpaB, 2a-CP1 | 6-IpaB, 2a-CP1, 3a-CP1, 6-CP1 |
| 2a-SseB, 3a-SseB, 3a-IpaB, 3a-CP1 | 6-IpaB, 2a-CP1, 3a-CP1, Sonnei-CP1 |
| 2a-SseB, 3a-SseB, 3a-IpaB, 6-CP1 | 6-IpaB, 2a-CP1, 6-CP1, Sonnei-CP1 |
| 2a-SseB, 3a-SseB, 3a-IpaB, Sonnei-CP1 | 6-IpaB, 3a-CP1, 6-CP1, Sonnei-CP1 |
| 2a-SseB, 3a-SseB, 6-IpaB, Sonnei-IpaB | Sonnei-IpaB, 2a-CP1, 3a-CP1, 6-CP1 |
| 2a-SseB, 3a-SseB, 6-IpaB, 2a-CP1 | Sonnei-IpaB, 2a-CP1, 3a-CP1, Sonnei-CP1 |
| 2a-SseB, 3a-SseB, 6-IpaB, 3a-CP1 | Sonnei-IpaB, 2a-CP1, 6-CP1, Sonnei-CP1 |
| 2a-SseB, 3a-SseB, 6-IpaB, 6-CP1 | Sonnei-IpaB, 3a-CP1, 6-CP1, Sonnei-CP1 |
| 2a-SseB, 3a-SseB, 6-IpaB, Sonnei-CP1 | 2a-CP1, 3a-CP1, 6-CP1, Sonnei-CP1 |
| 2a-SseB, 3a-SseB, Sonnei-IpaB, 2a-CP1 | Sonnei-SseB, 2a-IpaB, 3a-IpaB, 6-IpaB |
| 2a-SseB, 3a-SseB, Sonnei-IpaB, 3a-CP1 | Sonnei-SseB, 2a-IpaB, 3a-IpaB, Sonnei-IpaB |
| 2a-SseB, 3a-SseB, Sonnei-IpaB, 6-CP1 | Sonnei-SseB, 2a-IpaB, 3a-IpaB, 2a-CP1 |
| 2a-SseB, 3a-SseB, Sonnei-IpaB, Sonnei-CP1 | Sonnei-SseB, 2a-IpaB, 3a-IpaB, 3a-CP1 |
| 2a-SseB, 3a-SseB, 2a-CP1, 3a-CP1 | Sonnei-SseB, 2a-IpaB, 3a-IpaB, 6-CP1 |
| 2a-SseB, 3a-SseB, 2a-CP1, 6-CP1 | Sonnei-SseB, 2a-IpaB, 3a-IpaB, Sonnei-CP1 |
| 2a-SseB, 3a-SseB, 2a-CP1, Sonnei-CP1 | Sonnei-SseB, 2a-IpaB, 6-IpaB, Sonnei-IpaB |
| 2a-SseB, 3a-SseB, 3a-CP1, 6-CP1 | Sonnei-SseB, 2a-IpaB, 6-IpaB, 2a-CP1 |
| 2a-SseB, 3a-SseB, 3a-CP1, Sonnei-CP1 | Sonnei-SseB, 2a-IpaB, 6-IpaB, 3a-CP1 |
| 2a-SseB, 3a-SseB, 6-CP1, Sonnei-CP1 | Sonnei-SseB, 2a-IpaB, 6-IpaB, 6-CP1 |
| 2a-SseB, 6-SseB, Sonnei-SseB, 2a-IpaB | Sonnei-SseB, 2a-IpaB, 6-IpaB, Sonnei-CP1 |
| 2a-SseB, 6-SseB, Sonnei-SseB, 3a-IpaB | Sonnei-SseB, 2a-IpaB, Sonnei-IpaB, 2a-CP1 |
| 2a-SseB, 6-SseB, Sonnei-SseB, 6-IpaB | Sonnei-SseB, 2a-IpaB, Sonnei-IpaB, 3a-CP1 |
| 2a-SseB, 6-SseB, Sonnei-SseB, Sonnei-IpaB | Sonnei-SseB, 2a-IpaB, Sonnei-IpaB, 6-CP1 |
| 2a-SseB, 6-SseB, Sonnei-SseB, 2a-CP1 | Sonnei-SseB, 2a-IpaB, Sonnei-IpaB, Sonnei-CP1 |
| 2a-SseB, 6-SseB, Sonnei-SseB, 3a-CP1 | Sonnei-SseB, 2a-IpaB, 2a-CP1, 3a-CP1 |
| 2a-SseB, 6-SseB, Sonnei-SseB, 6-CP1 | Sonnei-SseB, 2a-IpaB, 2a-CP1, 6-CP1 |
| 2a-SseB, 6-SseB, Sonnei-SseB, Sonnei-CP1 | Sonnei-SseB, 2a-IpaB, 2a-CP1, Sonnei-CP1 |
| 2a-SseB, 6-SseB, 2a-IpaB, 3a-IpaB | Sonnei-SseB, 2a-IpaB, 3a-CP1, 6-CP1 |
| 2a-SseB, 6-SseB, 2a-IpaB, 6-IpaB | Sonnei-SseB, 2a-IpaB, 3a-CP1, Sonnei-CP1 |
| 2a-SseB, 6-SseB, 2a-IpaB, Sonnei-IpaB | Sonnei-SseB, 2a-IpaB, 6-CP1, Sonnei-CP1 |
| 2a-SseB, 6-SseB, 2a-IpaB, 2a-CP1 | Sonnei-SseB, 3a-IpaB, 6-IpaB, Sonnei-IpaB |
| 2a-SseB, 6-SseB, 2a-IpaB, 3a-CP1 | Sonnei-SseB, 3a-IpaB, 6-IpaB, 2a-CP1 |
| 2a-SseB, 6-SseB, 2a-IpaB, 6-CP1 | Sonnei-SseB, 3a-IpaB, 6-IpaB, 3a-CP1 |
| 2a-SseB, 6-SseB, 2a-IpaB, Sonnei-CP1 | Sonnei-SseB, 3a-IpaB, 6-IpaB, 6-CP1 |
| 2a-SseB, 6-SseB, 3a-IpaB, 6-IpaB | Sonnei-SseB, 3a-IpaB, 6-IpaB, Sonnei-CP1 |
| 2a-SseB, 6-SseB, 3a-IpaB, Sonnei-IpaB | Sonnei-SseB, 3a-IpaB, Sonnei-IpaB, 2a-CP1 |
| 2a-SseB, 6-SseB, 3a-IpaB, 2a-CP1 | Sonnei-SseB, 3a-IpaB, Sonnei-IpaB, 3a-CP1 |
| 2a-SseB, 6-SseB, 3a-IpaB, 3a-CP1 | Sonnei-SseB, 3a-IpaB, Sonnei-IpaB, 6-CP1 |
| 2a-SseB, 6-SseB, 3a-IpaB, 6-CP1 | Sonnei-SseB, 3a-IpaB, Sonnei-IpaB, Sonnei-CP1 |
| 2a-SseB, 6-SseB, 3a-IpaB, Sonnei-CP1 | Sonnei-SseB, 3a-IpaB, 2a-CP1, 3a-CP1 |
| 2a-SseB, 6-SseB, 6-IpaB, Sonnei-IpaB | Sonnei-SseB, 3a-IpaB, 2a-CP1, 6-CP1 |
| 2a-SseB, 6-SseB, 6-IpaB, 2a-CP1 | Sonnei-SseB, 3a-IpaB, 2a-CP1, Sonnei-CP1 |
| 2a-SseB, 6-SseB, 6-IpaB, 3a-CP1 | Sonnei-SseB, 3a-IpaB, 3a-CP1, 6-CP1 |
| 2a-SseB, 6-SseB, 6-IpaB, 6-CP1 | Sonnei-SseB, 3a-IpaB, 3a-CP1, Sonnei-CP1 |
| 2a-SseB, 6-SseB, 6-IpaB, Sonnei-CP1 | Sonnei-SseB, 3a-IpaB, 6-CP1, Sonnei-CP1 |
| 2a-SseB, 6-SseB, Sonnei-IpaB, 2a-CP1 | Sonnei-SseB, 6-IpaB, Sonnei-IpaB, 2a-CP1 |
| 2a-SseB, 6-SseB, Sonnei-IpaB, 3a-CP1 | Sonnei-SseB, 6-IpaB, Sonnei-IpaB, 3a-CP1 |
| 2a-SseB, 6-SseB, Sonnei-IpaB, 6-CP1 | Sonnei-SseB, 6-IpaB, Sonnei-IpaB, 6-CP1 |
| 2a-SseB, 6-SseB, Sonnei-IpaB, Sonnei-CP1 | Sonnei-SseB, 6-IpaB, Sonnei-IpaB, Sonnei-CP1 |
| 2a-SseB, 6-SseB, 2a-CP1, 3a-CP1 | Sonnei-SseB, 6-IpaB, 2a-CP1, 3a-CP1 |
| 2a-SseB, 6-SseB, 2a-CP1, 6-CP1 | Sonnei-SseB, 6-IpaB, 2a-CP1, 6-CP1 |
| 2a-SseB, 6-SseB, 2a-CP1, Sonnei-CP1 | Sonnei-SseB, 6-IpaB, 2a-CP1, Sonnei-CP1 |
| 2a-SseB, 6-SseB, 3a-CP1, 6-CP1 | Sonnei-SseB, 6-IpaB, 3a-CP1, 6-CP1 |
| 2a-SseB, 6-SseB, 3a-CP1, Sonnei-CP1 | Sonnei-SseB, 6-IpaB, 3a-CP1, Sonnei-CP1 |
| 2a-SseB, 6-SseB, 6-CP1, Sonnei-CP1 | Sonnei-SseB, 6-IpaB, 6-CP1, Sonnei-CP1 |
| 2a-SseB, Sonnei-SseB, 2a-IpaB, 3a-IpaB | Sonnei-SseB, Sonnei-IpaB, 2a-CP1, 3a-CP1 |
| 2a-SseB, Sonnei-SseB, 2a-IpaB, 6-IpaB | Sonnei-SseB, Sonnei-IpaB, 2a-CP1, 6-CP1 |
| 2a-SseB, Sonnei-SseB, 2a-IpaB, Sonnei-IpaB | Sonnei-SseB, Sonnei-IpaB, 2a-CP1, Sonnei-CP1 |
| 2a-SseB, Sonnei-SseB, 2a-IpaB, 2a-CP1 | Sonnei-SseB, Sonnei-IpaB, 3a-CP1, 6-CP1 |
| 2a-SseB, Sonnei-SseB, 2a-IpaB, 3a-CP1 | Sonnei-SseB, Sonnei-IpaB, 3a-CP1, Sonnei-CP1 |
| 2a-SseB, Sonnei-SseB, 2a-IpaB, 6-CP1 | Sonnei-SseB, Sonnei-IpaB, 6-CP1, Sonnei-CP1 |
| 2a-SseB, Sonnei-SseB, 2a-IpaB, Sonnei-CP1 | Sonnei-SseB, 2a-CP1, 3a-CP1, 6-CP1 |
| 2a-SseB, Sonnei-SseB, 3a-IpaB, 6-IpaB | Sonnei-SseB, 2a-CP1, 3a-CP1, Sonnei-CP1 |
| 2a-SseB, Sonnei-SseB, 3a-IpaB, Sonnei-IpaB | Sonnei-SseB, 2a-CP1, 6-CP1, Sonnei-CP1 |
| 2a-SseB, Sonnei-SseB, 3a-IpaB, 2a-CP1 | Sonnei-SseB, 3a-CP1, 6-CP1, Sonnei-CP1 |
| 2a-SseB, Sonnei-SseB, 3a-IpaB, 3a-CP1 | 2a-IpaB, 3a-IpaB, 6-IpaB, Sonnei-IpaB |
| 2a-SseB, Sonnei-SseB, 3a-IpaB, 6-CP1 | 2a-IpaB, 3a-IpaB, 6-IpaB, 2a-CP1 |
| 2a-SseB, Sonnei-SseB, 3a-IpaB, Sonnei-CP1 | 2a-IpaB, 3a-IpaB, 6-IpaB, 3a-CP1 |
| 2a-SseB, Sonnei-SseB, 6-IpaB, Sonnei-IpaB | 2a-IpaB, 3a-IpaB, 6-IpaB, 6-CP1 |
| 2a-SseB, Sonnei-SseB, 6-IpaB, 2a-CP1 | 2a-IpaB, 3a-IpaB, 6-IpaB, Sonnei-CP1 |
| 2a-SseB, Sonnei-SseB, 6-IpaB, 3a-CP1 | 2a-IpaB, 3a-IpaB, Sonnei-IpaB, 2a-CP1 |
| 2a-SseB, Sonnei-SseB, 6-IpaB, 6-CP1 | 2a-IpaB, 3a-IpaB, Sonnei-IpaB, 3a-CP1 |
| 2a-SseB, Sonnei-SseB, 6-IpaB, Sonnei-CP1 | 2a-IpaB, 3a-IpaB, Sonnei-IpaB, 6-CP1 |
| 2a-SseB, Sonnei-SseB, Sonnei-IpaB, 2a-CP1 | 2a-IpaB, 3a-IpaB, Sonnei-IpaB, Sonnei-CP1 |
| 2a-SseB, Sonnei-SseB, Sonnei-IpaB, 3a-CP1 | 2a-IpaB, 3a-IpaB, 2a-CP1, 3a-CP1 |
| 2a-SseB, Sonnei-SseB, Sonnei-IpaB, 6-CP1 | 2a-IpaB, 3a-IpaB, 2a-CP1, 6-CP1 |
| 2a-SseB, Sonnei-SseB, Sonnei-IpaB, Sonnei-CP1 | 2a-IpaB, 3a-IpaB, 2a-CP1, Sonnei-CP1 |
| 2a-SseB, Sonnei-SseB, 2a-CP1, 3a-CP1 | 2a-IpaB, 3a-IpaB, 3a-CP1, 6-CP1 |
| 2a-SseB, Sonnei-SseB, 2a-CP1, 6-CP1 | 2a-IpaB, 3a-IpaB, 3a-CP1, Sonnei-CP1 |
| 2a-SseB, Sonnei-SseB, 2a-CP1, Sonnei-CP1 | 2a-IpaB, 3a-IpaB, 6-CP1, Sonnei-CP1 |
| 2a-SseB, Sonnei-SseB, 3a-CP1, 6-CP1 | 2a-IpaB, 6-IpaB, Sonnei-IpaB, 2a-CP1 |
| 2a-SseB, Sonnei-SseB, 3a-CP1, Sonnei-CP1 | 2a-IpaB, 6-IpaB, Sonnei-IpaB, 3a-CP1 |
| 2a-SseB, Sonnei-SseB, 6-CP1, Sonnei-CP1 | 2a-IpaB, 6-IpaB, Sonnei-IpaB, 6-CP1 |
| 2a-SseB, 2a-IpaB, 3a-IpaB, 6-IpaB | 2a-IpaB, 6-IpaB, Sonnei-IpaB, Sonnei-CP1 |
| 2a-SseB, 2a-IpaB, 3a-IpaB, Sonnei-IpaB | 2a-IpaB, 6-IpaB, 2a-CP1, 3a-CP1 |
| 2a-SseB, 2a-IpaB, 3a-IpaB, 2a-CP1 | 2a-IpaB, 6-IpaB, 2a-CP1, 6-CP1 |
| 2a-SseB, 2a-IpaB, 3a-IpaB, 3a-CP1 | 2a-IpaB, 6-IpaB, 2a-CP1, Sonnei-CP1 |
| 2a-SseB, 2a-IpaB, 3a-IpaB, 6-CP1 | 2a-IpaB, 6-IpaB, 3a-CP1, 6-CP1 |
| 2a-SseB, 2a-IpaB, 3a-IpaB, Sonnei-CP1 | 2a-IpaB, 6-IpaB, 3a-CP1, Sonnei-CP1 |
| 2a-SseB, 2a-IpaB, 6-IpaB, Sonnei-IpaB | 2a-IpaB, 6-IpaB, 6-CP1, Sonnei-CP1 |
| 2a-SseB, 2a-IpaB, 6-IpaB, 2a-CP1 | 2a-IpaB, Sonnei-IpaB, 2a-CP1, 3a-CP1 |
| 2a-SseB, 2a-IpaB, 6-IpaB, 3a-CP1 | 2a-IpaB, Sonnei-IpaB, 2a-CP1, 6-CP1 |
| 2a-SseB, 2a-IpaB, 6-IpaB, 6-CP1 | 2a-IpaB, Sonnei-IpaB, 2a-CP1, Sonnei-CP1 |
| 2a-SseB, 2a-IpaB, 6-IpaB, Sonnei-CP1 | 2a-IpaB, Sonnei-IpaB, 3a-CP1, 6-CP1 |
| 2a-SseB, 2a-IpaB, Sonnei-IpaB, 2a-CP1 | 2a-IpaB, Sonnei-IpaB, 3a-CP1, Sonnei-CP1 |
| 2a-SseB, 2a-IpaB, Sonnei-IpaB, 3a-CP1 | 2a-IpaB, Sonnei-IpaB, 6-CP1, Sonnei-CP1 |
| 2a-SseB, 2a-IpaB, Sonnei-IpaB, 6-CP1 | 2a-IpaB, 2a-CP1, 3a-CP1, 6-CP1 |
| 2a-SseB, 2a-IpaB, Sonnei-IpaB, Sonnei-CP1 | 2a-IpaB, 2a-CP1, 3a-CP1, Sonnei-CP1 |
| 2a-SseB, 2a-IpaB, 2a-CP1, 3a-CP1 | 2a-IpaB, 2a-CP1, 6-CP1, Sonnei-CP1 |
| 2a-SseB, 2a-IpaB, 2a-CP1, 6-CP1 | 2a-IpaB, 3a-CP1, 6-CP1, Sonnei-CP1 |
| 2a-SseB, 2a-IpaB, 2a-CP1, Sonnei-CP1 | 6-SseB, Sonnei-SseB, 2a-IpaB, 3a-IpaB |
| 2a-SseB, 2a-IpaB, 3a-CP1, 6-CP1 | 6-SseB, Sonnei-SseB, 2a-IpaB, 6-IpaB |
| 2a-SseB, 2a-IpaB, 3a-CP1, Sonnei-CP1 | 6-SseB, Sonnei-SseB, 2a-IpaB, Sonnei-IpaB |
| 2a-SseB, 2a-IpaB, 6-CP1, Sonnei-CP1 | 6-SseB, Sonnei-SseB, 2a-IpaB, 2a-CP1 |
| 2a-SseB, 3a-IpaB, 6-IpaB, Sonnei-IpaB | 6-SseB, Sonnei-SseB, 2a-IpaB, 3a-CP1 |
| 2a-SseB, 3a-IpaB, 6-IpaB, 2a-CP1 | 6-SseB, Sonnei-SseB, 2a-IpaB, 6-CP1 |
| 2a-SseB, 3a-IpaB, 6-IpaB, 3a-CP1 | 6-SseB, Sonnei-SseB, 2a-IpaB, Sonnei-CP1 |
| 2a-SseB, 3a-IpaB, 6-IpaB, 6-CP1 | 6-SseB, Sonnei-SseB, 3a-IpaB, 6-IpaB |
| 2a-SseB, 3a-IpaB, 6-IpaB, Sonnei-CP1 | 6-SseB, Sonnei-SseB, 3a-IpaB, Sonnei-IpaB |
| 2a-SseB, 3a-IpaB, Sonnei-IpaB, 2a-CP1 | 6-SseB, Sonnei-SseB, 3a-IpaB, 2a-CP1 |
| 2a-SseB, 3a-IpaB, Sonnei-IpaB, 3a-CP1 | 6-SseB, Sonnei-SseB, 3a-IpaB, 3a-CP1 |
| 2a-SseB, 3a-IpaB, Sonnei-IpaB, 6-CP1 | 6-SseB, Sonnei-SseB, 3a-IpaB, 6-CP1 |
| 2a-SseB, 3a-IpaB, Sonnei-IpaB, Sonnei-CP1 | 6-SseB, Sonnei-SseB, 3a-IpaB, Sonnei-CP1 |
| 2a-SseB, 3a-IpaB, 2a-CP1, 3a-CP1 | 6-SseB, Sonnei-SseB, 6-IpaB, Sonnei-IpaB |
| 2a-SseB, 3a-IpaB, 2a-CP1, 6-CP1 | 6-SseB, Sonnei-SseB, 6-IpaB, 2a-CP1 |
| 2a-SseB, 3a-IpaB, 2a-CP1, Sonnei-CP1 | 6-SseB, Sonnei-SseB, 6-IpaB, 3a-CP1 |
| 2a-SseB, 3a-IpaB, 3a-CP1, 6-CP1 | 6-SseB, Sonnei-SseB, 6-IpaB, 6-CP1 |
| 2a-SseB, 3a-IpaB, 3a-CP1, Sonnei-CP1 | 6-SseB, Sonnei-SseB, 6-IpaB, Sonnei-CP1 |
| 2a-SseB, 3a-IpaB, 6-CP1, Sonnei-CP1 | 6-SseB, Sonnei-SseB, Sonnei-IpaB, 2a-CP1 |
| 2a-SseB, 6-IpaB, Sonnei-IpaB, 2a-CP1 | 6-SseB, Sonnei-SseB, Sonnei-IpaB, 3a-CP1 |
| 2a-SseB, 6-IpaB, Sonnei-IpaB, 3a-CP1 | 6-SseB, Sonnei-SseB, Sonnei-IpaB, 6-CP1 |
| 2a-SseB, 6-IpaB, Sonnei-IpaB, 6-CP1 | 6-SseB, Sonnei-SseB, Sonnei-IpaB, Sonnei-CP1 |
| 2a-SseB, 6-IpaB, Sonnei-IpaB, Sonnei-CP1 | 6-SseB, Sonnei-SseB, 2a-CP1, 3a-CP1 |
| 2a-SseB, 6-IpaB, 2a-CP1, 3a-CP1 | 6-SseB, Sonnei-SseB, 2a-CP1, 6-CP1 |
| 2a-SseB, 6-IpaB, 2a-CP1, 6-CP1 | 6-SseB, Sonnei-SseB, 2a-CP1, Sonnei-CP1 |
| 2a-SseB, 6-IpaB, 2a-CP1, Sonnei-CP1 | 6-SseB, Sonnei-SseB, 3a-CP1, 6-CP1 |
| 2a-SseB, 6-IpaB, 3a-CP1, 6-CP1 | 6-SseB, Sonnei-SseB, 3a-CP1, Sonnei-CP1 |
| 2a-SseB, 6-IpaB, 3a-CP1, Sonnei-CP1 | 6-SseB, Sonnei-SseB, 6-CP1, Sonnei-CP1 |
| 2a-SseB, 6-IpaB, 6-CP1, Sonnei-CP1 | 6-SseB, 2a-IpaB, 3a-IpaB, 6-IpaB |
| 2a-SseB, Sonnei-IpaB, 2a-CP1, 3a-CP1 | 6-SseB, 2a-IpaB, 3a-IpaB, Sonnei-IpaB |
| 2a-SseB, Sonnei-IpaB, 2a-CP1, 6-CP1 | 6-SseB, 2a-IpaB, 3a-IpaB, 2a-CP1 |
| 2a-SseB, Sonnei-IpaB, 2a-CP1, Sonnei-CP1 | 6-SseB, 2a-IpaB, 3a-IpaB, 3a-CP1 |
| 2a-SseB, Sonnei-IpaB, 3a-CP1, 6-CP1 | 6-SseB, 2a-IpaB, 3a-IpaB, 6-CP1 |
| 2a-SseB, Sonnei-IpaB, 3a-CP1, Sonnei-CP1 | 6-SseB, 2a-IpaB, 3a-IpaB, Sonnei-CP1 |
| 2a-SseB, Sonnei-IpaB, 6-CP1, Sonnei-CP1 | 6-SseB, 2a-IpaB, 6-IpaB, Sonnei-IpaB |
| 2a-SseB, 2a-CP1, 3a-CP1, 6-CP1 | 6-SseB, 2a-IpaB, 6-IpaB, 2a-CP1 |
| 2a-SseB, 2a-CP1, 3a-CP1, Sonnei-CP1 | 6-SseB, 2a-IpaB, 6-IpaB, 3a-CP1 |
| 2a-SseB, 2a-CP1, 6-CP1, Sonnei-CP1 | 6-SseB, 2a-IpaB, 6-IpaB, 6-CP1 |
| 2a-SseB, 3a-CP1, 6-CP1, Sonnei-CP1 | 6-SseB, 2a-IpaB, 6-IpaB, Sonnei-CP1 |
| 3a-SseB, 6-SseB, Sonnei-SseB, 2a-IpaB | 6-SseB, 2a-IpaB, Sonnei-IpaB, 2a-CP1 |
| 3a-SseB, 6-SseB, Sonnei-SseB, 3a-IpaB | 6-SseB, 2a-IpaB, Sonnei-IpaB, 3a-CP1 |
| 3a-SseB, 6-SseB, Sonnei-SseB, 6-IpaB | 6-SseB, 2a-IpaB, Sonnei-IpaB, 6-CP1 |
| 3a-SseB, 6-SseB, Sonnei-SseB, Sonnei-IpaB | 6-SseB, 2a-IpaB, Sonnei-IpaB, Sonnei-CP1 |
| 3a-SseB, 6-SseB, Sonnei-SseB, 2a-CP1 | 6-SseB, 2a-IpaB, 2a-CP1, 3a-CP1 |
| 3a-SseB, 6-SseB, Sonnei-SseB, 3a-CP1 | 6-SseB, 2a-IpaB, 2a-CP1, 6-CP1 |
| 3a-SseB, 6-SseB, Sonnei-SseB, 6-CP1 | 6-SseB, 2a-IpaB, 2a-CP1, Sonnei-CP1 |
| 3a-SseB, 6-SseB, Sonnei-SseB, Sonnei-CP1 | 6-SseB, 2a-IpaB, 3a-CP1, 6-CP1 |
| 3a-SseB, 6-SseB, 2a-IpaB, 3a-IpaB | 6-SseB, 2a-IpaB, 3a-CP1, Sonnei-CP1 |
| 3a-SseB, 6-SseB, 2a-IpaB, 6-IpaB | 6-SseB, 2a-IpaB, 6-CP1, Sonnei-CP1 |
| 3a-SseB, 6-SseB, 2a-IpaB, Sonnei-IpaB | 6-SseB, 3a-IpaB, 6-IpaB, Sonnei-IpaB |
| 3a-SseB, 6-SseB, 2a-IpaB, 2a-CP1 | 6-SseB, 3a-IpaB, 6-IpaB, 2a-CP1 |
| 3a-SseB, 6-SseB, 2a-IpaB, 3a-CP1 | 6-SseB, 3a-IpaB, 6-IpaB, 3a-CP1 |
| 3a-SseB, 6-SseB, 2a-IpaB, 6-CP1 | 6-SseB, 3a-IpaB, 6-IpaB, 6-CP1 |
| 3a-SseB, 6-SseB, 2a-IpaB, Sonnei-CP1 | 6-SseB, 3a-IpaB, 6-IpaB, Sonnei-CP1 |
| 3a-SseB, 6-SseB, 3a-IpaB, 6-IpaB | 6-SseB, 3a-IpaB, Sonnei-IpaB, 2a-CP1 |
| 3a-SseB, 6-SseB, 3a-IpaB, Sonnei-IpaB | 6-SseB, 3a-IpaB, Sonnei-IpaB, 3a-CP1 |
| 3a-SseB, 6-SseB, 3a-IpaB, 2a-CP1 | 6-SseB, 3a-IpaB, Sonnei-IpaB, 6-CP1 |
| 3a-SseB, 6-SseB, 3a-IpaB, 3a-CP1 | 6-SseB, 3a-IpaB, Sonnei-IpaB, Sonnei-CP1 |
| 3a-SseB, 6-SseB, 3a-IpaB, 6-CP1 | 6-SseB, 3a-IpaB, 2a-CP1, 3a-CP1 |
| 3a-SseB, 6-SseB, 3a-IpaB, Sonnei-CP1 | 6-SseB, 3a-IpaB, 2a-CP1, 6-CP1 |
| 3a-SseB, 6-SseB, 6-IpaB, Sonnei-IpaB | 6-SseB, 3a-IpaB, 2a-CP1, Sonnei-CP1 |
| 3a-SseB, 6-SseB, 6-IpaB, 2a-CP1 | 6-SseB, 3a-IpaB, 3a-CP1, 6-CP1 |
| 3a-SseB, 6-SseB, 6-IpaB, 3a-CP1 | 6-SseB, 3a-IpaB, 3a-CP1, Sonnei-CP1 |
| 3a-SseB, 6-SseB, 6-IpaB, 6-CP1 | 6-SseB, 3a-IpaB, 6-CP1, Sonnei-CP1 |
| 3a-SseB, 6-SseB, 6-IpaB, Sonnei-CP1 | 6-SseB, 6-IpaB, Sonnei-IpaB, 2a-CP1 |
| 3a-SseB, 6-SseB, Sonnei-IpaB, 2a-CP1 | 6-SseB, 6-IpaB, Sonnei-IpaB, 3a-CP1 |
| 3a-SseB, 6-SseB, Sonnei-IpaB, 3a-CP1 | 6-SseB, 6-IpaB, Sonnei-IpaB, 6-CP1 |
| 3a-SseB, 6-SseB, Sonnei-IpaB, 6-CP1 | 6-SseB, 6-IpaB, Sonnei-IpaB, Sonnei-CP1 |
| 3a-SseB, 6-SseB, Sonnei-IpaB, Sonnei-CP1 | 6-SseB, 6-IpaB, 2a-CP1, 3a-CP1 |
| 3a-SseB, 6-SseB, 2a-CP1, 3a-CP1 | 6-SseB, 6-IpaB, 2a-CP1, 6-CP1 |
| 3a-SseB, 6-SseB, 2a-CP1, 6-CP1 | 6-SseB, 6-IpaB, 2a-CP1, Sonnei-CP1 |
| 3a-SseB, 6-SseB, 2a-CP1, Sonnei-CP1 | 6-SseB, 6-IpaB, 3a-CP1, 6-CP1 |
| 3a-SseB, 6-SseB, 3a-CP1, 6-CP1 | 6-SseB, 6-IpaB, 3a-CP1, Sonnei-CP1 |
| 3a-SseB, 6-SseB, 3a-CP1, Sonnei-CP1 | 6-SseB, 6-IpaB, 6-CP1, Sonnei-CP1 |
| 3a-SseB, 6-SseB, 6-CP1, Sonnei-CP1 | 6-SseB, Sonnei-IpaB, 2a-CP1, 3a-CP1 |
| 3a-SseB, Sonnei-SseB, 2a-IpaB, 3a-IpaB | 6-SseB, Sonnei-IpaB, 2a-CP1, 6-CP1 |
| 3a-SseB, Sonnei-SseB, 2a-IpaB, 6-IpaB | 6-SseB, Sonnei-IpaB, 2a-CP1, Sonnei-CP1 |
| 3a-SseB, Sonnei-SseB, 2a-IpaB, Sonnei-IpaB | 6-SseB, Sonnei-IpaB, 3a-CP1, 6-CP1 |
| 3a-SseB, Sonnei-SseB, 2a-IpaB, 2a-CP1 | 6-SseB, Sonnei-IpaB, 3a-CP1, Sonnei-CP1 |
| 3a-SseB, Sonnei-SseB, 2a-IpaB, 3a-CP1 | 6-SseB, Sonnei-IpaB, 6-CP1, Sonnei-CP1 |
| 3a-SseB, Sonnei-SseB, 2a-IpaB, 6-CP1 | 6-SseB, 2a-CP1, 3a-CP1, 6-CP1 |
| 3a-SseB, Sonnei-SseB, 2a-IpaB, Sonnei-CP1 | 6-SseB, 2a-CP1, 3a-CP1, Sonnei-CP1 |
| 3a-SseB, Sonnei-SseB, 3a-IpaB, 6-IpaB | 6-SseB, 2a-CP1, 6-CP1, Sonnei-CP1 |
| 3a-SseB, Sonnei-SseB, 3a-IpaB, Sonnei-IpaB | 6-SseB, 3a-CP1, 6-CP1, Sonnei-CP1 |
| 3a-SseB, Sonnei-SseB, 3a-IpaB, 2a-CP1 | 3a-SseB, Sonnei-IpaB, 2a-CP1, 3a-CP1 |
| 3a-SseB, Sonnei-SseB, 3a-IpaB, 3a-CP1 | 3a-SseB, Sonnei-IpaB, 2a-CP1, 6-CP1 |
| 3a-SseB, Sonnei-SseB, 3a-IpaB, 6-CP1 | 3a-SseB, Sonnei-IpaB, 2a-CP1, Sonnei-CP1 |
| 3a-SseB, Sonnei-SseB, 3a-IpaB, Sonnei-CP1 | 3a-SseB, Sonnei-IpaB, 3a-CP1, 6-CP1 |
| 3a-SseB, Sonnei-SseB, 6-IpaB, Sonnei-IpaB | 3a-SseB, Sonnei-IpaB, 3a-CP1, Sonnei-CP1 |
| 3a-SseB, Sonnei-SseB, 6-IpaB, 2a-CP1 | 3a-SseB, Sonnei-IpaB, 6-CP1, Sonnei-CP1 |
| 3a-SseB, Sonnei-SseB, 6-IpaB, 3a-CP1 | 3a-SseB, 2a-CP1, 3a-CP1, 6-CP1 |
| 3a-SseB, Sonnei-SseB, 6-IpaB, 6-CP1 | 3a-SseB, 2a-CP1, 3a-CP1, Sonnei-CP1 |
| 3a-SseB, Sonnei-SseB, 6-IpaB, Sonnei-CP1 | 3a-SseB, 2a-CP1, 6-CP1, Sonnei-CP1 |
| 3a-SseB, Sonnei-SseB, Sonnei-IpaB, 2a-CP1 | 3a-SseB, 3a-CP1, 6-CP1, Sonnei-CP1 |
| 3a-SseB, Sonnei-SseB, Sonnei-IpaB, 3a-CP1 | 3a-SseB, 2a-IpaB, 3a-IpaB, 2a-CP1 |
| 3a-SseB, Sonnei-SseB, Sonnei-IpaB, 6-CP1 | 3a-SseB, 2a-IpaB, 3a-IpaB, 3a-CP1 |
| 3a-SseB, Sonnei-SseB, Sonnei-IpaB, Sonnei-CP1 | 3a-SseB, 2a-IpaB, 3a-IpaB, 6-CP1 |
| 3a-SseB, Sonnei-SseB, 2a-CP1, 3a-CP1 | 3a-SseB, 2a-IpaB, 3a-IpaB, Sonnei-CP1 |
| 3a-SseB, Sonnei-SseB, 2a-CP1, 6-CP1 | 3a-SseB, 2a-IpaB, 6-IpaB, Sonnei-IpaB |
| 3a-SseB, Sonnei-SseB, 2a-CP1, Sonnei-CP1 | 3a-SseB, 2a-IpaB, 6-IpaB, 2a-CP1 |
| 3a-SseB, Sonnei-SseB, 3a-CP1, 6-CP1 | 3a-SseB, 2a-IpaB, 6-IpaB, 3a-CP1 |
| 3a-SseB, Sonnei-SseB, 3a-CP1, Sonnei-CP1 | 3a-SseB, 2a-IpaB, 6-IpaB, 6-CP1 |
| 3a-SseB, Sonnei-SseB, 6-CP1, Sonnei-CP1 | 3a-SseB, 2a-IpaB, 6-IpaB, Sonnei-CP1 |
| 3a-SseB, 2a-IpaB, 3a-IpaB, 6-IpaB | 3a-SseB, 2a-IpaB, Sonnei-IpaB, 2a-CP1 |
| 3a-SseB, 2a-IpaB, 3a-IpaB, Sonnei-IpaB | 3a-SseB, 2a-IpaB, Sonnei-IpaB, 3a-CP1 |
| 3a-SseB, 2a-IpaB, 3a-IpaB, 2a-CP1 | 3a-SseB, 2a-IpaB, Sonnei-IpaB, 6-CP1 |
| 3a-SseB, 2a-IpaB, 3a-IpaB, 3a-CP1 | 3a-SseB, 2a-IpaB, Sonnei-IpaB, Sonnei-CP1 |
| 3a-SseB, 2a-IpaB, 3a-IpaB, 6-CP1 | 3a-SseB, 2a-IpaB, 2a-CP1, 3a-CP1 |
| 3a-SseB, 2a-IpaB, 3a-IpaB, Sonnei-CP1 | 3a-SseB, 2a-IpaB, 2a-CP1, 6-CP1 |
| 3a-SseB, 2a-IpaB, 6-IpaB, Sonnei-IpaB | 3a-SseB, 2a-IpaB, 2a-CP1, Sonnei-CP1 |
| 3a-SseB, 2a-IpaB, 6-IpaB, 2a-CP1 | 3a-SseB, 2a-IpaB, 3a-CP1, 6-CP1 |
| 3a-SseB, 2a-IpaB, 6-IpaB, 3a-CP1 | 3a-SseB, 2a-IpaB, 3a-CP1, Sonnei-CP1 |
| 3a-SseB, 2a-IpaB, 6-IpaB, 6-CP1 | 3a-SseB, 2a-IpaB, 6-CP1, Sonnei-CP1 |
| 3a-SseB, 2a-IpaB, 6-IpaB, Sonnei-CP1 | 3a-SseB, 3a-IpaB, 6-IpaB, Sonnei-IpaB |
| 3a-SseB, 2a-IpaB, Sonnei-IpaB, 2a-CP1 | 3a-SseB, 3a-IpaB, 6-IpaB, 2a-CP1 |
| 3a-SseB, 2a-IpaB, Sonnei-IpaB, 3a-CP1 | 3a-SseB, 3a-IpaB, 6-IpaB, 3a-CP1 |
| 3a-SseB, 2a-IpaB, Sonnei-IpaB, 6-CP1 | 3a-SseB, 3a-IpaB, 6-IpaB, 6-CP1 |
| 3a-SseB, 2a-IpaB, Sonnei-IpaB, Sonnei-CP1 | 3a-SseB, 3a-IpaB, 6-IpaB, Sonnei-CP1 |
| 3a-SseB, 2a-IpaB, 2a-CP1, 3a-CP1 | 3a-SseB, 3a-IpaB, Sonnei-IpaB, 2a-CP1 |
| 3a-SseB, 2a-IpaB, 2a-CP1, 6-CP1 | 3a-SseB, 3a-IpaB, Sonnei-IpaB, 3a-CP1 |
| 3a-SseB, 2a-IpaB, 2a-CP1, Sonnei-CP1 | 3a-SseB, 3a-IpaB, Sonnei-IpaB, 6-CP1 |
| 3a-SseB, 2a-IpaB, 3a-CP1, 6-CP1 | 3a-SseB, 3a-IpaB, Sonnei-IpaB, Sonnei-CP1 |
| 3a-SseB, 2a-IpaB, 3a-CP1, Sonnei-CP1 | 3a-SseB, 3a-IpaB, 2a-CP1, 3a-CP1 |
| 3a-SseB, 2a-IpaB, 6-CP1, Sonnei-CP1 | 3a-SseB, 3a-IpaB, 2a-CP1, 6-CP1 |
| 3a-SseB, 3a-IpaB, 6-IpaB, Sonnei-IpaB | 3a-SseB, 3a-IpaB, 2a-CP1, Sonnei-CP1 |
| 3a-SseB, 3a-IpaB, 6-IpaB, 2a-CP1 | 3a-SseB, 3a-IpaB, 3a-CP1, 6-CP1 |
| 3a-SseB, 3a-IpaB, 6-IpaB, 3a-CP1 | 3a-SseB, 3a-IpaB, 3a-CP1, Sonnei-CP1 |
| 3a-SseB, 3a-IpaB, 6-IpaB, 6-CP1 | 3a-SseB, 3a-IpaB, 6-CP1, Sonnei-CP1 |
| 3a-SseB, 3a-IpaB, 6-IpaB, Sonnei-CP1 | 3a-SseB, 6-IpaB, Sonnei-IpaB, 2a-CP1 |
| 3a-SseB, 3a-IpaB, Sonnei-IpaB, 2a-CP1 | 3a-SseB, 6-IpaB, Sonnei-IpaB, 3a-CP1 |
| 3a-SseB, 3a-IpaB, Sonnei-IpaB, 3a-CP1 | 3a-SseB, 6-IpaB, Sonnei-IpaB, 6-CP1 |
| 3a-SseB, 3a-IpaB, Sonnei-IpaB, 6-CP1 | 3a-SseB, 6-IpaB, Sonnei-IpaB, Sonnei-CP1 |
| 3a-SseB, 3a-IpaB, Sonnei-IpaB, Sonnei-CP1 | 3a-SseB, 6-IpaB, 2a-CP1, 3a-CP1 |
| 3a-SseB, 3a-IpaB, 2a-CP1, 3a-CP1 | 3a-SseB, 6-IpaB, 2a-CP1, 6-CP1 |
| 3a-SseB, 3a-IpaB, 2a-CP1, 6-CP1 | 3a-SseB, 6-IpaB, 2a-CP1, Sonnei-CP1 |
| 3a-SseB, 3a-IpaB, 2a-CP1, Sonnei-CP1 | 3a-SseB, 6-IpaB, 3a-CP1, 6-CP1 |
| 3a-SseB, 3a-IpaB, 3a-CP1, 6-CP1 | 3a-SseB, 6-IpaB, 3a-CP1, Sonnei-CP1 |
| 3a-SseB, 3a-IpaB, 3a-CP1, Sonnei-CP1 | 3a-SseB, 6-IpaB, 6-CP1, Sonnei-CP1 |
| 3a-SseB, 3a-IpaB, 6-CP1, Sonnei-CP1 | 3a-SseB, Sonnei-IpaB, 2a-CP1, 3a-CP1 |
| 3a-SseB, 6-IpaB, Sonnei-IpaB, 2a-CP1 | 3a-SseB, Sonnei-IpaB, 2a-CP1, 6-CP1 |
| 3a-SseB, 6-IpaB, Sonnei-IpaB, 3a-CP1 | 3a-SseB, Sonnei-IpaB, 2a-CP1, Sonnei-CP1 |
| 3a-SseB, 6-IpaB, Sonnei-IpaB, 6-CP1 | 3a-SseB, Sonnei-IpaB, 3a-CP1, 6-CP1 |
| 3a-SseB, 6-IpaB, Sonnei-IpaB, Sonnei-CP1 | 3a-SseB, Sonnei-IpaB, 3a-CP1, Sonnei-CP1 |
| 3a-SseB, 6-IpaB, 2a-CP1, 3a-CP1 | 3a-SseB, Sonnei-IpaB, 6-CP1, Sonnei-CP1 |
| 3a-SseB, 6-IpaB, 2a-CP1, 6-CP1 | 3a-SseB, 2a-CP1, 3a-CP1, 6-CP1 |
| 3a-SseB, 6-IpaB, 2a-CP1, Sonnei-CP1 | 3a-SseB, 2a-CP1, 3a-CP1, Sonnei-CP1 |
| 3a-SseB, 6-IpaB, 3a-CP1, 6-CP1 | 3a-SseB, 2a-CP1, 6-CP1, Sonnei-CP1 |
| 3a-SseB, 6-IpaB, 3a-CP1, Sonnei-CP1 | 3a-SseB, 3a-CP1, 6-CP1, Sonnei-CP1 |
| 3a-SseB, 6-IpaB, 6-CP1, Sonnei-CP1 | |
| 3a-SseB, Sonnei-IpaB, 2a-CP1, 3a-CP1 | |
| 3a-SseB, Sonnei-IpaB, 2a-CP1, 6-CP1 | |
| 3a-SseB, Sonnei-IpaB, 2a-CP1, Sonnei-CP1 | |
| 3a-SseB, Sonnei-IpaB, 3a-CP1, 6-CP1 | |
| 3a-SseB, Sonnei-IpaB, 3a-CP1, Sonnei-CP1 | |
| 3a-SseB, Sonnei-IpaB, 6-CP1, Sonnei-CP1 | |
| 3a-SseB, 2a-CP1, 3a-CP1, 6-CP1 | |
| 3a-SseB, 2a-CP1, 3a-CP1, Sonnei-CP1 | |
| 3a-SseB, 2a-CP1, 6-CP1, Sonnei-CP1 | |
| 3a-SseB, 3a-CP1, 6-CP1, Sonnei-CP1 | |
| 3a-SseB, 3a-IpaB, 2a-CP1, 6-CP1 | |
| 3a-SseB, 3a-IpaB, 2a-CP1, Sonnei-CP1 | |
| 3a-SseB, 3a-IpaB, 3a-CP1, 6-CP1 | |
| 3a-SseB, 3a-IpaB, 3a-CP1, Sonnei-CP1 | |
| 3a-SseB, 3a-IpaB, 6-CP1, Sonnei-CP1 | |
| 3a-SseB, 6-IpaB, Sonnei-IpaB, 2a-CP1 | |
| 3a-SseB, 6-IpaB, Sonnei-IpaB, 3a-CP1 | |
| 3a-SseB, 6-IpaB, Sonnei-IpaB, 6-CP1 | |
| 3a-SseB, 6-IpaB, Sonnei-IpaB, Sonnei-CP1 | |
| 3a-SseB, 6-IpaB, 2a-CP1, 3a-CP1 | |
| 3a-SseB, 6-IpaB, 2a-CP1, 6-CP1 | |
| 3a-SseB, 6-IpaB, 2a-CP1, Sonnei-CP1 | |
| 3a-SseB, 6-IpaB, 3a-CP1, 6-CP1 | |
| 3a-SseB, 6-IpaB, 3a-CP1, Sonnei-CP1 | |
| 3a-SseB, 6-IpaB, 6-CP1, Sonnei-CP1 | |

In some embodiments, a vaccine composition comprises two or more species of immunogenic complexes (e.g., in immunogenic compositions) in amounts such that the weight of polysaccharides in the vaccine composition from each immunogenic complex is about the same, e.g., present in a w/w ratio of about 1:1. In some embodiments, the weight of polysaccharides in the vaccine contributed by each immunogenic complex is about 0.20 micro grams. In some embodiments, the weight of polysaccharides in the vaccine contributed by each immunogenic complex is about 0.25 micro grams. In some embodiments, the weight of polysaccharides in the vaccine contributed by each immunogenic complex is about 0.5 micro grams. In some embodiments, the weight of polysaccharides in the vaccine contributed by each immunogenic complex is about 1 micro grams. In some embodiments, the weight of polysaccharides in the vaccine contributed by each immunogenic complex is about 1.5 micro grams. In some embodiments, the weight of polysaccharides in the vaccine contributed by each immunogenic complex is about 2 micro grams. In some embodiments, the weight of polysaccharides in the vaccine contributed by each immunogenic complex is about 2.5 micro grams. In some embodiments, the weight of polysaccharides in the vaccine contributed by each immunogenic complex is about 3 micro grams. In some embodiments, the weight of polysaccharides in the vaccine contributed by each immunogenic complex is about 3.5 micro grams. In some embodiments, the weight of polysaccharides in the vaccine contributed by each immunogenic complex is about 4 micro grams. In some embodiments, the weight of polysaccharides in the vaccine contributed by each immunogenic complex is about 4.5 micro grams. In some embodiments, the weight of polysaccharides in the vaccine contributed by each immunogenic complex is about 5 micro grams. In some embodiments, the weight of polysaccharides in the vaccine contributed by each immunogenic complex is about 5.5 micro grams. In some embodiments, the weight of polysaccharides in the vaccine contributed by each immunogenic complex is about 6 micro grams. In some embodiments, the weight of polysaccharides in the vaccine contributed by each immunogenic complex is about 7 micro grams. In some embodiments, the weight of polysaccharides in the vaccine contributed by each immunogenic complex is about 8 micro grams. In some embodiments, the weight of polysaccharides in the vaccine contributed by each immunogenic complex is about 9 micro grams. In some embodiments, the weight of polysaccharides in the vaccine contributed by each immunogenic complex is about 10 micro grams. In some embodiments, the weight of polysaccharides in the vaccine contributed by each immunogenic complex is about 11 micro grams. In some embodiments, the weight of polysaccharides in the vaccine contributed by each immunogenic complex is about 12 micro grams. In some embodiments, the weight of polysaccharides in the vaccine contributed by each immunogenic complex is more than 12 micro grams, e.g., 13 micro grams, 14 micro grams, 15 micro grams, 16 micro grams, 17 micro grams, 18 micro grams, 19 micro grams, 20 micro grams, 21 micro grams, 22 micro grams, 23 micro grams, 24 micro grams, 25 micro grams, or more.

In some embodiments, a vaccine composition comprises two or more species of immunogenic complexes (e.g., in immunogenic compositions) in amounts such that the weight of polysaccharides in the vaccine composition contributed by each immunogenic complex is different, e.g., present in a w/w ratio that is not about 1:1. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:2. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:3. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:4. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:5. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:6. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:7. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:8. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:9. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:10. In some embodiments, the vaccine composition comprises a mixture of immunogenic complexes, such that the weight of polysaccharide in a vaccine contributed by an immunogenic complex ranges from about 0.20 micro grams to about 6 micro grams. In some embodiments, the vaccine composition comprises a mixture of immunogenic complexes, such that the weight of polysaccharide in a vaccine contributed by an immunogenic complex ranges from about 0.20 micro grams to about 12 micro grams. In some embodiments, the vaccine composition comprises a mixture of immunogenic complexes, such that the weight of polysaccharides in the vaccine contributed by each immunogenic complex ranges from about 0.20 micro grams to about 20 micro grams. In some embodiments, the vaccine composition comprises a mixture of immunogenic complexes, such that the weight of polysaccharides in the vaccine contributed by each immunogenic complex ranges from about 0.20 micro grams to about 40 micro grams.

In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is about the same, e.g., present in a w/w protein:PS ratio of about 1:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 2:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 3:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 4:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 5:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 6:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 7:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 8:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 9:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 10:1.

In some embodiments, the combined weight of polysaccharides and polypeptides in the vaccine contributed by each immunogenic complex is about 0.20 micro grams. In some embodiments, the combined weight of polysaccharides and polypeptides in the vaccine contributed by each immunogenic complex is about 0.40 micro grams. In some embodiments, the combined weight of polysaccharides and polypeptides in the vaccine contributed by each immunogenic complex is about 1 micro grams. In some embodiments, the combined weight of polysaccharides and polypeptides in the vaccine contributed by each immunogenic complex is about 2 micro grams. In some embodiments, the combined weight of polysaccharides and polypeptides in the vaccine contributed by each immunogenic composition is about 3 micro grams. In some embodiments, the combined weight of polysaccharides and polypeptides in the vaccine contributed by each immunogenic composition is about 4 micro grams. In some embodiments, the combined weight of polysaccharides and polypeptides in the vaccine contributed by each immunogenic composition is about 5 micro grams. In some embodiments, the combined weight of polysaccharides and polypeptides in the vaccine contributed by each immunogenic composition is about 6 micro grams. In some embodiments, the combined weight of polysaccharides and polypeptides in the vaccine contributed by each immunogenic composition is about 7 micro grams. In some embodiments, the combined weight of polysaccharides and polypeptides in the vaccine contributed by each immunogenic composition is about 8 micro grams. In some embodiments, the combined weight of polysaccharides and polypeptides in the vaccine contributed by each immunogenic composition is about 9 micro grams. In some embodiments, the combined weight of polysaccharides and polypeptides in the vaccine contributed by each immunogenic composition is about 10 micro grams. In some embodiments, the combined weight of polysaccharides and polypeptides in the vaccine contributed by each immunogenic composition is about 11 micro grams. In some embodiments, the combined weight of polysaccharides and polypeptides in the vaccine contributed by each immunogenic composition is about 12 micro grams. In some embodiments, the combined weight of polysaccharides and polypeptides in the vaccine contributed by each immunogenic composition is about 14 micro grams. In some embodiments, the combined weight of polysaccharides and polypeptides in the vaccine contributed by each immunogenic composition is about 16 micro grams. In some embodiments, the combined weight of polysaccharides and polypeptides in the vaccine contributed by each immunogenic composition is about 18 micro grams. In some embodiments, the combined weight of polysaccharides and polypeptides in the vaccine contributed by each immunogenic composition is about 20 micro grams. In some embodiments, the combined weight of polysaccharides and polypeptides in the vaccine contributed by each immunogenic composition is about 21 micro grams. In some embodiments, the combined weight of polysaccharides and polypeptides in the vaccine contributed by each immunogenic composition is about 22 micro grams. In some embodiments, the combined weight of polysaccharides and polypeptides in the vaccine contributed by each immunogenic composition is about 23 micro grams. In some embodiments, the combined weight of polysaccharides and polypeptides in the vaccine contributed by each immunogenic composition is about 24 micro grams. In some embodiments, the combined weight of polysaccharides and polypeptides in the vaccine contributed by each immunogenic composition is about 25 micro grams. In some embodiments, the combined weight of polysaccharides and polypeptides in the vaccine contributed by each immunogenic composition is about 30 micro grams. In some embodiments, the combined weight of polysaccharides and polypeptides in the vaccine contributed by each immunogenic composition is about 40 micro grams. In some embodiments, the combined weight of polysaccharides and polypeptides in the vaccine contributed by each immunogenic composition is about 50 micro grams. In some embodiments, the combined weight of polysaccharides and polypeptides in the vaccine contributed by each immunogenic composition is about 60 micro grams. In some embodiments, the combined weight of polysaccharides and polypeptides in the vaccine contributed by each immunogenic composition is about 70 micro grams. In some embodiments, the combined weight of polysaccharides and polypeptides in the vaccine contributed by each immunogenic composition is about 80 micro grams. In some embodiments, the combined weight of polysaccharides and polypeptides in the vaccine contributed by each immunogenic composition is about 90 micro grams. In some embodiments, the combined weight of polysaccharides and polypeptides in the vaccine contributed by each immunogenic composition is about 100 micro grams. In some embodiments, the combined weight of polysaccharides and polypeptides in the vaccine contributed by each immunogenic composition is about 110 micro grams.

In some embodiments, a vaccine composition comprises two or more species of immunogenic complexes (e.g., in immunogenic compositions) in amounts such that the combined weight of polysaccharides and polypeptides in the vaccine composition contributed by each immunogenic complex is different, e.g., present in a w/w protein:PS ratio that is not about 1:1, e.g., a protein:PS ratio that is 2:1, 3:1, 4:1. 5:1. 6:1, 7:1, 8:1, 9:1, or 10:1. In some embodiments, the vaccine composition comprises a mixture of immunogenic complexes, such that the combined weight of polysaccharides and polypeptides in the vaccine contributed by each immunogenic complex ranges from about 0.4 micro grams to about 110 micro grams.

### Combined Vaccine Comprising Shigella-MAPS Immunogenic Complexes and Salmonella-MAPS Immunogenic Complexes

In some embodiments, a Shigella-MAPS immunogenic complex as disclosed herein can be combined in a composition with one more salmonella-MAPS immunogenic complexes. Salmonella-MAPS immunogenic complexes are disclosed in PCT application No: PCT/US22/80531 filed on Nov. 29, 2022, which is incorporated herein in its entirety by reference.

Without wishing to be bound by theory, in some embodiments, an immunogenic composition or vaccine as disclosed herein comprises at least one Shigella-MAPS immunogenic complex as disclosed herein, and at least one Salmonella-MAPS immunogenic complex as disclosed in PCT application No: PCT/US22/80531 filed on Nov. 29, 2022. Such an immunogenic composition or vaccine as disclosed herein comprises at least one Shigella-MAPS immunogenic complex as disclosed herein, and at least one Salmonella-MAPS immunogenic complex is referred to herein as a dual “Shigella-Salmonella MAPS vaccine”.

In some embodiments, a Shigella-Salmonella MAPS vaccine can comprise more than four Shigella-MAPS immunogenic complexes, and/or more than four Salmonella-MAPS immunogenic complexes. In some embodiments, Shigella-Salmonella MAPS vaccine can comprise different ratios of Shigella-MAPS immunogenic complexes to Salmonella-MAPS immunogenic complexes. For example, a dual Shigella-Salmonella MAPS vaccine can comprise any combination of: 1:1, 1:2, 1:3, 1:4, 1:5, 1:6, 1:7, 1:8, 1:9 or 1:10 of Shigella-MAPS immunogenic complexes to the number of Salmonella-MAPS immunogenic complexes. In some embodiments, a dual Shigella-Salmonella MAPS vaccine can comprise any combination of: 2:1, 2:2, 2:3, 2:4, 2:5, 2:6, 2:7, 2:8, 2:9 or 2:10 of the number of Shigella-MAPS immunogenic complexes to the number of Salmonella-MAPS immunogenic complexes. In some embodiments, a dual Shigella-Salmonella MAPS vaccine can comprise any combination of: 3:1, 3:2, 3:3, 3:4, 3:5, 3:6, 3:7, 3:8, 3:9 or 3:10 of the number of Shigella-MAPS immunogenic complexes to the number of Salmonella-MAPS immunogenic complexes. In some embodiments, a dual Shigella-Salmonella MAPS vaccine can comprise any combination of: 4:1, 4:2, 4:3, 4:4, 4:5, 4:6, 4:7, 4:8, 4:9 or 4:10 of the number of Shigella-MAPS immunogenic complexes to the number of Salmonella-MAPS immunogenic complexes. In some embodiments, a dual Shigella-Salmonella MAPS vaccine can comprise any combination of: 5:1, 5:2, 5:3, 5:4, 5:5, 5:6, 5:7, 5:8, 5:9 or 5:10 of the number of Shigella-MAPS immunogenic complexes to the number of Salmonella-MAPS immunogenic complexes. In some embodiments, a dual Shigella-Salmonella MAPS vaccine can comprise any combination of: 6:1, 6:2, 6:3, 6:4, 6:5, 6:6, 6:7, 6:8, 6:9 or 6:10 of the number of Shigella-MAPS immunogenic complexes to the number of Salmonella-MAPS immunogenic complexes. In some embodiments, a dual Shigella-Salmonella MAPS vaccine can comprise any combination of: 7:1, 7:2, 7:3, 7:4, 7:5, 7:6, 7:7, 7:8, 7:9 or 7:10 of the number of Shigella-MAPS immunogenic complexes to the number of Salmonella-MAPS immunogenic complexes. In some embodiments, a dual Shigella-Salmonella MAPS vaccine can comprise any combination of: 8:1, 8:2, 8:3, 8:4, 8:5, 8:6, 8:7, 8:8, 8:9 or 8:10 of the number of Shigella-MAPS immunogenic complexes to the number of Salmonella-MAPS immunogenic complexes. In some embodiments, a dual Shigella-Salmonella MAPS vaccine can comprise any combination of: 9:1, 9:2, 9:3, 9:4, 9:5, 9:6, 9:7, 9:8, 9:9 or 9:10 of the number of Shigella-MAPS immunogenic complexes to the number of Salmonella-MAPS immunogenic complexes. In some embodiments, a dual Shigella-Salmonella MAPS vaccine can comprise any combination of: 10:1, 10:2, 10:3, 10:4, 10:5, 10:6, 10:7, 10:8, 10:9 or 10:10 of the number of Shigella-MAPS immunogenic complexes to the number of Salmonella-MAPS immunogenic complexes.

In some embodiments, a Shigella-Salmonella MAPS vaccine comprises at least two Shigella-MAPS immunogenic complexes and at least two Salmonella-MAPS immunogenic complexes. For example, in some embodiments, a Shigella-Salmonella MAPS vaccine can comprise any two Shigella-MAPS immunogenic complexes selected from any disclosed in Table 2A herein, and at least two Salmonella-MAPS immunogenic complexes selected from any of Table 3A disclosed herein.

In some embodiments, a Shigella-Salmonella MAPS vaccine comprises at least three Shigella-MAPS immunogenic complexes and at least three Salmonella-MAPS immunogenic complexes. For example, in some embodiments, a Shigella-Salmonella MAPS vaccine can comprise any three Shigella-MAPS immunogenic complexes selected from any disclosed in Table 2B herein, and at least two Salmonella-MAPS immunogenic complexes selected from any of Table 3B disclosed herein.

In some embodiments, a Shigella-Salmonella MAPS vaccine comprises at least four Shigella-MAPS immunogenic complexes and at least four Salmonella-MAPS immunogenic complexes. For example, in some embodiments, a Shigella-Salmonella MAPS vaccine can comprise any four Shigella-MAPS immunogenic complexes selected from any disclosed in Table 2C herein, and at least four Salmonella-MAPS immunogenic complexes selected from any of the combinations disclosed in Table 3C disclosed herein.

In some embodiments, an exemplary Shigella-Salmonella MAPS vaccine comprises at least 2, or at least 3, or all 4 Shigella-MAPS complexes selected from the group of: S. flexneri-2a-IpaB; S. flexneri-6-IpaB; S. flexneri-3a-IpaB; S. sonnei-IpaB, and at least 2, or at least 3, or all 4 Salmonella-MAPS complexes selected from the group of: S. typhimurium-SseB, S. enteritidis-SseB, S. typhi Vi-CP1, S. paratyphi A-CP1.

In some embodiments, an exemplary Shigella-Salmonella MAPS vaccine comprises at least 2, or at least 3, or all 4 Shigella-MAPS complexes selected from the group of: S. flexneri-2a-IpaB; S. flexneri-6-IpaB; S. flexneri-3a-IpaB; S. sonnei-IpaB, and at least 2, or at least 3, or all 4 Salmonella-MAPS complexes selected from the group of: S. typhimurium-SseB, S. enteritidis-SseB, S. typhi Vi-SseB, S. paratyphi A-SseB.

In some embodiments, a Shigella-MAPS immunogenic complex as disclosed herein, or a bivalent (2V) Shigella-MAPS vaccine disclosed in Table 2A, or a multivalent (3V) Shigella-MAPS vaccine disclosed in Table 2B or a quadrivalent (4V) Shigella-MAPS vaccine disclosed in Table 2C can be combined with at least one Salmonella-MAPS immunogenic complex as disclosed in PCT application No: PCT/US22/80531 filed on Nov. 29, 2022. Without being limited to theory, a Shigella-MAPS vaccine disclosed in any of Table 2A, 2B or 2C can be combined with any of the 2-valent, or 3-valent, or 4-valent Salmonella-MAPS vaccine disclosed in Tables 3A, 3B and 3C respectively herein.

Table 3A. Exemplary combinations of Salmonella-MAPS immunogenic complexes for a bivalent (2V) Salmonella-MAPS vaccine composition comprising two Salmonella-MAPS immunogenic complexes in the vaccine, which can be combined with one or more Shigella-MAPS immunogenic complexes.

TABLE 3A
2V Salmonella-MAPS immunogenic compositions

| | |
|---|---|
| Typhimurium-SseB, Enteritidis-SseB | Enteritidis-SseB, Vi-CP1 |
| Typhimurium-SseB, Vi-SseB | Enteritidis-SseB, ParaOSP-CP1 |
| Typhimurium-SseB, ParaOSP-SseB | Vi-SseB, ParaOSP-SseB |
| Typhimurium-SseB, Typhimurium-IpaB | Vi-SseB, Typhimurium-IpaB |
| Typhimurium-SseB, Enteritidis-IpaB | Vi-SseB, Enteritidis-IpaB |
| Typhimurium-SseB, Vi-IpaB | Vi-SseB, Vi-IpaB |
| Typhimurium-SseB, ParaOSP-IpaB | Vi-SseB, ParaOSP-IpaB |
| Typhimurium-SseB, Typhimurium-CP1 | Vi-SseB, Typhimurium-CP1 |
| Typhimurium-SseB, Enteritidis-CP1 | Vi-SseB, Enteritidis-CP1 |
| Typhimurium-SseB, Vi-CP1 | Vi-SseB, Vi-CP1 |
| Typhimurium-SseB, ParaOSP-CP1 | Vi-SseB, ParaOSP-CP1 |
| Enteritidis-SseB, Vi-SseB | ParaOSP-SseB, Typhimurium-IpaB |
| Enteritidis-SseB, ParaOSP-SseB | ParaOSP-SseB, Enteritidis-IpaB |
| Enteritidis-SseB, Typhimurium-IpaB | ParaOSP-SseB, Vi-IpaB |
| Enteritidis-SseB, Enteritidis-IpaB | ParaOSP-SseB, ParaOSP-IpaB |
| Enteritidis-SseB, Vi-IpaB | ParaOSP-SseB, Typhimurium-CP1 |
| Enteritidis-SseB, ParaOSP-IpaB | ParaOSP-SseB, Enteritidis-CP1 |
| Enteritidis-SseB, Typhimurium-CP1 | ParaOSP-SseB, Vi-CP1 |
| Enteritidis-SseB, Enteritidis-CP1 | ParaOSP-SseB, ParaOSP-CP1 |
| Typhimurium-IpaB, ParaOSP-CP1 | Typhimurium-IpaB, Enteritidis-IpaB |
| Enteritidis-IpaB, Vi-IpaB | Typhimurium-IpaB, Vi-IpaB |
| Enteritidis-IpaB, ParaOSP-IpaB | Typhimurium-IpaB, ParaOSP-IpaB |
| Enteritidis-IpaB, Typhimurium-CP1 | Typhimurium-IpaB, Typhimurium-CP1 |
| Enteritidis-IpaB, Enteritidis-CP1 | Typhimurium-IpaB, Enteritidis-CP1 |
| Enteritidis-IpaB, Vi-CP1 | Typhimurium-IpaB, Vi-CP1 |
| Enteritidis-IpaB, ParaOSP-CP1 | ParaOSP-IpaB, Typhimurium-CP1 |
| Vi-IpaB, ParaOSP-IpaB | ParaOSP-IpaB, Enteritidis-CP1 |
| Vi-IpaB, Typhimurium-CP1 | ParaOSP-IpaB, Vi-CP1 |
| Vi-IpaB, Enteritidis-CP1 | ParaOSP-IpaB, ParaOSP-CP1 |
| Vi-IpaB, Vi-CP1 | Typhimurium-CP1, Enteritidis-CP1 |
| Vi-IpaB, ParaOSP-CP1 | Typhimurium-CP1, Vi-CP1 |
| | Typhimurium-CP1, ParaOSP-CP1 |
| | Enteritidis-CP1, Vi-CP1 |
| | Enteritidis-CP1, ParaOSP-CP1 |
| | Vi-CP1, ParaOSP-CP1 |

In some embodiments, an immunogenic vaccine comprises at one Shigella-MAPS immunogenic complex as disclosed herein, and least three Salmonella-MAPS immunogenic complexes as disclosed in PCT application No: PCT/US22/80531 filed on Nov. 29, 2022, wherein the three immunogenic compositions are selected from any of Typhimurium-SseB, Enteritidis-SseB, Vi-SseB, ParaOSP-SseB, Typhimurium-IpaB, Enteritidis-IpaB, Vi-IpaB, ParaOSP-IpaB, Typhimurium-CP1, Enteritidis-CP1, Vi-CP1, ParaOSP-CP. Exemplary combinations are shown in Table 3B.

Table 3B. Exemplary combinations of Salmonella-MAPS immunogenic complexes for a multivalent (3V) Salmonella-MAPS immunogenic compositions, comprising three Salmonella-MAPS immunogenic complexes, which can be combined with one or more Shigella-MAPS immunogenic complexes as disclosed herein.

TABLE 3B
3V Salmonella MAPS immunogenic compositions

| | |
|---|---|
| Typhimurium-SseB, Enteritidis-SseB, Vi-SseB | Enteritidis-SseB, Vi-SseB, ParaOSP-SseB |
| Typhimurium-SseB, Enteritidis-SseB, ParaOSP-SseB | Enteritidis-SseB, Vi-SseB, Typhimurium-IpaB |
| Typhimurium-SseB, Enteritidis-SseB, Typhimurium-IpaB | Enteritidis-SseB, Vi-SseB, Enteritidis-IpaB |
| Typhimurium-SseB, Enteritidis-SseB, Enteritidis-IpaB | Enteritidis-SseB, Vi-SseB, Vi-IpaB |
| Typhimurium-SseB, Enteritidis-SseB, Vi-IpaB | Enteritidis-SseB, Vi-SseB, ParaOSP-IpaB |
| Typhimurium-SseB, Enteritidis-SseB, ParaOSP-IpaB | Enteritidis-SseB, Vi-SseB, Typhimurium-CP1 |
| Typhimurium-SseB, Enteritidis-SseB, Typhimurium-CP1 | Enteritidis-SseB, Vi-SseB, Enteritidis-CP1 |
| Typhimurium-SseB, Enteritidis-SseB, Enteritidis-CP1 | Enteritidis-SseB, Vi-SseB, Vi-CP1 |
| Typhimurium-SseB, Enteritidis-SseB, Vi-CP1 | Enteritidis-SseB, Vi-SseB, ParaOSP-CP1 |
| Typhimurium-SseB, Enteritidis-SseB, ParaOSP-CP1 | Enteritidis-SseB, ParaOSP-SseB, Typhimurium-IpaB |
| Typhimurium-SseB, Vi-SseB, ParaOSP-SseB | Enteritidis-SseB, ParaOSP-SseB, Enteritidis-IpaB |
| Typhimurium-SseB, Vi-SseB, Typhimurium-IpaB | Enteritidis-SseB, ParaOSP-SseB, Vi-IpaB |
| Typhimurium-SseB, Vi-SseB, Enteritidis-IpaB | Enteritidis-SseB, ParaOSP-SseB, ParaOSP-IpaB |
| Typhimurium-SseB, Vi-SseB, Vi-IpaB | Enteritidis-SseB, ParaOSP-SseB, Typhimurium-CP1 |
| Typhimurium-SseB, Vi-SseB, ParaOSP-IpaB | Enteritidis-SseB, ParaOSP-SseB, Enteritidis-CP1 |
| Typhimurium-SseB, Vi-SseB, Typhimurium-CP1 | Enteritidis-SseB, ParaOSP-SseB, Vi-CP1 |
| Typhimurium-SseB, Vi-SseB, Enteritidis-CP1 | Enteritidis-SseB, ParaOSP-SseB, ParaOSP-CP1 |
| Typhimurium-SseB, Vi-SseB, Vi-CP1 | Enteritidis-SseB, Typhimurium-IpaB, Enteritidis-IpaB |
| Typhimurium-SseB, Vi-SseB, ParaOSP-CP1 | Enteritidis-SseB, Typhimurium-IpaB, Vi-IpaB |
| Typhimurium-SseB, ParaOSP-SseB, Typhimurium-IpaB | Enteritidis-SseB, Typhimurium-IpaB, ParaOSP-IpaB |
| Typhimurium-SseB, ParaOSP-SseB, Enteritidis-IpaB | Enteritidis-SseB, Typhimurium-IpaB, Typhimurium-CP1 |
| Typhimurium-SseB, ParaOSP-SseB, Vi-IpaB | Enteritidis-SseB, Typhimurium-IpaB, Enteritidis-CP1 |
| Typhimurium-SseB, ParaOSP-SseB, ParaOSP-IpaB | Enteritidis-SseB, Typhimurium-IpaB, Vi-CP1 |
| Typhimurium-SseB, ParaOSP-SseB, Typhimurium-CP1 | Enteritidis-SseB, Typhimurium-IpaB, ParaOSP-CP1 |
| Typhimurium-SseB, ParaOSP-SseB, Enteritidis-CP1 | Enteritidis-SseB, Enteritidis-IpaB, Vi-IpaB |
| Typhimurium-SseB, ParaOSP-SseB, Vi-CP1 | Enteritidis-SseB, Enteritidis-IpaB, ParaOSP-IpaB |
| Typhimurium-SseB, ParaOSP-SseB, ParaOSP-CP1 | Enteritidis-SseB, Enteritidis-IpaB, Typhimurium-CP1 |
| Typhimurium-SseB, Typhimurium-IpaB, Enteritidis-IpaB | Enteritidis-SseB, Enteritidis-IpaB, Enteritidis-CP1 |
| Typhimurium-SseB, Typhimurium-IpaB, Vi-IpaB | Enteritidis-SseB, Enteritidis-IpaB, Vi-CP1 |
| Typhimurium-SseB, Typhimurium-IpaB, ParaOSP-IpaB | Enteritidis-SseB, Enteritidis-IpaB, ParaOSP-CP1 |
| Typhimurium-SseB, Typhimurium-IpaB, Typhimurium-CP1 | Enteritidis-SseB, Vi-IpaB, ParaOSP-IpaB |
| Typhimurium-SseB, Typhimurium-IpaB, Enteritidis-CP1 | Enteritidis-SseB, Vi-IpaB, Typhimurium-CP1 |
| Typhimurium-SseB, Typhimurium-IpaB, Vi-CP1 | Enteritidis-SseB, Vi-IpaB, Enteritidis-CP1 |
| Typhimurium-SseB, Typhimurium-IpaB, ParaOSP-CP1 | Enteritidis-SseB, Vi-IpaB, Vi-CP1 |
| Typhimurium-SseB, Enteritidis-IpaB, Vi-IpaB | Enteritidis-SseB, Vi-IpaB, ParaOSP-CP1 |
| Typhimurium-SseB, Enteritidis-IpaB, ParaOSP-IpaB | Enteritidis-SseB, ParaOSP-IpaB, Typhimurium-CP1 |
| Typhimurium-SseB, Enteritidis-IpaB, Typhimurium-CP1 | Enteritidis-SseB, ParaOSP-IpaB, Enteritidis-CP1 |
| Typhimurium-SseB, Enteritidis-IpaB, Enteritidis-CP1 | Enteritidis-SseB, ParaOSP-IpaB, Vi-CP1 |
| Typhimurium-SseB, Enteritidis-IpaB, Vi-CP1 | Enteritidis-SseB, ParaOSP-IpaB, ParaOSP-CP1 |
| Typhimurium-SseB, Enteritidis-IpaB, ParaOSP-CP1 | Enteritidis-SseB, Typhimurium-CP1, Enteritidis-CP1 |
| Typhimurium-SseB, Vi-IpaB, ParaOSP-IpaB | Enteritidis-SseB, Typhimurium-CP1, Vi-CP1 |
| Typhimurium-SseB, Vi-IpaB, Typhimurium-CP1 | Enteritidis-SseB, Typhimurium-CP1, ParaOSP-CP1 |
| Typhimurium-SseB, Vi-IpaB, Enteritidis-CP1 | Enteritidis-SseB, Enteritidis-CP1, Vi-CP1 |
| Typhimurium-SseB, Vi-IpaB, Vi-CP1 | Enteritidis-SseB, Enteritidis-CP1, ParaOSP-CP1 |
| Typhimurium-SseB, Vi-IpaB, ParaOSP-CP1 | Enteritidis-SseB, Vi-CP1, ParaOSP-CP1 |
| Typhimurium-SseB, ParaOSP-IpaB, Typhimurium-CP1 | ParaOSP-SseB, Typhimurium-IpaB, Enteritidis-IpaB |
| Typhimurium-SseB, ParaOSP-IpaB, Enteritidis-CP1 | ParaOSP-SseB, Typhimurium-IpaB, Vi-IpaB |
| Typhimurium-SseB, ParaOSP-IpaB, Vi-CP1 | ParaOSP-SseB, Typhimurium-IpaB, ParaOSP-IpaB |
| Typhimurium-SseB, ParaOSP-IpaB, ParaOSP-CP1 | ParaOSP-SseB, Typhimurium-IpaB, Typhimurium-CP1 |
| Typhimurium-SseB, Typhimurium-CP1, Enteritidis-CP1 | ParaOSP-SseB, Typhimurium-IpaB, Enteritidis-CP1 |
| Typhimurium-SseB, Typhimurium-CP1, Vi-CP1 | ParaOSP-SseB, Typhimurium-IpaB, Vi-CP1 |
| Typhimurium-SseB, Typhimurium-CP1, ParaOSP-CP1 | ParaOSP-SseB, Typhimurium-IpaB, ParaOSP-CP1 |
| Typhimurium-SseB, Enteritidis-CP1, Vi-CP1 | ParaOSP-SseB, Enteritidis-IpaB, Vi-IpaB |
| Typhimurium-SseB, Enteritidis-CP1, ParaOSP-CP1 | ParaOSP-SseB, Enteritidis-IpaB, ParaOSP-IpaB |
| Typhimurium-SseB, Vi-CP1, ParaOSP-CP1 | ParaOSP-SseB, Enteritidis-IpaB, Typhimurium-CP1 |
| Vi-SseB, ParaOSP-SseB, Typhimurium-IpaB | ParaOSP-SseB, Enteritidis-IpaB, Enteritidis-CP1 |
| Vi-SseB, ParaOSP-SseB, Enteritidis-IpaB | ParaOSP-SseB, Enteritidis-IpaB, Vi-CP1 |
| Vi-SseB, ParaOSP-SseB, Vi-IpaB | ParaOSP-SseB, Enteritidis-IpaB, ParaOSP-CP1 |
| Vi-SseB, ParaOSP-SseB, ParaOSP-IpaB | ParaOSP-SseB, Vi-IpaB, ParaOSP-IpaB |
| Vi-SseB, ParaOSP-SseB, Typhimurium-CP1 | ParaOSP-SseB, Vi-IpaB, Typhimurium-CP1 |
| Vi-SseB, ParaOSP-SseB, Enteritidis-CP1 | ParaOSP-SseB, Vi-IpaB, Enteritidis-CP1 |
| Vi-SseB, ParaOSP-SseB, Vi-CP1 | ParaOSP-SseB, Vi-IpaB, Vi-CP1 |
| Vi-SseB, ParaOSP-SseB, ParaOSP-CP1 | ParaOSP-SseB, Vi-IpaB, ParaOSP-CP1 |
| Vi-SseB, Typhimurium-IpaB, Enteritidis-IpaB | ParaOSP-SseB, ParaOSP-IpaB, Typhimurium-CP1 |
| Vi-SseB, Typhimurium-IpaB, Vi-IpaB | ParaOSP-SseB, ParaOSP-IpaB, Enteritidis-CP1 |
| Vi-SseB, Typhimurium-IpaB, ParaOSP-IpaB | ParaOSP-SseB, ParaOSP-IpaB, Vi-CP1 |
| Vi-SseB, Typhimurium-IpaB, Typhimurium-CP1 | ParaOSP-SseB, ParaOSP-IpaB, ParaOSP-CP1 |
| Vi-SseB, Typhimurium-IpaB, Enteritidis-CP1 | ParaOSP-SseB, Typhimurium-CP1, Enteritidis-CP1 |
| Vi-SseB, Typhimurium-IpaB, Vi-CP1 | ParaOSP-SseB, Typhimurium-CP1, Vi-CP1 |
| Vi-SseB, Typhimurium-IpaB, ParaOSP-CP1 | ParaOSP-SseB, Typhimurium-CP1, ParaOSP-CP1 |
| Vi-SseB, Enteritidis-IpaB, Vi-IpaB | ParaOSP-SseB, Enteritidis-CP1, Vi-CP1 |
| Vi-SseB, Enteritidis-IpaB, ParaOSP-IpaB | ParaOSP-SseB, Enteritidis-CP1, ParaOSP-CP1 |
| Vi-SseB, Enteritidis-IpaB, Typhimurium-CP1 | Typhimurium-IpaB, Enteritidis-IpaB, Vi-IpaB |
| Vi-SseB, Enteritidis-IpaB, Enteritidis-CP1 | Typhimurium-IpaB, Enteritidis-IpaB, ParaOSP-IpaB |
| Vi-SseB, Enteritidis-IpaB, Vi-CP1 | Typhimurium-IpaB, Enteritidis-IpaB, Typhimurium-CP1 |
| Vi-SseB, Enteritidis-IpaB, ParaOSP-CP1 | Typhimurium-IpaB, Enteritidis-IpaB, Enteritidis-CP1 |
| Vi-SseB, Vi-IpaB, ParaOSP-IpaB | Typhimurium-IpaB, Enteritidis-IpaB, Vi-CP1 |
| Vi-SseB, Vi-IpaB, Typhimurium-CP1 | Typhimurium-IpaB, Enteritidis-IpaB, ParaOSP-CP1 |
| Vi-SseB, Vi-IpaB, Enteritidis-CP1 | Typhimurium-IpaB, Vi-IpaB, ParaOSP-IpaB |
| Vi-SseB, Vi-IpaB, Vi-CP1 | Typhimurium-IpaB, Vi-IpaB, Typhimurium-CP1 |
| Vi-SseB, Vi-IpaB, ParaOSP-CP1 | Typhimurium-IpaB, Vi-IpaB, Enteritidis-CP1 |
| Vi-SseB, ParaOSP-IpaB, Typhimurium-CP1 | Typhimurium-IpaB, Vi-IpaB, Vi-CP1 |
| Vi-SseB, ParaOSP-IpaB, Enteritidis-CP1 | Typhimurium-IpaB, Vi-IpaB, ParaOSP-CP1 |
| Vi-SseB, ParaOSP-IpaB, Vi-CP1 | Typhimurium-IpaB, ParaOSP-IpaB, Typhimurium-CP1 |
| Vi-SseB, ParaOSP-IpaB, ParaOSP-CP1 | Typhimurium-IpaB, ParaOSP-IpaB, Enteritidis-CP1 |
| Vi-SseB, Typhimurium-CP1, Enteritidis-CP1 | Typhimurium-IpaB, ParaOSP-IpaB, Vi-CP1 |
| Vi-SseB, Typhimurium-CP1, Vi-CP1 | Typhimurium-IpaB, ParaOSP-IpaB, ParaOSP-CP1 |
| Vi-SseB, Typhimurium-CP1, ParaOSP-CP1 | Typhimurium-IpaB, Typhimurium-CP1, Enteritidis-CP1 |
| Vi-SseB, Enteritidis-CP1, Vi-CP1 | Typhimurium-IpaB, Typhimurium-CP1, Vi-CP1 |
| Vi-SseB, Enteritidis-CP1, ParaOSP-CP1 | Typhimurium-IpaB, Typhimurium-CP1, ParaOSP-CP1 |
| Vi-SseB, Vi-CP1, ParaOSP-CP1 | Typhimurium-IpaB, Enteritidis-CP1, Vi-CP1 |
| ParaOSP-SseB, Vi-CP1, ParaOSP-CP1 | Typhimurium-IpaB, Enteritidis-CP1, ParaOSP-CP1 |
| Vi-IpaB, ParaOSP-IpaB, Typhimurium-CP1 | Typhimurium-IpaB, Vi-CP1, ParaOSP-CP1 |
| Vi-IpaB, ParaOSP-IpaB, Enteritidis-CP1 | ParaOSP-IpaB, Typhimurium-CP1, Enteritidis-CP1 |
| Vi-IpaB, ParaOSP-IpaB, Vi-CP1 | ParaOSP-IpaB, Typhimurium-CP1, Vi-CP1 |
| Vi-IpaB, ParaOSP-IpaB, ParaOSP-CP1 | ParaOSP-IpaB, Typhimurium-CP1, ParaOSP-CP1 |
| Vi-IpaB, Typhimurium-CP1, Enteritidis-CP1 | ParaOSP-IpaB, Enteritidis-CP1, Vi-CP1 |
| Vi-IpaB, Typhimurium-CP1, Vi-CP1 | ParaOSP-IpaB, Enteritidis-CP1, ParaOSP-CP1 |
| Vi-IpaB, Typhimurium-CP1, ParaOSP-CP1 | ParaOSP-IpaB, Vi-CP1, ParaOSP-CP1 |
| Vi-IpaB, Enteritidis-CP1, Vi-CP1 | Typhimurium-CP1, Enteritidis-CP1, Vi-CP1 |
| Vi-IpaB, Enteritidis-CP1, ParaOSP-CP1 | Typhimurium-CP1, Enteritidis-CP1, ParaOSP-CP1 |
| Vi-IpaB, Vi-CP1, ParaOSP-CP1 | Typhimurium-CP1, Vi-CP1, ParaOSP-CP1 |
| Enteritidis-IpaB, Vi-IpaB, ParaOSP-IpaB | Enteritidis-CP1, Vi-CP1, ParaOSP-CP1 |
| Enteritidis-IpaB, Vi-IpaB, Typhimurium-CP1 | |
| Enteritidis-IpaB, Vi-IpaB, Enteritidis-CP1 | |
| Enteritidis-IpaB, Vi-IpaB, Vi-CP1 | |
| Enteritidis-IpaB, Vi-IpaB, ParaOSP-CP1 | |
| Enteritidis-IpaB, ParaOSP-IpaB, Typhimurium-CP1 | |
| Enteritidis-IpaB, ParaOSP-IpaB, Enteritidis-CP1 | |
| Enteritidis-IpaB, ParaOSP-IpaB, Vi-CP1 | |
| Enteritidis-IpaB, ParaOSP-IpaB, ParaOSP-CP1 | |
| Enteritidis-IpaB, Typhimurium-CP1, Enteritidis-CP1 | |
| Enteritidis-IpaB, Typhimurium-CP1, Vi-CP1 | |
| Enteritidis-IpaB, Typhimurium-CP1, ParaOSP-CP1 | |
| Enteritidis-IpaB, Enteritidis-CP1, Vi-CP1 | |
| Enteritidis-IpaB, Enteritidis-CP1, ParaOSP-CP1 | |
| Enteritidis-IpaB, Vi-CP1, ParaOSP-CP1 | |

In some embodiments, an immunogenic vaccine comprises at least four immunogenic compositions as described herein, wherein the four immunogenic compositions are selected from any of Typhimurium-SseB, Enteritidis-SseB, Vi-SseB, ParaOSP-SseB, Typhimurium-IpaB, Enteritidis-IpaB, Vi-IpaB, ParaOSP-IpaB, Typhimurium-CP1, Enteritidis-CP1, Vi-CP1, ParaOSP-CP1. Exemplary combinations are shown in Table 3C.

In one embodiment, a quadrivalent vaccine comprises the following four immunogenic complexes: S Typhimurium-SseB, S. Enteritidis-SseB, Vi-CP1 and ParaOSP-CP1 (referred to herein in the Examples as “Salmonella-MAPS 1”). In one embodiment, a quadrivalent vaccine comprises the following four immunogenic complexes: S. Typhimurium-SseB, S. Enteritidis-SseB, Vi-SseB and ParaOSP-SseB (referred to herein in the Examples as “Salmonella-MAPS 2”).

Table 3C. Exemplary combinations of Salmonella-MAPS immunogenic complexes for a quadrivalent (4V) vaccine composition comprising four Salmonella-MAPS immunogenic complexes in the vaccine, which can be combined with one or more Shigella-MAPS immunogenic complexes.

TABLE 3C
4V Salmonella-MAPS vaccines

| | |
|---|---|
| Typhimurium-SseB, Enteritidis-SseB, Vi- | Enteritidis-IpaB, Vi-IpaB, ParaOSP- |
| SseB, ParaOSP-SseB | IpaB, Typhimurium-CP1 |
| Typhimurium-SseB, Enteritidis-SseB, Vi- | Enteritidis-IpaB, Vi-IpaB, ParaOSP- |
| SseB, Typhimurium-IpaB | IpaB, Enteritidis-CP1 |
| Typhimurium-SseB, Enteritidis-SseB, Vi- | Enteritidis-IpaB, Vi-IpaB, ParaOSP-IpaB, Vi-CP1 |
| SseB, Enteritidis-IpaB | Enteritidis-IpaB, Vi-IpaB, ParaOSP-IpaB, ParaOSP- |
| Typhimurium-SseB, Enteritidis-SseB, Vi-SseB, Vi-IpaB | CP1 |
| Typhimurium-SseB, Enteritidis-SseB, Vi- | Enteritidis-IpaB, Vi-IpaB, Typhimurium- |
| SseB, ParaOSP-IpaB | CP1, Enteritidis-CP1 |
| Typhimurium-SseB, Enteritidis-SseB, Vi- | Enteritidis-IpaB, Vi-IpaB, Typhimurium-CP1, Vi- |
| SseB, Typhimurium-CP1 | CP1 |
| Typhimurium-SseB, Enteritidis-SseB, Vi- | Enteritidis-IpaB, Vi-IpaB, Typhimurium- |
| SseB, Enteritidis-CP1 | CP1, ParaOSP-CP1 |
| Typhimurium-SseB, Enteritidis-SseB, Vi-SseB, Vi-CP1 | Enteritidis-IpaB, Vi-IpaB, Enteritidis-CP1, Vi-CP1 |
| Typhimurium-SseB, Enteritidis-SseB, Vi- | Enteritidis-IpaB, Vi-IpaB, Enteritidis- |
| SseB, ParaOSP-CP1 | CP1, ParaOSP-CP1 |
| Typhimurium-SseB, Enteritidis-SseB, ParaOSP- | Enteritidis-IpaB, Vi-IpaB, Vi-CP1, ParaOSP-CP1 |
| SseB, Typhimurium-IpaB | Enteritidis-IpaB, ParaOSP-IpaB, Typhimurium- |
| Typhimurium-SseB, Enteritidis-SseB, ParaOSP- | CP1, Enteritidis-CP1 |
| SseB, Enteritidis-IpaB | Enteritidis-IpaB, ParaOSP-IpaB, Typhimurium- |
| Typhimurium-SseB, Enteritidis-SseB, ParaOSP- | CP1, Vi-CP1 |
| SseB, Vi-IpaB | Enteritidis-IpaB, ParaOSP-IpaB, Typhimurium- |
| Typhimurium-SseB, Enteritidis-SseB, ParaOSP- | CP1, ParaOSP-CP1 |
| SseB, ParaOSP-IpaB | Enteritidis-IpaB, ParaOSP-IpaB, Enteritidis- |
| Typhimurium-SseB, Enteritidis-SseB, ParaOSP- | CP1, Vi-CP1 |
| SseB, Typhimurium-CP1 | Enteritidis-IpaB, ParaOSP-IpaB, Enteritidis- |
| Typhimurium-SseB, Enteritidis-SseB, ParaOSP- | CP1, ParaOSP-CP1 |
| SseB, Enteritidis-CP1 | Enteritidis-IpaB, ParaOSP-IpaB, Vi-CP1, ParaOSP- |
| Typhimurium-SseB, Enteritidis-SseB, ParaOSP- | CP1 |
| SseB, Vi-CP1 | Enteritidis-IpaB, Typhimurium-CP1, Enteritidis- |
| Typhimurium-SseB, Enteritidis-SseB, ParaOSP- | CP1, Vi-CP1 |
| SseB, ParaOSP-CP1 | Enteritidis-IpaB, Typhimurium-CP1, Enteritidis- |
| Typhimurium-SseB, Enteritidis-SseB, Typhimurium- | CP1, ParaOSP-CP1 |
| IpaB, Enteritidis-IpaB | Enteritidis-IpaB, Typhimurium-CP1, Vi- |
| Typhimurium-SseB, Enteritidis-SseB, Typhimurium- | CP1, ParaOSP-CP1 |
| IpaB, Vi-IpaB | Enteritidis-IpaB, Enteritidis-CP1, Vi- |
| Typhimurium-SseB, Enteritidis-SseB, Typhimurium- | CP1, ParaOSP-CP1 |
| IpaB, ParaOSP-IpaB | Vi-IpaB, ParaOSP-IpaB, Typhimurium- |
| Typhimurium-SseB, Enteritidis-SseB, Typhimurium- | CP1, Enteritidis-CP1 |
| IpaB, Typhimurium-CP1 | Vi-IpaB, ParaOSP-IpaB, Typhimurium-CP1, Vi- |
| Typhimurium-SseB, Enteritidis-SseB, Typhimurium- | CPI |
| IpaB, Enteritidis-CP1 | Vi-IpaB, ParaOSP-IpaB, Typhimurium- |
| Typhimurium-SseB, Enteritidis-SseB, Typhimurium- | CP1, ParaOSP-CP1 |
| IpaB, Vi-CP1 | Vi-IpaB, ParaOSP-IpaB, Enteritidis-CP1, Vi-CP1 |
| Typhimurium-SseB, Enteritidis-SseB, Typhimurium- | Vi-IpaB, ParaOSP-IpaB, Enteritidis-CP1, ParaOSP- |
| IpaB, ParaOSP-CP1 | CP1 |
| Typhimurium-SseB, Enteritidis-SseB, Enteritidis- | Vi-IpaB, ParaOSP-IpaB, Vi-CP1, ParaOSP-CP1 |
| IpaB, Vi-IpaB | Vi-IpaB, Typhimurium-CP1, Enteritidis-CP1, Vi- |
| Typhimurium-SseB, Enteritidis-SseB, Enteritidis- | CP1 |
| IpaB, ParaOSP-IpaB | Vi-IpaB, Typhimurium-CP1, Enteritidis- |
| Typhimurium-SseB, Enteritidis-SseB, Enteritidis- | CP1, ParaOSP-CP1 |
| IpaB, Typhimurium-CP1 | Vi-IpaB, Typhimurium-CP1, Vi-CP1, ParaOSP- |
| Typhimurium-SseB, Enteritidis-SseB, Enteritidis- | CP1 |
| IpaB, Enteritidis-CP1 | Vi-IpaB, Enteritidis-CP1, Vi-CP1, ParaOSP-CP1 |
| Typhimurium-SseB, Enteritidis-SseB, Enteritidis- | ParaOSP-IpaB, Typhimurium-CP1, Enteritidis- |
| IpaB, Vi-CP1 | CP1, Vi-CP1 |
| Typhimurium-SseB, Enteritidis-SseB, Enteritidis- | ParaOSP-IpaB, Typhimurium-CP1, Enteritidis- |
| IpaB, ParaOSP-CP1 | CP1, ParaOSP-CP1 |
| Typhimurium-SseB, Enteritidis-SseB, Vi- | ParaOSP-IpaB, Typhimurium-CP1, Vi- |
| IpaB, ParaOSP-IpaB | CP1, ParaOSP-CP1 |
| Typhimurium-SseB, Enteritidis-SseB, Vi- | ParaOSP-IpaB, Enteritidis-CP1, Vi-CP1, ParaOSP- |
| IpaB, Typhimurium-CP1 | CP1 |
| Typhimurium-SseB, Enteritidis-SseB, Vi- | Typhimurium-CP1, Enteritidis-CP1, Vi- |
| IpaB, Enteritidis-CP1 | CP1, ParaOSP-CP1 |
| Typhimurium-SseB, Enteritidis-SseB, Vi-IpaB, Vi-CP1 | ParaOSP-SseB, Typhimurium-IpaB, Enteritidis- |
| Typhimurium-SseB, Enteritidis-SseB, Vi- | IpaB, Vi-IpaB |
| IpaB, ParaOSP-CP1 | ParaOSP-SseB, Typhimurium-IpaB, Enteritidis- |
| Typhimurium-SseB, Enteritidis-SseB, ParaOSP- | IpaB, ParaOSP-IpaB |
| IpaB, Typhimurium-CP1 | ParaOSP-SseB, Typhimurium-IpaB, Enteritidis- |
| Typhimurium-SseB, Enteritidis-SseB, ParaOSP- | IpaB, Typhimurium-CP1 |
| IpaB, Enteritidis-CP1 | ParaOSP-SseB, Typhimurium-IpaB, Enteritidis- |
| Typhimurium-SseB, Enteritidis-SseB, ParaOSP- | IpaB, Enteritidis-CP1 |
| IpaB, Vi-CP1 | ParaOSP-SseB, Typhimurium-IpaB, Enteritidis- |
| Typhimurium-SseB, Enteritidis-SseB, ParaOSP- | IpaB, Vi-CP1 |
| IpaB, ParaOSP-CP1 | ParaOSP-SseB, Typhimurium-IpaB, Enteritidis- |
| Typhimurium-SseB, Enteritidis-SseB, Typhimurium- | IpaB, ParaOSP-CP1 |
| CP1, Enteritidis-CP1 | ParaOSP-SseB, Typhimurium-IpaB, Vi- |
| Typhimurium-SseB, Enteritidis-SseB, Typhimurium- | IpaB, ParaOSP-IpaB |
| CP1, Vi-CP1 | ParaOSP-SseB, Typhimurium-IpaB, Vi- |
| Typhimurium-SseB, Enteritidis-SseB, Typhimurium- | IpaB, Typhimurium-CP1 |
| CP1, ParaOSP-CP1 | ParaOSP-SseB, Typhimurium-IpaB, Vi- |
| Typhimurium-SseB, Enteritidis-SseB, Enteritidis- | IpaB, Enteritidis-CP1 |
| CP1, Vi-CP1 | ParaOSP-SseB, Typhimurium-IpaB, Vi-IpaB, Vi- |
| Typhimurium-SseB, Enteritidis-SseB, Enteritidis- | CP1 |
| CP1, ParaOSP-CP1 | ParaOSP-SseB, Typhimurium-IpaB, Vi- |
| Typhimurium-SseB, Enteritidis-SseB, Vi-CP1, ParaOSP- | IpaB, ParaOSP-CP1 |
| CP1 | ParaOSP-SseB, Typhimurium-IpaB, ParaOSP- |
| Typhimurium-SseB, Vi-SseB, ParaOSP- | IpaB, Typhimurium-CP1 |
| SseB, Typhimurium-IpaB | ParaOSP-SseB, Typhimurium-IpaB, ParaOSP- |
| Typhimurium-SseB, Vi-SseB, ParaOSP- | IpaB, Enteritidis-CP1 |
| SseB, Enteritidis-IpaB | ParaOSP-SseB, Typhimurium-IpaB, ParaOSP- |
| Typhimurium-SseB, Vi-SseB, ParaOSP-SseB, Vi-IpaB | IpaB, Vi-CP1 |
| Typhimurium-SseB, Vi-SseB, ParaOSP-SseB, ParaOSP- | ParaOSP-SseB, Typhimurium-IpaB, ParaOSP- |
| IpaB | IpaB, ParaOSP-CP1 |
| Typhimurium-SseB, Vi-SseB, ParaOSP- | ParaOSP-SseB, Typhimurium-IpaB, Typhimurium- |
| SseB, Typhimurium-CP1 | CP1, Enteritidis-CP1 |
| Typhimurium-SseB, Vi-SseB, ParaOSP- | ParaOSP-SseB, Typhimurium-IpaB, Typhimurium- |
| SseB, Enteritidis-CP1 | CP1, Vi-CP1 |
| Typhimurium-SseB, Vi-SseB, ParaOSP-SseB, Vi-CP1 | ParaOSP-SseB, Typhimurium-IpaB, Typhimurium- |
| Typhimurium-SseB, Vi-SseB, ParaOSP-SseB, ParaOSP- | CP1, ParaOSP-CP1 |
| CP1 | ParaOSP-SseB, Typhimurium-IpaB, Enteritidis- |
| Typhimurium-SseB, Vi-SseB, Typhimurium- | CP1, Vi-CP1 |
| IpaB, Enteritidis-IpaB | ParaOSP-SseB, Typhimurium-IpaB, Enteritidis- |
| Typhimurium-SseB, Vi-SseB, Typhimurium-IpaB, Vi- | CP1, ParaOSP-CP1 |
| IpaB | ParaOSP-SseB, Typhimurium-IpaB, Vi- |
| Typhimurium-SseB, Vi-SseB, Typhimurium- | CP1, ParaOSP-CP1 |
| IpaB, ParaOSP-IpaB | ParaOSP-SseB, Enteritidis-IpaB, Vi- |
| Typhimurium-SseB, Vi-SseB, Typhimurium- | IpaB, ParaOSP-IpaB |
| IpaB, Typhimurium-CP1 | ParaOSP-SseB, Enteritidis-IpaB, Vi- |
| Typhimurium-SseB, Vi-SseB, Typhimurium- | IpaB, Typhimurium-CP1 |
| IpaB, Enteritidis-CP1 | ParaOSP-SseB, Enteritidis-IpaB, Vi- |
| Typhimurium-SseB, Vi-SseB, Typhimurium-IpaB, Vi- | IpaB, Enteritidis-CP1 |
| CP1 | ParaOSP-SseB, Enteritidis-IpaB, Vi-IpaB, Vi-CP1 |
| Typhimurium-SseB, Vi-SseB, Typhimurium- | ParaOSP-SseB, Enteritidis-IpaB, Vi- |
| IpaB, ParaOSP-CP1 | IpaB, ParaOSP-CP1 |
| Typhimurium-SseB, Vi-SseB, Enteritidis-IpaB, Vi-IpaB | ParaOSP-SseB, Enteritidis-IpaB, ParaOSP- |
| Typhimurium-SseB, Vi-SseB, Enteritidis- | IpaB, Typhimurium-CP1 |
| IpaB, ParaOSP-IpaB | ParaOSP-SseB, Enteritidis-IpaB, ParaOSP- |
| Typhimurium-SseB, Vi-SseB, Enteritidis- | IpaB, Enteritidis-CP1 |
| IpaB, Typhimurium-CP1 | ParaOSP-SseB, Enteritidis-IpaB, ParaOSP- |
| Typhimurium-SseB, Vi-SseB, Enteritidis- | IpaB, Vi-CP1 |
| IpaB, Enteritidis-CP1 | ParaOSP-SseB, Enteritidis-IpaB, ParaOSP- |
| Typhimurium-SseB, Vi-SseB, Enteritidis-IpaB, Vi-CP1 | IpaB, ParaOSP-CP1 |
| Typhimurium-SseB, Vi-SseB, Enteritidis- | ParaOSP-SseB, Enteritidis-IpaB, Typhimurium- |
| IpaB, ParaOSP-CP1 | CP1, Enteritidis-CP1 |
| Typhimurium-SseB, Vi-SseB, Vi-IpaB, ParaOSP-IpaB | ParaOSP-SseB, Enteritidis-IpaB, Typhimurium- |
| Typhimurium-SseB, Vi-SseB, Vi-IpaB, Typhimurium- | CP1, Vi-CP1 |
| CP1 | ParaOSP-SseB, Enteritidis-IpaB, Typhimurium- |
| Typhimurium-SseB, Vi-SseB, Vi-IpaB, Enteritidis-CP1 | CP1, ParaOSP-CP1 |
| Typhimurium-SseB, Vi-SseB, Vi-IpaB, Vi-CP1 | ParaOSP-SseB, Enteritidis-IpaB, Enteritidis- |
| Typhimurium-SseB, Vi-SseB, Vi-IpaB, ParaOSP-CP1 | CP1, Vi-CP1 |
| Typhimurium-SseB, Vi-SseB, ParaOSP- | ParaOSP-SseB, Enteritidis-IpaB, Enteritidis- |
| IpaB, Typhimurium-CP1 | CP1, ParaOSP-CP1 |
| Typhimurium-SseB, Vi-SseB, ParaOSP- | ParaOSP-SseB, Enteritidis-IpaB, Vi-CP1, ParaOSP- |
| IpaB, Enteritidis-CP1 | CP1 |
| Typhimurium-SseB, Vi-SseB, ParaOSP-IpaB, Vi-CP1 | ParaOSP-SseB, Vi-IpaB, ParaOSP- |
| Typhimurium-SseB, Vi-SseB, ParaOSP-IpaB, ParaOSP- | IpaB, Typhimurium-CP1 |
| CP1 | ParaOSP-SseB, Vi-IpaB, ParaOSP- |
| Typhimurium-SseB, Vi-SseB, Typhimurium- | IpaB, Enteritidis-CP1 |
| CP1, Enteritidis-CP1 | ParaOSP-SseB, Vi-IpaB, ParaOSP-IpaB, Vi-CP1 |
| Typhimurium-SseB, Vi-SseB, Typhimurium-CP1, Vi- | ParaOSP-SseB, Vi-IpaB, ParaOSP-IpaB, ParaOSP- |
| CP1 | CP1 |
| Typhimurium-SseB, Vi-SseB, Typhimurium- | ParaOSP-SseB, Vi-IpaB, Typhimurium- |
| CP1, ParaOSP-CP1 | CP1, Enteritidis-CP1 |
| Typhimurium-SseB, Vi-SseB, Enteritidis-CP1, Vi-CP1 | ParaOSP-SseB, Vi-IpaB, Typhimurium-CP1, Vi- |
| Typhimurium-SseB, Vi-SseB, Enteritidis-CP1, ParaOSP- | CP1 |
| CP1 | ParaOSP-SseB, Vi-IpaB, Typhimurium- |
| Typhimurium-SseB, Vi-SseB, Vi-CP1, ParaOSP-CP1 | CP1, ParaOSP-CP1 |
| Typhimurium-SseB, ParaOSP-SseB, Typhimurium- | ParaOSP-SseB, Vi-IpaB, Enteritidis-CP1, Vi-CP1 |
| IpaB, Enteritidis-IpaB | ParaOSP-SseB, Vi-IpaB, Enteritidis-CP1, ParaOSP- |
| Typhimurium-SseB, ParaOSP-SseB, Typhimurium- | CP1 |
| IpaB, Vi-IpaB | ParaOSP-SseB, Vi-IpaB, Vi-CP1, ParaOSP-CP1 |
| Typhimurium-SseB, ParaOSP-SseB, Typhimurium- | ParaOSP-SseB, ParaOSP-IpaB, Typhimurium- |
| IpaB, ParaOSP-IpaB | CP1, Enteritidis-CP1 |
| Typhimurium-SseB, ParaOSP-SseB, Typhimurium- | ParaOSP-SseB, ParaOSP-IpaB, Typhimurium- |
| IpaB, Typhimurium-CP1 | CP1, Vi-CP1 |
| Typhimurium-SseB, ParaOSP-SseB, Typhimurium- | ParaOSP-SseB, ParaOSP-IpaB, Typhimurium- |
| IpaB, Enteritidis-CP1 | CP1, ParaOSP-CP1 |
| Typhimurium-SseB, ParaOSP-SseB, Typhimurium- | ParaOSP-SseB, ParaOSP-IpaB, Enteritidis-CP1, Vi- |
| IpaB, Vi-CP1 | CP1 |
| Typhimurium-SseB, ParaOSP-SseB, Typhimurium- | ParaOSP-SseB, ParaOSP-IpaB, Enteritidis- |
| IpaB, ParaOSP-CP1 | CP1, ParaOSP-CP1 |
| Typhimurium-SseB, ParaOSP-SseB, Enteritidis- | ParaOSP-SseB, ParaOSP-IpaB, Vi-CP1, ParaOSP- |
| IpaB, Vi-IpaB | CP1 |
| Typhimurium-SseB, ParaOSP-SseB, Enteritidis- | ParaOSP-SseB, Typhimurium-CP1, Enteritidis- |
| IpaB, ParaOSP-IpaB | CP1, Vi-CP1 |
| Typhimurium-SseB, ParaOSP-SseB, Enteritidis- | ParaOSP-SseB, Typhimurium-CP1, Enteritidis- |
| IpaB, Typhimurium-CP1 | CP1, ParaOSP-CP1 |
| Typhimurium-SseB, ParaOSP-SseB, Enteritidis- | ParaOSP-SseB, Typhimurium-CP1, Vi- |
| IpaB, Enteritidis-CP1 | CP1, ParaOSP-CP1 |
| Typhimurium-SseB, ParaOSP-SseB, Enteritidis- | ParaOSP-SseB, Enteritidis-CP1, Vi-CP1, ParaOSP- |
| IpaB, Vi-CP1 | CP1 |
| Typhimurium-SseB, ParaOSP-SseB, Enteritidis- | Typhimurium-IpaB, Enteritidis-IpaB, Vi- |
| IpaB, ParaOSP-CP1 | IpaB, ParaOSP-IpaB |
| Typhimurium-SseB, ParaOSP-SseB, Vi-IpaB, ParaOSP- | Typhimurium-IpaB, Enteritidis-IpaB, Vi- |
| IpaB | IpaB, Typhimurium-CP1 |
| Typhimurium-SseB, ParaOSP-SseB, Vi- | Typhimurium-IpaB, Enteritidis-IpaB, Vi- |
| IpaB, Typhimurium-CP1 | IpaB, Enteritidis-CP1 |
| Typhimurium-SseB, ParaOSP-SseB, Vi- | Typhimurium-IpaB, Enteritidis-IpaB, Vi-IpaB, Vi- |
| IpaB, Enteritidis-CP1 | CP1 |
| Typhimurium-SseB, ParaOSP-SseB, Vi-IpaB, Vi-CP1 | Typhimurium-IpaB, Enteritidis-IpaB, Vi- |
| Typhimurium-SseB, ParaOSP-SseB, Vi-IpaB, ParaOSP- | IpaB, ParaOSP-CP1 |
| CP1 | Typhimurium-IpaB, Enteritidis-IpaB, ParaOSP- |
| Typhimurium-SseB, ParaOSP-SseB, ParaOSP- | IpaB, Typhimurium-CP1 |
| IpaB, Typhimurium-CP1 | Typhimurium-IpaB, Enteritidis-IpaB, ParaOSP- |
| Typhimurium-SseB, ParaOSP-SseB, ParaOSP- | IpaB, Enteritidis-CP1 |
| IpaB, Enteritidis-CP1 | Typhimurium-IpaB, Enteritidis-IpaB, ParaOSP- |
| Typhimurium-SseB, ParaOSP-SseB, ParaOSP-IpaB, Vi- | IpaB, Vi-CP1 |
| CP1 | Typhimurium-IpaB, Enteritidis-IpaB, ParaOSP- |
| Typhimurium-SseB, ParaOSP-SseB, ParaOSP- | IpaB, ParaOSP-CP1 |
| IpaB, ParaOSP-CP1 | Typhimurium-IpaB, Enteritidis- |
| Typhimurium-SseB, ParaOSP-SseB, Typhimurium- | IpaB, Typhimurium-CP1, Enteritidis-CP1 |
| CP1, Enteritidis-CP1 | Typhimurium-IpaB, Enteritidis- |
| Typhimurium-SseB, ParaOSP-SseB, Typhimurium- | IpaB, Typhimurium-CP1, Vi-CP1 |
| CP1, Vi-CP1 | Typhimurium-IpaB, Enteritidis- |
| Typhimurium-SseB, ParaOSP-SseB, Typhimurium- | IpaB, Typhimurium-CP1, ParaOSP-CP1 |
| CP1, ParaOSP-CP1 | Typhimurium-IpaB, Enteritidis-IpaB, Enteritidis- |
| Typhimurium-SseB, ParaOSP-SseB, Enteritidis-CP1, Vi- | CP1, Vi-CP1 |
| CP1 | Typhimurium-IpaB, Enteritidis-IpaB, Enteritidis- |
| Typhimurium-SseB, ParaOSP-SseB, Enteritidis- | CP1, ParaOSP-CP1 |
| CP1, ParaOSP-CP1 | Typhimurium-IpaB, Enteritidis-IpaB, Vi- |
| Typhimurium-SseB, ParaOSP-SseB, Vi-CP1, ParaOSP- | CP1, ParaOSP-CP1 |
| CP1 | Typhimurium-IpaB, Vi-IpaB, ParaOSP- |
| Typhimurium-SseB, Typhimurium-IpaB, Enteritidis- | IpaB, Typhimurium-CP1 |
| IpaB, Vi-IpaB | Typhimurium-IpaB, Vi-IpaB, ParaOSP- |
| Typhimurium-SseB, Typhimurium-IpaB, Enteritidis- | IpaB, Enteritidis-CP1 |
| IpaB, ParaOSP-IpaB | Typhimurium-IpaB, Vi-IpaB, ParaOSP-IpaB, Vi- |
| Typhimurium-SseB, Typhimurium-IpaB, Enteritidis- | CP1 |
| IpaB, Typhimurium-CP1 | Typhimurium-IpaB, Vi-IpaB, ParaOSP- |
| Typhimurium-SseB, Typhimurium-IpaB, Enteritidis- | IpaB, ParaOSP-CP1 |
| IpaB, Enteritidis-CP1 | Typhimurium-IpaB, Vi-IpaB, Typhimurium- |
| Typhimurium-SseB, Typhimurium-IpaB, Enteritidis- | CP1, Enteritidis-CP1 |
| IpaB, Vi-CP1 | Typhimurium-IpaB, Vi-IpaB, Typhimurium- |
| Typhimurium-SseB, Typhimurium-IpaB, Enteritidis- | CP1, Vi-CP1 |
| IpaB, ParaOSP-CP1 | Typhimurium-IpaB, Vi-IpaB, Typhimurium- |
| Typhimurium-SseB, Typhimurium-IpaB, Vi- | CP1, ParaOSP-CP1 |
| IpaB, ParaOSP-IpaB | Typhimurium-IpaB, Vi-IpaB, Enteritidis-CP1, Vi- |
| Typhimurium-SseB, Typhimurium-IpaB, Vi- | CP1 |
| IpaB, Typhimurium-CP1 | Typhimurium-IpaB, Vi-IpaB, Enteritidis- |
| Typhimurium-SseB, Typhimurium-IpaB, Vi- | CP1, ParaOSP-CP1 |
| IpaB, Enteritidis-CP1 | Typhimurium-IpaB, Vi-IpaB, Vi-CP1, ParaOSP- |
| Typhimurium-SseB, Typhimurium-IpaB, Vi-IpaB, Vi- | CP1 |
| CP1 | Typhimurium-IpaB, ParaOSP-IpaB, Typhimurium- |
| Typhimurium-SseB, Typhimurium-IpaB, Vi- | CP1, Enteritidis-CP1 |
| IpaB, ParaOSP-CP1 | Typhimurium-IpaB, ParaOSP-IpaB, Typhimurium- |
| Typhimurium-SseB, Typhimurium-IpaB, ParaOSP- | CP1, Vi-CP1 |
| IpaB, Typhimurium-CP1 | Typhimurium-IpaB, ParaOSP-IpaB, Typhimurium- |
| Typhimurium-SseB, Typhimurium-IpaB, ParaOSP- | CP1, ParaOSP-CP1 |
| IpaB, Enteritidis-CP1 | Typhimurium-IpaB, ParaOSP-IpaB, Enteritidis- |
| Typhimurium-SseB, Typhimurium-IpaB, ParaOSP- | CP1, Vi-CP1 |
| IpaB, Vi-CP1 | Typhimurium-IpaB, ParaOSP-IpaB, Enteritidis- |
| Typhimurium-SseB, Typhimurium-IpaB, ParaOSP- | CP1, ParaOSP-CP1 |
| IpaB, ParaOSP-CP1 | Typhimurium-IpaB, ParaOSP-IpaB, Vi- |
| Typhimurium-SseB, Typhimurium-IpaB, Typhimurium- | CP1, ParaOSP-CP1 |
| CP1, Enteritidis-CP1 | Typhimurium-IpaB, Typhimurium- |
| Typhimurium-SseB, Typhimurium-IpaB, Typhimurium- | CP1, Enteritidis-CP1, Vi-CP1 |
| CP1, Vi-CP1 | Typhimurium-IpaB, Typhimurium- |
| Typhimurium-SseB, Typhimurium-IpaB, Typhimurium- | CP1, Enteritidis-CP1, ParaOSP-CP1 |
| CP1, ParaOSP-CP1 | Typhimurium-IpaB, Typhimurium-CP1, Vi- |
| Typhimurium-SseB, Typhimurium-IpaB, Enteritidis- | CP1, ParaOSP-CP1 |
| CP1, Vi-CP1 | Typhimurium-IpaB, Enteritidis-CP1, Vi- |
| Typhimurium-SseB, Typhimurium-IpaB, Enteritidis- | CP1, ParaOSP-CP1 |
| CP1, ParaOSP-CP1 | Vi-SseB, ParaOSP-SseB, Typhimurium- |
| Typhimurium-SseB, Typhimurium-IpaB, Vi- | IpaB, Enteritidis-IpaB |
| CP1, ParaOSP-CP1 | Vi-SseB, ParaOSP-SseB, Typhimurium-IpaB, Vi- |
| Typhimurium-SseB, Enteritidis-IpaB, Vi- | IpaB |
| IpaB, ParaOSP-IpaB | Vi-SseB, ParaOSP-SseB, Typhimurium- |
| Typhimurium-SseB, Enteritidis-IpaB, Vi- | IpaB, ParaOSP-IpaB |
| IpaB, Typhimurium-CP1 | Vi-SseB, ParaOSP-SseB, Typhimurium- |
| Typhimurium-SseB, Enteritidis-IpaB, Vi- | IpaB, Typhimurium-CP1 |
| IpaB, Enteritidis-CP1 | Vi-SseB, ParaOSP-SseB, Typhimurium- |
| Typhimurium-SseB, Enteritidis-IpaB, Vi-IpaB, Vi-CP1 | IpaB, Enteritidis-CP1 |
| Typhimurium-SseB, Enteritidis-IpaB, Vi- | Vi-SseB, ParaOSP-SseB, Typhimurium-IpaB, Vi- |
| IpaB, ParaOSP-CP1 | CP1 |
| Typhimurium-SseB, Enteritidis-IpaB, ParaOSP- | Vi-SseB, ParaOSP-SseB, Typhimurium- |
| IpaB, Typhimurium-CP1 | IpaB, ParaOSP-CP1 |
| Typhimurium-SseB, Enteritidis-IpaB, ParaOSP- | Vi-SseB, ParaOSP-SseB, Enteritidis-IpaB, Vi-IpaB |
| IpaB, Enteritidis-CP1 | Vi-SseB, ParaOSP-SseB, Enteritidis- |
| Typhimurium-SseB, Enteritidis-IpaB, ParaOSP- | IpaB, ParaOSP-IpaB |
| IpaB, Vi-CP1 | Vi-SseB, ParaOSP-SseB, Enteritidis- |
| Typhimurium-SseB, Enteritidis-IpaB, ParaOSP- | IpaB, Typhimurium-CP1 |
| IpaB, ParaOSP-CP1 | Vi-SseB, ParaOSP-SseB, Enteritidis- |
| Typhimurium-SseB, Enteritidis-IpaB, Typhimurium- | IpaB, Enteritidis-CP1 |
| CP1, Enteritidis-CP1 | Vi-SseB, ParaOSP-SseB, Enteritidis-IpaB, Vi-CP1 |
| Typhimurium-SseB, Enteritidis-IpaB, Typhimurium- | Vi-SseB, ParaOSP-SseB, Enteritidis- |
| CP1, Vi-CP1 | IpaB, ParaOSP-CP1 |
| Typhimurium-SseB, Enteritidis-IpaB, Typhimurium- | Vi-SseB, ParaOSP-SseB, Vi-IpaB, ParaOSP-IpaB |
| CP1, ParaOSP-CP1 | Vi-SseB, ParaOSP-SseB, Vi-IpaB, Typhimurium- |
| Typhimurium-SseB, Enteritidis-IpaB, Enteritidis- | CP1 |
| CP1, Vi-CP1 | Vi-SseB, ParaOSP-SseB, Vi-IpaB, Enteritidis-CP1 |
| Typhimurium-SseB, Enteritidis-IpaB, Enteritidis- | Vi-SseB, ParaOSP-SseB, Vi-IpaB, Vi-CP1 |
| CP1, ParaOSP-CP1 | Vi-SseB, ParaOSP-SseB, Vi-IpaB, ParaOSP-CP1 |
| Typhimurium-SseB, Enteritidis-IpaB, Vi-CP1, ParaOSP- | Vi-SseB, ParaOSP-SseB, ParaOSP- |
| CP1 | IpaB, Typhimurium-CP1 |
| Typhimurium-SseB, Vi-IpaB, ParaOSP- | Vi-SseB, ParaOSP-SseB, ParaOSP- |
| IpaB, Typhimurium-CP1 | IpaB, Enteritidis-CP1 |
| Typhimurium-SseB, Vi-IpaB, ParaOSP- | Vi-SseB, ParaOSP-SseB, ParaOSP-IpaB, Vi-CP1 |
| IpaB, Enteritidis-CP1 | Vi-SseB, ParaOSP-SseB, ParaOSP-IpaB, ParaOSP- |
| Typhimurium-SseB, Vi-IpaB, ParaOSP-IpaB, Vi-CP1 | CP1 |
| Typhimurium-SseB, Vi-IpaB, ParaOSP-IpaB, ParaOSP- | Vi-SseB, ParaOSP-SseB, Typhimurium- |
| CP1 | CP1, Enteritidis-CP1 |
| Typhimurium-SseB, Vi-IpaB, Typhimurium- | Vi-SseB, ParaOSP-SseB, Typhimurium-CP1, Vi- |
| CP1, Enteritidis-CP1 | CP1 |
| Typhimurium-SseB, Vi-IpaB, Typhimurium-CP1, Vi- | Vi-SseB, ParaOSP-SseB, Typhimurium- |
| CP1 | CP1, ParaOSP-CP1 |
| Typhimurium-SseB, Vi-IpaB, Typhimurium- | Vi-SseB, ParaOSP-SseB, Enteritidis-CP1, Vi-CP1 |
| CP1, ParaOSP-CP1 | Vi-SseB, ParaOSP-SseB, Enteritidis-CP1, ParaOSP- |
| Typhimurium-SseB, Vi-IpaB, Enteritidis-CP1, Vi-CP1 | CP1 |
| Typhimurium-SseB, Vi-IpaB, Enteritidis-CP1, ParaOSP- | Vi-SseB, ParaOSP-SseB, Vi-CP1, ParaOSP-CP1 |
| CP1 | Vi-SseB, Typhimurium-IpaB, Enteritidis-IpaB, Vi- |
| Typhimurium-SseB, Vi-IpaB, Vi-CP1, ParaOSP-CP1 | IpaB |
| Typhimurium-SseB, ParaOSP-IpaB, Typhimurium- | Vi-SseB, Typhimurium-IpaB, Enteritidis- |
| CP1, Enteritidis-CP1 | IpaB, ParaOSP-IpaB |
| Typhimurium-SseB, ParaOSP-IpaB, Typhimurium- | Vi-SseB, Typhimurium-IpaB, Enteritidis- |
| CP1, Vi-CP1 | IpaB, Typhimurium-CP1 |
| Typhimurium-SseB, ParaOSP-IpaB, Typhimurium- | Vi-SseB, Typhimurium-IpaB, Enteritidis- |
| CP1, ParaOSP-CP1 | IpaB, Enteritidis-CP1 |
| Typhimurium-SseB, ParaOSP-IpaB, Enteritidis-CP1, Vi- | Vi-SseB, Typhimurium-IpaB, Enteritidis-IpaB, Vi- |
| CP1 | CP1 |
| Typhimurium-SseB, ParaOSP-IpaB, Enteritidis- | Vi-SseB, Typhimurium-IpaB, Enteritidis- |
| CP1, ParaOSP-CP1 | IpaB, ParaOSP-CP1 |
| Typhimurium-SseB, ParaOSP-IpaB, Vi-CP1, ParaOSP- | Vi-SseB, Typhimurium-IpaB, Vi-IpaB, ParaOSP- |
| CP1 | IpaB |
| Typhimurium-SseB, Typhimurium-CP1, Enteritidis- | Vi-SseB, Typhimurium-IpaB, Vi- |
| CP1, Vi-CP1 | IpaB, Typhimurium-CP1 |
| Typhimurium-SseB, Typhimurium-CP1, Enteritidis- | Vi-SseB, Typhimurium-IpaB, Vi-IpaB, Enteritidis- |
| CP1, ParaOSP-CP1 | CP1 |
| Typhimurium-SseB, Typhimurium-CP1, Vi- | Vi-SseB, Typhimurium-IpaB, Vi-IpaB, Vi-CP1 |
| CP1, ParaOSP-CP1 | Vi-SseB, Typhimurium-IpaB, Vi-IpaB, ParaOSP- |
| Typhimurium-SseB, Enteritidis-CP1, Vi-CP1, ParaOSP- | CP1 |
| CP1 | Vi-SseB, Typhimurium-IpaB, ParaOSP- |
| Enteritidis-SseB, Vi-SseB, ParaOSP- | IpaB, Typhimurium-CP1 |
| SseB, Typhimurium-IpaB | Vi-SseB, Typhimurium-IpaB, ParaOSP- |
| Enteritidis-SseB, Vi-SseB, ParaOSP-SseB, Enteritidis- | IpaB, Enteritidis-CP1 |
| IpaB | Vi-SseB, Typhimurium-IpaB, ParaOSP-IpaB, Vi- |
| Enteritidis-SseB, Vi-SseB, ParaOSP-SseB, Vi-IpaB | CP1 |
| Enteritidis-SseB, Vi-SseB, ParaOSP-SseB, ParaOSP- | Vi-SseB, Typhimurium-IpaB, ParaOSP- |
| IpaB | IpaB, ParaOSP-CP1 |
| Enteritidis-SseB, Vi-SseB, ParaOSP- | Vi-SseB, Typhimurium-IpaB, Typhimurium- |
| SseB, Typhimurium-CP1 | CP1, Enteritidis-CP1 |
| Enteritidis-SseB, Vi-SseB, ParaOSP-SseB, Enteritidis- | Vi-SseB, Typhimurium-IpaB, Typhimurium- |
| CP1 | CP1, Vi-CP1 |
| Enteritidis-SseB, Vi-SseB, ParaOSP-SseB, Vi-CP1 | Vi-SseB, Typhimurium-IpaB, Typhimurium- |
| Enteritidis-SseB, Vi-SseB, ParaOSP-SseB, ParaOSP- | CP1, ParaOSP-CP1 |
| CP1 | Vi-SseB, Typhimurium-IpaB, Enteritidis-CP1, Vi- |
| Enteritidis-SseB, Vi-SseB, Typhimurium- | CP1 |
| IpaB, Enteritidis-IpaB | Vi-SseB, Typhimurium-IpaB, Enteritidis- |
| Enteritidis-SseB, Vi-SseB, Typhimurium-IpaB, Vi-IpaB | CP1, ParaOSP-CP1 |
| Enteritidis-SseB, Vi-SseB, Typhimurium- | Vi-SseB, Typhimurium-IpaB, Vi-CP1, ParaOSP- |
| IpaB, ParaOSP-IpaB | CP1 |
| Enteritidis-SseB, Vi-SseB, Typhimurium- | Vi-SseB, Enteritidis-IpaB, Vi-IpaB, ParaOSP-IpaB |
| IpaB, Typhimurium-CP1 | Vi-SseB, Enteritidis-IpaB, Vi-IpaB, Typhimurium- |
| Enteritidis-SseB, Vi-SseB, Typhimurium- | CP1 |
| IpaB, Enteritidis-CP1 | Vi-SseB, Enteritidis-IpaB, Vi-IpaB, Enteritidis-CP1 |
| Enteritidis-SseB, Vi-SseB, Typhimurium-IpaB, Vi-CP1 | Vi-SseB, Enteritidis-IpaB, Vi-IpaB, Vi-CP1 |
| Enteritidis-SseB, Vi-SseB, Typhimurium- | Vi-SseB, Enteritidis-IpaB, Vi-IpaB, ParaOSP-CP1 |
| IpaB, ParaOSP-CP1 | Vi-SseB, Enteritidis-IpaB, ParaOSP- |
| Enteritidis-SseB, Vi-SseB, Enteritidis-IpaB, Vi-IpaB | IpaB, Typhimurium-CP1 |
| Enteritidis-SseB, Vi-SseB, Enteritidis-IpaB, ParaOSP- | Vi-SseB, Enteritidis-IpaB, ParaOSP- |
| IpaB | IpaB, Enteritidis-CP1 |
| Enteritidis-SseB, Vi-SseB, Enteritidis- | Vi-SseB, Enteritidis-IpaB, ParaOSP-IpaB, Vi-CP1 |
| IpaB, Typhimurium-CP1 | Vi-SseB, Enteritidis-IpaB, ParaOSP- |
| Enteritidis-SseB, Vi-SseB, Enteritidis-IpaB, Enteritidis- | IpaB, ParaOSP-CP1 |
| CP1 | Vi-SseB, Enteritidis-IpaB, Typhimurium- |
| Enteritidis-SseB, Vi-SseB, Enteritidis-IpaB, Vi-CP1 | CP1, Enteritidis-CP1 |
| Enteritidis-SseB, Vi-SseB, Enteritidis-IpaB, ParaOSP- | Vi-SseB, Enteritidis-IpaB, Typhimurium-CP1, Vi- |
| CP1 | CP1 |
| Enteritidis-SseB, Vi-SseB, Vi-IpaB, ParaOSP-IpaB | Vi-SseB, Enteritidis-IpaB, Typhimurium- |
| Enteritidis-SseB, Vi-SseB, Vi-IpaB, Typhimurium-CP1 | CP1, ParaOSP-CP1 |
| Enteritidis-SseB, Vi-SseB, Vi-IpaB, Enteritidis-CP1 | Vi-SseB, Enteritidis-IpaB, Enteritidis-CP1, Vi-CP1 |
| Enteritidis-SseB, Vi-SseB, Vi-IpaB, Vi-CP1 | Vi-SseB, Enteritidis-IpaB, Enteritidis- |
| Enteritidis-SseB, Vi-SseB, Vi-IpaB, ParaOSP-CP1 | CP1, ParaOSP-CP1 |
| Enteritidis-SseB, Vi-SseB, ParaOSP- | Vi-SseB, Enteritidis-IpaB, Vi-CP1, ParaOSP-CP1 |
| IpaB, Typhimurium-CP1 | Vi-SseB, Vi-IpaB, ParaOSP-IpaB, Typhimurium- |
| Enteritidis-SseB, Vi-SseB, ParaOSP-IpaB, Enteritidis- | CP1 |
| CP1 | Vi-SseB, Vi-IpaB, ParaOSP-IpaB, Enteritidis-CP1 |
| Enteritidis-SseB, Vi-SseB, ParaOSP-IpaB, Vi-CP1 | Vi-SseB, Vi-IpaB, ParaOSP-IpaB, Vi-CP1 |
| Enteritidis-SseB, Vi-SseB, ParaOSP-IpaB, ParaOSP-CP1 | Vi-SseB, Vi-IpaB, ParaOSP-IpaB, ParaOSP-CP1 |
| Enteritidis-SseB, Vi-SseB, Typhimurium- | Vi-SseB, Vi-IpaB, Typhimurium-CP1, Enteritidis- |
| CP1, Enteritidis-CP1 | CP1 |
| Enteritidis-SseB, Vi-SseB, Typhimurium-CP1, Vi-CP1 | Vi-SseB, Vi-IpaB, Typhimurium-CP1, Vi-CP1 |
| Enteritidis-SseB, Vi-SseB, Typhimurium-CP1, ParaOSP- | Vi-SseB, Vi-IpaB, Typhimurium-CP1, ParaOSP- |
| CP1 | CP1 |
| Enteritidis-SseB, Vi-SseB, Enteritidis-CP1, Vi-CP1 | Vi-SseB, Vi-IpaB, Enteritidis-CP1, Vi-CP1 |
| Enteritidis-SseB, Vi-SseB, Enteritidis-CP1, ParaOSP- | Vi-SseB, Vi-IpaB, Enteritidis-CP1, ParaOSP-CP1 |
| CP1 | Vi-SseB, Vi-IpaB, Vi-CP1, ParaOSP-CP1 |
| Enteritidis-SseB, Vi-SseB, Vi-CP1, ParaOSP-CP1 | Vi-SseB, ParaOSP-IpaB, Typhimurium- |
| Enteritidis-SseB, ParaOSP-SseB, Typhimurium- | CP1, Enteritidis-CP1 |
| IpaB, Enteritidis-IpaB | Vi-SseB, ParaOSP-IpaB, Typhimurium-CP1, Vi- |
| Enteritidis-SseB, ParaOSP-SseB, Typhimurium- | CP1 |
| IpaB, Vi-IpaB | Vi-SseB, ParaOSP-IpaB, Typhimurium- |
| Enteritidis-SseB, ParaOSP-SseB, Typhimurium- | CP1, ParaOSP-CP1 |
| IpaB, ParaOSP-IpaB | Vi-SseB, ParaOSP-IpaB, Enteritidis-CP1, Vi-CP1 |
| Enteritidis-SseB, ParaOSP-SseB, Typhimurium- | Vi-SseB, ParaOSP-IpaB, Enteritidis-CP1, ParaOSP- |
| IpaB, Typhimurium-CP1 | CP1 |
| Enteritidis-SseB, ParaOSP-SseB, Typhimurium- | Vi-SseB, ParaOSP-IpaB, Vi-CP1, ParaOSP-CP1 |
| IpaB, Enteritidis-CP1 | Vi-SseB, Typhimurium-CP1, Enteritidis-CP1, Vi- |
| Enteritidis-SseB, ParaOSP-SseB, Typhimurium- | CP1 |
| IpaB, Vi-CP1 | Vi-SseB, Typhimurium-CP1, Enteritidis- |
| Enteritidis-SseB, ParaOSP-SseB, Typhimurium- | CP1, ParaOSP-CP1 |
| IpaB, ParaOSP-CP1 | Vi-SseB, Typhimurium-CP1, Vi-CP1, ParaOSP- |
| Enteritidis-SseB, ParaOSP-SseB, Enteritidis-IpaB, Vi- | CP1 |
| IpaB | Vi-SseB, Enteritidis-CP1, Vi-CP1, ParaOSP-CP1 |
| Enteritidis-SseB, ParaOSP-SseB, Enteritidis- | Enteritidis-SseB, ParaOSP-IpaB, Typhimurium- |
| IpaB, ParaOSP-IpaB | CP1, Enteritidis-CP1 |
| Enteritidis-SseB, ParaOSP-SseB, Enteritidis- | Enteritidis-SseB, ParaOSP-IpaB, Typhimurium- |
| IpaB, Typhimurium-CP1 | CP1, Vi-CP1 |
| Enteritidis-SseB, ParaOSP-SseB, Enteritidis- | Enteritidis-SseB, ParaOSP-IpaB, Typhimurium- |
| IpaB, Enteritidis-CP1 | CP1, ParaOSP-CP1 |
| Enteritidis-SseB, ParaOSP-SseB, Enteritidis-IpaB, Vi- | Enteritidis-SseB, ParaOSP-IpaB, Enteritidis- |
| CP1 | CP1, Vi-CP1 |
| Enteritidis-SseB, ParaOSP-SseB, Enteritidis- | Enteritidis-SseB, ParaOSP-IpaB, Enteritidis- |
| IpaB, ParaOSP-CP1 | CP1, ParaOSP-CP1 |
| Enteritidis-SseB, ParaOSP-SseB, Vi-IpaB, ParaOSP- | Enteritidis-SseB, ParaOSP-IpaB, Vi-CP1, ParaOSP- |
| IpaB | CP1 |
| Enteritidis-SseB, ParaOSP-SseB, Vi- | Enteritidis-SseB, Typhimurium-CP1, Enteritidis- |
| IpaB, Typhimurium-CP1 | CP1, Vi-CP1 |
| Enteritidis-SseB, ParaOSP-SseB, Vi-IpaB, Enteritidis- | Enteritidis-SseB, Typhimurium-CP1, Enteritidis- |
| CP1 | CP1, ParaOSP-CP1 |
| Enteritidis-SseB, ParaOSP-SseB, Vi-IpaB, Vi-CP1 | Enteritidis-SseB, Typhimurium-CP1, Vi- |
| Enteritidis-SseB, ParaOSP-SseB, Vi-IpaB, ParaOSP-CP1 | CP1, ParaOSP-CP1 |
| Enteritidis-SseB, ParaOSP-SseB, ParaOSP- | Enteritidis-SseB, Enteritidis-CP1, Vi- |
| IpaB, Typhimurium-CP1 | CP1, ParaOSP-CP1 |
| Enteritidis-SseB, ParaOSP-SseB, ParaOSP- | Enteritidis-SseB, Typhimurium-IpaB, Enteritidis- |
| IpaB, Enteritidis-CP1 | IpaB, Typhimurium-CP1 |
| Enteritidis-SseB, ParaOSP-SseB, ParaOSP-IpaB, Vi-CP1 | Enteritidis-SseB, Typhimurium-IpaB, Enteritidis- |
| Enteritidis-SseB, ParaOSP-SseB, ParaOSP- | IpaB, Enteritidis-CP1 |
| IpaB, ParaOSP-CP1 | Enteritidis-SseB, Typhimurium-IpaB, Enteritidis- |
| Enteritidis-SseB, ParaOSP-SseB, Typhimurium- | IpaB, Vi-CP1 |
| CP1, Enteritidis-CP1 | Enteritidis-SseB, Typhimurium-IpaB, Enteritidis- |
| Enteritidis-SseB, ParaOSP-SseB, Typhimurium-CP1, Vi- | IpaB, ParaOSP-CP1 |
| CP1 | Enteritidis-SseB, Typhimurium-IpaB, Vi- |
| Enteritidis-SseB, ParaOSP-SseB, Typhimurium- | IpaB, ParaOSP-IpaB |
| CP1, ParaOSP-CP1 | Enteritidis-SseB, Typhimurium-IpaB, Vi- |
| Enteritidis-SseB, ParaOSP-SseB, Enteritidis-CP1, Vi- | IpaB, Typhimurium-CP1 |
| CP1 | Enteritidis-SseB, Typhimurium-IpaB, Vi- |
| Enteritidis-SseB, ParaOSP-SseB, Enteritidis- | IpaB, Enteritidis-CP1 |
| CP1, ParaOSP-CP1 | Enteritidis-SseB, Typhimurium-IpaB, Vi-IpaB, Vi- |
| Enteritidis-SseB, ParaOSP-SseB, Vi-CP1, ParaOSP-CP1 | CP1 |
| Enteritidis-SseB, Typhimurium-IpaB, Enteritidis- | Enteritidis-SseB, Typhimurium-IpaB, Vi- |
| IpaB, Vi-IpaB | IpaB, ParaOSP-CP1 |
| Enteritidis-SseB, Typhimurium-IpaB, Enteritidis- | Enteritidis-SseB, Typhimurium-IpaB, ParaOSP- |
| IpaB, ParaOSP-IpaB | IpaB, Typhimurium-CP1 |
| Enteritidis-SseB, Typhimurium-IpaB, Enteritidis- | Enteritidis-SseB, Typhimurium-IpaB, ParaOSP- |
| IpaB, Typhimurium-CP1 | IpaB, Enteritidis-CP1 |
| Enteritidis-SseB, Typhimurium-IpaB, Enteritidis- | Enteritidis-SseB, Typhimurium-IpaB, ParaOSP- |
| IpaB, Enteritidis-CP1 | IpaB, Vi-CP1 |
| Enteritidis-SseB, Typhimurium-IpaB, Enteritidis- | Enteritidis-SseB, Typhimurium-IpaB, ParaOSP- |
| IpaB, Vi-CP1 | IpaB, ParaOSP-CP1 |
| Enteritidis-SseB, Typhimurium-IpaB, Enteritidis- | Enteritidis-SseB, Typhimurium- |
| IpaB, ParaOSP-CP1 | IpaB, Typhimurium-CP1, Enteritidis-CP1 |
| Enteritidis-SseB, Typhimurium-IpaB, Vi- | Enteritidis-SseB, Typhimurium- |
| IpaB, ParaOSP-IpaB | IpaB, Typhimurium-CP1, Vi-CP1 |
| Enteritidis-SseB, Typhimurium-IpaB, Vi- | Enteritidis-SseB, Typhimurium- |
| IpaB, Typhimurium-CP1 | IpaB, Typhimurium-CP1, ParaOSP-CP1 |
| Enteritidis-SseB, Typhimurium-IpaB, Vi- | Enteritidis-SseB, Typhimurium-IpaB, Enteritidis- |
| IpaB, Enteritidis-CP1 | CP1, Vi-CP1 |
| Enteritidis-SseB, Typhimurium-IpaB, Vi-IpaB, Vi-CP1 | Enteritidis-SseB, Typhimurium-IpaB, Enteritidis- |
| Enteritidis-SseB, Typhimurium-IpaB, Vi- | CP1, ParaOSP-CP1 |
| IpaB, ParaOSP-CP1 | Enteritidis-SseB, Typhimurium-IpaB, Vi- |
| Enteritidis-SseB, Typhimurium-IpaB, ParaOSP- | CP1, ParaOSP-CP1 |
| IpaB, Typhimurium-CP1 | Enteritidis-SseB, Enteritidis-IpaB, Vi- |
| Enteritidis-SseB, Typhimurium-IpaB, ParaOSP- | IpaB, ParaOSP-IpaB |
| IpaB, Enteritidis-CP1 | Enteritidis-SseB, Enteritidis-IpaB, Vi- |
| Enteritidis-SseB, Typhimurium-IpaB, ParaOSP- | IpaB, Typhimurium-CP1 |
| IpaB, Vi-CP1 | Enteritidis-SseB, Enteritidis-IpaB, Vi- |
| Enteritidis-SseB, Typhimurium-IpaB, ParaOSP- | IpaB, Enteritidis-CP1 |
| IpaB, ParaOSP-CP1 | Enteritidis-SseB, Enteritidis-IpaB, Vi-IpaB, Vi-CP1 |
| Enteritidis-SseB, Typhimurium-IpaB, Typhimurium- | Enteritidis-SseB, Enteritidis-IpaB, Vi- |
| CP1, Enteritidis-CP1 | IpaB, ParaOSP-CP1 |
| Enteritidis-SseB, Typhimurium-IpaB, Typhimurium- | Enteritidis-SseB, Enteritidis-IpaB, ParaOSP- |
| CP1, Vi-CP1 | IpaB, Typhimurium-CP1 |
| Enteritidis-SseB, Typhimurium-IpaB, Typhimurium- | Enteritidis-SseB, Enteritidis-IpaB, ParaOSP- |
| CP1, ParaOSP-CP1 | IpaB, Enteritidis-CP1 |
| Enteritidis-SseB, Typhimurium-IpaB, Enteritidis- | Enteritidis-SseB, Enteritidis-IpaB, ParaOSP- |
| CP1, Vi-CP1 | IpaB, Vi-CP1 |
| Enteritidis-SseB, Typhimurium-IpaB, Enteritidis- | Enteritidis-SseB, Enteritidis-IpaB, ParaOSP- |
| CP1, ParaOSP-CP1 | IpaB, ParaOSP-CP1 |
| Enteritidis-SseB, Typhimurium-IpaB, Vi-CP1, ParaOSP- | Enteritidis-SseB, Enteritidis-IpaB, Typhimurium- |
| CP1 | CP1, Enteritidis-CP1 |
| Enteritidis-SseB, Enteritidis-IpaB, Vi-IpaB, ParaOSP- | Enteritidis-SseB, Enteritidis-IpaB, Typhimurium- |
| IpaB | CP1, Vi-CP1 |
| Enteritidis-SseB, Enteritidis-IpaB, Vi- | Enteritidis-SseB, Enteritidis-IpaB, Typhimurium- |
| IpaB, Typhimurium-CP1 | CP1, ParaOSP-CP1 |
| Enteritidis-SseB, Enteritidis-IpaB, Vi-IpaB, Enteritidis- | Enteritidis-SseB, Enteritidis-IpaB, Enteritidis- |
| CP1 | CP1, Vi-CP1 |
| Enteritidis-SseB, Enteritidis-IpaB, Vi-IpaB, Vi-CP1 | Enteritidis-SseB, Enteritidis-IpaB, Enteritidis- |
| Enteritidis-SseB, Enteritidis-IpaB, Vi-IpaB, ParaOSP- | CP1, ParaOSP-CP1 |
| CP1 | Enteritidis-SseB, Enteritidis-IpaB, Vi- |
| Enteritidis-SseB, Enteritidis-IpaB, ParaOSP- | CP1, ParaOSP-CP1 |
| IpaB, Typhimurium-CP1 | Enteritidis-SseB, Vi-IpaB, ParaOSP- |
| Enteritidis-SseB, Enteritidis-IpaB, ParaOSP- | IpaB, Typhimurium-CP1 |
| IpaB, Enteritidis-CP1 | Enteritidis-SseB, Vi-IpaB, ParaOSP- |
| Enteritidis-SseB, Enteritidis-IpaB, ParaOSP-IpaB, Vi- | IpaB, Enteritidis-CP1 |
| CP1 | Enteritidis-SseB, Vi-IpaB, ParaOSP-IpaB, Vi-CP1 |
| Enteritidis-SseB, Enteritidis-IpaB, ParaOSP- | Enteritidis-SseB, Vi-IpaB, ParaOSP- |
| IpaB, ParaOSP-CP1 | IpaB, ParaOSP-CP1 |
| Enteritidis-SseB, Enteritidis-IpaB, Typhimurium- | Enteritidis-SseB, Vi-IpaB, Typhimurium- |
| CP1, Enteritidis-CP1 | CP1, Enteritidis-CP1 |
| Enteritidis-SseB, Enteritidis-IpaB, Typhimurium- | Enteritidis-SseB, Vi-IpaB, Typhimurium-CP1, Vi- |
| CP1, Vi-CP1 | CP1 |
| Enteritidis-SseB, Enteritidis-IpaB, Typhimurium- | Enteritidis-SseB, Vi-IpaB, Typhimurium- |
| CP1, ParaOSP-CP1 | CP1, ParaOSP-CP1 |
| Enteritidis-SseB, Enteritidis-IpaB, Enteritidis-CP1, Vi- | Enteritidis-SseB, Vi-IpaB, Enteritidis-CP1, Vi-CP1 |
| CP1 | Enteritidis-SseB, Vi-IpaB, Enteritidis- |
| Enteritidis-SseB, Enteritidis-IpaB, Enteritidis- | CP1, ParaOSP-CP1 |
| CP1, ParaOSP-CP1 | Enteritidis-SseB, Vi-IpaB, Vi-CP1, ParaOSP-CP1 |
| Enteritidis-SseB, Enteritidis-IpaB, Vi-CP1, ParaOSP- | Enteritidis-SseB, ParaOSP-IpaB, Typhimurium- |
| CP1 | CP1, Enteritidis-CP1 |
| Enteritidis-SseB, Vi-IpaB, ParaOSP- | Enteritidis-SseB, ParaOSP-IpaB, Typhimurium- |
| IpaB, Typhimurium-CP1 | CP1, Vi-CP1 |
| Enteritidis-SseB, Vi-IpaB, ParaOSP-IpaB, Enteritidis- | Enteritidis-SseB, ParaOSP-IpaB, Typhimurium- |
| CP1 | CP1, ParaOSP-CP1 |
| Enteritidis-SseB, Vi-IpaB, ParaOSP-IpaB, Vi-CP1 | Enteritidis-SseB, ParaOSP-IpaB, Enteritidis- |
| Enteritidis-SseB, Vi-IpaB, ParaOSP-IpaB, ParaOSP-CP1 | CP1, Vi-CP1 |
| Enteritidis-SseB, Vi-IpaB, Typhimurium- | Enteritidis-SseB, ParaOSP-IpaB, Enteritidis- |
| CP1, Enteritidis-CP1 | CP1, ParaOSP-CP1 |
| Enteritidis-SseB, Vi-IpaB, Typhimurium-CP1, Vi-CP1 | Enteritidis-SseB, ParaOSP-IpaB, Vi-CP1, ParaOSP- |
| Enteritidis-SseB, Vi-IpaB, Typhimurium-CP1, ParaOSP- | CP1 |
| CP1 | Enteritidis-SseB, Typhimurium-CP1, Enteritidis- |
| Enteritidis-SseB, Vi-IpaB, Enteritidis-CP1, Vi-CP1 | CP1, Vi-CP1 |
| Enteritidis-SseB, Vi-IpaB, Enteritidis-CP1, ParaOSP- | Enteritidis-SseB, Typhimurium-CP1, Enteritidis- |
| CP1 | CP1, ParaOSP-CP1 |
| Enteritidis-SseB, Vi-IpaB, Vi-CP1, ParaOSP-CP1 | Enteritidis-SseB, Typhimurium-CP1, Vi- |
| Enteritidis-SseB, ParaOSP-IpaB, Typhimurium- | CP1, ParaOSP-CP1 |
| CP1, Enteritidis-CP1 | Enteritidis-SseB, Enteritidis-CP1, Vi- |
| Enteritidis-SseB, ParaOSP-IpaB, Typhimurium-CP1, Vi- | CP1, ParaOSP-CP1 |
| CP1 | |
| Enteritidis-SseB, ParaOSP-IpaB, Typhimurium- | |
| CP1, ParaOSP-CP1 | |
| Enteritidis-SseB, ParaOSP-IpaB, Enteritidis-CP1, Vi- | |
| CP1 | |
| Enteritidis-SseB, ParaOSP-IpaB, Enteritidis- | |
| CP1, ParaOSP-CP1 | |
| Enteritidis-SseB, ParaOSP-IpaB, Vi-CP1, ParaOSP-CP1 | |
| Enteritidis-SseB, Typhimurium-CP1, Enteritidis- | |
| CP1, Vi-CP1 | |
| Enteritidis-SseB, Typhimurium-CP1, Enteritidis- | |
| CP1, ParaOSP-CP1 | |
| Enteritidis-SseB, Typhimurium-CP1, Vi-CP1, ParaOSP- | |
| CP1 | |
| Enteritidis-SseB, Enteritidis-CP1, Vi-CP1, ParaOSP-CP1 | |
| Enteritidis-SseB, Enteritidis-IpaB, Typhimurium- | |
| CP1, Vi-CP1 | |
| Enteritidis-SseB, Enteritidis-IpaB, Typhimurium- | |
| CP1, ParaOSP-CP1 | |
| Enteritidis-SseB, Enteritidis-IpaB, Enteritidis-CP1, Vi- | |
| CP1 | |
| Enteritidis-SseB, Enteritidis-IpaB, Enteritidis- | |
| CP1, ParaOSP-CP1 | |
| Enteritidis-SseB, Enteritidis-IpaB, Vi-CP1, ParaOSP- | |
| CP1 | |
| Enteritidis-SseB, Vi-IpaB, ParaOSP- | |
| IpaB, Typhimurium-CP1 | |
| Enteritidis-SseB, Vi-IpaB, ParaOSP-IpaB, Enteritidis- | |
| CP1 | |
| Enteritidis-SseB, Vi-IpaB, ParaOSP-IpaB, Vi-CP1 | |
| Enteritidis-SseB, Vi-IpaB, ParaOSP-IpaB, ParaOSP-CP1 | |
| Enteritidis-SseB, Vi-IpaB, Typhimurium- | |
| CP1, Enteritidis-CP1 | |
| Enteritidis-SseB, Vi-IpaB, Typhimurium-CP1, Vi-CP1 | |
| Enteritidis-SseB, Vi-IpaB, Typhimurium-CP1, ParaOSP- | |
| CP1 | |
| Enteritidis-SseB, Vi-IpaB, Enteritidis-CP1, Vi-CP1 | |
| Enteritidis-SseB, Vi-IpaB, Enteritidis-CP1, ParaOSP- | |
| CP1 | |
| Enteritidis-SseB, Vi-IpaB, Vi-CP1, ParaOSP-CP1 | |

### Conjugated Immunogenic Complexes; Immunogenic and Vaccine Compositions Comprising Same

In some embodiments, one or more polypeptides (e.g., antigenic polypeptides) of immunogenic complexes are conjugated to one or more polysaccharides. In some embodiments, one or more conjugated polysaccharides comprise a O specific polysaccharide of Shigella. In some embodiments, one or more polypeptides of conjugated immunogenic complex comprise an antigenic polypeptide of S. enterica, S. pneumoniae, or Shigella flexneri. In some embodiments, an antigenic polypeptide of a conjugated immunogenic complex is or comprises a fusion protein. In some such embodiments, a fusion protein of a conjugated immunogenic complex is or comprises at least one of: SseB, IpaB, or the CP1 fusion protein.

### Uses of Immunogenic and Vaccine Compositions

In some embodiments, an immunogenic complex described herein that includes one or more antigenic polysaccharides is characterized in that one or more of the opsonization potential, or immune response to one or more antigenic polysaccharides is increased relative to a predetermined level, as measured by ELISA and or by a functional antibody assay. In some embodiments, one or more of the opsonization potential, immune response to the one or more antigenic polysaccharides is increased at least 1-fold, 2-fold, 3-fold, 4-fold, or 5-fold relative to a predetermined level, as measured by ELISA and or by a functional antibody assay. In some embodiments, the predetermined level is a pre-immune level. In some embodiments, the predetermined level is a pre-immune level. In some embodiments, one or more polypeptide antigens are carrier proteins for one or more antigenic polysaccharides.

In some embodiments, an immunogenic complex described herein, upon administration to a subject, induces an immune response against one or more pathogens in the subject at a level greater than a composition comprising an antigenic polysaccharide alone. In some embodiments, an immunogenic complex described herein, upon administration to a subject, induces an immune response against one or more pathogens in the subject at a level greater than a composition comprising a polypeptide antigen alone. In some embodiments, an immunogenic complex described herein, upon administration to a subject, induces a protective immune response.

In some embodiments, an immunogenic complex described herein, upon administration to a subject, induces an immune response against Shigella. In some embodiments, an immunogenic complex described herein, upon administration to a subject, induces an immune response against one or more serotypes of Shigella. In some embodiments, such an immune response may be directed against one or more (e.g., 1, 2, 3, 4, 5, 6, 7, 8, 9, 10 or more) serotypes of Shigella, wherein an immunogenic complex described herein includes polysaccharide(s) present in at least one or more (e.g., 1, 2, 3, 4, 5, 6, 7, 8, 9, 10 or more) of such serotype(s). In some embodiments, such an immune response may be directed against one or more (e.g., 1, 2, 3, 4, 5, 6, 7, 8, 9, 10 or more) serotypes of Shigella, wherein an immunogenic complex described herein does not include polysaccharide(s) present in at least one or more (e.g., 1, 2, 3, 4, 5, 6, 7, 8, 9, 10 or more) of such serotype(s). In some embodiments, such an immune response may be directed against two or more (e.g., 2, 3, 4, 5, 6, 7, 8, 9, 10 or more) serotypes of Shigella, wherein an immunogenic complex described herein (i) includes polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s). In some embodiments, an immunogenic complex described herein, upon administration to a subject, induces a protective immune response against one or more serotypes of Shigella. In some embodiments, such a protective response may be directed against one or more (e.g., 1, 2, 3, 4, 5, 6, 7, 8, 9, 10 or more) serotypes of Shigella, wherein an immunogenic complex described herein includes polysaccharide(s) present in at least one or more (e.g., 1, 2, 3, 4, 5, 6, 7, 8, 9, 10 or more) of such serotype(s). In some embodiments, such a protective response may be directed against one or more (e.g., 1, 2, 3, 4, 5, 6, 7, 8, 9, 10 or more) serotypes of Shigella, wherein an immunogenic complex described herein does not include polysaccharide(s) present in at least one or more (e.g., 1, 2, 3, 4, 5, 6, 7, 8, 9, 10 or more) of such serotype(s). In some embodiments, such a protective response may be directed against two or more (e.g., 2, 3, 4, 5, 6, 7, 8, 9, 10 or more) serotypes of Shigella, wherein an immunogenic complex described herein (i) includes polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s).

In some embodiments, the immune response is an antibody or B cell response. In some embodiments, the immune response is a T cell response. In some embodiments, the immune response is an innate immune response. In some embodiments, the immune response is a CD4+ T cell response, including Tₕ1, Tₕ2, or Tₕ17 response, or a CD8+ T cell response, or a CD4+ and CD8+ T cell response, or a CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response.

In some embodiments, the immune response is to the polysaccharide. In some embodiments, the immune response is to the antigenic polypeptide (also referred to as a carrier protein), e.g., to any one or more of antigenic polypeptides SseB, IpaB, SP1500 or SP785 in the immunogenic composition. In some embodiments, there is an immune response is to the polysaccharide and to the antigenic polypeptide (also referred to as a carrier protein), e.g., to any one or more of antigenic polypeptides SseB, IpaB, SP1500 or SP785 in the immunogenic composition.

In some embodiments, an immunogenic complex described herein, upon administration to a subject, induces antibody production against one or more pathogens in the subject at a level greater than a composition comprising an antigenic polysaccharide alone. In some embodiments, an immunogenic complex described herein, upon administration to a subject, induces antibody production against one or more pathogens in the subject at level greater than a composition comprising a polypeptide antigen alone.

In some embodiments, an immunogenic composition or vaccine described herein, upon administration to a subject, induces an immune response against one or more pathogens in the subject at a level greater than a composition comprising an antigenic polysaccharide alone. In some embodiments, an immunogenic composition or vaccine described herein, upon administration to a subject, induces an immune response against one or more pathogens in the subject at a level greater than a composition comprising a polypeptide antigen alone. In some embodiments, an immunogenic complex described herein, upon administration to a subject, induces a protective immune response.

The Shigella immunogenic compositions and vaccines described herein may be used for prophylactic and/or therapeutic treatment of Shigella. Accordingly, this application provides a method for immunizing a subject suffering from or susceptible to Shigella infection, comprising administering an immunologically effective amount of any of the immunogenic compositions or vaccine formulations described herein. The subject receiving the vaccination may be a male or a female, and may be an infant, child, adolescent, or adult. In some embodiments, the subject being treated is a human. In other embodiments, the subject is a non-human animal. In some embodiments, an immunogenic complex described herein, upon administration to a subject, induces a protective immune response against one or more serotypes of Shigella.

In prophylactic embodiments, a vaccine composition (e.g., ones as described and/or utilized herein) is administered to a subject to induce an immune response that can help protect against the establishment of Shigella, for example by protecting against colonization, the first and necessary step in disease. In some embodiments, such an immune response may be directed against one or more (e.g., 1, 2, 3, 4, 5, 6, 7, 8, 9, 10 or more) serotypes of Shigella, wherein a vaccine composition described herein includes polysaccharide(s) present in at least one or more (e.g., 1, 2, 3, 4, 5, 6, 7, 8, 9, 10 or more) of such serotype(s). In some embodiments, such an immune response may be directed against one or more (e.g., 1, 2, 3, 4, 5, 6, 7, 8, 9, 10 or more) serotypes of Shigella, wherein a vaccine composition described herein does not include polysaccharide(s) present in at least one or more (e.g., 1, 2, 3, 4, 5, 6, 7, 8, 9, 10 or more) of such serotype(s) (non-vaccine types, NVTs). In some embodiments, such an immune response may be directed against two or more (e.g., 2, 3, 4, 5, 6, 7, 8, 9, 10 or more) serotypes of Shigella, wherein a vaccine composition described herein (i) includes polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s). Thus, in some aspects, the method inhibits infection by Shigella in a noncolonized or uninfected subject. In another aspect, the method may reduce the duration of colonization in a subject who is already colonized.

In therapeutic embodiments, the vaccine may be administered to a subject suffering from Shigella infection, in an amount sufficient to treat the subject. Treating the subject, in this case, refers to reducing Shigella symptoms and/or bacterial load and/or sequelae in an infected subject. In some embodiments, treating the subject refers to reducing the duration of symptoms or sequelae, or reducing the intensity of symptoms or sequelae. In some embodiments, the vaccine reduces transmissibility of Shigella from the vaccinated subject. In certain embodiments, the reductions described above are at least 25%, 30%, 40%, 50%, 60%, 70%, 80%, or 90%.

In therapeutic embodiments, the vaccine is administered to a subject post infection. The vaccine may be administered shortly after infection, e.g. before symptoms or sequelae manifest, or may be administered during or after manifestation of symptoms or sequelae.

In some embodiments, the vaccine compositions of the invention confer protective immunity, allowing a vaccinated subject to exhibit delayed onset of symptoms or sequelae, or reduced severity of symptoms or sequelae, as the result of his or her exposure to the vaccine. In certain embodiments, the reduction in severity of symptoms or sequelae is at least 25%, 40%, 50%, 60%, 70%, 80%, or 90%. In particular embodiments, vaccinated subjects may display no symptoms or sequelae upon contact with Shigella, do not become colonized by Shigella, or both. Protective immunity is typically achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes, that result in sIGA production by B lymphocytes on mucosa-lined tissues of the body. Humoral immunity is typically the result of IgG antibodies and IgM antibodies in serum. Cellular immunity can be achieved through cytotoxic T lymphocytes or through delayed-type hypersensitivity that involves macrophages and T lymphocytes, as well as other mechanisms involving T cells without a requirement for antibodies. In particular, cellular immunity may be mediated by Tₕ1 or Tₕ17 cells.

In some embodiments, an immunogenic composition or vaccine described herein, upon administration to a subject, induces an immune response against Shigella. In some embodiments, an immunogenic composition or vaccine described herein, upon administration to a subject, induces an immune response against one or more (e.g., 1, 2, 3, 4, 5, 6, 7, 8, 9, 10, 11, 12, 13, 14, 15, 16, 17, 18, 19, 20, 21, 22, 23, 24, or more) serotypes of Shigella. In some embodiments, an immunogenic composition or vaccine described herein, upon administration to a subject, induces an immune response against all serotypes of Shigella comprised in such immunogenic composition or vaccine. In some embodiments, an immunogenic complex described herein, upon administration to a subject, induces a protective immune response against one or more (e.g., 1,2,3,4, 5, 6, 7, 8, 9, 10, 11, 12, 13, 14, 15, 16, 17, 18, 19, 20,21,22, 23,24, or more) serotypes of Shigella. In some embodiments, an immunogenic complex described herein, upon administration to a subject, induces a protective immune response against all serotypes of Shigella comprised in such immunogenic composition or vaccine.

In some embodiments, the immune response is an antibody or B cell response. In some embodiments, the immune response is a T cell response. In some embodiments, the immune response is an innate immune response. In some embodiments, the immune response is a CD4+ T cell response, including Tₕ1, Tₕ2, or Tₕ17 response, or a CD8+ T cell response, or a CD4+ and CD8+ T cell response, or CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response.

In some embodiments, an immunogenic composition or vaccine described herein, upon administration to a subject, induces an antibody or B cell response against one or more pathogens in the subject at a level greater than a composition comprising an antigenic polysaccharide alone. In some embodiments, an immunogenic composition or vaccine described herein, upon administration to a subject, induces an antibody or B cell response against one or more pathogens in the subject at level greater than a composition comprising a polypeptide antigen alone. In some embodiments, the immune response is a protective immune response.

In some embodiments, an immunogenic composition or vaccine described herein, upon administration to a subject, induces a T cell response against one or more pathogens in the subject at a level greater than a composition comprising an antigenic polysaccharide alone. In some embodiments, an immunogenic composition or vaccine described herein, upon administration to a subject, induces a T cell response against one or more pathogens in the subject at level greater than a composition comprising a polypeptide antigen alone. In some embodiments, the immune response is a protective immune response.

In some embodiments, upon administration to a subject, an immunogenic composition or vaccine described herein treats or prevents infection by Shigella. In some embodiments, upon administration to a subject, an immunogenic composition or vaccine described herein inhibits or reduces the rate of occurrence of infection by Shigella. In some embodiments, upon administration to a subject, an immunogenic composition or vaccine described herein reduces the severity of infection by Shigella. In some embodiments, upon administration to a subject, an immunogenic composition or vaccine described herein inhibits transmission of Shigella from the subject to another subject.

In some embodiments, an immunogenic composition or vaccine described herein, upon administration to a subject, elicits immunogenicity against one or more of Shigella serotypes selected from Shigella flexneri or Shigella sonnei, e.g., Shigella flexneri serotype selected from: 2a, 3a or 6, or Shigella sonnei.

In some embodiments, a fusion protein described herein does not have, or has minimal, hemolytic activity. For example, in some embodiments, the hemolytic activity of a fusion protein described herein can be established by turbidimetry (OD₄₂₀) after incubation of the fusion protein at different dilutions with red blood cells (e.g., sheep erythrocytes), to determine the protein concentration at which 50% of the red blood cells are lysed. In some such embodiments, the hemolytic activity of a fusion protein described herein can be characterized by an OD₄₂₀ of less than 0.4 or lower, including, e.g., less than 0.3, less than 0.25, less than 0.2, or lower, for a given protein concentration.

In some embodiments, polypeptides of Shigella and fusion proteins described herein, and fragments and variants thereof, are immunogenic. These polypeptides and fusion proteins may be immunogenic in mammals, for example mice, rats, guinea pigs, or humans. An antigenic polypeptide or fusion protein is typically one capable of raising a significant immune response in an assay or in a subject. The immune response may be innate, humoral, cell-mediated, or mucosal (combining elements of innate, humoral and cell mediated immunity). For instance, an antigenic polypeptide or fusion protein may increase the amount of IL-17 produced by T cells. Alternatively or additionally, an antigenic polypeptide or fusion protein may (i) induce production of antibodies, e.g., neutralizing antibodies, that bind to the polypeptide and/or the whole bacteria, (ii) induce Tₕ17 immunity, (iii) activate the CD4+ T cell response, for example by increasing the number of CD4+ T cells and/or increasing localization of CD4+ T cells to the site of infection or reinfection, (iv) activate the CD8+ T cell response, for example by increasing the number of CD8+ T cells and/or increasing localization of CD8+ T cells to the site of infection or reinfection, (v) activate both the CD4+ and the CD8+ response, (vi) activate CD4−/CD8− immunity, (vii) induce Tₕ1 immunity, (viii) induce anti-microbial peptides, (ix) activate innate immunity, or any combination of the foregoing. In some embodiments, an antigenic polypeptide or fusion protein elicits production of a detectable amount of antibody specific to that antigen.

In some embodiments, a fusion protein described herein is an antigen or has antigenic properties. In some embodiments, a fusion protein described herein is a carrier protein or has carrier properties. In some embodiments, a fusion protein described herein is both an antigen and a carrier protein. In some embodiments, a fusion protein described herein has both carrier properties and antigenic properties.

In some embodiments, a fusion protein described herein is an antigen of an immunogenic complex (e.g., a Multiple Antigen Presenting System (MAPS) complex as described in WO 2012/155007 and in U.S. Pat. No. 11,013,793, the entire contents of which are incorporated herein by reference for the purposes indicated herein). In some embodiments, a fusion protein described herein is a carrier protein of an immunogenic complex. In some embodiments, a fusion protein described herein is both a carrier protein and an antigen of an immunogenic complex.

In some embodiments, polypeptides of the fusion proteins described herein have less than 20%, 30%, 40%, 50%, 60% or 70% identity to human auto-antigens and/or gut commensal bacteria (e.g., certain Bacteroides, Clostridium, Fusobacterium, Eubacterium, Ruminococcus, Peptococcus, Peptostreptococcus, Bifidobacterium, Escherichia, and Lactobacillus species). Examples of human autoantigens include insulin, proliferating cell nuclear antigen, cytochrome P450, and myelin basic protein.

A polypeptide included in a fusion protein described herein may comprise one or more immunogenic portions and one or more nonimmunogenic portions. The immunogenic portions may be identified by various methods, including protein microarrays, ELISPOT/ELISA techniques, and/or specific assays on different deletion mutants (e.g., fragments) of the polypeptide in question. Immunogenic portions may also be identified by computer algorithms. Some such algorithms, like EpiMatrix (produced by EpiVax), use a computational matrix approach. Other computational tools for identifying antigenic epitopes include PEPVAC (Promiscuous EPitope-based VACcine, hosted by Dana Farber Cancer Institute on the world wide web at immunax.dfci.harvard.edu/PEPVAC), MHCPred (which uses a partial least squares approach and is hosted by The Jenner Institute on the world wide web at www.jenner.ac.uk/MHCPred), and Immune Epitope Database algorithms on the World Wide Web at tools.immuneepitope.org. An antigenic fragment of a polypeptide described herein comprises at least one immunogenic portion, as measured experimentally or identified by algorithm (for example, the SYFPEITHI algorithm found at syfpeithi.de).

### Antibody Compositions

Some embodiments provide for an antibody composition comprising antibodies raised in a mammal immunized with an immunogenic complex of the invention. In some embodiments, an antibody comprises at least one antibody selected from the group consisting of mAbs and anti-idiotype antibodies. In some embodiments, an antibody composition comprises an isolated gamma globulin fraction. In some embodiments, an antibody composition comprises polyclonal antibodies. In some embodiments, the antibody composition is administered to a subject. In some embodiments, the antibody composition administered to a subject confers passive immunization.

### Vaccine Formulations

Optimal amounts of components for a particular vaccine can be ascertained by standard studies involving observation of appropriate immune responses in subjects. Following an initial vaccination, subjects can receive one or several booster immunizations adequately spaced in time.

The immunogenic complexes described herein, and/or preparations thereof may be formulated in a unit dosage form for ease of administration and uniformity of dosage. The specific therapeutically effective dose level for any particular subject or organism may depend upon a variety of factors including the severity or degree of risk of infection; the activity of the specific vaccine or vaccine composition employed; other characteristics of the specific vaccine or vaccine composition employed; the age, body weight, general health, sex of the subject, diet of the subject, pharmacokinetic condition of the subject, the time of administration (e.g., with regard to other activities of the subject such as eating, sleeping, receiving other medicines including other vaccine doses, etc.), route of administration, rate of excretion of the specific vaccine or vaccine composition employed; vaccines used in combination or coincidental with the vaccine composition employed; and like factors well known in the medical arts.

Immunogenic complexes for use in accordance with the present disclosure may be formulated into compositions (e.g., pharmaceutical compositions) according to known techniques. Vaccine preparation is generally described in Vaccine Design (Powell and Newman, 1995). For example, an immunologically amount of a vaccine product can be formulated together with one or more organic or inorganic, liquid or solid, pharmaceutically suitable carrier materials. Preparation of pneumococcal polysaccharide and conjugate vaccines is described, for example, in U.S. Ser. No. 11/395,593, filed Mar. 31, 2006, the contents of which are incorporated herein by reference.

In general, pharmaceutically acceptable carrier(s) include solvents, dispersion media, and the like, which are compatible with pharmaceutical administration. For example, materials that can serve as pharmaceutically acceptable carriers include, but are not limited to sugars such as lactose, glucose, dextrose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; polyols such as glycerol, propylene glycol, and liquid polyethylene glycol; esters such as ethyl oleate and ethyl laurate; agar; buffering agents such as magnesium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as preservatives, and antioxidants can also be present in the composition, according to the judgment of the formulator (Martin, 1975).

Vaccines may be formulated by combining one or more of the immunogenic complexes disclosed herein with carriers and/or other optional components by any available means including, for example, conventional mixing, granulating, dissolving, lyophilizing, or similar processes.

Vaccine compositions useful in the provided methods may be lyophilized up until they are about to be used, at which point they are extemporaneously reconstituted with diluent. In some embodiments, vaccine components or compositions are lyophilized in the presence of one or more other components (e.g., adjuvants), and are extemporaneously reconstituted with saline solution. Alternatively, individual components, or sets of components may be separately lyophilized and/or stored (e.g., in a vaccination kit), the components being reconstituted and either mixed prior to use or administered separately to the subject.

Lyophilization can produce a more stable composition (for instance by preventing or reducing breakdown of polysaccharide antigens). Lyophilizing of vaccines or vaccine components is well known in the art. Typically, a liquid vaccine or vaccine component is freeze dried, often in the presence of an anti-caking agent (such as, for example, sugars such as sucrose or lactose). In some embodiments, the anti-caking agent is present, for example, at an initial concentration of 10-200 mg/ml. Lyophilization typically occurs over a series of steps, for instance a cycle starting at −69° C., gradually adjusting to −24° C. over 3 h, then retaining this temperature for 18 h, then gradually adjusting to −16° C. over 1 h, then retaining this temperature for 6 h, then gradually adjusting to +34° C. over 3 h, and finally retaining this temperature over 9 h.

In some embodiments, a vaccine is a liquid. In some embodiments the liquid is a reconstituted lyophylate. In some embodiments a vaccine has a pH of about 5, about 6, about 7, or about 8. In some embodiments a vaccine has a pH between about 5 and about 7.5. In some embodiments a vaccine has a pH between 5 and 7.5. In some embodiments a vaccine has a pH between about 5.3 and about 6.3. In some embodiments a vaccine has a pH between 5.3 and 6.3. In some embodiments a vaccine has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

Vaccines or vaccine components for use in accordance with the present invention may be incorporated into liposomes, cochleates, biodegradable polymers such as poly-lactide, poly-glycolide and poly-lactide-co-glycolides, or immune-stimulating complexes (ISCOMs).

In certain situations, it may be desirable to prolong the effect of a vaccine or for use in accordance with the present invention, for example by slowing the absorption of one or more vaccine components. Such delay of absorption may be accomplished, for example, by the use of a liquid suspension of crystalline or amorphous material with poor water solubility. The rate of absorption of the product then depends upon its rate of dissolution, which in turn, may depend upon size and form. Alternatively, or additionally, delayed absorption may be accomplished by dissolving or suspending one or more vaccine components in an oil vehicle. Injectable depot forms can also be employed to delay absorption. Such depot forms can be prepared by forming microcapsule matrices of one or more vaccine components a biodegradable polymers network. Depending upon the ratio of polymer to vaccine component, and the nature of the particular polymer(s) employed, the rate of release can be controlled.

Examples of biodegradable polymers that can be employed in accordance with the present invention include, for example, poly(orthoesters) and poly(anhydrides). One particular exemplary polymer is polylactide-polyglycolide.

Depot injectable formulations may also be prepared by entrapping the product in liposomes or microemulsions, which are compatible with body tissues.

Polymeric delivery systems can also be employed in non-depot formulations including, for example, oral formulations. For example, biodegradable, biocompatible polymers such as ethylene vinyl acetate, polyanhydrides, polyglycolic acid, collagen, polyorthoesters, and polylactic acid, etc., can be used in oral formulations. Polysaccharide antigens or conjugates may be formulated with such polymers, for example to prepare particles, microparticles, extrudates, solid dispersions, admixtures, or other combinations in order to facilitate preparation of useful formulations (e.g., oral).

Vaccines for use in accordance with the present invention include immunogenic compositions, and may additionally include one or more additional active agents (i.e., agents that exert a biological effect—not inert ingredients). For example, it is common in vaccine preparation to include one or more adjuvants. It will be appreciated that such additional agents may be formulated together with one or more other vaccine components, or may be maintained separately and combined at or near the time of administration. In some embodiments, such additional components may be administered separately from some or all of the other vaccine components, within an appropriate time window for the relevant effect to be achieved.

### Adjuvants

The vaccine formulations and immunogenic compositions described herein may include an adjuvant. Adjuvants, generally, are agents that enhance the immune response to an antigen. Adjuvants can be broadly separated into two classes, based on their principal mechanisms of action: vaccine delivery systems and immunostimulatory adjuvants (see, e.g., Singh et al, 2003). In most vaccine formulations, the adjuvant provides a signal to the immune system so that it generates a response to the antigen, and the antigen is required for driving the specificity of the response to the pathogen. Vaccine delivery systems are often particulate formulations, e.g., emulsions, microparticles, immune-stimulating complexes (ISCOMs), nanoparticles, which may be, for example, particles and/or matrices, and liposomes. In contrast, immunostimulatory adjuvants are sometimes from or derived from pathogens and can represent pathogen associated molecular patterns (PAMP), e.g., lipopolysaccharides (LPS), monophosphoryl lipid A (MPL), or CpG-containing DNA, which activate cells of the innate immune system.

Alternatively, adjuvants may be classified as organic and inorganic. Inorganic adjuvants include alum salts such as aluminum phosphate, amorphous aluminum hydroxyphosphate sulfate, and aluminum hydroxide, which are commonly used in human vaccines. Organic adjuvants comprise organic molecules including macromolecules. Nonlimiting examples of organic adjuvants include cholera toxin/toxoids, other enterotoxins/toxoids or labile toxins/toxoids of Gram-negative bacteria, interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), and tumor necrosis factor (TNF).

In a specific embodiment, vaccine formulations and immunogenic compositions described herein may include aluminum phosphate. In particular, in addition to the antigens and the adjuvants described above, a vaccine formulation or immunogenic composition may include one or more additional components. In some embodiments, the vaccine formulation comprises aluminum phosphate (referred to herein as alum phosphate, or AP). In some embodiments, a vaccine formulation comprising Shigella-MAPS also comprise aluminum phosphate (referred to herein as alum phosphate, or AP). In some embodiments, the amount of alum phosphate is determined by one of ordinary skill in the art. In some embodiments, the amount of alum phosphate is 250 μg per 500 μl injection (25 μg polysaccharide). In some embodiments, a vaccine formulation or immunogenic composition comprises 250 μg of alum phosphate per 500 μl injection. In some embodiments, the alum phosphate is in a buffer comprising 20 mM Histadine, pH 6, 150 mM NaCl, 0.02% tween 80.

Adjuvants may also be classified by the response they induce. In some embodiments, the adjuvant induces the generation, proliferation, or activation of Tₕ1 cells or Tₕ2 cells. In other embodiments, the adjuvant induces the generation, proliferation, or activation of B cells. In yet other embodiments, the adjuvant induces the activation of antigen-presenting cells. These categories are not mutually exclusive; in some cases, an adjuvant activates more than one type of cell.

In certain embodiments, the adjuvant induces the generation, proliferation, or activation of Tₕ17 cells. The adjuvant may promote the CD4+ or CD8+ T cells to secrete IL-17. In some embodiments, an adjuvant that induces the generation, proliferation, or activation of Tₕ17 cells is one that produces at least a 2-fold, and in some cases a 10-fold, experimental sample to control ratio in the following assay. In the assay, an experimenter compares the IL-17 levels secreted by two populations of cells: (1) cells from animals immunized with the adjuvant and a polypeptide known to induce Tₕ17 generation, proliferation, or activation, and (2) cells from animals treated with the adjuvant and an irrelevant (control) polypeptide. An adjuvant that induces the generation, proliferation, or activation of Tₕ17 cells may cause the cells of population (1) to produce more than 2-fold, or more than 10-fold more IL-17 than the cells of population (2). IL-17 may be measured, for example, by ELISA or ELISPOT. Certain toxins, such as cholera toxin and labile toxin (produced by enterotoxigenic E. coli, or ETEC), activate a Tₕ17 response. Thus, in some embodiments, the adjuvant is a toxin or toxoid. Cholera toxin was successfully used in the mouse model to induce protective immunity in conjunction with certain polypeptides from Table 1 (see Examples 5-8). One form of labile toxin is produced by Intercell. Mutant derivates of labile toxin (toxoids) that are active as adjuvants but significantly less toxic can be used as well. Exemplary detoxified mutant derivatives of labile toxin include mutants lacking AE)P-ribosyltransferase activity. Particular detoxified mutant derivatives of labile toxin include LTK7 (Douce et al, 1995) and LTK63 (Williams et al, 2004), LT-G192 (Douce et al, 1999), and LTR72 (Giuliani et al, 1998).

In some embodiments, the adjuvant comprises a VLP (virus-like particle). One such adjuvant platform, Alphavirus replicons, induces the activation of Tₕ17 cells using alphavirus and is produced by Alphavax. In certain embodiments of the Alphavirus replicon system, alphavirus may be engineered to express an antigen of interest, a cytokine of interest (for example, IL-17 or a cytokine that stimulates IL-17 production), or both, and may be produced in a helper cell line. More detailed information may be found in U.S. Pat. Nos. 5,643,576 and 6,783,939. In some embodiments, a vaccine formulation is administered to a subject in combination with a nucleic acid encoding a cytokine.

Certain classes of adjuvants activate toll-like receptors (TLRs) in order to activate a Tₕ17 response. TLRs are well known proteins that may be found on leukocyte membranes, and recognize foreign antigens (including microbial antigens). Administering a known TLR ligand together with an antigen of interest (for instance, as a fusion protein) can promote the development of an immune response specific to the antigen of interest. One exemplary adjuvant that activates TLRs comprises Monophosphoryl Lipid A (MPL). Traditionally, MPL has been produced as a detoxified lipopolysaccharide (LPS) endotoxin obtained from Gram-negative bacteria, such as S. minnesota. In particular, sequential acid and base hydrolysis of LPS produces an immunoactive lipid A fraction (which is MPL), and lacks the saccharide groups and all but one of the phosphates present in LPS. A number of synthetic TLR agonists (in particular, TLR-4 agonists) are disclosed in Evans et al, 2003. Like MPL adjuvants, these synthetic compounds activate the innate immune system via TLR. Another type of TLR agonist is a synthetic phospholipid dimer, for example E6020 (Ishizaka et al, 2007). Various TLR agonists (including TLR-4 agonists) have been produced and/or sold by, for example, the Infectious Disease Research Institute (IRDI), Corixa, Esai, Avanti Polar Lipids, Inc., and Sigma Aldrich. Another exemplary adjuvant that activates TLRs comprises a mixture of MPL, Trehalose Dicoynomycolate (TDM), and dioctadecyldimethylammonium bromide (DDA). Another TLR-activating adjuvant is R848 (resiquimod).

In some embodiments, the adjuvant is or comprises a saponin. Typically, the saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C.

In some embodiments, the vaccine composition as disclosed herein comprises an AddaSO3 adjuvant. In some embodiments, the vaccine composition as disclosed herein comprises a R848 adjuvant. In some embodiments, the vaccine composition as disclosed herein comprises an ODN2395 adjuvant. In some embodiments, the vaccine composition as disclosed herein comprises an Alum phos adjuvant. In some embodiments, the vaccine composition as disclosed herein comprises an AddaSO3 alum adjuvant. In some embodiments, the vaccine composition as disclosed herein comprises a R848 alum adjuvant. In some embodiments, the vaccine composition as disclosed herein comprises an ODN alum adjuvant. In some embodiments, the vaccine composition as disclosed herein comprises an Alum 2PE adjuvant.

In some embodiments, the pharmaceutical composition as disclosed herein comprises an AddaSO3 adjuvant. In some embodiments, the pharmaceutical composition as disclosed herein comprises a R848 adjuvant. In some embodiments, the pharmaceutical composition as disclosed herein comprises an ODN2395 adjuvant. In some embodiments, the pharmaceutical composition as disclosed herein comprises an Alum phos adjuvant. In some embodiments, the pharmaceutical composition as disclosed herein comprises an AddaSO3 alum adjuvant. In some embodiments, the pharmaceutical composition as disclosed herein comprises a R848 alum adjuvant. In some embodiments, the pharmaceutical composition as disclosed herein comprises an ODN alum adjuvant. In some embodiments, the pharmaceutical composition as disclosed herein comprises an Alum 2PE adjuvant.

In some embodiments, the immunogenic composition as disclosed herein comprises an AddaSO3 adjuvant. In some embodiments, the immunogenic composition as disclosed herein comprises a R848 adjuvant. In some embodiments, the immunogenic composition as disclosed herein comprises an ODN2395 adjuvant. In some embodiments, the immunogenic composition as disclosed herein comprises an Alum phos adjuvant. In some embodiments, the immunogenic composition as disclosed herein comprises an AddaSO3 alum adjuvant. In some embodiments, the immunogenic composition as disclosed herein comprises a R848 alum adjuvant. In some embodiments, the immunogenic composition as disclosed herein comprises an ODN alum adjuvant. In some embodiments, the immunogenic composition as disclosed herein comprises an Alum 2PE adjuvant.

In some embodiments, the adjuvant can comprise of at least one of AddaSO3, R848, ODN2395, Alum phosphate, AddaSO3 alum, R848 alum, ODN alum, or Alum 2PE, at least two of AddaSO3, R848, ODN2395, Alum phosphate, AddaSO3 alum, R848 alum, ODN alum, or Alum 2PE, at least three of AddaSO3, R848, ODN2395, Alum phosphate, AddaSO3 alum, R848 alum, ODN alum, or Alum 2PE, at least four of AddaSO3, R848, ODN2395, Alum phosphate, AddaSO3 alum, R848 alum, ODN alum, or Alum 2PE, at least five of AddaSO3, R848, ODN2395, Alum phosphate, AddaSO3 alum, R848 alum, ODN alum, or Alum 2PE, at least six of AddaSO3, R848, ODN2395, Alum phosphate, AddaSO3 alum, R848 alum, ODN alum, or Alum 2PE, at least seven of AddaSO3, R848, ODN2395, Alum phosphate, AddaSO3 alum, R848 alum, ODN alum, or Alum 2PE, or at least eight of AddaSO3, R848, ODN2395, Alum phosphate, AddaSO3 alum, R848 alum, ODN alum, or Alum 2PE.

In certain embodiments, combinations of adjuvants are used. Three exemplary combinations of adjuvants are MPL and alum, E6020 and alum, and MPL and an ISCOM.

Adjuvants may be covalently or non-covalently bound to antigens. In some embodiments, the adjuvant may comprise a protein which induces inflammatory responses through activation of antigen-presenting cells (APCs). In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to T cells and initiation of T cell responses (e.g., see Wu et al, 2005).

In some embodiments, immunogenic complexes described herein are formulated and/or administered in combination with an adjuvant. In some embodiments, the adjuvant is selected from the group consisting of aluminum phosphate, aluminum hydroxide, and phosphate aluminum hydroxide. In some embodiments, the adjuvant comprises aluminum phosphate. In some embodiments, the adjuvant is aluminum phosphate.

Typically, the same adjuvant or mixture of adjuvants is present in each dose of a vaccine. Optionally, however, an adjuvant may be administered with the first dose of vaccine and not with subsequent doses (i.e., booster shots). Alternatively, a strong adjuvant may be administered with the first dose of vaccine and a weaker adjuvant or lower dose of the strong adjuvant may be administered with subsequent doses. The adjuvant can be administered before the administration of the antigen, concurrent with the administration of the antigen or after the administration of the antigen to a subject (sometimes within 1, 2, 6, or 12 hours, and sometimes within 1, 2, or 5 days). Certain adjuvants are appropriate for human subjects, non-human animals, or both.

Vaccines for use in accordance with the present invention may include, or be administered concurrently with, other antimicrobial therapy. For example, such vaccines may include or be administered with one or more agents that kills or retards growth of a pathogen. Such agents include, for example, penicillin, vancomycin, erythromycin, azithromycin, and clarithromycin, cefotaxime, ceftriaxone, levoflaxin, gatifloxacin.

Alternatively or additionally, vaccines for use in accordance with the present invention may include, or be administered with, one or more other vaccines or therapies. For example, one or more non-pneumococcal antigens may be included in or administered with the vaccines.

### Additional Components and Excipients

In addition to the antigens and the adjuvants described above, a vaccine formulation or immunogenic composition may include one or more additional components. In some embodiments, the vaccine formulation comprises aluminum phosphate (referred to herein as alum phosphate, or AP). In some embodiments, a vaccine formulation comprising shigella-MAPS aluminum phosphate (referred to herein as alum phosphate, or AP). In some embodiments, the amount of alum phosphate is determined by one of ordinary skill in the art. In some embodiments, the amount of alum phosphate is 250 μg per 500 μl injection (25 μg polysaccharide). In some embodiments, a vaccine formulation or immunogenic composition comprises 250 μg of alum phosphate per 500 μl injection. In some embodiments, the alum phosphate is in a buffer comprising 20 mM Histadine, pH 6, 150 mM NaCl, 0.02% tween 80.

In certain embodiments, the vaccine formulation or immunogenic composition may include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In certain embodiments, the vaccine formulation or immunogenic composition includes one or more buffers such as a mixture of sodium bicarbonate and ascorbic acid. In some embodiments, the vaccine formulation may be administered in saline, such as phosphate buffered saline (PBS), or distilled water.

In certain embodiments, the vaccine formulation or immunogenic composition includes one or more surfactants, for example, but not limited to, polysorbate 80 (TWEEN 80), polysorbate 20 (TWEEN 20), Polyethylene glycol p-(1,1,3,3-tetramethylbutyl)-phenyl ether (TRITON X-100), and 4-(1,1,3,3-Tetramethylbutyl)phenol polymer with formaldehyde and oxirane (TYTOXAPOT). A surfactant can be ionic or non-ionic.

In certain embodiments, the vaccine formulation or immunogenic composition includes one or more salts such as sodium chloride, ammonium chloride, calcium chloride, or potassium chloride.

In certain embodiments, a preservative is included in the vaccine or immunogenic composition. In other embodiments, no preservative is used. A preservative is most often used in multi-dose vaccine vials, and is less often needed in single-dose vaccine vials. In certain embodiments, the preservative is 2-phenoxyethanol, methyl and propyl parabens, benzyl alcohol, and/or sorbic acid.

### Methods of Administration

In some embodiments, immunogenic complexes are administered to a subject at risk of developing pneumococcal disease, e.g.. an infant, a toddler, a juvenile, or an older adult. In some embodiments the subject is a human. In some embodiments the human is between about 2 weeks of age and about 6 weeks of age. In some embodiments the human is between about 6 weeks of age and about 6 years of age. In some embodiments the human is between about 6 years of age and about 18 years of age. In some embodiments the human is between about 18 years of age and about 50 years of age. In some embodiments the human is about 50 years of age or older. In some embodiments, immunogenic complexes are administered to a subject at elevated risk of developing shigellosis, e.g., immunocompromised subjects, subjects having sickle cell disease or other hemoglobinopathies, congenital or acquired asplenia, splenic dysfunction, chronic renal failure or nephrotic syndrome, diseases associated with treatment with immunosuppressive drugs or radiation therapy (including malignant neoplasm, leukemia, lymphomas, Hodgkin's disease, or solid organ transplantation), congenital or acquired immunodeficiency, HIV infection, cerebrospinal fluid leaks, cochlear implant(s), chronic heart disease, chronic lung disease, diabetes mellitus, alcoholism, chronic liver disease, cigarette smoking, asthma, generalized malignancy, multiple myeloma, or solid organ transplantation. It will be appreciated that a subject can be considered at risk for developing a disease without having been diagnosed with any symptoms of the disease. For example, if the subject is known to have been, or to be intended to be, in situations with relatively high risk of infection, such as at a refugee camp or other cramped living quarters and/or with poor hygiene and waste removal, that subject will be considered at risk for developing the disease.

Any effective route of administration may be utilized such as, for example, oral, nasal, enteral, parenteral, intramuscular or intravenous, subcutaneous, transdermal, intradermal, rectal, vaginal, topical, ocular, pulmonary, or by contact application. In some embodiments, vaccine compositions may be injected (e.g., via intramuscular, intraperitoneal, intradermal and/or subcutaneous routes); or delivered via the mucosa (e.g., to the oral/alimentary, respiratory, and/or genitourinary tracts). Intranasal administration of vaccines may be particularly useful in some contexts, for example for treatment of shigellosis or pneumonia or otitis media (as nasopharyngeal carriage of shigella can be more effectively prevented, thus attenuating infection at its earliest stage). In some embodiments of the invention, it may be desirable to administer different doses of a vaccine by different routes; in some embodiments, it may be desirable to administer different components of one dose via different routes. In some embodiments, an immunogenic composition or vaccine disclosed herein is administered intramuscularly. In some embodiments, an immunogenic composition or vaccine disclosed herein is administered subcutaneously.

In some embodiments of the present invention, pharmaceutical compositions (e.g., vaccines) are administered intradermally. Conventional technique of intradermal injection, the “Mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced while providing a slight pressure to elevate it under the skin. The liquid is then injected very slowly thereby forming a bleb or bump on the skin surface, followed by slow withdrawal of the needle.

Devices that are specifically designed to administer liquid agents into or across the skin have been described, for example the devices described in WO 99/34850 and EP 1092444, also the jet injection devices described for example in WO 01/13977; U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 97/37705 and WO 97/13537. Other methods of intradermal administration of the vaccine preparations may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

As described above, pharmaceutical compositions (e.g., vaccines) may be administered as a single dose or as multiple doses. It will be appreciated that an administration is a single “dose” so long as all relevant components are administered to a subject within a window of time; it is not necessary that every component be present in a single composition. For example, administration of two different immunogenic compositions, within a period of less than 24 h, is considered a single dose. To give but one example, immunogenic compositions having different antigenic components may be administered in separate compositions, but as part of a single dose. As noted above, such separate compositions may be administered via different routes or via the same route. Alternatively or additionally, in embodiments wherein a vaccine comprises a combination of immunogenic compositions and additional types of active agents, immunogenic compositions may be administered via one route, and a second active agent may be administered by the same route or by a different route.

Pharmaceutical compositions (e.g., vaccines) are administered in such amounts and for such time as is necessary to achieve a desired result. In certain embodiments of the present invention, a vaccine composition comprises an immunologically effective amount of at least immunogenic composition. The exact amount required to achieve an immunologically effective amount may vary, depending on the immunogenic composition, and from subject to subject, depending on the species, age, and general condition of the subject, the stage of the disease, the particular pharmaceutical mixture, its mode of administration, and the like.

The amount of polypeptide antigen(s), polysaccharide antigen(s) or conjugate(s) in each pharmaceutical composition (e.g., vaccine) dose is selected to allow the vaccine, when administered as described herein, to induce an appropriate immune-protective response without significant, adverse side effects.

### Combination Prophylaxis or Combination Therapy

In some embodiments, an immunogenic complex, immunogenic composition, vaccine, or pharmaceutical composition disclosed herein may be administered in combination with another agent.

### Dosing

In some embodiments, administration of a vaccine (e.g., a vaccine composition) described herein may involve the delivery of a single dose. In some embodiments, administration may involve an initial dose followed by one or several additional immunization doses, adequately spaced. Such additional immunization doses can be referred to as boosters. In some embodiments, a booster (or second or subsequent) immunization dose is administered 2 weeks, or 3 weeks, or about 1 month, or about 2 months, or about 6 months or about 1 year after the preceding dose (where the proceeding dose can be initial dose or a second or third dose, or booster dose).

The present disclosure provides immunization methods that involve administering at least one dose of a vaccine to an infant subject. In some embodiments, the infant subject is 18 months old or younger. In some embodiments, the infant subject is 12 months old or younger.

The present disclosure provides immunization methods that involve administering at least one dose of a vaccine to a toddler subject. In some embodiments, the toddler subject is 5 years old or younger. In some embodiments, the toddler subject is 4 years old or younger.

The present disclosure provides immunization methods that involve administering at least one dose of a vaccine to a juvenile subject. In some embodiments, the juvenile subject is 18 years old or younger. In some embodiments, the juvenile subject is 15 years old or younger.

The present disclosure provides immunization methods that involve administering at least one dose of a vaccine to an adult subject. In some embodiments, the adult subject is older than about 50 years of age. In some embodiments, the adult subject is older than about 65 years of age.

Immunization schedules of the present disclosure are provided to induce an immune response (e.g., an immunoprotective response) in a subject sufficient to reduce at least one measure selected from the group consisting of incidence, prevalence, frequency, and/or severity of at least one infection, disease, or disorder, and/or at least one surrogate marker of the infection, disease, or disorder, in a population and/or subpopulation of the subject(s). A supplemental immunization schedule is one which has this effect relative to the standard schedule which it supplements. A supplemental schedule may call for additional administrations and/or supra-immunogenic doses of the immunogenic compositions disclosed herein, found in the standard schedule, or for the administration of vaccines not part of the standard schedule. A full immunization schedule of the present invention may comprise both a standard schedule and a supplemental schedule. Exemplary sample vaccination schedules are provided for illustrative purposes. Detailed descriptions of methods to assess immunogenic response discussed herein allow one to develop alterations to the sample immunization schedules without undue experimentation.

It will be appreciated by those skilled in the art that a variety of possible combinations and sub-combinations of the various conditions of timing of the first administration, shortest interval, largest interval and total number of administrations (in absolute terms, or within a stated period) exist, and all of these combinations and sub-combinations should be considered to be within the inventor's contemplation though not explicitly enumerated here.

### Assays for Determining Immune Response

In some embodiments, a method of assessing the immunogenicity of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by opsonophagocytic killing (OPK), serum bactericidal killing (SBA), agglutination, motility, cytotoxicity, or adherence; and in vivo assays in animal models of shigella disease (e.g. shigellosis, pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the shigella pathogens that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the potency of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by OPK, serum bactericidal killing (SBA), internalization, activity neutralization, agglutination, motility, cytotoxicity, or adherence; and in vivo assays in animal models of shigella disease (e.g. shigellosis, pneumonia, bacteremia, meningitis, sepsis, otitis media, nasopharyngeal colonization).

In some embodiments, a method of assessing the immunogenicity of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by opsonophagocytic killing (OPK), serum bactericidal killing (SBA), agglutination, motility, cytotoxicity, or adherence; and in vivo assays in animal models of shigella disease (e.g. shigellosis, pneumonia, bacteremia, meningitis, sepsis, otitis media, nasopharyngeal colonization).

Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the shigella pathogens that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the potency of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by OPK, serum bactericidal killing (SBA), internalization, activity neutralization, agglutination, motility, cytotoxicity, or adherence; and in vivo assays in animal models of shigella disease (e.g. shigellosis, pneumonia, bacteremia, meningitis, sepsis, otitis media, nasopharyngeal colonization). Parameters of in vivo assays include bacterial clearance or reduction from mucosal surfaces or bloodstream, reduction or prevention of bacteremia, meningitis, sepsis, or otitis media, reduction or prevention of colonization of the nasopharynx, reduction of mortality, and passive and active protection following challenge with the shigella pathogens that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the immunogenicity of a vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by OPK, serum bactericidal killing (SBA), agglutination, motility, cytotoxicity, or adherence; and in vivo assays in animal models of shigella disease (e.g. shigellosis, pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the shigella pathogens that are the targets of the immunogenic composition. In some embodiments, the immune response is compared to a control composition. In some embodiments, a control composition may comprise an antigenic polysaccharide present in the vaccine composition and not comprise an antigenic polypeptide present in the vaccine composition. In some embodiments, a control composition may comprise an antigenic polypeptide present in the vaccine composition and not comprise an antigenic polysaccharide present in the vaccine composition. In some embodiments, a control composition may comprise an adjuvant present in the vaccine composition, and not comprise an antigenic polysaccharide and/or an immunogenic polypeptide present in the vaccine composition.

In some embodiments, a method of assessing the potency of a vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by OPK, serum bactericidal killing (SBA), agglutination, motility, cytotoxicity, or adherence; and in vivo assays in animal models of shigella disease (e.g. shigellosis, pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the shigella pathogens that are the targets of the immunogenic composition. In some embodiments, the immune response is compared to a control composition. In some embodiments, a control composition may comprise an antigenic polysaccharide present in the vaccine composition and not comprise an antigenic polypeptide present in the vaccine composition. In some embodiments, a control composition may comprise an antigenic polypeptide present in the vaccine composition and not comprise an antigenic polysaccharide present in the vaccine composition. In some embodiments, a control composition may comprise an adjuvant present in the vaccine composition, and not comprise an antigenic polysaccharide and/or an immunogenic polypeptide present in the vaccine composition.

In some embodiments, a method of assessing the immunogenicity of a pharmaceutical composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by OPK, serum bactericidal killing (SBA), agglutination, motility, cytotoxicity, or adherence; and in vivo assays in animal models of shigella disease (e.g. shigellosis, pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the shigella pathogens that are the targets of the immunogenic composition. In some embodiments, the immune response is compared to a control composition. In some embodiments, a control composition may comprise an antigenic polysaccharide present in the pharmaceutical composition and not comprise an antigenic polypeptide present in the pharmaceutical composition. In some embodiments, a control composition may comprise an antigenic polypeptide present in the pharmaceutical composition and not comprise an antigenic polysaccharide present in the pharmaceutical composition. In some embodiments, a control composition may comprise an adjuvant present in the pharmaceutical composition, and not comprise an antigenic polysaccharide and/or an immunogenic polypeptide present in the pharmaceutical composition.

In some embodiments, a method of assessing the potency of a pharmaceutical composition described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by OPK, serum bactericidal killing (SBA), agglutination, motility, cytotoxicity, or adherence; and in vivo assays in animal models of shigella disease (e.g. shigella, pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the shigella pathogens that are the targets of the immunogenic composition. In some embodiments, the immune response is compared to a control composition. In some embodiments, a control composition may comprise an antigenic polysaccharide present in the pharmaceutical composition and not comprise an antigenic polypeptide present in the pharmaceutical composition. In some embodiments, a control composition may comprise an antigenic polypeptide present in the pharmaceutical composition and not comprise an antigenic polysaccharide present in the pharmaceutical composition. In some embodiments, a control composition may comprise an adjuvant present in the pharmaceutical composition, and not comprise an antigenic polysaccharide and/or an immunogenic polypeptide present in the pharmaceutical composition.

In some embodiments, a method of assessing the immunogenicity and/or potency of an immunogenic complex comprises evaluating an immune response to immunogenic or vaccine compositions comprising one or more immunogenic complexes. In some embodiments, the method of assessing the immunogenicity and/or potency of an immunogenic complex described herein comprises evaluating, measuring, and/or comparing an immune response using one or more in vitro bioassays, including B cell and T cell responses such as antibody levels by ELISA, multiplex ELISA, MSD, Luminex, flow cytometry, Tₕ1/Tₕ17 cell response, cytokine level measurement and functional antibody levels as measured by OPK, serum bactericidal killing (SBA), agglutination, motility, cytotoxicity, or adherence; and in vivo assays in animal models of shigella disease (e.g. shigellosis, pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction in mortality, and passive and active protection following challenge with the shigella pathogens that are the targets of the immunogenic composition.

Generally speaking, it may be desirable to assess humoral responses, cellular responses, and/or interactions between the two. Where humoral responses are being assessed, antibody titers and/or types (e.g., total IgG, IgG1, IgG2, IgM, IgA, etc.) to specific pathogen polysaccharides or polypeptides (either serotype-specific or conserved across two or more serotypes) may be determined, for example before and/or after administration of an initial or a boosting dose of vaccine (and/or as compared with antibody levels in the absence of antigenic stimulation). Cellular responses may be assessed by monitoring reactions such as delayed type hypersensitivity responses, etc. to the carrier protein. Cellular responses can also be measured directly by evaluating the response of peripheral blood mononuclear cells (PBMCs) monocytes to stimulation with the antigens of interest. Precursor and memory B cell populations may be assessed in enzyme linked immunospot (ELISpot) assays directed against specific pathogen polysaccharides or polypeptides.

Any of a variety of assays may be employed to detect levels and/or activity of antibodies in subject sera. Suitable assays include, for example, ligand binding assays, such as radioimmunoassay (RIAs), ELISAs, and multiplex assays (Luminex, Bioplex, MSD); functional assays, such as opsonophagocytic assays or internalization assays; and in vivo assays in animal models of shigella disease (e.g. shigellosis, pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction in mortality, and passive and active protection following challenge with the shigella pathogens that are the targets of the immunogenic composition.

The RIA method detects specific antibodies through incubation of sera with radio-labeled polysaccharides or polypeptides in suspension (e.g., Schiffiman et al, 1980). The antigenantibody complexes are then precipitated with ammonium sulfate and the radiolabeled pellets assayed for counts per minute (cpm).

In the ELISA detection method, specific antibodies from the sera of vaccinated subjects are quantitated by incubation with polysaccharides or polypeptides (either serotypespecific or conserved across two or more serotypes) which have been adsorbed to a solid support (e.g., Koskela and Leinonen (1981); Kojima et al, 1990; Concepcion and Frasch, 2001). The bound antibody is detected using enzyme-conjugated secondary detection antibodies. The ELISA also allows isotyping and subclassing of the immune response (i.e., IgM vs. IgG or IgG1 vs. IgG2) by using isotype- or subclass-specific secondary antibodies and can be adapted to evaluate the avidity of the antibodies (Anttila et al, 1998; Romero-Steiner et al, 2005). Multiplex assays (e.g., Luminex) facilitate simultaneous detection of antibodies to multiple antigens. O specific polysaccharide(s) or polypeptides are conjugated to spectrally distinct microspheres that are mixed and incubated with serum. The antibodies bound to the polysaccharides or polypeptides on the coated microspheres are detected using a secondary antibody (e.g., R-Phycoerythrin-conjugated goat anti-human IgG).

An approach for assessing functional antibody in serum is an opsonophagocytic assay (OPA) or a concentrated opsonophagocytic assay (COPA), which quantitates only the antibodies that can opsonize the bacteria, leading to ingestion and killing of the bacteria. The standard assay utilizes a human phagocytic effector cell, a source of complement, bacteria, and diluted sera. The assay readout is the serum endpoint titer at which there is >50% killing compared to bacteria incubated with complement and human cells alone (Romero-Steiner et al, 1997). This killing OPA can also be multiplexed by utilizing target strains of pathogen that carry different antibiotic resistance markers (Kim et al, 2003). Another type of multiplex opsonic assay is a nonkilling assay in which the uptake by phagocytic effector cells of fluorescent stained encapsulated pathogen or fluorescent microspheres conjugated with antigenic polysaccharides or polypeptides from a target pathogen in the presence of diluted sera plus a complement source is evaluated by flow cytometry (Martinez et al, 1999). Opsonic activity of serum antibody plus complement can also be evaluated by measuring the oxidative response of phagocytic human effector cells to ingested pathogen (Munro et al. 1985; Ojo-Amaize et al. 1995).

Certain in vivo model systems can be used to evaluate the protection afforded by serum antibodies induced by vaccines of the present invention. In such passive protection systems, mice or rats are challenged with the pathogen plus diluted sera, and the endpoint titer of the sera which provides protection against shigellosis, pneumonia, bacteremia, colonization of organs or tissues, or mortality is determined (Stack et al. 1998; Saeland et al. 2000).

In some embodiments, efficacy of vaccination may be determined by assaying one or more cytokine levels by stimulating T cells from a subject after vaccination. The one or more cytokine levels may be compared to the one or more cytokine levels in the same subject before vaccination. Increased levels of the one or more cytokine, such as a 1.5 fold, 2-fold, 5-fold, 10-fold, 20-fold, 50-fold or 100-fold or more increase over pre-immunization cytokine levels, would indicate an increased response to the vaccine. In some embodiments, the one or more cytokines are selected from GM-CSP; IL-Iα; IL-1β; IL-2; IL-3; IL-4; IL-5; IL-6; IL-7; IL-8; IL-10; IL-12; IL-17A, IL-17F or other members of the IL-17 family; IL-22; IL-23; IFN-α; IFN-β; IFN-γ; MIP-1α; MIP-1β; TGF-β; TNFα, or TNF-β. In a non-limiting example, efficacy of vaccination may be determined by assaying IL-17 levels (particularly IL-17 A) by stimulating T cells from a subject after vaccination. The IL-17 levels may be compared to IL-17 levels in the same subject before vaccination. Increased IL-17 (e.g., IL-17A) levels, such as a 1.5 fold, 2-fold, 5-fold, 10-fold, 20-fold, 50-fold or 100-fold or more increase, would indicate an increased response to the vaccine.

In some embodiments, one may assay neutrophils in the presence of T cells or antibodies from the patient for shigella killing. Increased shigella killing, such as a 1.5 fold, 2-fold, 5-fold, 10-fold, 20-fold, 50-fold or 100-fold or more increase, would indicate an increased response to the vaccine. For example, one may measure Tₕ17 cell activation, where increased Tₕ17 cell activation, such as a 1.5 fold, 2-fold, 5-fold, 10-fold, 20-fold, 50-fold or 100-fold or more increase, correlates with an increased response to the vaccine. In another nonlimiting example, one may measure Tₕ1 cell activation, where increased Tₕ1 cell activation, such as a 1.5 fold, 2-fold, 5-fold, 10-fold, 20-fold, 50-fold or 100-fold or more increase, correlates with an increased response to the vaccine. One may also measure levels of an antibody specific to the vaccine, where increased levels of the specific antibody, such as a 1.5 fold, 2-fold, 5-fold, 10-fold, 20-fold, 50-fold or 100-fold or more increase, are correlated with increased vaccine efficacy. In certain embodiments, two or more of these assays are used. For example, one may measure IL-17 levels and the levels of vaccine-specific antibody.

Alternatively, one may follow epidemiological markers such as incidence of, severity of, or duration of shigella infection in vaccinated individuals compared to unvaccinated individuals.

Vaccine efficacy may also be assayed in various model systems such as the mouse challenge model. For instance, BALB/c or C57BL/6 strains of mice may be used. After administering the test vaccine to a subject (as a single dose or multiple doses), the experimenter administers a challenge dose of Shigella sp. In some cases, a challenge dose administered intranasally is sufficient to cause Shigella sp.colonization (especially nasal colonization) in an unvaccinated animal, and in some cases a challenge dose administered via aspiration is sufficient to cause sepsis and a high rate of lethality in unvaccinated animals. In some cases, a challenge dose administered via intraperitoneal injection is sufficient to cause sepsis and a high rate of lethality in unvaccinated animals. In some cases, a challenge dose administered via intravenous injection is sufficient to cause sepsis and a high rate of lethality in unvaccinated animals. One can then measure the reduction in colonization or the reduction in lethality in vaccinated animals.

Certain in vivo model systems can be used to evaluate the protection afforded by serum antibodies induced by vaccines of the present invention. In such passive protection systems, mice or rats are challenged with the pathogen plus diluted sera, and the endpoint titer of the sera which provides protection against bacteremia, colonization of organs or tissues, or mortality is determined (Stack et al. 1998; Saeland et al. 2000).

### Kits

The present disclosure also provides for kits for producing an immunogenic complex as disclosed herein which is useful for an investigator to tailor an immunogenic complex with their preferred antigens, e.g., for research purposes to assess the effect of an antigen, or a combination of antigens on immune response. Such kits can be prepared from readily available materials and reagents. For example, such kits can comprise any one or more of the following materials: a container comprising a polysaccharide cross-linked with a plurality of first affinity molecules; a container comprising a complementary affinity molecule which associates with the first affinity molecule, wherein the complementary affinity molecule associates with an antigen or carrier protein; a container comprising an antigen; a container comprising a carrier protein; a container comprising an antigen associated with a complementary affinity molecule; a container comprising a carrier protein associated with a complementary affinity molecule.

In another embodiment, the kit comprises a container comprising a polysaccharide; a container comprising a plurality of first affinity molecules; and a container comprising a cross-linking reagent for cross-linking the first affinity molecules to the polysaccharide, for example, but not limited to, CDAP (1-cyano-4-dimethylaminopyridinium tetrafluoroborate), and EDC (1-Ethyl-3-[3-dimethylaminopropyl]carbodiimide hydrochloride).

In another embodiment, the kit comprises a container comprising an antigen or carrier protein, and a container comprising a complementary affinity molecule which associates with a first affinity molecule. In some embodiments, the kit further comprises a means to attach the complementary affinity molecule to the antigen or carrier protein, where the means can be by a cross-linking reagent or by some intermediary fusion protein.

In some embodiments, the kit can comprise at least one co-stimulation factor which can be added to the polymer. In some embodiments, the kit comprises a cross-linking reagent, for example, but not limited to, CDAP (1-cyano-4-dimethylaminopyridinium tetrafluoroborate); EDC (1-Ethyl-3-[3-dimethylaminopropyl]carbodiimide hydrochloride); sodium cyanoborohydride; cyanogen bromide; and ammonium bicarbonate/iodoacetic acid, for linking the co-factor to the polymer.

A variety of kits and components can be prepared for use in the methods described herein, depending upon the intended use of the kit, the particular target antigen and the needs of the user.

Exemplary embodiments of the various aspects described herein can be illustrated by the following numbered embodiments:

Embodiment 1: A vaccine comprising an immunogenic complex, wherein the immunogenic complex comprises:
(a) a biotinylated polysaccharide antigen; and
(b) a fusion protein comprising:
   (ii.) a biotin-binding moiety; and
   (iii.) at least one polypeptide antigen;
      wherein the biotinylated polysaccharide antigen comprises a 0-specific polysaccharide (OSP) of Shigella, and further wherein the biotinylated polysaccharide antigen is non-covalently associated with the biotin-binding moiety of the fusion protein to form an immunogenic complex.

Embodiment 2: The vaccine of Embodiment 1, wherein the biotinylated polysaccharide antigen comprises a polysaccharide of Shigella selected from S. flexneri 3a, S. flexneri 2a, S. flexneri 6 or S. sonnei.

Embodiment 3: The vaccine of Embodiment 1 or Embodiment 2, wherein the at least one polypeptide antigen of the fusion protein is or comprises a polypeptide antigen from Salmonella, Shigella, or Streptococcus pneumoniae.

Embodiment 4: The vaccine of any of Embodiments 1-3, wherein the at least one polypeptide antigen of the fusion protein comprises a Salmonella SseB polypeptide or antigenic fragment thereof.

Embodiment 5: The vaccine of any of Embodiments 1-3, wherein the at least one polypeptide antigen of the fusion protein comprises a Shigella IpaB polypeptide or antigenic fragment thereof.

Embodiment 6: The vaccine of any of Embodiments 1-3, wherein the at least one polypeptide antigen of the fusion protein comprises a Streptococcus pneumoniae SP1500 polypeptide or antigenic fragment thereof, a Streptococcus pneumoniae SP0785 polypeptide or antigenic fragment thereof, or both.

Embodiment 7: The vaccine of any one of the preceding Embodiments, wherein the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

Embodiment 8: The vaccine of any one of the preceding claims, wherein the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

Embodiment 9: The vaccine of any one of the preceding claims, wherein the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8 or SEQ ID NO: 9, or a combination of SEQ ID NO: 8 and SEQ ID NO: 9.

Embodiment 10: The vaccine of any one of the preceding claims, wherein the biotin-binding moiety is a polypeptide comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 3 or a biotin-binding fragment thereof.

Embodiment 11: An immunogenic composition (e.g., a vaccine) comprising a plurality of different species of immunogenic complexes, wherein the different species comprise:
a plurality of biotinylated polysaccharide antigens comprising polysaccharide antigens of one or more Shigella serotypes; and
a plurality of fusion proteins, each fusion protein comprising:
a biotin-binding moiety; and
a polypeptide antigen,
wherein each of the plurality of biotinylated polysaccharide antigens is non-covalently associated with the biotin-binding moiety of one or more of the plurality of fusion proteins to form an immunogenic complex.

Embodiment 12: The immunogenic composition of Embodiment 11, wherein the different species each comprise a distinct polysaccharide antigen of one or more Shigella serotypes and/or a distinct polypeptide antigen.

Embodiment 13: The immunogenic composition of Embodiment 11 or 12, wherein the one or more Shigella serotypes is or comprises S. flexneri 3a, S. flexneri 2a, S. flexneri 6 or S. sonnei, or combinations thereof.

Embodiment 14: The immunogenic composition of any one of Embodiments 11-13, wherein the polypeptide antigen is or comprises a polypeptide antigen from Salmonella, Shigella, and/or Streptococcus pneumoniae.

Embodiment 15: The immunogenic composition of Embodiment 14, wherein the polypeptide antigen is or comprises: an SseB polypeptide antigen of Salmonella, an IpaB polypeptide antigen of Shigella, and/or a polypeptide antigen comprising an SP1500 polypeptide and/or an SP0785 polypeptide, of S. pneumoniae.

Embodiment 16: The immunogenic composition of any one of Embodiments 11-15, comprising at least two different species of immunogenic complexes, wherein the different species comprise:
a first biotinylated polysaccharide antigen comprising a polysaccharide of S. flexneri 2a non-covalently complexed with a first fusion protein; and
a second biotinylated polysaccharide antigen comprising a polysaccharide of S. flexneri 3a non-covalently complexed with a second fusion protein,
   wherein the first fusion protein and the second fusion protein each independently comprise:
a biotin-binding moiety; and
a polypeptide antigen,
   wherein each of the first and second biotinylated polysaccharide antigens is non-covalently associated with the biotin-binding moiety of the respective fusion protein to form an immunogenic complex.

Embodiment 17: The immunogenic composition of Embodiment 16, wherein the polypeptide antigen of the first fusion protein and of the second fusion protein is or comprises an SseB polypeptide antigen of Salmonella.

Embodiment 18: The immunogenic composition of Embodiment 17, wherein the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof.

Embodiment 19: The immunogenic composition of any one of Embodiments 11-15, comprising at least two different species of immunogenic complexes, wherein the different species comprise:
a first biotinylated polysaccharide antigen comprising a polysaccharide of S. flexneri 2a non-covalently complexed with a first fusion protein; and
a second biotinylated polysaccharide antigen comprising a polysaccharide of S. sonnei non-covalently complexed with a second fusion protein,
   wherein the first fusion protein and the second fusion protein each independently comprise:
a biotin-binding moiety; and
a polypeptide antigen,
   wherein each of the first and second biotinylated polysaccharide antigens is non-covalently associated with the biotin-binding moiety of the respective fusion protein to form an immunogenic complex.

Embodiment 20: The immunogenic composition of claim 19, wherein the polypeptide antigen of the first fusion protein and of the second fusion protein is or comprises an SP1500 polypeptide, an SP0785 polypeptide, or both, of S. pneumoniae, and/or the fusion protein is CP1.

Embodiment 21: The immunogenic composition of claim 20, wherein the SP1500 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, the SP0785 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, and the CP1 fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 6, or an immunogenic fragment thereof.

Embodiment 22: The immunogenic composition of any one of Embodiments 11-15, comprising at least four different species of immunogenic complexes, wherein the different species comprise:
a first biotinylated polysaccharide antigen comprising a polysaccharide of S. flexneri 2a non-covalently complexed with a first fusion protein; and
a second biotinylated polysaccharide antigen comprising a polysaccharide of S. flexneri 3a non-covalently complexed with a second fusion protein,
a third biotinylated polysaccharide antigen comprising a polysaccharide of S. flexneri 6 non-covalently complexed with a third fusion protein; and
a fourth biotinylated polysaccharide antigen comprising a polysaccharide of S. sonnei A non-covalently complexed with a fourth fusion protein,
   wherein the first fusion protein, the second fusion protein, the third fusion protein, and the fourth fusion protein each independently comprise:
a biotin-binding moiety; and
a polypeptide antigen,
   wherein each of the first, second, third, and fourth biotinylated polysaccharide antigens is non-covalently associated with the biotin-binding moiety of the respective fusion protein to form an immunogenic complex.

Embodiment 23: The immunogenic composition of Embodiment 22, wherein the polypeptide antigen of the first fusion protein and of the second fusion protein is or comprises an SseB polypeptide antigen of Salmonella, and the polypeptide antigen of the third fusion protein and of the fourth fusion protein is or comprises an SP1500 polypeptide, an SP0785 polypeptide, or both, of S. pneumoniae, and/or the fusion protein is CP1.

Embodiment 24: The immunogenic composition of claim 23, wherein the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof, the SP1500 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, the SP0785 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 9, or an immunogenic fragment thereof, and the CP1 fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 6, or an immunogenic fragment thereof.

Embodiment 25: The immunogenic composition of any one of Embodiments 11-15, comprising at least four different species of immunogenic complexes, wherein the different species comprise:
a first biotinylated polysaccharide antigen comprising a polysaccharide of S. flexneri 2a non-covalently complexed with a first fusion protein; and
a second biotinylated polysaccharide antigen comprising a polysaccharide of S. flexneri 3a non-covalently complexed with a second fusion protein,
a third biotinylated polysaccharide antigen comprising a polysaccharide of S. flexneri 6 non-covalently complexed with a third fusion protein; and
a fourth biotinylated polysaccharide antigen comprising a polysaccharide of S. sonnei non-covalently complexed with a fourth fusion protein,
   wherein the first fusion protein, the second fusion protein, the third fusion protein, and the fourth fusion protein each independently comprise:
a biotin-binding moiety; and
a polypeptide antigen,
   wherein each of the first, second, third, and fourth biotinylated polysaccharide antigens is non-covalently associated with the biotin-binding moiety of the respective fusion protein to form an immunogenic complex.

Embodiment 26: The immunogenic composition of Embodiment 25, wherein the polypeptide antigen of the first fusion protein, the second fusion protein, the third fusion protein, and the fourth fusion protein is or comprises an SseB polypeptide antigen of Salmonella.

Embodiment 27: The immunogenic composition of Embodiment 26, wherein the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof.

Embodiment 28: A vaccine comprising a plurality of different species of immunogenic complexes, wherein the different species comprise:
a biotinylated polysaccharide antigen of Shigella serotype S. flexneri 2a non-covalently complexed with a fusion protein;
a biotinylated polysaccharide antigen of Shigella serotype S. flexneri 3a non-covalently complexed with a fusion protein;
   wherein each fusion protein comprises
a biotin-binding moiety; and
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
   wherein each of the biotinylated polysaccharide antigens is non-covalently associated with the biotin-binding moiety of at least one fusion protein to form an immunogenic complex.

Embodiment 29: The vaccine of Embodiment 28, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

Embodiment 30: The vaccine of Embodiment 28, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

Embodiment 31: The vaccine of Embodiment 28, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8, SEQ ID NO: 9, or both SEQ ID NO: 8 and SEQ ID NO:9.

Embodiment 32: A vaccine comprising a plurality of different species of immunogenic complexes, wherein the different species comprise:
a biotinylated polysaccharide antigen of Shigella serotype S. flexneri 6 non-covalently complexed with a fusion protein;
a biotinylated polysaccharide antigen of Shigella serotype S. sonnei non-covalently complexed with a fusion protein;
   wherein each fusion protein comprises
a biotin-binding moiety; and
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and wherein each of the biotinylated polysaccharide antigens is non-covalently associated with the biotin-binding moiety of at least one fusion protein to form an immunogenic complex.

Embodiment 33: The vaccine of Embodiment 32, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

Embodiment 34: The vaccine of Embodiment 32, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

Embodiment 35: The vaccine of Embodiment 32, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8, SEQ ID NO: 9, or both SEQ ID NO: 8 and SEQ ID NO:9.

Embodiment 36: A vaccine comprising a plurality of different species of immunogenic complexes, wherein the different species comprise:
a biotinylated polysaccharide antigen of Shigella serotype S. flexneri 2a non-covalently complexed with a fusion protein;
a biotinylated polysaccharide antigen of Shigella serotype S. flexneri 3a non-covalently complexed with a fusion protein;
a biotinylated polysaccharide antigen of Shigella serotype S. flexneri 6 non-covalently complexed with a fusion protein;
a biotinylated polysaccharide antigen of Shigella serotype S. sonnei non-covalently complexed with a fusion protein;
   wherein each fusion protein comprises
a biotin-binding moiety; and
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
   wherein each of the biotinylated polysaccharide antigens is non-covalently associated with the biotin-binding moiety of at least one fusion protein to form an immunogenic complex.

Embodiment 37: The vaccine of Embodiment 36, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

Embodiment 38: The vaccine of Embodiment 36, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

Embodiment 39: The vaccine of Embodiment 36, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8, SEQ ID NO: 9, or both SEQ ID NO: 8 and SEQ ID NO:9.

Embodiment 40: A vaccine comprising a plurality of different species of immunogenic complexes, wherein the different species comprise:
a biotinylated polysaccharide antigen of Shigella serotype S. Flexneri 2a non-covalently complexed with a fusion protein;
a biotinylated polysaccharide antigen of Shigella serotype S. Flexneri 3a non-covalently complexed with a fusion protein;
a biotinylated polysaccharide antigen of Shigella serotype S. Flexneri 6 non-covalently complexed with a fusion protein;
a biotinylated polysaccharide antigen of Shigella serotype S. Sonnei non-covalently complexed with a fusion protein;
   wherein each fusion protein comprises
a biotin-binding moiety;
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
a second polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
   wherein each of the biotinylated polysaccharide antigens is non-covalently associated with the biotin-binding moiety of at least one fusion protein to form an immunogenic complex.

Embodiment 41: The vaccine of Embodiment 40, wherein the vaccine comprises a stoichiometrically equal ratio, by weight, of each of the polysaccharide antigens of the different species.

Embodiment 42: The vaccine of Embodiment 40, wherein the vaccine comprises at least one of the polysaccharide antigens of the different species at a stoichiometrically different ratio, by weight.

Embodiment 43: The vaccine of any of Embodiments 28-42, wherein the vaccine comprises a stoichiometrically different ratio, by weight, of each of the polysaccharide antigens of the different species.

Embodiment 44: The vaccine of any one of the preceding Embodiments, wherein the biotin-binding moiety is a polypeptide comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 3.

Embodiment 45: An immunogenic complex comprising a biotinylated polysaccharide antigen of Shigella non-covalently associated with a fusion protein, wherein the fusion protein comprises a biotin-binding moiety and at least one polypeptide antigen.

Embodiment 46: The immunogenic complex of Embodiment 45, wherein the biotinylated polysaccharide antigen comprises a polysaccharide of Shigella having a serotype selected from S. flexneri 2a, flexneri 3a, S. flexneri 6 or S. sonnei.

Embodiment 47: The immunogenic complex of either Embodiment 45 or 46, wherein the fusion protein comprises SseB, IpaB, or an SP1500 polypeptide, an SP0785 polypeptide, or both.

Embodiment 48: The immunogenic complex of claim 47, wherein the fusion protein comprises:
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an antigenic fragment thereof,
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5 or an antigenic fragment thereof; or
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof.

Embodiment 49: The immunogenic complex of any one of claims 45-48, comprising a ratio of fusion protein to polysaccharide antigen of about 1:1, about 2:1, about 3:1, about 4:1, about 5:1, about 6:1, about 7:1, about 8:1, about 9:1, or about 10:1, by weight.

Embodiment 50: A vaccine comprising one or more immunogenic complexes of any one of Embodiments 45-49.

Embodiment 51: A pharmaceutical composition comprising the vaccine of any one of Embodiments 1-44 and 46, and a pharmaceutically acceptable carrier.

Embodiment 52: A pharmaceutical composition comprising the immunogenic complex of any one of Embodiments 45-49, and a pharmaceutically acceptable carrier.

Embodiment 53: The pharmaceutical composition of Embodiment 51 or 52, further comprising one or more adjuvants.

Embodiment 54: The pharmaceutical composition of Embodiment 53, wherein the one or more adjuvants is or comprises a co-stimulation factor.

Embodiment 55: The pharmaceutical composition of Embodiment 53 or 54, wherein the one or more adjuvants are selected from the group consisting of aluminum phosphate, aluminum hydroxide, and phosphated aluminum hydroxide.

Embodiment 56: The pharmaceutical composition of any one of Embodiments 53-55, wherein the one or more adjuvants is or comprises aluminum phosphate.

Embodiment 57: The pharmaceutical composition of any one of Embodiments 51-56, wherein the pharmaceutical composition is formulated for injection.

Embodiment 58: The pharmaceutical composition of any one of Embodiments 51-57, wherein upon administration to a subject, the pharmaceutical composition induces an immune response.

Embodiment 59: The pharmaceutical composition of Embodiment 58, wherein the immune response is to (i) at least one polysaccharide antigen of the vaccine or immunogenic complex, and/or (ii) at least one polypeptide antigen of the vaccine or immunogenic complex.

Embodiment 60: A method of making a vaccine, comprising non-covalently complexing a plurality of biotinylated polysaccharide antigens with a plurality of fusion proteins, wherein each fusion protein comprises at least one polypeptide antigen selected SseB, IpaB, SP0785 or SP1500;
wherein the plurality of biotinylated polysaccharide antigens comprises polysaccharides of one or more Shigella serotypes selected from Typhimurium, Enteritidis, Typhi, and Paratyphi A.

Embodiment 61: A method of immunizing a subject against Shigella infection and/or colonization comprising administering to the subject an immunologically effective amount of the vaccine of any one of Embodiments 1-44 and 46.

Embodiment 62: A method of immunizing a subject against Shigella infection and/or colonization comprising administering to the subject an immunologically effective amount of the immunogenic complex of any one of Embodiments 45-49.

Embodiment 63: A method of immunizing a subject against Shigella infection and/or colonization comprising administering to the subject an immunologically effective amount of the pharmaceutical composition of any one of Embodiments 51-58.

Embodiment 64: The method of any one of Embodiments 61-63, wherein the vaccine, immunogenic composition, or pharmaceutical composition induces an immune response.

Embodiment 65: The method of any one of Embodiments 61-64, wherein the immune response is to at least one polysaccharide antigen or at least one polypeptide of a fusion protein.

Embodiment 66: The method of any one of Embodiments 61-64, wherein the subject is immunized against Shigella infection and/or colonization with one dose of a vaccine.

Embodiment 67: The method of any one of Embodiments 61-64, wherein the subject is immunized against Shigella infection and/or colonization with two doses of a vaccine.

Embodiment 68: The method of any one of Embodiments 61-64, wherein the subject is immunized against Shigella infection and/or colonization with three doses of a vaccine.

Embodiment 69: A fusion protein comprising a rhizavidin protein and at least one peptide or polypeptide antigen, wherein the rhizavidin protein comprises amino acids of SEQ ID NO: 3, or 85% sequence identity to amino acids of SEQ ID NO: 3, and Salmonella peptide or polypeptide comprises a fragment of at least 20 amino acids of the SseB protein, or the Shigella peptide or polypeptide comprises a fragment of at least 20 amino acids of the IpaB protein.

Embodiment 70: The fusion protein of Embodiment 69, wherein the SseB protein comprises at least SEQ ID NO: 4 or a protein of at least 20 amino acids that has at least 85% sequence identity to SEQ ID NO: 4.

Embodiment 71: The fusion protein of Embodiment 69, wherein the IpaB protein comprises at least SEQ ID NO: 5 or a protein of at least 20 amino acids that has at least 85% sequence identity to SEQ ID NO: 5.

Embodiment 72: The fusion protein of Embodiment 69, wherein the fusion protein comprises at least SEQ ID NO: 1.

Embodiment 73: The fusion protein of Embodiment 69, wherein the fusion protein comprises at least SEQ ID NO: 2.

Embodiment 74: The fusion protein of any of Embodiments 69-73, wherein the fusion protein comprises a Salmonella peptide or polypeptide comprises a fragment of at least 20 amino acids of the SseB protein, and the Shigella peptide or polypeptide comprises a fragment of at least 20 amino acids of the IpaB protein.

Embodiment 75: The fusion protein of any of Embodiments 69-74, wherein the fusion protein is selected from any fusion protein in Table 1.

Embodiment 76: A vaccine composition comprising at least two immunogenic complexes selected from any combination of Shigella-MAPS immunogenic complexes listed in Table 2A, and at least two or more immunogenic complexes selected from any of the Salmonella-MAPS immunogenic complexes listed in any of Tables 3A, 3B or 3C.

Embodiment 77: The vaccine composition of claim 75, comprising at least three immunogenic complexes selected from any combination of Shigella-MAPS immunogenic complexes listed in Table 2B, and at least two or more immunogenic complexes selected from any of the Salmonella-MAPS immunogenic complexes listed in any of Tables 3A, 3B or 3C.

Embodiment 78: The vaccine composition of claim 75, comprising at least four immunogenic complexes selected from any combination of Shigella-MAPS immunogenic complexes listed in Table 2C, and at least two or more immunogenic complexes selected from any of the Salmonella-MAPS immunogenic complexes listed in any of Tables 3A, 3B or 3C.

Embodiment 79: A fusion protein comprising, in any order:
a biotin-binding moiety and
a IpaB polypeptide.
   wherein the fusion protein comprises (i) an amino acid sequence that is at least 80% identical to SEQ ID NO: 3 (Rhavi) and (ii) an amino acid sequence that is at least 80% identical to SEQ ID NO: 5 (IpaB).

Embodiment 80: The fusion protein of Embodiment 79, wherein the biotin-binding moiety comprises an amino acid sequence that is at least 80%, 85%, 90% identical to SEQ ID NO: 3 or a biotin binding portion thereof.

Embodiment 81: The fusion protein of claim 79, wherein the IpaB polypeptide comprises an amino acid sequence that is at least 80%, 85%, 90% identical to SEQ ID NO: 5.

Embodiment 82: The fusion protein of claim 79, wherein the fusion protein comprises, in order of N-to C-terminal:
a. a biotin-binding moiety comprises an amino acid sequence of SEQ ID NO: 3, or an amino acid sequence that is at least 80%, 85%, 90% identical to SEQ ID NO:3 or a biotin binding portion thereof, and
b. a IpaB polypeptide comprising an amino acid sequence of SEQ ID NO: 5, or an amino acid sequence an amino acid sequence that is at least 80%, 85%, 90% identical to SEQ ID NO: 5

Embodiment 83: The fusion protein of Embodiment 79, wherein the fusion protein comprises, in order of N- to C-terminal:
a. a IpaB polypeptide comprising an amino acid sequence of SEQ ID NO: 5, or an amino acid sequence that is at least 80%, 85%, 90% identical to SEQ ID NO:5, and
b. a biotin-binding moiety comprises an amino acid sequence of SEQ ID NO: 3, or an amino acid sequence that is at least 80%, 85%, 90% identical to SEQ ID NO:3 or a biotin binding portion thereof.

Embodiment 84: The fusion protein of Embodiment 79, wherein the fusion protein comprises an amino acid sequence that is at least 80%, 85%, or 90% identical to SEQ ID NO: 2.

Embodiment 85: A fusion protein comprising, in any order:
a biotin-binding moiety and
a SseB polypeptide.
   wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB).

Embodiment 86: The fusion protein of Embodiment 85, wherein the biotin-binding moiety comprises an amino acid sequence that is at least 80%, 85%, 90% identical to SEQ ID NO: 3 or a biotin binding portion thereof.

Embodiment 87: The fusion protein of Embodiment 85, wherein the SseB polypeptide comprises an amino acid sequence that is at least 80%, 85%, 90% identical to SEQ ID NO: 4.

Embodiment 88: The fusion protein of Embodiment 85, wherein the fusion protein comprises, in order of N- to C-terminal:
a. a biotin-binding moiety comprises an amino acid sequence of SEQ ID NO: 3, or an amino acid sequence that is at least 80%, 85%, 90% identical to SEQ ID NO:3 or a biotin binding portion thereof, and
b. a SseB polypeptide comprising an amino acid sequence of SEQ ID NO: 4, or an amino acid sequence an amino acid sequence that is at least 80%, 85%, 90% identical to SEQ ID NO: 4.

Embodiment 89: The fusion protein of claim 85, wherein the fusion protein comprises, in order of N-to C-terminal:
a. a SseB polypeptide comprising an amino acid sequence of SEQ ID NO: 4, or an amino acid sequence that is at least 80%, 85%, 90% identical to SEQ ID NO: 4, and
b. a biotin-binding moiety comprises an amino acid sequence of SEQ ID NO: 3, or an amino acid sequence that is at least 80%, 85%, 90% identical to SEQ ID NO:3 or a biotin binding portion thereof.

Embodiment 90: The fusion protein of Embodiment 85, wherein the fusion protein comprises an amino acid sequence that is at least 80%, 85%, or 90% identical to SEQ ID NO: 1.

Embodiment 91: The fusion protein of any of Embodiments 1-90, further comprising at least one additional antigenic polypeptide, selected from any of: SP1500, SP875, IpaB or SseB.

Embodiment 92: The fusion protein of Embodiment 91, wherein the SP1500 polypeptide comprises an amino acid sequence of SEQ ID NO: 9, or an amino acid sequence that is at least 80%, 85%, 90% identical to SEQ ID NO: 9 or a biotin binding portion thereof.

Embodiment 93: The fusion protein of Embodiment 91, wherein the SP785 polypeptide comprises an amino acid sequence of SEQ ID NO: 8, or an amino acid sequence that is at least 80%, 85%, 90% identical to SEQ ID NO:8 or a biotin binding portion thereof.

Embodiment 94: The fusion protein of Embodiment 91, wherein the SseB polypeptide comprises an amino acid sequence of SEQ ID NO: 4 as disclosed in US patent Publication 2020/0087361, or an amino acid sequence that is at least 80%, 85%, 90% identical to SEQ ID NO:4 or a biotin binding portion thereof.

Embodiment 95: The fusion protein of any of Embodiments 79-94, comprising a linker or spacer positioned between the biotin-binding moiety and the IpaB polypeptide, or the biotin-binding moiety and the SseB polypeptide, wherein the linker comprises the amino acid sequence selected from: GGGSS, GGGGSSS, TDPNSSS, SSS, AAA, or any of SEQ ID NO: 37-52 as disclosed in U.S. application Ser. No. 16/568,646.

Embodiment 96: The fusion protein of any of Embodiments 79-95, wherein a biotin-binding moiety comprises an amino acid sequence of at least 80%, or 90%, or 95% sequence identity to SEQ ID NO: 3 that has any one or more of the amino acid modifications: N80, T108, N118, S119A, N138A.

Embodiment 97: The fusion protein of any of Embodiments 79-96, wherein a biotin-binding moiety comprises an amino acid sequence of at least 80%, or 90%, or 95%, 98%, 99% or 100% sequence identity to SEQ ID NO: 55, wherein the biotin binding moiety has at least one or more of the amino acid modifications: N80, T108, N118, S119A, N138A.

Embodiment 98: An immunogenic composition comprising at least at least one species of immunogenic complex, wherein each species of the immunogenic complex comprises:
(a) a biotinylated polysaccharide antigen comprising biotin and a 0-specific polysaccharide (OSP) antigen from a Shigella spp serogroup, and
(b) a fusion protein comprising:
   (i) a biotin-binding moiety; and
   (ii) at least one polypeptide antigen comprising a IpaB polypeptide,
      wherein the fusion protein comprises (i) an amino acid sequence that is at least 80% identical to SEQ ID NO: 3 (Rhavi) and (ii) an amino acid sequence that has at least 80% identical to SEQ ID NO: 5(IpaB), or an amino acid sequence that has at least 80% sequence identity to SEQ ID NO: 4 (SseB), and
      wherein in each species of the immunogenic complexes, the biotinylated OSP antigen is non-covalently associated with the biotin-binding moiety of the fusion protein.

Embodiment 99: The immunogenic composition of Embodiment 98, wherein the Shigella spp. serogroup is selected from: Shigella flexeri or Shigella sonnei.

Embodiment 100: The immunogenic composition of Embodiment 98, wherein the Shigella spp. serogroup is Serogroup A (e.g., Serogroup A comprises of 15 different serotypes; an exemplary example is S. dysenteriae), Serogroup B (comprises of 9 serotypes; an exemplary example is S. flexneri), Serogroup C (comprises of 19 serotypes; an exemplary example is S. boydii) or Serogroup D (comprises of one serotype, S. sonnei).

Embodiment 101: The immunogenic composition of Embodiment 98, wherein the O-specific polysaccharide (OSP) antigen is selected from a distinct Shigella flexeri serotype is selected from any of: 1a, 1b, 1c, 2a, 2b, 3a, 3b, 4a, 4b, 5a, 5b, 6, X, Xv, Y, and F6.

Embodiment 102: The immunogenic composition of Embodiment 98, wherein the O-specific polysaccharide (OSP) antigen is selected from a Shigella flexeri serotype selected from: 2a, 3a or 6, or Shigella sonnei.

Embodiment 103: The immunogenic composition of Embodiment 99, wherein the Shigella sonnei is strain 53G.

Embodiment 104: The immunogenic composition of any of Embodiments 98-103, comprising at least one species of immunogenic complex selected from:
a. an immunogenic complex comprising (a) a biotinylated polysaccharide antigen comprising biotin and a polysaccharide antigen from Shigella flexeri serotype 2a, and (b) a fusion protein;
b. an second immunogenic complex comprising (a) a biotinylated polysaccharide antigen comprising biotin and a polysaccharide antigen from Shigella flexeri serotype 3a, and (b) a fusion protein;
c. an third immunogenic complex comprising (a) a biotinylated polysaccharide antigen comprising biotin and a polysaccharide antigen from Shigella flexeri serotype 6, and (b) a fusion protein; and
d. an fourth immunogenic complex comprising (a) a biotinylated polysaccharide antigen comprising biotin and a polysaccharide antigen from Shigalla sonnei, and (b) a fusion protein.

Embodiment 105: The immunogenic composition of Embodiment 98, comprising at least 2, or at least 3 immunogenic complexes selected from any of:
a. an immunogenic complex comprising (a) a biotinylated polysaccharide antigen comprising biotin and a polysaccharide antigen from Shigella flexeri serotype 2a, and (b) a fusion protein;
b. an second immunogenic complex comprising (a) a biotinylated polysaccharide antigen comprising biotin and a polysaccharide antigen from Shigella flexeri serotype 3a, and (b) a fusion protein;
c. an third immunogenic complex comprising (a) a biotinylated polysaccharide antigen comprising biotin and a polysaccharide antigen from Shigella flexeri serotype 6, and (b) a fusion protein; and
d. an fourth immunogenic complex comprising (a) a biotinylated polysaccharide antigen comprising biotin and a polysaccharide antigen from Shigalla sonnei, and (b) a fusion protein.

Embodiment 106: The immunogenic composition of any of claims 98-105, comprising:
a. a first immunogenic complex comprising (a) a biotinylated polysaccharide antigen comprising biotin and a polysaccharide antigen from Shigella flexeri serotype 2a, and (b) a fusion protein;
b. a second immunogenic complex comprising (a) a biotinylated polysaccharide antigen comprising biotin and a polysaccharide antigen from Shigella flexeri serotype 3a, and (b) a fusion protein;
c. a third immunogenic complex comprising (a) a biotinylated polysaccharide antigen comprising biotin and a polysaccharide antigen from Shigella flexeri serotype 6, and (b) a fusion protein; and
d. a fourth immunogenic complex comprising (a) a biotinylated polysaccharide antigen comprising biotin and a polysaccharide antigen from Shigalla sonnei, and (b) a fusion protein.

Embodiment 107: The immunogenic composition of any of Embodiments 98-106 wherein the OMP antigen is a high molecular weight (HMW) antigen.

Embodiment 108: The immunogenic composition of any of Embodiments 98-107, comprising at least one additional species of immunogenic complex, wherein the additional species of immunogenic complex comprises:
(a) a biotinylated polysaccharide antigen comprising biotin and a 0-specific polysaccharide (OSP) antigen from a Samonella spp, and
(b) a fusion protein comprising:
   (i) a biotin-binding moiety; and
   (ii) at least one polypeptide antigen comprising a IpaB polypeptide,
      wherein the fusion protein comprises (i) an amino acid sequence that is at least 80% identical to SEQ ID NO: 3 (Rhavi) and (ii) an amino acid sequence that is at least 80% identical to SEQ ID NO: 5(IpaB), and
      wherein in each species of the immunogenic complexes, the biotinylated OSP antigen is non-covalently associated with the biotin-binding moiety of the fusion protein.

Embodiment 109: The immunogenic composition of any of Embodiments 98-108, wherein the OSP is from a Salmonella serogroup A, B, C2 and C3, D and E.

Embodiment 110: The immunogenic composition of Embodiment 109, wherein the OSP is from any of serotypes: Salmonella kentucky strain IS98, Salmonella enterica serovar Toucra 048 (also known as S. enterica subspecies enterica serovar Toucra), Salmonella enterica serovars Enteritidis and Typhimurium or Salmonella arizonae 045 (Arizona 11).

Embodiment 111: The immunogenic composition of any of Embodiments 98-110, wherein the fusion protein is defined by any of Embodiments 79-97.

Embodiments 112: A pharmaceutical composition comprising the immunogenic composition of any of Embodiments 98-110, and a pharmaceutically acceptable carrier.

Embodiment 113: The pharmaceutical composition of Embodiment 111, further comprising one or more adjuvants.

Embodiment 114: The pharmaceutical composition of Embodiment 111, wherein the one or more adjuvants is or comprises a co-stimulation factor.

Embodiment 115: The pharmaceutical composition of Embodiment 114, wherein the one or more adjuvants are selected from the group consisting of aluminum phosphate, aluminum hydroxide, and phosphated aluminum hydroxide.

Embodiment 116: A vaccine comprising at least one immunogenic composition of any of Embodiments 98-110, or a fusion protein of any of Embodiments 79-97, and a pharmaceutically acceptable carrier.

Embodiment 117: A method of making a multivalent vaccine, comprising mixing two or more species of immunogenic complexes of any of Embodiments 97-110 in a single formulation.

Embodiment 118: The method of Embodiment 117, comprising mixing four or more species of immunogenic complexes in a single formulation, wherein the four or more immunogenic complexes are disclosed in any of Embodiments 104-106.

Embodiment 119: The method of Embodiment 117, wherein each species of the immunogenic complexes comprises least one fusion protein is a fusion protein selected from any of Embodiments 79-97.

Embodiment 120: Use of the immunogenic composition of any of Embodiments 98-110, or the vaccine of Embodiment 116 to induce an immune response to a subject.

Embodiment 121: Use of a vaccine composition of Embodiment 116, or a fusion protein of any of Embodiments 79-97 to induce an immune response in a subject.

Embodiment 122: A method to induce an immune response to a subject, comprising administering to the subject a pharmaceutical composition comprising the immunogenic composition of any of Embodiments 89-110, or the vaccine of Embodiment 116.

Embodiment 123: A method to induce an immune response in a subject, comprising administering a vaccine comprising a fusion protein of any of Embodiments 79-97.

Embodiment 124: The method of Embodiment 123, wherein the immune response is an antibody or B cell response.

Embodiment 125: The method of Embodiments 123-124, wherein the immune response is a CD4+ T cell response, including Tₕ1, Tₕ2, or Tₕ17 response, or a CD8+ T cell response, or CD4+/CD8+ T cell response.

Embodiment 126: The method of Embodiments 123-125, wherein the immune response is:
an antibody or B cell response; and
a T cell response.

Embodiment 127: The method of Embodiments 123-126, wherein the immune response is to: at least the first antigenic polysaccharide, or the second antigenic polysaccharides, or both the first and second antigenic polysaccharide, or at least one polypeptide antigen.

Embodiment 128: The method of Embodiments 123-127, wherein the immune response is an antibody or B cell response to at least the first antigenic polysaccharide and/or second antigenic polysaccharide, and a CD4+ T cell response, including Tₕ 1, Tₕ2, or Tₕ 17 response, or a CD8+ T cell response, or CD4+/CD8+ T cell response to at least one polypeptide antigen.

Embodiment 129: The method of claims 123-128, wherein the immune response is an antibody or B cell response to at least the first antigenic polysaccharide and/or the second antigenic polysaccharide, and an antibody or B cell response and a CD4+ T cell response, including Tₕ1, Tₕ2, or Tₕ 17 response, or a CD8+ T cell response, or CD4+/CD8+ T cell response to at least one polypeptide antigen.

Embodiment 130: The method of claims 123-129, wherein the immune response protects the subject from at least one serotype of Shigella.

Embodiment 131: The method of claim 130, wherein the immune response protects the subject from at least one serotype of Shigella, selected from Shigella flexneri serotypes 2a, 3a, 6 or Shigella sonnei.

Embodiment 132: The immunogenic composition of any of Embodiments 98-110, wherein herein the immunogenic composition, upon administration to a subject, elicits (i) an immune response to the at least the OSP antigenic polysaccharide and (ii) an immune response to at least one of the polypeptide antigens, e.g., IpaB or SseB, or both, in the subject.

Embodiment 133: The immunogenic composition of any of Embodiments 98-110, wherein the immune response to the OSP antigenic polysaccharide, and/or at least one of the one polypeptide antigens (e.g., IpaB and/or SseB) comprises an antibody or B cell response.

Embodiment 134: The immunogenic composition of any of Embodiments 98-110, wherein the immune response to the OSP antigenic polysaccharide, and/or at least one of the one polypeptide antigens (e.g., IpaB and/or SseB) comprises a T cell response.

Embodiment 135: The immunogenic composition of any of Embodiments 98-110, wherein the immune response to the OSP antigenic polysaccharide, and/or at least one of the one polypeptide antigens (e.g., IpaB and/or SseB) comprises a CD4+ T cell response, including Tₕ1, Tₕ2, or Tₕ17 response, or a CD8+ T cell response, or a CD4+/CD8+ T cell response.

Embodiment 136: The immunogenic composition of any of Embodiments 98-110, wherein the immune response to the OSP antigenic polysaccharide, and/or at least one of the one polypeptide antigens (e.g., IpaB and/or SseB) comprises:
an antibody or B cell response; and
a T cell response.

Embodiment 137: The immunogenic composition of any of Embodiments 98-110, wherein the immunogenic composition, upon administration to a subject, elicits at least (i) an antibody or B cell response to the OSP antigenic polysaccharide, and (ii) an antibody or B cell response to at least one of polypeptide antigen (e.g., IpaB and/or SseB), in the subject.

Embodiment 138: The immunogenic composition of any of Embodiments 98-110, wherein the immunogenic composition, upon administration to a subject, elicits at least (i) an antibody or B cell response to the OSP antigenic polysaccharide, and (ii) an antibody or B cell response and a CD4+ T cell response, including Tₕ1, Tₕ2, or Tₕ17 response, or a CD8+ T cell response, or a CD4+/CD8+ T cell response to at least one of polypeptide antigens (e.g., IpaB and/or SseB) in the subject.

Embodiment 139: The immunogenic composition of any of Embodiments 98-110, wherein the immunogenic composition, upon administration to a subject, elicits at least (i) an antibody or B cell response to the at least the OMP antigenic polysaccharide, and (ii) a CD4+ T cell response, including Tₕ1, Tₕ2, or Tₕ17 response, or a CD8+ T cell response, or a CD4+/CD8+ T cell response to at least one of polypeptide antigen, e.g., IpaB, in the subject.

Embodiment 140: A heterologous nucleic acid sequence encoding a fusion protein comprising, in any order:
a. a biotin-binding moiety and
b. a IpaB polypeptide,
   wherein the fusion protein comprises (i) an amino acid sequence that is at least 80% identical to SEQ ID NO: 3 (Rhavi) and (ii) an amino acid sequence that is at least 80% identical to SEQ ID NO: 5(IpaB).

Embodiment 141: A heterologous nucleic acid sequence encoding a fusion protein comprising, in any order:
a. a biotin-binding moiety and
b. a SseB polypeptide,
   wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB).

Embodiment 142: The heterologous nucleic acid of Embodiment 140 or 141, wherein the nucleic acid sequence comprises a nucleic acid sequence of SEQ ID NO: 60 or a nucleic acid sequence at least 85% sequence identity to SEQ ID NO: 60, and a nucleic acid sequence of SEQ ID NO: 46 or a nucleic acid sequence at least 85% sequence identity to SEQ ID NO: 46.

Embodiment 143: The heterologous nucleic acid of Embodiment 140, wherein the nucleic acid sequence comprises SEQ ID NO: 45, or a nucleic acid sequence having at least 85% sequence identity to SEQ ID NO: 45.

Embodiment 144: The heterologous nucleic acid of any of Embodiments 140-143, wherein the heterologous nucleic acid further comprises a nucleic acid that encodes at least one linker, or a nucleic acid that encodes an expression tag, or both.

Embodiment 145: The nucleic acid sequence of Embodiment 144, wherein the nucleic acid sequence encoding at least one linker is located between SEQ ID NO: 60 and SEQ ID NO: 46, or sequences at having least 85% to SEQ ID NO: 60 or 46, thereof.

Embodiment 146: The nucleic acid sequence of Embodiment 144, wherein the nucleic acid sequence encoding at least one linker encodes a linker having an amino acid sequence selected from any of: GGGSS, GGGGSSS or AAA and any of: SEQ ID NOS: 39-60 and SEQ ID NO: 62, 63, 108, 109 as disclosed in U.S. application Ser. No. 16/568,646.

Embodiment 147: The nucleic acid sequence of Embodiment 140, wherein the heterologous nucleic acid is selected from one of the following, in the following order:
a. a nucleic acid sequence of SEQ ID NO: 60 or a nucleic acid sequence at least 85% sequence identity to SEQ ID NO: 60, and a nucleic acid sequence of SEQ ID NO: 46 or a nucleic acid sequence at least 85% sequence identity to SEQ ID NO: 46, or
b. a nucleic acid sequence of SEQ ID NO: 46 or a nucleic acid sequence at least 85% sequence identity to SEQ ID NO: 46, and a nucleic acid sequence of SEQ ID NO: 60 or a nucleic acid sequence at least 85% sequence identity to SEQ ID NO: 60.

Embodiment 148: A cell transfected with the nucleic acid sequence of Embodiment 140 or 141.

Embodiment 149: The cell of Embodiment 148, wherein the cell is transfected with the nucleic acid sequence of Embodiment 143.

Embodiment 150: The cell of Embodiment 148, wherein the cell is transfected with the nucleic acid sequence of Embodiment 142 or 147.

Embodiment 151: The cell of any of Embodiments 148-149, wherein the cell is an expression host cell.

Embodiment 152: The cell of Embodiment 151, wherein the expression host cell is selected from the group consisting of: E. coli, an insect cell line or a mammalian cell line.

Embodiment 153: The cell of Embodiment 152, wherein the insect cell line is baculovirus expression system.

Embodiment 154: The cell of Embodiment 152, wherein the mammalian cell line is a human cell line or Chinese Hamster ovary (CHO) cell line.

Embodiment 155: The cell of Embodiment 152, wherein the heterologous nucleic acid sequence is codon-optimized to improve expression in the host cell.

Embodiment 156: The cell of Embodiment 152, wherein the nucleic acid sequence further comprises a nucleic acid sequence encoding one or more of: polyadenylation sequence or termination sequence, a signal sequence.

Embodiment 157: An expression vector comprising the nucleic acid sequence of any of Embodiments 140-147.

Embodiment 158: The expression vector of Embodiment 157, comprising the nucleic acid sequence of claim 143.

Embodiment 159: The expression vector of Embodiment 157, comprising the nucleic acid sequence of Embodiment 142 or 147.

Embodiment 160: The expression vector of any of Embodiments 148-156, comprising the expression vector of Embodiment 157.

Embodiment 161: The expression vector of any of Embodiments 148-156, comprising the expression vector of claim 158.

Embodiment 162: The expression vector any of Embodiments 148-156, comprising the expression vector of Embodiment 142 or 147.

Embodiment 163: The expression vector of any of Embodiments 140-147, wherein the nucleic acid encodes a fusion protein comprising a biotin-binding moiety having an amino acid sequence of at least 80%, or 90%, or 95% sequence identity to SEQ ID NO: 3 that has any one or more of the amino acid modifications: N80, T108, N118, S119A, N138A.

Embodiment 164: The expression vector of any of Embodiments 140-147 or 163, wherein a biotin-binding moiety comprises an amino acid sequence of at least 80%, or 90%, or 95%, 98%, 99% or 100% sequence identity to SEQ ID NO: 55, wherein the biotin binding moiety has at least one or more of the amino acid modifications: N80, T108, N118, S119A, N138A.

### Certain Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastrical, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: In its broadest sense, the term “amino acid”, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Non-standard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kDa tetrameric agents comprised of two identical heavy chain polypeptides (about 50 kDa each) and two identical light chain polypeptides (about 25 kDa each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi-or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies*); Small Modular ImmunoPharmaceuticals (“SMIPs”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR©s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Antigen: The term “antigen”, as used herein, refers to (i) an agent that induces an immune response; and/or (ii) an agent that binds to a T cell receptor (e.g., when presented by an MHC molecule) or to an antibody. In some embodiments, an antigen induces a humoral response (e.g., including production of antigen-specific antibodies); in some embodiments, an antigen induces a cellular response (e.g., involving T cells whose receptors specifically interact with the antigen). In some embodiments, an antigen induces a humoral response and a cellular response. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)), etc. In some embodiments, an antigen is or comprises a polypeptide. In some embodiments, an antigen is or comprises a polysaccharide. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen. In some embodiments, an antigen is a polypeptide or a polysaccharide that, upon administration to a subject, induces a specific and/or clinically relevant immune response to such polypeptide or polysaccharide. In some embodiments, an antigen is selected to induce a specific and/or clinically relevant immune response to such polypeptide or polysaccharide.

Associated with: Two entities are “associated” with one another, as that term is used herein, if the presence, level and/or form of one is correlated with that of the other. In some embodiments, two or more entities are physically “associated” with one another if they interact, directly or indirectly, so that they are and/or remain in physical proximity with one another. In some embodiments, two or more entities that are physically associated with one another are covalently linked to one another. In some embodiments, two or more entities that are physically associated with one another are not covalently linked to one another but are non-covalently associated, for example by means of affinity interactions, electrostatic interactions, hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Carrier protein: As used herein, the term “carrier protein” refers to a protein or peptide that is coupled, complexed, or otherwise associated with a hapten (e.g., a small peptide or lipid) or less immunogenic antigen (e.g., a polysaccharide) and that induces or improves an immune response to such a coupled, or complexed, or otherwise associated hapten (e.g., a small peptide or lipid) or less immunogenic antigen (e.g., a polysaccharide). In some embodiments, such an immune response is or comprises a response to a hapten or less immunogenic antigen that is coupled, complexed, or otherwise associated with such a carrier protein. In some embodiments, such an immune response is or comprises a response to both a carrier protein and a hapten or less immunogenic antigen that is coupled, complexed, or otherwise associated with such a carrier protein. In some embodiments, no significant immune response to a carrier protein itself occurs. In some embodiments, immune response to a carrier protein may be detected; in some such embodiments, immune response to such a carrier protein is strong. In some embodiments, a carrier protein is coupled, complexed, or otherwise associated with one or more other molecules.

Colonization: As used herein, the term “colonization” generally refers to the ability of a microbe to grow at a target site or surface. For example, the term “colonization” refers to the ability of a microbe (e.g., a bacterium) to grow at an anatomical site (e.g., a mucosal membrane, gastrointestinal tract, injury site, organ, etc.) of a host.

Combination therapy: As used herein, the term “combination therapy” refers to those situations in which a subject is exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Derivative: As used herein, the term “derivative”, or grammatical equivalents thereof, refers to a structural analogue of a reference substance. That is, a “derivative” is a substance that shows significant structural similarity with the reference substance, for example sharing a core or consensus structure, but also differs in certain discrete ways. Such a substance would be said to be “derived from” said reference substance. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, R-sheet character, coiled-coil character, random coil character, etc.), and/or by a particular functional feature (e.g., binding activity, enzymatic activity, folding activity, signaling activity, etc.).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Fragment: A “fragment” of a material or entity as described herein has a structure that includes a discrete portion of the whole, but lacks one or more moieties found in the whole. In some embodiments, a fragment consists of such a discrete portion. In some embodiments, a fragment includes a discrete portion of the whole which discrete portion shares one or more functional characteristics found in the whole. In some embodiments, a fragment consists of such a discrete portion. In some embodiments, a fragment consists of or comprises a characteristic structural element or moiety found in the whole. In some embodiments, a fragment of a polymer, e.g., a polypeptide or polysaccharide, comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, or 99% of the monomeric units (e.g., residues) found in the whole polymer. The whole material or entity may in some embodiments be referred to as the “parent” of the whole.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98% or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In some embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, at least 99.5%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, inhibit or reduce: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Immunologically effective amount or immunologically effective dose: As used herein, “immunologically effective amount” or “immunologically effective dose” refers to an amount of an antigenic or immunogenic substance, e.g., an antigen, immunogen, immunogenic complex, immunogenic composition, vaccine, or pharmaceutical composition, which when administered to a subject, either in a single dose or as part of a series of doses, that is sufficient to enhance a subject's own immune response against a subsequent exposure to a pathogen. In some embodiments, the pathogen is Shigella. In some embodiments, the immune response is against one or more different serotypes of Shigella. In some embodiments, the immune response is against two or more different serotypes of Shigella. In some embodiments, the immune response is against four or more different serotypes of Shigella. An immunologically effective amount may vary based on the subject to be treated, the species of the subject, the degree of immune response desired to induce, etc. In some embodiments, an immunologically effective amount is sufficient for treatment or protection of a subject having or at risk of having disease. In some embodiments, an immunologically effective amount refers to a non-toxic but sufficient amount that can be an amount to treat, attenuate, or prevent infection and/or disease (e.g., bacterial infection, pneumococcal infection, bacterial colonization, pneumococcal colonization, complications associated with bacterial infection, complications associated with pneumococcal infection, etc.) in any subject. In some embodiments, an immunologically effective amount is sufficient to induce an immunoprotective response upon administration to a subject.

Immunoprotective response or protective response: As used herein, “immunoprotective response” or “protective response” refers to an immune response that mediates antigen or immunogen-induced immunological memory. In some embodiments, an immunoprotective response is induced by the administration of a substance, e.g., an antigen, immunogen, immunogenic complex, immunogenic composition, vaccine, or pharmaceutical composition to a subject. In some embodiments, immunoprotection involves one or more of active immune surveillance, a more rapid and effective response upon immune activation as compared to a response observed in a naïve subject, efficient clearance of the activating agent or pathogen, followed by rapid resolution of inflammation. In some embodiments, an immunoprotective response is an adaptive immune response. In some embodiments, an immunoprotective response is sufficient to protect an immunized subject from productive infection by a particular pathogen or pathogens to which a vaccine is directed (e.g., Shigella infection).

Immunization: As used herein, “immunization”, or grammatical equivalents thereof, refers to a process of inducing an immune response to an infectious organism or agent in a subject (“active immunization”), or alternatively, providing immune system components against an infectious organism or agent to a subject (“passive immunization”). In some embodiments, immunization involves the administration of one or more antigens, immunogens, immunogenic complexes, vaccines, immune molecules such as antibodies, immune sera, immune cells such as T cells or B cells, or pharmaceutical compositions to a subject. In some embodiments, immunization is performed by administering an immunologically effective amount of a substance, e.g., an antigen, immunogen, immunogenic complex, immunogenic composition, vaccine, immune molecule such as an antibody, immune serum, immune cell such as a T cell or B cell, or pharmaceutical composition to a subject. In some embodiments, immunization results in an immunoprotective response in the subject. In some embodiments, active immunization is performed by administering to a subject an antigenic or immunogenic substance, e.g., an antigen, immunogen, immunogenic complex, vaccine, or pharmaceutical composition. In some embodiments, passive immunization is performed by administering to a subject an immune system component, e.g., an immune molecule such as an antibody, immune serum, or immune cell such as a T cell or B cell.

Isolated: As used herein, the term “isolated”, or grammatical equivalents thereof, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polysaccharide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide or polysaccharide that is chemically synthesized or is synthesized in a cellular system different from that which produces it in nature is considered to be an “isolated” polypeptide or polysaccharide. Alternatively or additionally, in some embodiments, a polypeptide or polysaccharide that has been subjected to one or more purification techniques may be considered to be an “isolated” polypeptide or polysaccharide to the extent that it has been separated from other components a) with which it is associated in nature; and/or b) with which it was associated when initially produced.

Linker: As used herein, the term “linker” is used to refer to an entity that connects two or more elements to form a multi-element agent. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Plurality: As used herein, the term “plurality” includes at least 2 or more, including, e.g., at least 3, at least 4, at least 5, at least 6, at least 7, at least 8, at least 9, at least 10, or more.

Polysaccharide: The term “polysaccharide” as used herein refers to a polymeric carbohydrate molecule composed of long chains of monosaccharide units bound together by glycosidic, phosphodiester, or other linkages, and on hydrolysis give the constituent monosaccharides or oligosaccharides. Polysaccharides range in structure from linear to highly branched. Examples include storage polysaccharides such as starch and glycogen, structural polysaccharides such as cellulose and chitin and microbial polysaccharides, and antigenic polysaccharides found in microorganisms including, but not limited to, O specific polysaccharides (CPS), O polysaccharides (OPS), core O polysaccharides (COPS), and lipopolysaccharides (LPS).

Polypeptide: The term “polypeptide”, as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Prevention: The term “prevent” or “prevention”, as used herein in connection with a disease, disorder, and/or medical condition, refers to reducing the risk of developing the disease, disorder and/or condition, and/or a delay of onset, and/or reduction in frequency and/or severity of one or more characteristics or symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete when onset of a disease, disorder or condition has been delayed for a predefined period of time.

Protein: As used herein, the term “protein” encompasses a polypeptide. Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant: As used herein, the term “recombinant” is intended to refer to polypeptides that are designed, engineered, prepared, expressed, created, manufactured, and/or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, subject, population, sample, sequence or value of interest is compared with a reference or control agent, animal, subject, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a “response” to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Subject or tumor response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of biomarkers in a sample obtained from a subject, cytology, and/or histology. The exact response criteria can be selected in any appropriate manner, provided that when comparing groups of subjects and/or tumors, the groups to be compared are assessed based on the same or comparable criteria for determining response rate. One of ordinary skill in the art will be able to select appropriate criteria.

Risk: As will be understood from context, “risk” of a disease, disorder, and/or condition refers to a likelihood that a particular subject will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments, risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from subjects comparable to a particular subject. In some embodiments, relative risk is 0, 1, 2, 3, 4, 5, 6, 7, 8, 9, 10, or more.

Serotype: As used herein, the term “serotype”, also referred to as a serovar, refers to a distinct variation within a species of bacteria or virus or among immune cells of different subjects. These microorganisms, viruses, or cells are classified together based on their cell surface antigens, allowing the epidemiologic classification of organisms to the sub-species level. A group of serovars with common antigens may be referred to as a serogroup or sometimes serocomplex.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

Susceptible to: A subject who is “susceptible to” a disease, disorder, or condition is at risk for developing the disease, disorder, or condition. In some embodiments, a subject who is susceptible to a disease, disorder, or condition does not display any symptoms of the disease, disorder, or condition. In some embodiments, a subject who is susceptible to a disease, disorder, or condition has not been diagnosed with the disease, disorder, and/or condition. In some embodiments, a subject who is susceptible to a disease, disorder, or condition is a subject who has been exposed to conditions associated with development of the disease, disorder, or condition. In some embodiments, a risk of developing a disease, disorder, and/or condition is a population-based risk (e.g., family members of subjects suffering from the disease, disorder, or condition).

Symptoms are reduced: As used herein, “symptoms are reduced” when one or more symptoms of a particular disease, disorder or condition is reduced in magnitude (e.g., intensity, severity, etc.) and/or frequency, e.g., to a stastistically and/or clinically significant or relevant level. For purposes of clarity, a delay in the onset of a particular symptom is considered one form of reducing the frequency of that symptom.

Treatment: As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vaccination: As used herein, the term “vaccination” refers to the administration of a composition intended to generate an immune response, for example to a disease-causing agent. For the purposes of the present invention, vaccination can be administered before, during, and/or after exposure to a disease-causing agent, and in some embodiments, before, during, and/or shortly after exposure to the agent. In some embodiments, vaccination includes multiple administrations, appropriately spaced in time, of a vaccinating composition. In some embodiments, vaccination initiates immunization.

### EXAMPLES

### Materials and Methods

Aluminum phosphate (alum) was from Brenntag North America (2% Alhydrogel). Adipic acid dihydrazide (ADH), 1-Ethyl-3-[3-dimethylaminopropyl]carbodiimide Hydrochloride (EDC) and N-hydroxysulfosuccinimide (NHS), and 1-cyano-4-dimethylaminopyridinium tetrafluoroborate (CDAP) were purchased from Thermo Fisher. Restriction endonucleases and T7 shuffle express competent cells were purchased from New England Biolabs. Plasmid pETDuet was purchased from Novagen. All other reagents were obtained from Sigma. For the Salmonella-MAPS immunogenic complexes: Vi polysaccharide was obtained from Dr. Szu from NIH {Szu, 1989 #13}.

### Protein Purification

DNA fragments encoding of His-SseB and SseA or His-IpaB and IpgC were synthesized and cloned into a pETDuet vector containing Rhavi by restriction enzyme digestion and ligation. Alternatively, nucleic acid sequencess encoding of SseB and SseA-His or IpaB and IpgC-His were synthesized and cloned into a pETDuet vector comprising a nucleic acid encoding Rhavi by restriction enzyme digestion and ligation. Sequence-confirmed plasmids were transformed E. coli T7 shuffle express cells and transformants containing the relevant cloned proteins were grown to OD600=1 at 25° C., and protein expression was induced with 0.2 mM IPTG at 16° C. overnight. Cells were spun down and pellets were resuspended in lysis buffer (20 mM Tris-HCl, 500 mM NaCl, pH8.0) and then lysed by sonication. Supernatant was loaded over a Ni-NTA column and SseA or IpgC was washed off column with 0.05% LDAO in lysis buffer; proteins Rhavi-SseB and Rhavi-IpaB were eluted in imidazole buffer. Protein-containing elutions were combined, purified over a gel-filtration column for dimer fractions.

Purification of OSP from Shigella flexneri or Shigella sonnei

20 clinical strains of Shigella (obtained from University of Maryland) were grown and pellets harvested. Pellets were boiled at 3% acetic acid for 3 hrs, and supernatant was dialyzed against H₂O and separated on FPLC. OSP were purified by acid hydrolysis and TFF buffer exchange. Shigella strains with the highest molecular weight OSPs were selected for scale up. OSP from 20 clinical strains of Shigella (obtained from University of Maryland) were purified by acid hydrolysis and size exclusion column. Shigella Sonnei strain 53G was chosen as the OSP of other clinical stains had low MW. All OSPs were confirmed by NMR. For the Salmonella-MAPS immunogenic complexes: OSPs were purified from S. Paratyphi 9150 (ATCC), clinical strains of S. Typhimurium and S. Enteritidis using a protocol established previously with modifications {Micoli, 2013}. Briefly, bacteria were resuspended in 2% acetic acid and boiled for 2 hours at 100° C. Supernatant was dialyzed against water extensively and then lyophilized. Resuspended OSP was treated with DNase and Proteinase K and then loaded on a gel-filtration column to separate the OSP with larger sizes. Final OSP was lyophilized and frozen in −80° C.

### Biotinylation of OSP

OSP from shigella or salmonella was biotinylated with CDAP using protocol as described previously {Lu, 2009}{Zhang, 2013}. Biotinylated OSP was dialyzed against saline extensively before being used for MAPS assembly. MAPS was assembled at a 3:1 (w:w) protein: polysaccharide ratio and purified with size exclusion columns. Protein concentration was determined by the BCA method (Pierce), and OSP concentration was determined using the Anthrone method {Roe, 1955}.

### Antigen Preparations and Immunization

For immunization experiments with Shigella-MAPS or Salmonella-MAPS, vaccines were mixed with aluminum phosphate (alum) at the indicated concentration in a 5 ml tube, which was then tumbled overnight at 4° C. to allow for adsorption one day before immunization. MAPS assembly was carried out by combining known ratio of polysaccharide (PS): Protein in end-over-end rotation at 4° C. overnight. The MAPS immunogenic complex was purified on FPLC. Free protein and PS were removed by TFF. Final MAPS immunogenic complexes have a ratio of 4:1 protein: PS ratio. Rabbit immunization experiments were carried out at Cocalico Biologicals Inc.

### Enzyme-Linked Immunosorbent Assay (ELISA)

IgG antibody titers against Vi and ParaOSP were measured using the methods described previously {Konadu, 1996}{Lu, 2012}.

### Bacterial Killing Assays

Killing assay was performed as described previously {Lu, 2012 #1013}{Gondwe, #51; Hale, 2006 #163; Gondwe, 2010 #51} with Salmonella typhimurium strains carrying an empty vector (Strain C5) or expressing Vi polysaccharide on the surface (Strain C5.507) {Lu, 2012 #1013}. Bactericidal assays for S. Paratyphi A, S. Typhimurium and S. Enteritidis were carried out as described previously using ATCC 9150 strain or clinical strains {Boyd, 2014}.

### Example 1. MAPS Vaccine Against Serogroups Shigella flexneri and Shigella sonnei

Shigella serogroups Shigella flexneri and shigella sonnei are the most common serogroups causing invasive shigellosis in low-income countries (LIC). O-specific polysaccharide (OSP) and pathogen-specific proteins are potential targets. MAPS vaccines present a few advantages: ease of manufacture and tech transfer, and low cost of goods. Additionally, previous work using pneumococcal MAPS in older adults showed higher or similar functional PS antibody levels compared to currently available PCV13 or PPSV23 vaccines. MAPS vaccines could also confer additional protection from included pathogen-specific antigens.

### Selection of Carrier Proteins and Detergent Selection

The carrier function of three proteins, SseB or IpaB fused to rhizavidin (Rhavi), or CP1, was compared. Next, the antibody titer and bactericidal activity was evaluated following immunization of rabbits with monovalent MAPS. The immunogenicity of bivalent and quadrivalent Shigella-MAPS or Salmonella MAPS in rabbits was also evaluated.

The components of high molecular weight OSP from strains Shigella flexneri 2a, Shigella flexneri 3a, Shigella flexneri 6, and Shigella sonnei strain 53G, the fusion protein selected is Rhavi-IpaB, aluminum phosphate is 0.5 mg/ml, and the excipient is histidine buffered saline with 0.02% Tween 80. The Shigella OSP structures are described in FIG. 1. The selection of strains for OSP purification is as follows: small scale preparations of 20 clinical strain from the University of Maryland were grown and pellets were harvested and boiled in 3% acetic acid for 3 hours. The supernatant was dialyzed against H2O and separated on FPLC and the strains expressing the highest molecular weight OSP were selected for scale up. 53G was chosen for S. sonnei as the clinical strains were all low molecular weight due to the lack of the invasive plasmid. All OSPs confirmed by NMR.

As used herein, “monovalent MAPS” refers to a vaccine containing one PS and one carrier protein. “Bivalent MAPS” refers to a mixture of two monovalent MAPS vaccines, also referred to as multivalent or 2V. “Quadrivalent MAPS”, also referred to as “4V” refers to a mixture of four monovalent MAPS vaccines.

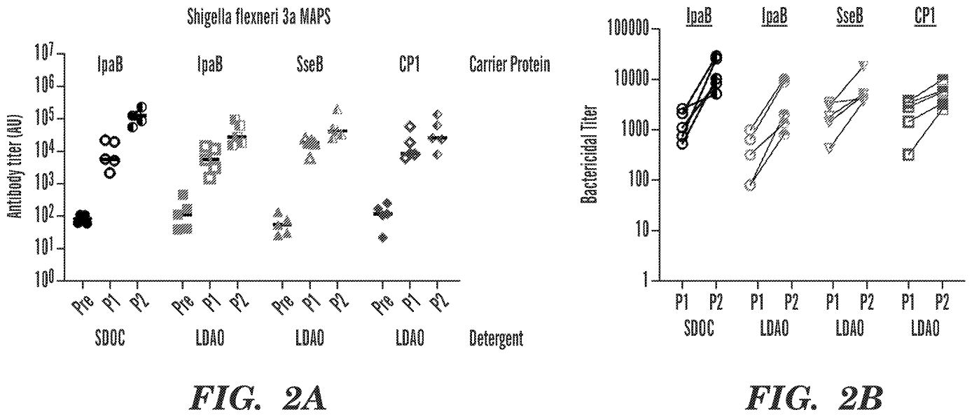

Rabbits were immunized on days 0 (Pre) and 21 with Shigella-MAPS with three carrier proteins: Rhavi-SseB, Rhavi-IpaB and CP1. The dosage was 5 μg/dose/rabbit for each MAPS injection. S. flaxneri-3a-MAPS comprising the OSP polysaccharide and a polypeptide antigen selected (also referred to herein as carrier protein) from IpaB, SseB or CP1 were assessed and the IgG antibody levels (AI) assessed (see FIG. 2A) as well as the bactericidal titer (FIG. 2B) after elution with SDOC (sodium deoxycholate) or LDAO (Dodecyldimethylaminoxid).

### Example 2

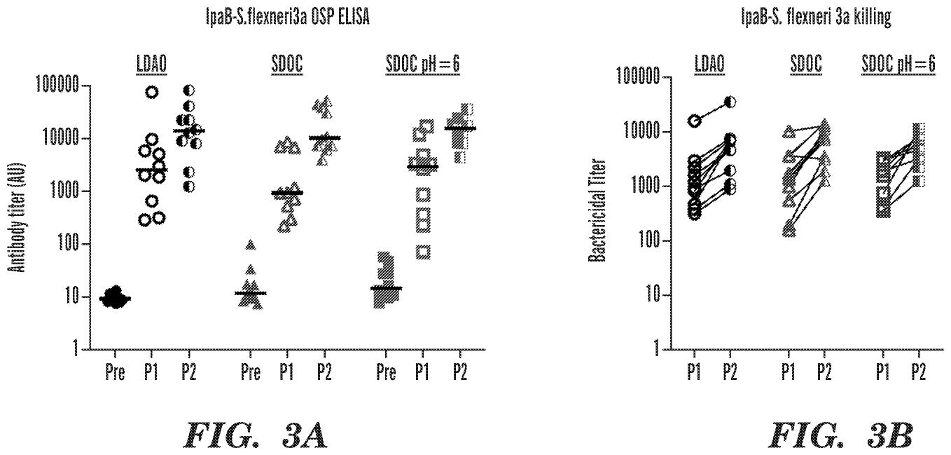

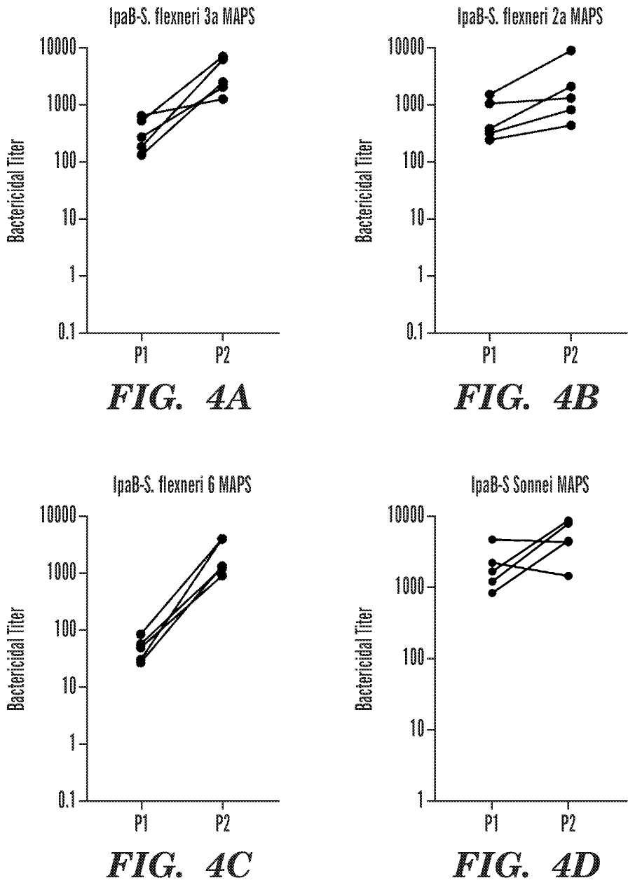

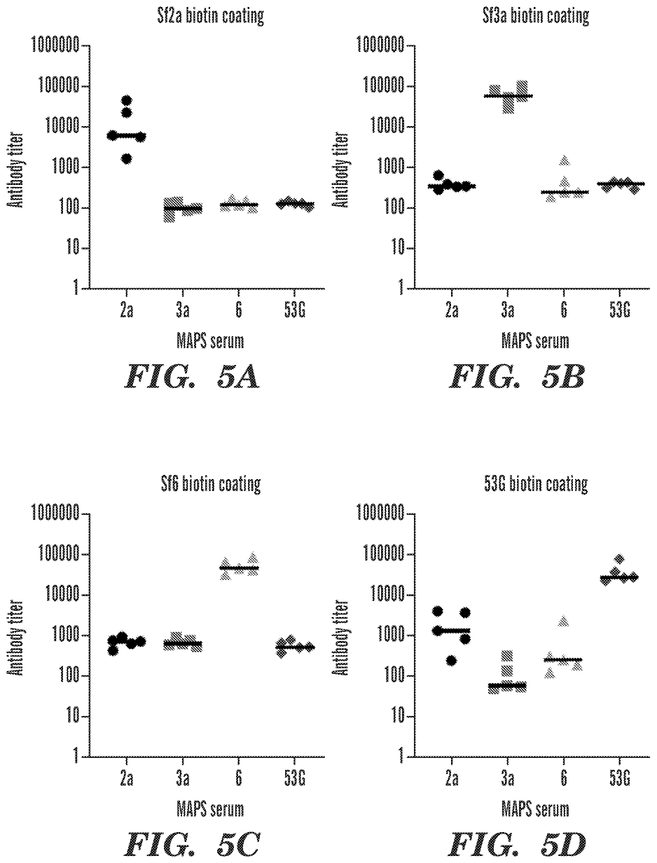

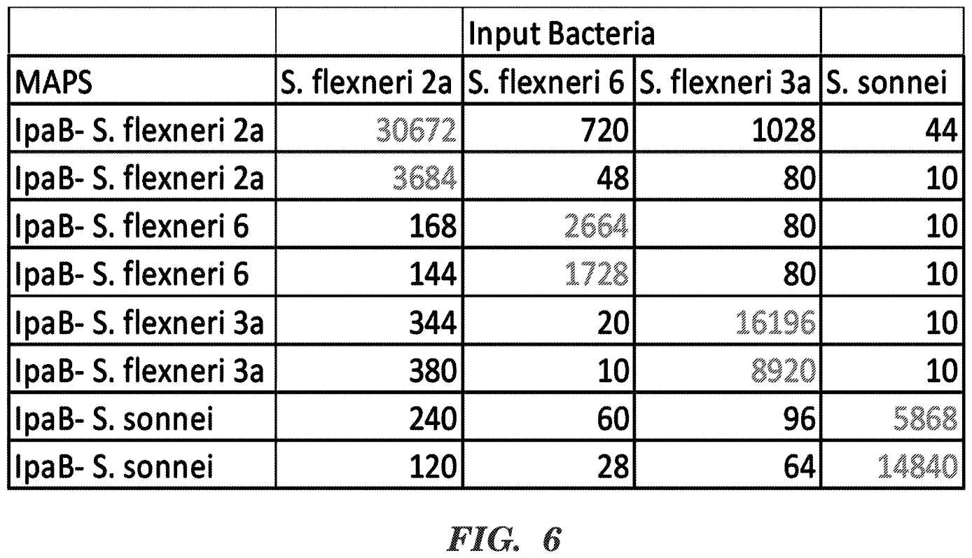

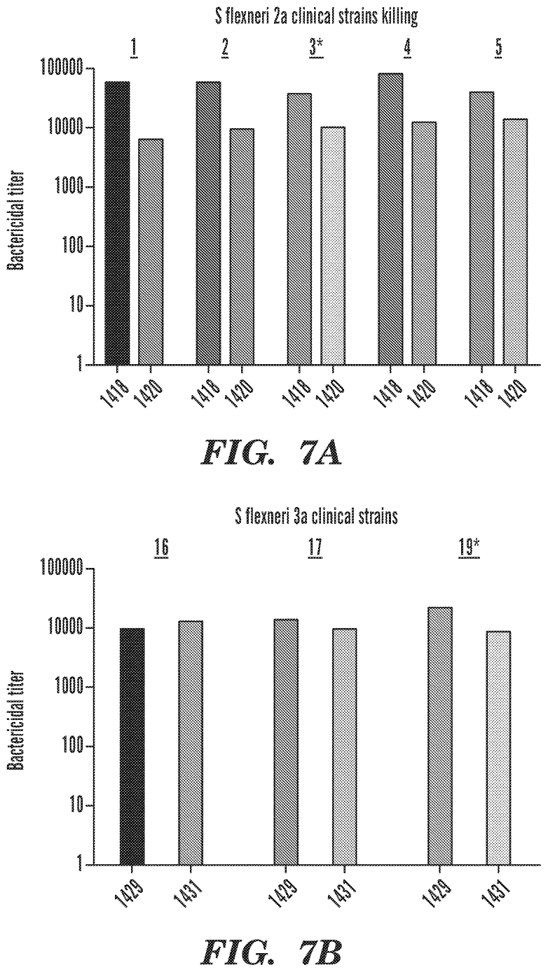

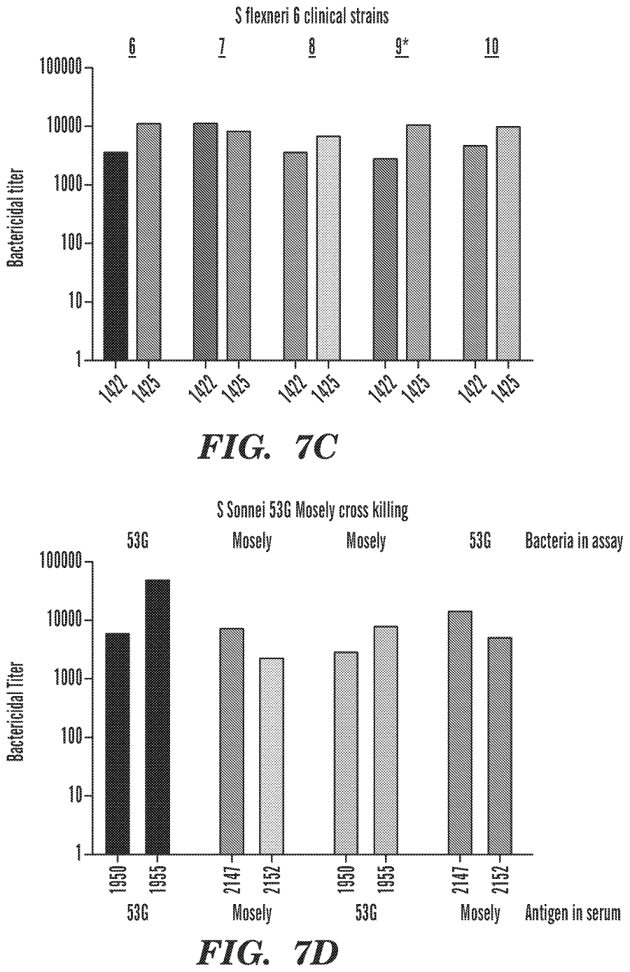

OSP antibody and function was assessed after elution with SDOC (sodium deoxycholate) or LDAO (Dodecyldimethylaminoxid) (FIG. 3A-3B) after rabbits were immunized with Shigella-MAPS comprising the S. flexneri-3a-polysaccharide and the IpaB carrier protein. The bacterial titer for each of the four Shigella-MAPS complexes for each shigella strains was assessed (FIGS. 4A-4D), and each anti-OSP antibody generated from each of the shigella-MAPS complexes is specific to each Shigella serotype (FIG. 5A-5D), with little to no cross-serotype killing (FIG. 6 and FIG. 7A-7D).

Bactericidal Titers Following Immunization with Shigella-MAPS

As is shown in FIG. 2A-2B, sera from mice immunized with S. Flexneri 3a-MAPS was collected prior to first immunization (Pre), three weeks after the first immunization (P1), and three weeks after the second immunization (P2). Antibody against OSP (FIG. 2A) was measured by ELISA. Rhavi-IpaB induced the highest OSP antibody. Rhavi-IpaB had excellent carrier function and was immunogenic in a Shigella MAPS.

Bactericidal activity was carried out using protocols described in the Materials and Methods, below. Titer was calculated from the sera dilution factor that kills 50% of the bacteria in the assay. As is shown in FIG. 2B, Rhavi-IpaB and Rhavi-SseB were both carrier proteins that resulted in high bactericidal titers. As is shown in FIG. 3A, the antibody against S. Flexneri 3a OSP correlated well with bactericidal titers (FIG. 3B. High bacterial titers were also detected S. Flexneri 3a-MAPS, S. Flexneri 2a-MAPS, S. Flexneri 6-MAPS and S. Sonnei-MAPS, each comprising the IpaB carrier protein. The bacterial titer for each of the four Shigella-MAPS complexes for each shigella strains was assessed, and each anti-OSP antibody generated from each of the shigella-MAPS complexes is specific to each Shigella serotype (FIG. 4A-4D), with little to no cross-serotype killing (FIG. 6 and FIG. 7A-7D).

### Example 3: Bivalent Shigella-MAPS and Quadrivalent Shigella-MAPS Vaccines Using Polysaccharides from S. Flexneri 3a-MAPS, S. Flexneri 2a-MAPS, S. Flexneri 6-MAPS and S. Sonnei-MAPS

### Quadrivalent Shigella MAPS Vaccines

Example 2 shows results from exemplary quadrivalent Shigella-MAPS vaccines. It is envisioned any number of combinations of S. flexneri 3a-MAPS, S. flexneri 2a-MAPS, S. flexneri 6-MAPS and S. sonnei-MAPS immunogenic complexes can be assembled or combined by a person of ordinary skill in the art to form bivalent (2V), multivalent, 3V, quadrivalent (4V) Shigella-MAPS vaccines. Without being limited to theory, exemplary combinations for 2V, 3V and 4V Shigella-MAPS vaccines are disclosed in Tables 1A-IC herein. It is envisioned that other combinations can be compiled to generate Shigella-MAPS vaccines that comprise between 1-50 species of immunogenic complexes. It is envisioned that other combinations can be compiled to generate Shigella-Salmonella-MAPS vaccines that comprise between 1-50 species of immunogenic complexes that are Shigella-MAPS immunogenic complexes or Salmonella-MAPS immunogenic complexes.

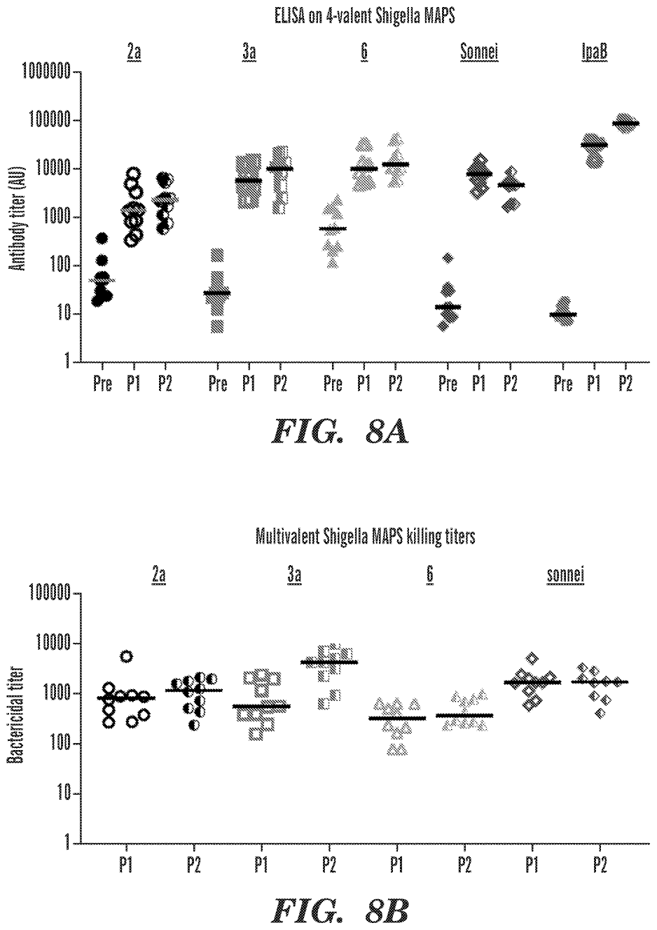

The method of FIG. 8A-8B quadrivalent Shigella MAPS vaccine includes IpaB-S. flexneri 2a, IpaB-S. flexneri 6, IpaB-S. flexneri 3a, and IpaB-S. sonnei 53G, alum phosphate, His buffer with Tween-80, pH 6. There is 5 μg each polysaccharide per dose. There are two immunizations, three weeks apart.

As shown in FIG. 8A-8B, a robust immune response to the OSP (as detected by anti-OSP IgG) was detected with the quadrivalent Shigella-MAPS was detected at doses as low as 5 μg polysaccharide. Moreover, the immune response was enhanced by a second (or booster) administration (P2) as detected by increased antibody titer (AU) (FIG. 8A-8B).

The quadrivalent Shigella-MAPS vaccines demonstrated effective bactericidal activity, as detected by the killing titers as assessed with bactericidal assays (FIG. 8B).

The process of MAPS production in the laboratory setting include bacteria grown in shaking flasks. The carrier protein which is 6His-tagged purified by Ni-NTA column and a size-exclusion column. The OSP is purified by acid hydrolysis and size-exclusion column. MAPS is assembled by combining a known ratio of polysaccharide:protein in end-over-end rotation at 4° C. overnight. The complex is then purified on FPLC.

The process of MAPS production for GLP includes bacteria grown in bioreactors with chemically defined medium. The carrier protein (non-His-tagged) is purified by attaching His-tagged chaperon to Ni-NTA column and eluted with 0.05% sDOC. OSP is purified by acid hydrolysis and TFF buffer exchange. MAPS are assembled by combining a known ration of polysaccharide:protein in a bottle with stir bars. Free protein and polysaccharide are removed by TFF. The assays used for GLP production include protein quantification (BCA), polysaccharide quantification (Anthrone for S. flexneri, HEPAC for S. sonnei), ID test for drug substance, drug product and final MAPS (monoclonal antibody for 4 PS, polyclonal antibody for IpaB), SEC MALS (size and free protein and free PS), Triton precipitation (free PS), endotoxin kit from Pierce (LPS), ICP-MS (Vaxform; alum content), Vaxform (pH, appearance, osmolality), sterility (outsourced), residues (sDOC; cyanide). The materials used for GLP production include OSPs (about 20 mg each for scale up; 100 mg for GLP production), protein over 90% purity (200 mg for scale up; 1600 mg for GLP production), biotin-OSP (biotinylation level need to be optimized to minimize free biotin in the MAPS), CDAP, Amine-Peg3-biotin, and alum phosphate. The final MAPS will have a 4:1 protein:PS ratio.

### Example 4. Quadrivalent Salmonella-MAPS Vaccine Against Salmonella Serovars with Typhimurium and Enteritidism, S. Typhi Vi and Paratyphi a OSP

In some embodiments, a vaccine or immunogenic composition as disclosed herein comprises a quadrivalent (4V) Shigella-MAPS vaccine as disclosed in Examples 1-2, and a quadrivalent (4V) salmonella-MAPS vaccine, thereby resulting in an 8-valent (8V) Shigella-Salmonella vaccine.

Example 3 shows results from exemplary quadrivalent Salmonella-MAPS vaccine, as disclosed in PCT application No: PCT/US22/80531 filed on Nov. 29, 2022, which is incorporated herein in its entirety by reference.

It is envisioned any number of combinations of S. Typhimurium, S. Enteritidis, S. Typhi and S. Paratyphi A MAPS immunogenic complexes can be assembled or combined by a person of ordinary skill in the art to form bivalent (2V), multivalent, 3V, quadrivalent (4V) Salmonella-MAPS vaccines, for use in the methods and immunogenic compositions as disclosed herein. Without being limited to theory, exemplary combinations for 2V, 3V and 4V salmonella-MAPS vaccines are disclosed in Tables 1A-IC in PCT application No: PCT/US22/80531 filed on Nov. 29, 2022. It is envisioned that other combinations can be compiled to generate Salmonella-MAPS vaccines that comprise between 1-50 species of immunogenic complexes.

The following quadrivalent (4V) MAPS vaccines were generated against Salmonella strains, and are exemplary 4V salmonella-MAPS vaccines, and assessed in further experiments.

### Quadrivalent MAPS Generate Robust Antibodies Against PS

Quadrivalent Salmonella-MAPS comprising (“Salmonella-MAPS1”):
i. S. Typhimurium-SseB
ii. S. Enteritidis-SseB
iii. S. Typhi Vi-CP1
iv. S. Paratyphi A-CP1

Quadrivalent Salmonella MAPS comprising (“Salmonella-MAPS2”):
i. S. Typhimurium-SseB
ii. S. Enteritidis-SseB
iii. S. Typhi Vi-SseB
iv. S. Paratyphi A-SseB

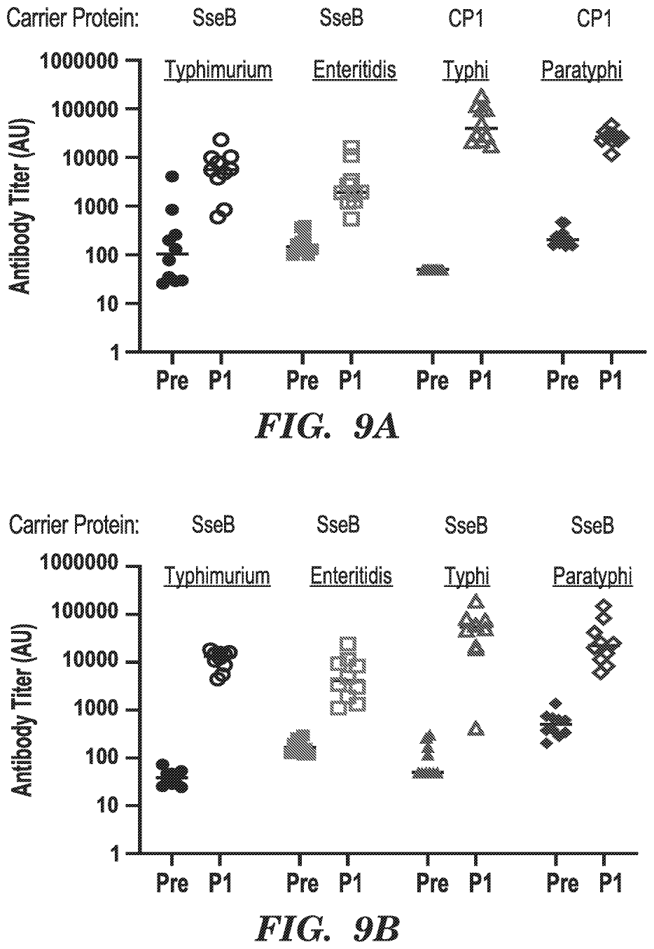

As is shown in FIG. 9A-9B, quadrivalent salmonella-MAPS comprising SseB made with Typhimurium and Enteritidis OSP, or salmonella-MAPS comprising CP1 with Vi and Paratyphi A OSP, and quadrivalent salmonella-MAPS comprising SseB carrier protein and Typhimurium and Enteritidis OSP, Vi or Paratyphi A OSP (FIG. 9B) were very immunogenic. Antibody against each OSP or Vi was measured by ELISA.

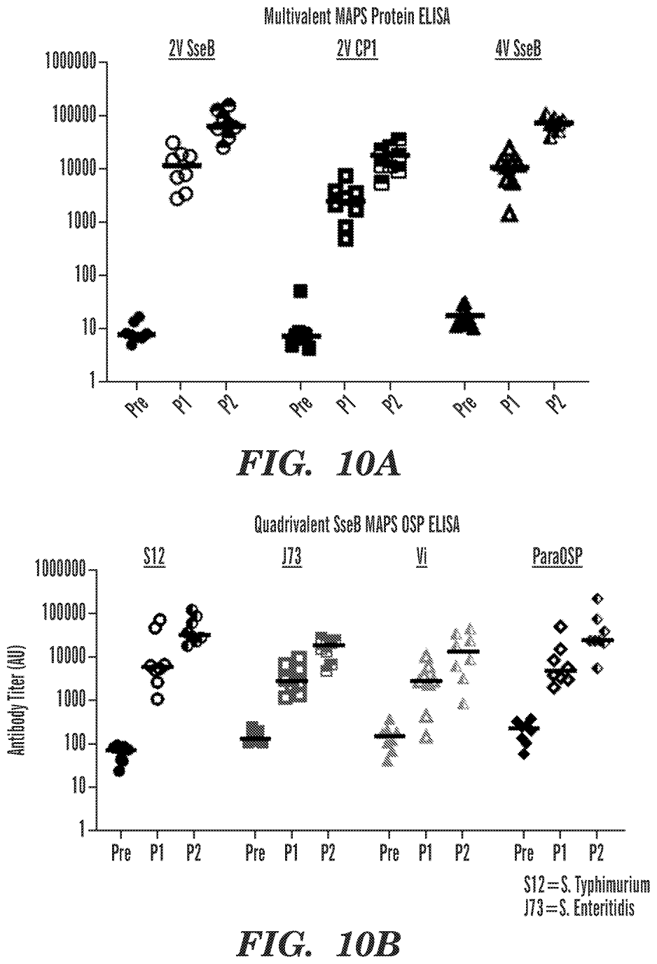

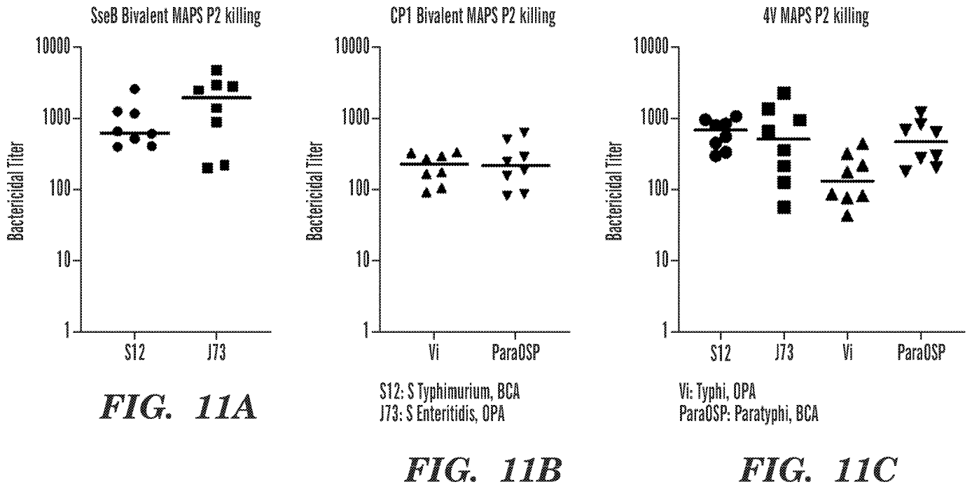

The efficacy of the bivalent and quadrivalent Salmonella-MAPS was assessed in vivo in rabbits, and the bivalent Salmonella-MAPS comprising polysaccharides from both S. Typhi (comprising the Vi polysaccharide) and S. Paratyphi A (comprising the OSP polysaccharide) and comprising the carrier protein SseB or CP1 induced a robust immune response after P1, which was enhanced after by a second immunization P2 (FIG. 10A), and similar increase in antibody titer (AI) was seen with the quadrivalent salmonella-MAPS with the SseB carrier protein comprising polysaccharides from: S. Typhimurium (S12), S. Enteritidis (J13), S. Typhi and S. Paratyphi A (FIG. 10B). The bivalent (FIGS. 11A-11B) and the quadrivalent salmonella MAPS (FIG. 11C) also were effective at killing bacteria of each serovers type.

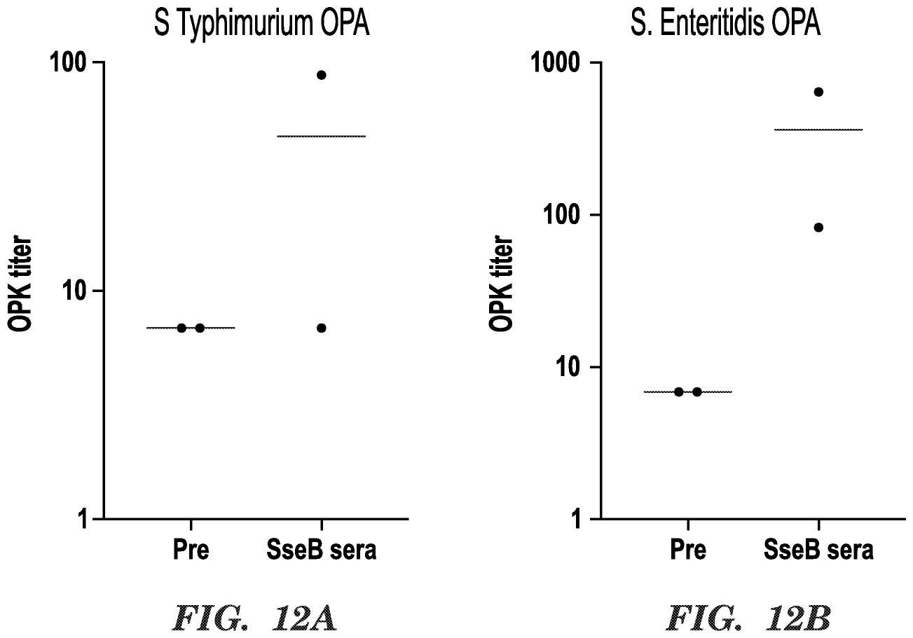

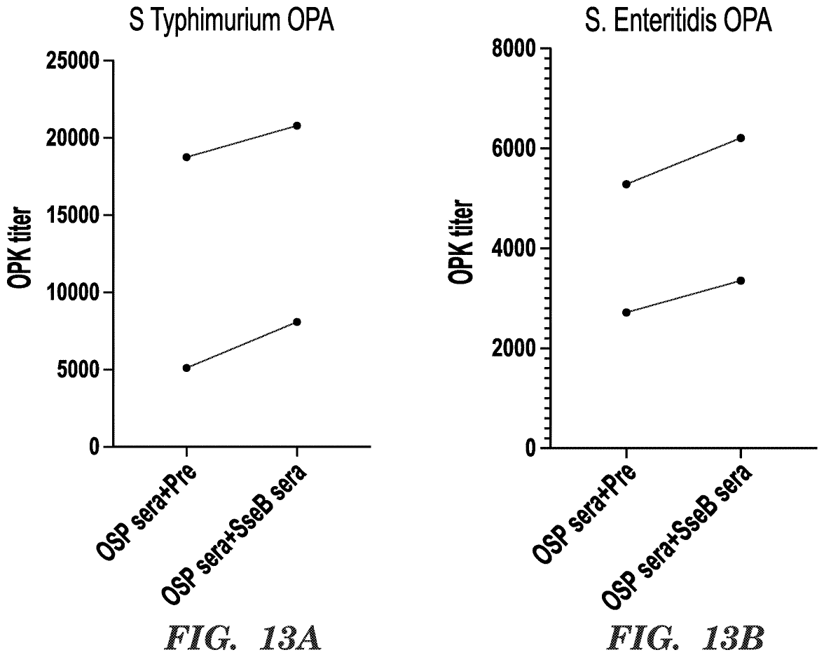

Opsonophagocytic killing assays (OPA) or OPK titers demonstrated the effective killing of Typhimurium (S12) and S. Enteritidis (J13) with SseB antisera (FIG. 12A-12B) or OSP antisera, and that the addition of SseB antisera enhanced killing by the OSP antisera (FIG. 13A-13B).

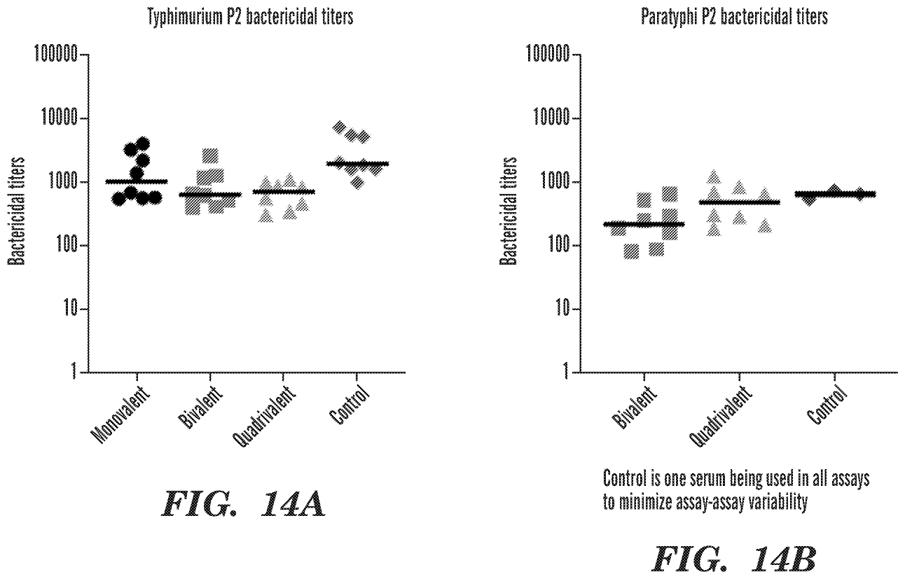

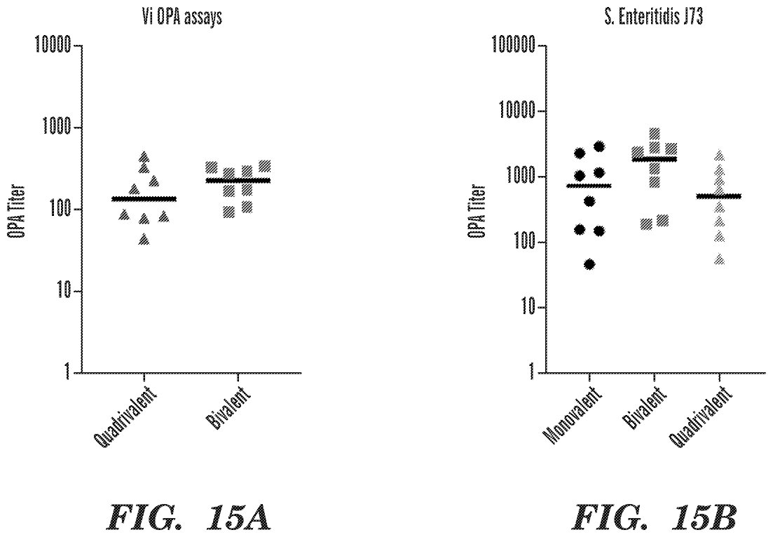

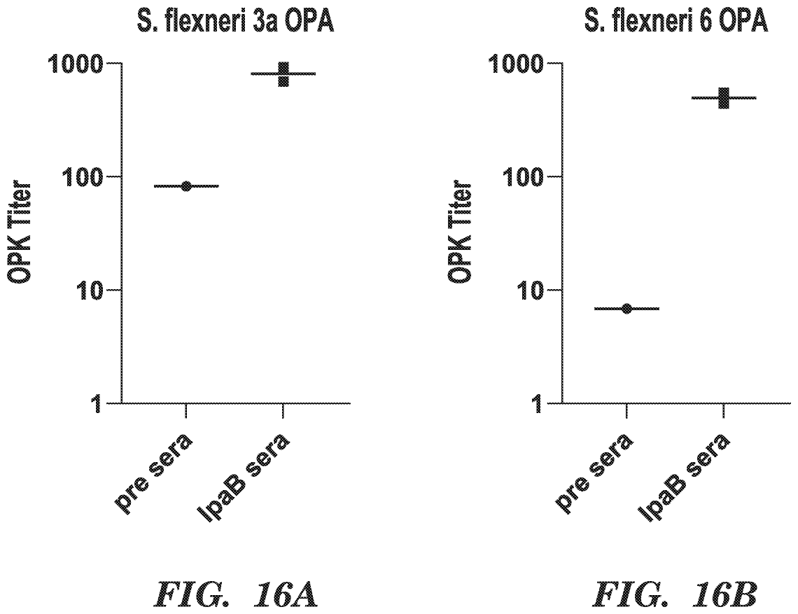

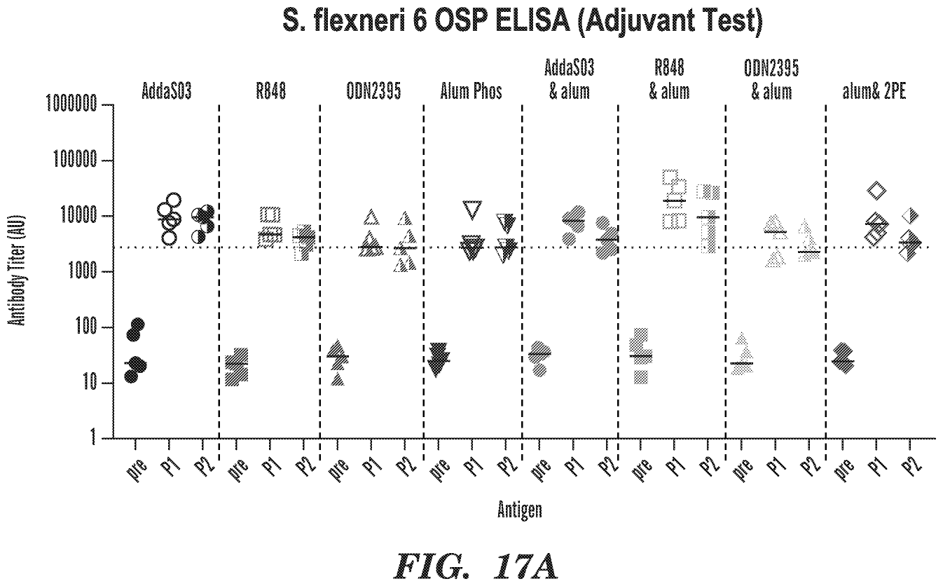

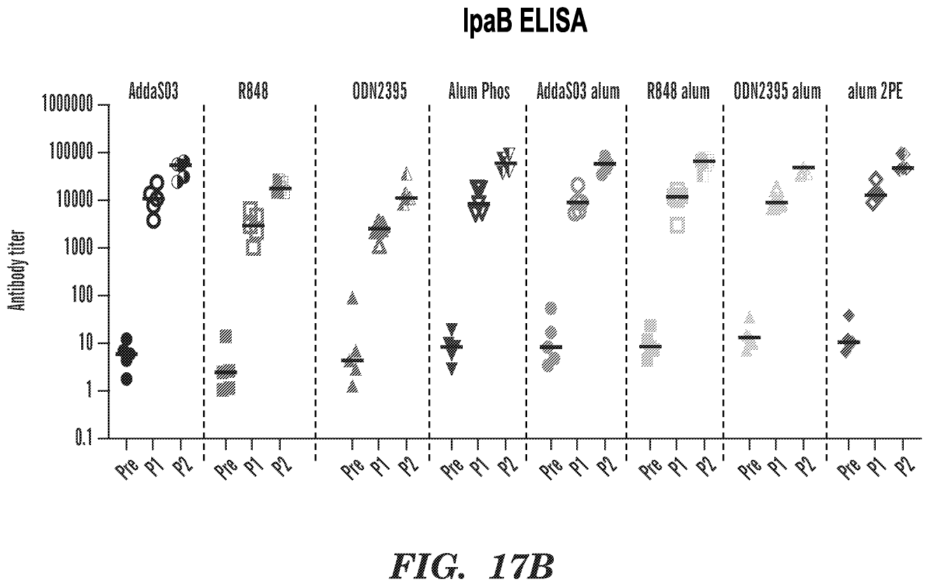

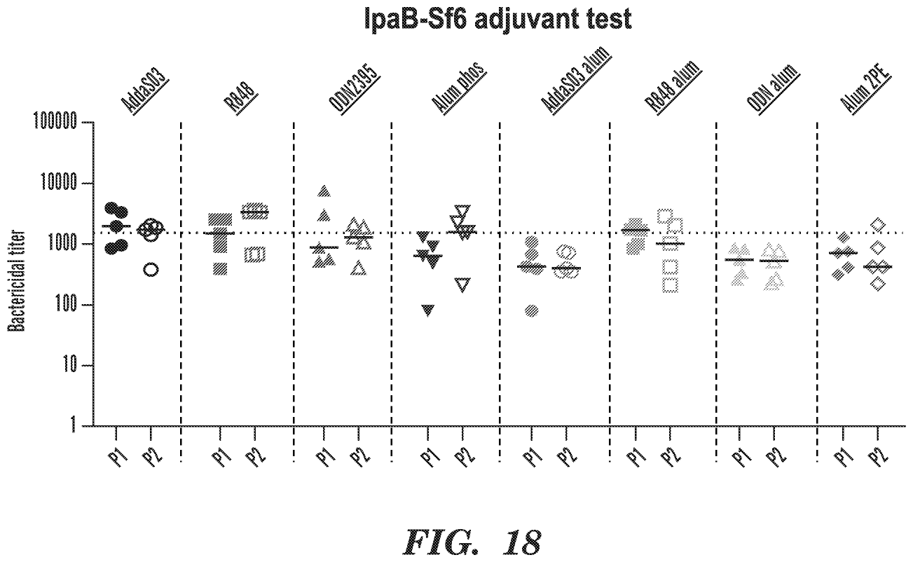

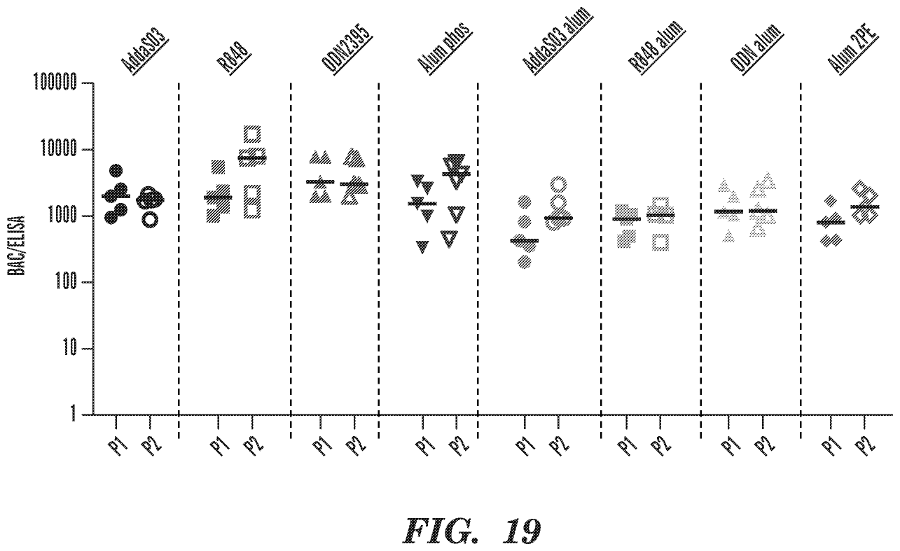

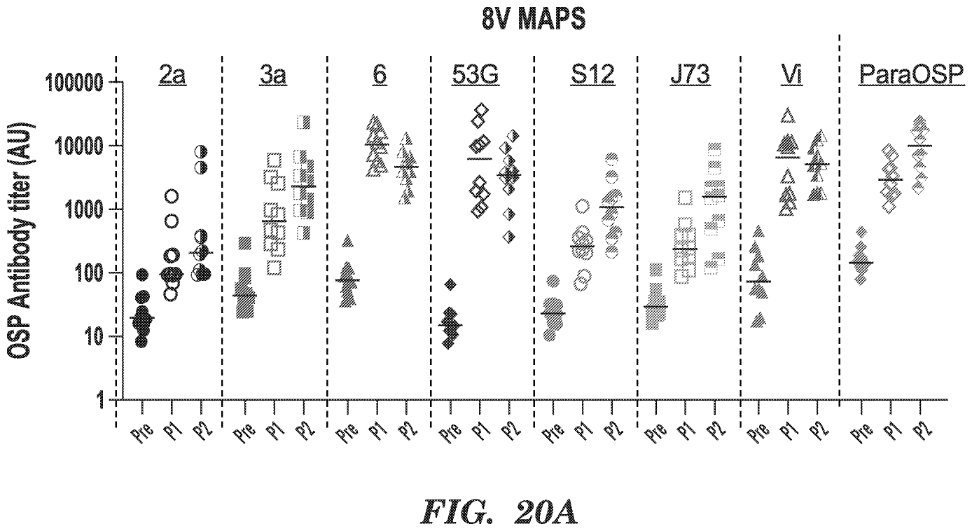

A comparison of the effective killing of S. Typhimurium or S. Paratyphi A after P2 administration of monovalent, bivalent and quadrivalent salmonella-MAPS shows that all MAPS complexes assessed were effective at reducing bacteria as compared to the control (FIG. 14A-14B). Similar robust immune response was seen with the monovalent, bivalent and quadrivalent salmonella-MAPS vaccines with S. Typhi and S. Enteritidis (FIG. 15A-15B).

The results presented herein demonstrate that (1) shigella-MAPS made with polysaccharides from the serovars selected from: S. Flexneri 3a-MAPS, S. Flexneri 2a-MAPS, S. Flexneri 6-MAPS and S. Sonnei-OSP generate robust antibody titers with high bactericidal killing activity against their respective serovars; (2) bivalent shigella-MAPS and quadrivalent shigella—MAPS show similar activity as monovalent shigella—MAPS; and (3) SseB fused with rhizavidin, or IpaB fused with rhizavidin are excellent carrier proteins and are immunogenic.

### Example 5: Comparing the 8-Valent and Quadrivalent MAPS-Shigella Vaccine and MAPS-Salmonella Vaccine

The following 8-valent (8V) MAPS vaccines were generated against Shigella and Salmonella strains, and are exemplary 8V Shigella-MAPS vaccines and Salmonella-MAPS vaccines, and assessed in further experiments.

8-valent MAPS-Shigella vaccines comprising:
i. IpaB-S. flexneri 2a
ii. IpaB-S. flexneri 3a
iii. IpaB-S. flexneri 6
iv. IpaB-S. sonnei 53G

8-valent MAPS-Salmonella vaccines comprising:
v. SseB-S. Typhimurium S12
vi. SseB-S. Enteritidis J73
vii. SseB-S. Typhi Vi
viii. SseB-S. Paratyphi A

8-valent MAPS vaccines also included alum phosphate. There is 5 μg each polysaccharide per dose. There are two immunizations, three weeks apart. Rabbits were also immunized with Salmonella and Shigella quadrivalent MAPS-vaccines at the same time to compare.

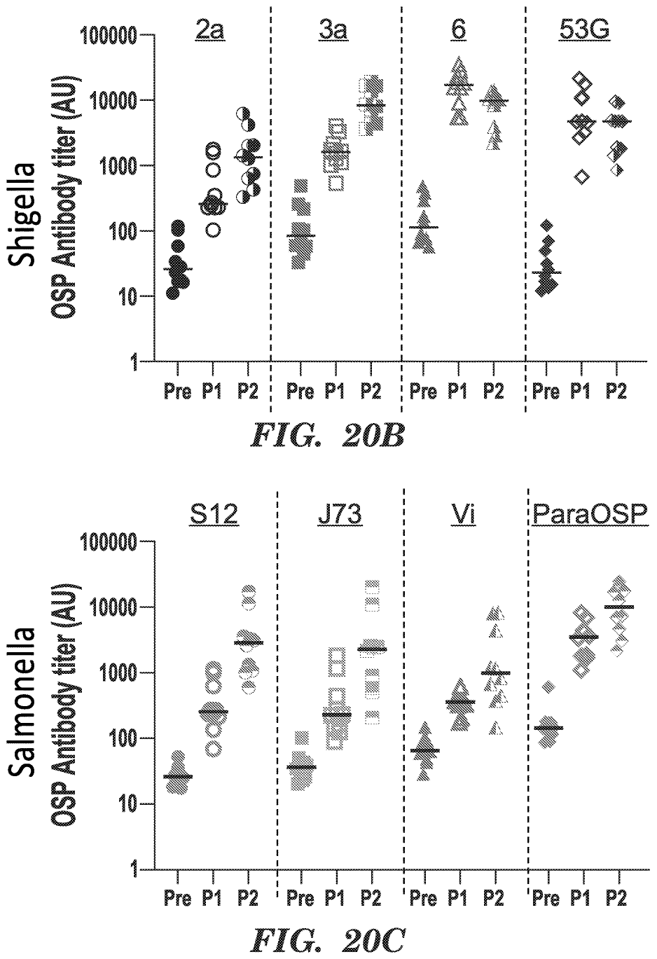

The efficacy of the 8-valent Shigella-MAPS and Salmonella-MAPS was assessed in vivo in rabbits. The 8-valent Shigella-MAPS and Salmonella MAPS induced a robust immune response after P1, which was enhanced after by a second immunization P2 (FIG. 20A). Similar increase in antibody titer (AU) was seen with the quadrivalent Shigella-MAPS vaccine with the IpaB carrier protein comprising polysaccharides from S. flexneri 2a, S. flexneri 3a, S. flexneri 6, and S. sonnei (FIG. 20B) and quadrivalent salmonella-MAPS vaccine (FIG. 20C) with the SseB carrier protein comprising polysaccharides from: S. Typhimurium (S12), S. Enteritidis (J13), S. Typhi and S. Paratyphi A (FIG. 20C). Antibody against each OSP or Vi was measured by ELISA.

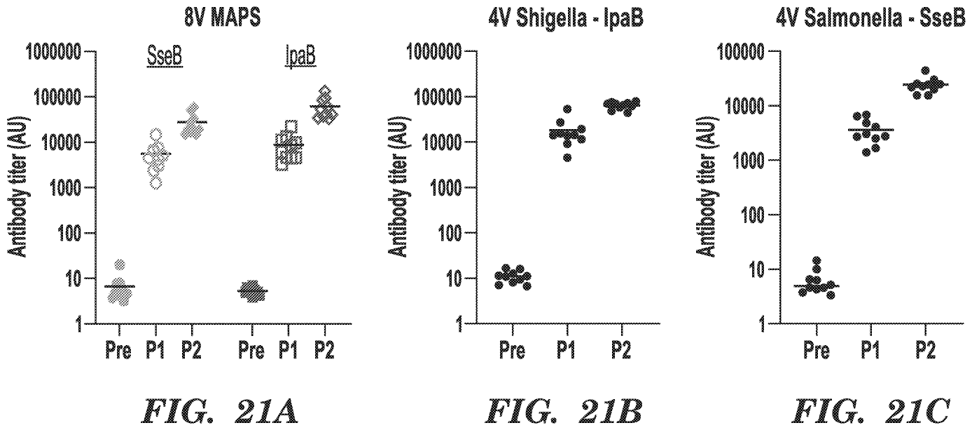

The protein antibody titer for 8-valent MAPS-Shigella and MAPS-Salmonella vaccine (FIG. 21A) is comparable to quadrivalent MAPS-Shigella (FIG. 21B) and MAPS-Salmonella vaccine (FIG. 21C).

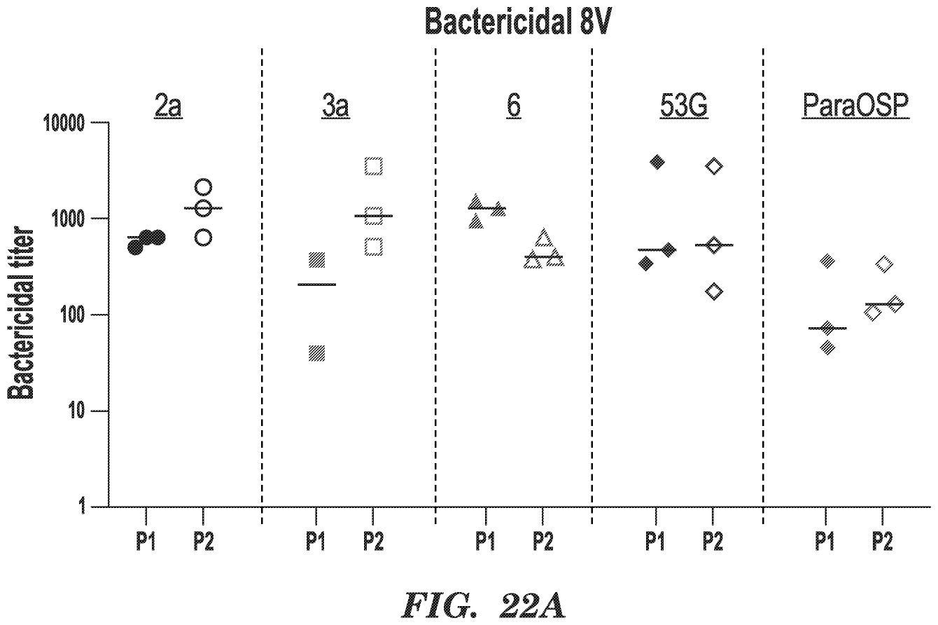

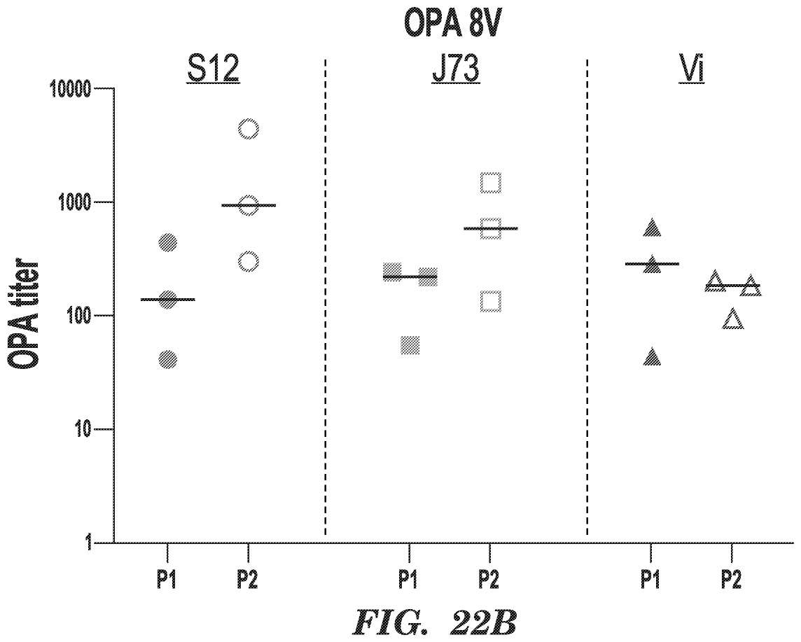

The 8-valent Shigella-MAPS vaccines demonstrated effective bactericidal activity, as detected by the killing titers as assessed with bactericidal assays (FIG. 22A). Opsonophagocytic killing assays (OPA) or OPK titers demonstrated the effective killing of 8-valent MAPS-Salmonella vaccines with high, medium, and low antibody titer antisera (FIG. 22B).

### REFERENCES

All the references cited herein and throughout the application are incorporated by reference in their entirety.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A vaccine comprising an immunogenic complex, wherein the immunogenic complex comprises:
(a) a biotinylated polysaccharide antigen; and
(b) a fusion protein comprising:
ii. a biotin-binding moiety; and
iii. at least one polypeptide antigen;
wherein the biotinylated polysaccharide antigen comprises a 0-specific polysaccharide (OSP) of Shigella, and further wherein the biotinylated polysaccharide antigen is non-covalently associated with the biotin-binding moiety of the fusion protein to form an immunogenic complex.

2. The vaccine of claim 1, wherein the biotinylated polysaccharide antigen comprises a polysaccharide of Shigella selected from S. flexneri 3a, S. flexneri 2a, S. flexneri 6 or S. sonnei.

3. The vaccine of claim 1 or claim 2, wherein the at least one polypeptide antigen of the fusion protein is or comprises a polypeptide antigen from Salmonella, Shigella, or Streptococcus pneumoniae.

4. The vaccine of any of claims 1-3, wherein the at least one polypeptide antigen of the fusion protein comprises a Salmonella SseB polypeptide or antigenic fragment thereof.

5. The vaccine of any of claims 1-3, wherein the at least one polypeptide antigen of the fusion protein comprises a Shigella IpaB polypeptide or antigenic fragment thereof.

6. The vaccine of any of claims 1-3, wherein the at least one polypeptide antigen of the fusion protein comprises a Streptococcus pneumoniae SP1500 polypeptide or antigenic fragment thereof, a Streptococcus pneumoniae SP0785 polypeptide or antigenic fragment thereof, or both.

7. The vaccine of any one of the preceding claims, wherein the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

8. The vaccine of any one of the preceding claims, wherein the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

9. The vaccine of any one of the preceding claims, wherein the at least one polypeptide antigen of the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8 or SEQ ID NO: 9, or a combination of SEQ ID NO: 8 and SEQ ID NO: 9.

10. The vaccine of any one of the preceding claims, wherein the biotin-binding moiety is a polypeptide comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 3 or a biotin-binding fragment thereof.

11. An immunogenic composition (e.g., a vaccine) comprising a plurality of different species of immunogenic complexes, wherein the different species comprise:
a plurality of biotinylated polysaccharide antigens comprising polysaccharide antigens of one or more Shigella serotypes; and
a plurality of fusion proteins, each fusion protein comprising:
a biotin-binding moiety; and
a polypeptide antigen,
wherein each of the plurality of biotinylated polysaccharide antigens is non-covalently associated with the biotin-binding moiety of one or more of the plurality of fusion proteins to form an immunogenic complex.

12. The immunogenic composition of claim 11, wherein the different species each comprise a distinct polysaccharide antigen of one or more Shigella serotypes and/or a distinct polypeptide antigen.

13. The immunogenic composition of claim 11 or 12, wherein the one or more Shigella serotypes is or comprises S. flexneri 3a, S. flexneri 2a, S. flexneri 6 or S. sonnei, or combinations thereof.

14. The immunogenic composition of any one of claims 11-13, wherein the polypeptide antigen is or comprises a polypeptide antigen from Salmonella, Shigella, and/or Streptococcus pneumoniae.

15. The immunogenic composition of claim 14, wherein the polypeptide antigen is or comprises: an SseB polypeptide antigen of Salmonella, an IpaB polypeptide antigen of Shigella, and/or a polypeptide antigen comprising an SP1500 polypeptide and/or an SP0785 polypeptide, of S. pneumoniae.

16. The immunogenic composition of any one of claims 11-15, comprising at least two different species of immunogenic complexes, wherein the different species comprise:
a first biotinylated polysaccharide antigen comprising a polysaccharide of S. flexneri 2a non-covalently complexed with a first fusion protein; and
a second biotinylated polysaccharide antigen comprising a polysaccharide of S. flexneri 3a non-covalently complexed with a second fusion protein,
wherein the first fusion protein and the second fusion protein each independently comprise:
a biotin-binding moiety; and
a polypeptide antigen,
wherein each of the first and second biotinylated polysaccharide antigens is non-covalently associated with the biotin-binding moiety of the respective fusion protein to form an immunogenic complex.

17. The immunogenic composition of claim 16, wherein the polypeptide antigen of the first fusion protein and of the second fusion protein is or comprises an SseB polypeptide antigen of Salmonella.

18. The immunogenic composition of claim 17, wherein the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof.

19. The immunogenic composition of any one of claims 11-15, comprising at least two different species of immunogenic complexes, wherein the different species comprise:
a first biotinylated polysaccharide antigen comprising a polysaccharide of Flexneri 2a non-covalently complexed with a first fusion protein; and
a second biotinylated polysaccharide antigen comprising a polysaccharide of S. Sonnei non-covalently complexed with a second fusion protein,
wherein the first fusion protein and the second fusion protein each independently comprise:
a biotin-binding moiety; and
a polypeptide antigen,
wherein each of the first and second biotinylated polysaccharide antigens is non-covalently associated with the biotin-binding moiety of the respective fusion protein to form an immunogenic complex.

20. The immunogenic composition of claim 19, wherein the polypeptide antigen of the first fusion protein and of the second fusion protein is or comprises an SP1500 polypeptide, an SP0785 polypeptide, or both, of S. pneumoniae, and/or the fusion protein is CP1.

21. The immunogenic composition of claim 20, wherein the SP1500 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, the SP0785 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, and the CP1 fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 6, or an immunogenic fragment thereof.

22. The immunogenic composition of any one of claims 11-15, comprising at least four different species of immunogenic complexes, wherein the different species comprise:
a first biotinylated polysaccharide antigen comprising a polysaccharide of S. Flexneri 2a non-covalently complexed with a first fusion protein; and
a second biotinylated polysaccharide antigen comprising a polysaccharide of S. Flexneri 3a non-covalently complexed with a second fusion protein,
a third biotinylated polysaccharide antigen comprising a polysaccharide of S. Flexneri 6 non-covalently complexed with a third fusion protein; and
a fourth biotinylated polysaccharide antigen comprising a polysaccharide of S. Sonnei A non-covalently complexed with a fourth fusion protein,
wherein the first fusion protein, the second fusion protein, the third fusion protein, and the fourth fusion protein each independently comprise:
a biotin-binding moiety; and
a polypeptide antigen,
wherein each of the first, second, third, and fourth biotinylated polysaccharide antigens is non-covalently associated with the biotin-binding moiety of the respective fusion protein to form an immunogenic complex.

23. The immunogenic composition of claim 22, wherein the polypeptide antigen of the first fusion protein and of the second fusion protein is or comprises an SseB polypeptide antigen of Salmonella, and the polypeptide antigen of the third fusion protein and of the fourth fusion protein is or comprises an SP1500 polypeptide, an SP0785 polypeptide, or both, of S. pneumoniae, and/or the fusion protein is CP1.

24. The immunogenic composition of claim 23, wherein the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof, the SP1500 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 8, or an immunogenic fragment thereof, the SP0785 polypeptide antigen of S. pneumoniae is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 9, or an immunogenic fragment thereof, and the CP1 fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 6, or an immunogenic fragment thereof.

25. The immunogenic composition of any one of claims 11-15, comprising at least four different species of immunogenic complexes, wherein the different species comprise:
a first biotinylated polysaccharide antigen comprising a polysaccharide of S. flexneri 2a non-covalently complexed with a first fusion protein; and
a second biotinylated polysaccharide antigen comprising a polysaccharide of S. flexneri 3a non-covalently complexed with a second fusion protein,
a third biotinylated polysaccharide antigen comprising a polysaccharide of S. flexneri 6 non-covalently complexed with a third fusion protein; and
a fourth biotinylated polysaccharide antigen comprising a polysaccharide of S. sonnei non-covalently complexed with a fourth fusion protein,
wherein the first fusion protein, the second fusion protein, the third fusion protein, and the fourth fusion protein each independently comprise:
a biotin-binding moiety; and
a polypeptide antigen,
wherein each of the first, second, third, and fourth biotinylated polysaccharide antigens is non-covalently associated with the biotin-binding moiety of the respective fusion protein to form an immunogenic complex.

26. The immunogenic composition of claim 25, wherein the polypeptide antigen of the first fusion protein, the second fusion protein, the third fusion protein, and the fourth fusion protein is or comprises an SseB polypeptide antigen of Salmonella.

27. The immunogenic composition of claim 26, wherein the SseB polypeptide antigen of Salmonella is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an immunogenic fragment thereof.

28. A vaccine comprising a plurality of different species of immunogenic complexes, wherein the different species comprise:
a biotinylated polysaccharide antigen of Shigella serotype S. Flexneri 2a non-covalently complexed with a fusion protein;
a biotinylated polysaccharide antigen of Shigella serotype S. Flexneri 3a non-covalently complexed with a fusion protein;
wherein each fusion protein comprises
a biotin-binding moiety; and
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
wherein each of the biotinylated polysaccharide antigens is non-covalently associated with the biotin-binding moiety of at least one fusion protein to form an immunogenic complex.

29. The vaccine of claim 28, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

30. The vaccine of claim 28, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

31. The vaccine of claim 28, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8, SEQ ID NO: 9, or both SEQ ID NO: 8 and SEQ ID NO:9.

32. A vaccine comprising a plurality of different species of immunogenic complexes, wherein the different species comprise:
a biotinylated polysaccharide antigen of Shigella serotype S. Flexneri 6 non-covalently complexed with a fusion protein;
a biotinylated polysaccharide antigen of Shigella serotype S. Sonnei non-covalently complexed with a fusion protein;
wherein each fusion protein comprises
a biotin-binding moiety; and
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
wherein each of the biotinylated polysaccharide antigens is non-covalently associated with the biotin-binding moiety of at least one fusion protein to form an immunogenic complex.

33. The vaccine of claim 32, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

34. The vaccine of claim 32, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

35. The vaccine of claim 32, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8, SEQ ID NO: 9, or both SEQ ID NO: 8 and SEQ ID NO:9.

36. A vaccine comprising a plurality of different species of immunogenic complexes, wherein the different species comprise:
a biotinylated polysaccharide antigen of Shigella serotype S. flexneri 2a non-covalently complexed with a fusion protein;
a biotinylated polysaccharide antigen of Shigella serotype S. flexneri 3a non-covalently complexed with a fusion protein;
a biotinylated polysaccharide antigen of Shigella serotype S. flexneri 6 non-covalently complexed with a fusion protein;
a biotinylated polysaccharide antigen of Shigella serotype S. sonnei non-covalently complexed with a fusion protein;
wherein each fusion protein comprises
a biotin-binding moiety; and
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
wherein each of the biotinylated polysaccharide antigens is non-covalently associated with the biotin-binding moiety of at least one fusion protein to form an immunogenic complex.

37. The vaccine of claim 36, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4.

38. The vaccine of claim 36, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5.

39. The vaccine of claim 36, wherein the first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to either SEQ ID NO: 8, SEQ ID NO: 9, or both SEQ ID NO: 8 and SEQ ID NO:9.

40. A vaccine comprising a plurality of different species of immunogenic complexes, wherein the different species comprise:
a biotinylated polysaccharide antigen of Shigella serotype S. flexneri 2a non-covalently complexed with a fusion protein;
a biotinylated polysaccharide antigen of Shigella serotype S. flexneri 3a non-covalently complexed with a fusion protein;
a biotinylated polysaccharide antigen of Shigella serotype S. flexneri 6 non-covalently complexed with a fusion protein;
a biotinylated polysaccharide antigen of Shigella serotype S. sonnei non-covalently complexed with a fusion protein;
wherein each fusion protein comprises
a biotin-binding moiety;
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO: 9, or an antigenic fragment thereof, and
a second polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof, and
wherein each of the biotinylated polysaccharide antigens is non-covalently associated with the biotin-binding moiety of at least one fusion protein to form an immunogenic complex.

41. The vaccine of claim 40, wherein the vaccine comprises a stoichiometrically equal ratio, by weight, of each of the polysaccharide antigens of the different species.

42. The vaccine of claim 40, wherein the vaccine comprises at least one of the polysaccharide antigens of the different species at a stoichiometrically different ratio, by weight.

43. The vaccine of any of claims 28-42, wherein the vaccine comprises a stoichiometrically different ratio, by weight, of each of the polysaccharide antigens of the different species.

44. The vaccine of any one of the preceding claims, wherein the biotin-binding moiety is a polypeptide comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 3.

45. An immunogenic complex comprising a biotinylated polysaccharide antigen of Shigella non-covalently associated with a fusion protein, wherein the fusion protein comprises a biotin-binding moiety and at least one polypeptide antigen.

46. The immunogenic complex of claim 45, wherein the biotinylated polysaccharide antigen comprises a polysaccharide of Shigella having a serotype selected from S. flexneri 2a, S. flexneri 3a, S. flexneri 6 or S. sonnei.

47. The immunogenic complex of either claim 45 or 46, wherein the fusion protein comprises SseB, IpaB, or an SP1500 polypeptide, an SP0785 polypeptide, or both.

48. The immunogenic complex of claim 47, wherein the fusion protein comprises:
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4 or an antigenic fragment thereof,
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5 or an antigenic fragment thereof; or
a first polypeptide antigen comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO: 4, SEQ ID NO: 5, or either SEQ ID NO: 8, SEQ ID NO: 9, or both SEQ ID NO: 8 and SEQ ID NO:9, or an antigenic fragment thereof.

49. The immunogenic complex of any one of claims 45-48, comprising a ratio of fusion protein to polysaccharide antigen of about 1:1, about 2:1, about 3:1, about 4:1, about 5:1, about 6:1, about 7:1, about 8:1, about 9:1, or about 10:1, by weight.

50. A vaccine comprising one or more immunogenic complexes of any one of claims 45-49.

51. A pharmaceutical composition comprising the vaccine of any one of claims 1-44 and 46, and a pharmaceutically acceptable carrier.

52. A pharmaceutical composition comprising the immunogenic complex of any one of claims 45-49, and a pharmaceutically acceptable carrier.

53. The pharmaceutical composition of claim 51 or 52, further comprising one or more adjuvants.

54. The pharmaceutical composition of claim 53, wherein the one or more adjuvants is or comprises a co-stimulation factor.

55. The pharmaceutical composition of claim 53 or 54, wherein the one or more adjuvants are selected from the group consisting of aluminum phosphate, aluminum hydroxide, and phosphated aluminum hydroxide.

56. The pharmaceutical composition of any one of claims 53-55, wherein the one or more adjuvants is or comprises aluminum phosphate.

57. The pharmaceutical composition of any one of claims 51-56, wherein the pharmaceutical composition is formulated for injection.

58. The pharmaceutical composition of any one of claims 51-57, wherein upon administration to a subject, the pharmaceutical composition induces an immune response.

59. The pharmaceutical composition of claim 58, wherein the immune response is to (i) at least one polysaccharide antigen of the vaccine or immunogenic complex, and/or (ii) at least one polypeptide antigen of the vaccine or immunogenic complex.

60. A method of making a vaccine, comprising non-covalently complexing a plurality of biotinylated polysaccharide antigens with a plurality of fusion proteins, wherein each fusion protein comprises at least one polypeptide antigen selected SseB, IpaB, SP0785 or SP1500;
wherein the plurality of biotinylated polysaccharide antigens comprises polysaccharides of one or more Shigella serotypes selected from Typhimurium, Enteritidis, Typhi, and Paratyphi A.

61. A method of immunizing a subject against Shigella infection and/or colonization comprising administering to the subject an immunologically effective amount of the vaccine of any one of claims 1-44 and 46.

62. A method of immunizing a subject against Shigella infection and/or colonization comprising administering to the subject an immunologically effective amount of the immunogenic complex of any one of claims 45-49.

63. A method of immunizing a subject against Shigella infection and/or colonization comprising administering to the subject an immunologically effective amount of the pharmaceutical composition of any one of claims 51-58.

64. The method of any one of claims 61-63, wherein the vaccine, immunogenic composition, or pharmaceutical composition induces an immune response.

65. The method of any one of claims 61-64, wherein the immune response is to at least one polysaccharide antigen or at least one polypeptide of a fusion protein.

66. The method of any one of claims 61-64, wherein the subject is immunized against Shigella infection and/or colonization with one dose of a vaccine.

67. The method of any one of claims 61-64, wherein the subject is immunized against Shigella infection and/or colonization with two doses of a vaccine.

68. The method of any one of claims 61-64, wherein the subject is immunized against Shigella infection and/or colonization with three doses of a vaccine.

69. A fusion protein comprising a rhizavidin protein and at least one peptide or polypeptide antigen, wherein the rhizavidin protein comprises amino acids of SEQ ID NO: 3, or 85% sequence identity to amino acids of SEQ ID NO: 3, and Salmonella peptide or polypeptide comprises a fragment of at least 20 amino acids of the SseB protein, or the Shigella peptide or polypeptide comprises a fragment of at least 20 amino acids of the IpaB protein.

70. The fusion protein of claim 69, wherein the SseB protein comprises at least SEQ ID NO: 4 or a protein of at least 20 amino acids that has at least 85% sequence identity to SEQ ID NO: 4.

71. The fusion protein of claim 69, wherein the IpaB protein comprises at least SEQ ID NO: 5 or a protein of at least 20 amino acids that has at least 85% sequence identity to SEQ ID NO: 5.

72. The fusion protein of claim 69, wherein the fusion protein comprises at least SEQ ID NO: 1.

73. The fusion protein of claim 69, wherein the fusion protein comprises at least SEQ ID NO: 2.

74. The fusion protein of any of claims 69-73, wherein the fusion protein comprises a Salmonella peptide or polypeptide comprises a fragment of at least 20 amino acids of the SseB protein, and the Shigella peptide or polypeptide comprises a fragment of at least 20 amino acids of the IpaB protein.

75. The fusion protein of any of claims 69-74, wherein the fusion protein is selected from any fusion protein in Table 1.

76. A vaccine composition comprising at least two immunogenic complexes selected from any combination of Shigella-MAPS immunogenic complexes listed in Table 2A, and at least two or more immunogenic complexes selected from any of the Salmonella-MAPS immunogenic complexes listed in any of Tables 3A, 3B or 3C.

77. The vaccine composition of claim 75, comprising at least three immunogenic complexes selected from any combination of Shigella-MAPS immunogenic complexes listed in Table 2B, and at least two or more immunogenic complexes selected from any of the Salmonella-MAPS immunogenic complexes listed in any of Tables 3A, 3B or 3C.

78. The vaccine composition of claim 75, comprising at least four immunogenic complexes selected from any combination of Shigella-MAPS immunogenic complexes listed in Table 2C, and at least two or more immunogenic complexes selected from any of the Salmonella-MAPS immunogenic complexes listed in any of Tables 3A, 3B or 3C.

79. A fusion proteincomprising, in any order:
a. a biotin-binding moiety and
b.a IpaB polypeptide.
wherein the fusion protein comprises (i) an amino acid sequence that is at least 80% identical to SEQ ID NO: 3 (Rhavi) and (ii) an amino acid sequence that is at least 80% identical to SEQ ID NO: 5 (IpaB).

80. The fusion protein of claim 79, wherein the biotin-binding moiety comprises an amino acid sequence that is at least 80%, 85%, 90% identical to SEQ ID NO: 3 or a biotin binding portion thereof.

81. The fusion protein of claim 79, wherein the IpaB polypeptide comprises an amino acid sequence that is at least 80%, 85%, 90% identical to SEQ ID NO: 5.

82. The fusion protein of claim 79, wherein the fusion protein comprises, in order of N- to C-terminal:
a.a biotin-binding moiety comprises an amino acid sequence of SEQ ID NO: 3, or an amino acid sequence that is at least 80%, 85%, 90% identical to SEQ ID NO:3 or a biotin binding portion thereof, and
b.a IpaB polypeptide comprising an amino acid sequence of SEQ ID NO: 5, or an amino acid sequence an amino acid sequence that is at least 80%, 85%, 90% identical to SEQ ID NO: 5.

83. The fusion protein of claim 79, wherein the fusion protein comprises, in order of N- to C-terminal:
a.a IpaB polypeptide comprising an amino acid sequence of SEQ ID NO: 5, or an amino acid sequence that is at least 80%, 85%, 90% identical to SEQ ID NO:5, and
b.a biotin-binding moiety comprises an amino acid sequence of SEQ ID NO: 3, or an amino acid sequence that is at least 80%, 85%, 90% identical to SEQ ID NO:3 or a biotin binding portion thereof.

84. The fusion protein of claim 79, wherein the fusion protein comprises an amino acid sequence that is at least 80%, 85%, or 90% identical to SEQ ID NO: 2.

85. A fusion protein comprising, in any order:
a. a biotin-binding moiety and
b.a SseB polypeptide.
wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB).

86. The fusion protein of claim 85, wherein the biotin-binding moiety comprises an amino acid sequence that is at least 80%, 85%, 90% identical to SEQ ID NO: 3 or a biotin binding portion thereof.

87. The fusion protein of claim 85, wherein the SseB polypeptide comprises an amino acid sequence that is at least 80%, 85%, 90% identical to SEQ ID NO: 4.

88. The fusion protein of claim 85, wherein the fusion protein comprises, in order of N- to C-terminal:
a.a biotin-binding moiety comprises an amino acid sequence of SEQ ID NO: 3, or an amino acid sequence that is at least 80%, 85%, 90% identical to SEQ ID NO:3 or a biotin binding portion thereof, and
b.a SseB polypeptide comprising an amino acid sequence of SEQ ID NO: 4, or an amino acid sequence an amino acid sequence that is at least 80%, 85%, 90% identical to SEQ ID NO: 4.

89. The fusion protein of claim 85, wherein the fusion protein comprises, in order of N- to C-terminal:
a.a SseB polypeptide comprising an amino acid sequence of SEQ ID NO: 4, or an amino acid sequence that is at least 80%, 85%, 90% identical to SEQ ID NO: 4, and
b.a biotin-binding moiety comprises an amino acid sequence of SEQ ID NO: 3, or an amino acid sequence that is at least 80%, 85%, 90% identical to SEQ ID NO:3 or a biotin binding portion thereof.

90. The fusion protein of claim 85, wherein the fusion protein comprises an amino acid sequence that is at least 80%, 85%, or 90% identical to SEQ ID NO: 1.

91. The fusion protein of any of claims 1-90, further comprising at least one additional antigenic polypeptide, selected from any of: SP1500, SP875, IpaB or SseB.

92. The fusion protein of claim 91, wherein the SP1500 polypeptide comprises an amino acid sequence of SEQ ID NO: 9, or an amino acid sequence that is at least 80%, 85%, 90% identical to SEQ ID NO: 9 or a biotin binding portion thereof.

93. The fusion protein of claim 91, wherein the SP785 polypeptide comprises an amino acid sequence of SEQ ID NO: 8, or an amino acid sequence that is at least 80%, 85%, 90% identical to SEQ ID NO:8 or a biotin binding portion thereof.

94. The fusion protein of claim 91, wherein the SseB polypeptide comprises an amino acid sequence of SEQ ID NO: 4 as disclosed in US patent Publication 2020/0087361, or an amino acid sequence that is at least 80%, 85%, 90% identical to SEQ ID NO:4 or a biotin binding portion thereof.

95. The fusion protein of any of claims 79-94, comprising a linker or spacer positioned between the biotin-binding moiety and the IpaB polypeptide, or the biotin-binding moiety and the SseB polypeptide, wherein the linker comprises the amino acid sequence selected from: GGGSS, GGGGSSS, TDPNSSS, SSS, AAA, or any of SEQ ID NO: 37-52 as disclosed in U.S. application Ser. No. 16/568,646.

96. The fusion protein of any of claims 79-95, wherein a biotin-binding moiety comprises an amino acid sequence of at least 80%, or 90%, or 95% sequence identity to SEQ ID NO: 3 that has any one or more of the amino acid modifications: N80, T108, N118, S119A, N138A.

97. The fusion protein of any of claims 79-96, wherein a biotin-binding moiety comprises an amino acid sequence of at least 80%, or 90%, or 95%, 98%, 99% or 100% sequence identity to SEQ ID NO: 55, wherein the biotin binding moiety has at least one or more of the amino acid modifications: N80, T108, N118, S119A, N138A.

98. An immunogenic composition comprising at least at least one species of immunogenic complex,
wherein each species of the immunogenic complex comprises:
(a) a biotinylated polysaccharide antigen comprising biotin and a 0-specific polysaccharide (OSP) antigen from a Shigella spp serogroup, and
(b) a fusion protein comprising:
(i) a biotin-binding moiety; and
(ii) at least one polypeptide antigen comprising a IpaB polypeptide,
wherein the fusion protein comprises (i) an amino acid sequence that is at least 80% identical to SEQ ID NO: 3 (Rhavi) and (ii) an amino acid sequence that has at least 80% identical to SEQ ID NO: 5(IpaB), or an amino acid sequence that has at least 80% sequence identity to SEQ ID NO: 4 (SseB), and
wherein in each species of the immunogenic complexes, the biotinylated OSP antigen is non-covalently associated with the biotin-binding moiety of the fusion protein.

99. The immunogenic composition of claim 98, wherein the Shigella spp. serogroup is selected from:
Shigella Flexeri or Shigella sonnei.

100. The immunogenic composition of claim 98, wherein the Shigella spp. serogroup is Serogroup A (e.g., Serogroup A comprises of 15 different serotypes; an exemplary example is S. dysenteriae), Serogroup B (comprises of 9 serotypes; an exemplary example is S. flexneri), Serogroup C (comprises of 19 serotypes; an exemplary example is S. boydii) or Serogroup D (comprises of one serotype, S. sonnei).

101. The immunogenic composition of claim 98, wherein the O-specific polysaccharide (OSP) antigen is selected from a distinct Shigella flexeri serotype is selected from any of: 1a, 1b, 1c, 2a, 2b, 3a, 3b, 4a, 4b, 5a, 5b, 6, X, Xv, Y, and F6.

102. The immunogenic composition of claim 98, wherein the O-specific polysaccharide (OSP) antigen is selected from a Shigella flexeri serotype selected from: 2a, 3a or 6, or Shigella sonnei.

103. The immunogenic composition of claim 99, wherein the Shigella sonnei is strain 53G.

104. The immunogenic composition of any of claims 98-103, comprising at least one species of immunogenic complex selected from:
a. an immunogenic complex comprising (a) a biotinylated polysaccharide antigen comprising biotin and a polysaccharide antigen from Shigella flexeri serotype 2a, and (b) a fusion protein;
b.an second immunogenic complex comprising (a) a biotinylated polysaccharide antigen comprising biotin and a polysaccharide antigen from Shigella flexeri serotype 3a, and (b) a fusion protein;
c. an third immunogenic complex comprising (a) a biotinylated polysaccharide antigen comprising biotin and a polysaccharide antigen from Shigella flexeri serotype 6, and (b) a fusion protein; and
d.an fourth immunogenic complex comprising (a) a biotinylated polysaccharide antigen comprising biotin and a polysaccharide antigen from Shigalla Sonnei, and (b) a fusion protein.

105. The immunogenic composition of claim 98, comprising at least 2, or at least 3 immunogenic complexes selected from any of:
a. an immunogenic complex comprising (a) a biotinylated polysaccharide antigen comprising biotin and a polysaccharide antigen from Shigella flexeri serotype 2a, and (b) a fusion protein;
b.an second immunogenic complex comprising (a) a biotinylated polysaccharide antigen comprising biotin and a polysaccharide antigen from Shigella flexeri serotype 3a, and (b) a fusion protein;
c. an third immunogenic complex comprising (a) a biotinylated polysaccharide antigen comprising biotin and a polysaccharide antigen from Shigella flexeri serotype 6, and (b) a fusion protein; and
d.an fourth immunogenic complex comprising (a) a biotinylated polysaccharide antigen comprising biotin and a polysaccharide antigen from Shigalla Sonnei, and (b) a fusion protein.

106. The immunogenic composition of any of claims 98-105, comprising:
a. a first immunogenic complex comprising (a) a biotinylated polysaccharide antigen comprising biotin and a polysaccharide antigen from Shigella flexeri serotype 2a, and (b) a fusion protein;
b.a second immunogenic complex comprising (a) a biotinylated polysaccharide antigen comprising biotin and a polysaccharide antigen from Shigella flexeri serotype 3a, and (b) a fusion protein;
c. a third immunogenic complex comprising (a) a biotinylated polysaccharide antigen comprising biotin and a polysaccharide antigen from Shigella flexeri serotype 6, and (b) a fusion protein; and
d.a fourth immunogenic complex comprising (a) a biotinylated polysaccharide antigen comprising biotin and a polysaccharide antigen from Shigalla Sonnei, and (b) a fusion protein.

107. The immunogenic composition of any of claims 98-106 wherein the OMP antigen is a high molecular weight (HMW) antigen.

108. The immunogenic composition of any of claims 98-107, comprising at least one additional species of immunogenic complex, wherein the additional species of immunogenic complex comprises:
a. (a) a biotinylated polysaccharide antigen comprising biotin and a 0-specific polysaccharide (OSP) antigen from a Samonella spp, and
(b) a fusion protein comprising:
(i) a biotin-binding moiety; and
(ii) at least one polypeptide antigen comprising a IpaB polypeptide,
wherein the fusion protein comprises (i) an amino acid sequence that is at least 80% identical to SEQ ID NO: 3 (Rhavi) and (ii) an amino acid sequence that is at least 80% identical to SEQ ID NO: 5(IpaB), and
wherein in each species of the immunogenic complexes, the biotinylated OSP antigen is non-covalently associated with the biotin-binding moiety of the fusion protein.

109. The immunogenic composition of any of claims 98-108, wherein the OSP is from a Salmonella serogroup A, B, C2 and C3, D and E.

110. The immunogenic composition of claim 109, wherein the OSP is from any of serotypes: Salmonella kentucky strain 1S98, Salmonella enterica serovar Toucra 048 (also known as S. enterica subspecies enterica serovar Toucra), Salmonella enterica serovars Enteritidis and Typhimurium or Salmonella arizonae 045 (Arizona 11).

111. The immunogenic composition of any of claims 98-110, wherein the fusion protein is defined by any of claims 79-97.

112. A pharmaceutical composition comprising the immunogenic composition of any of claims 98-110, and a pharmaceutically acceptable carrier.

113. The pharmaceutical composition of claim 111, further comprising one or more adjuvants.

114. The pharmaceutical composition of claim 111, wherein the one or more adjuvants is or comprises a co-stimulation factor.

115. The pharmaceutical composition of claim 114, wherein the one or more adjuvants are selected from the group consisting of aluminum phosphate, aluminum hydroxide, and phosphated aluminum hydroxide.

116. A vaccine comprising at least one immunogenic composition of any of claims 98-110, or a fusion protein of any of claims 79-97, and a pharmaceutically acceptable carrier.

117. A method of making a multivalent vaccine, comprising mixing two or more species of immunogenic complexes of any of claims 97-110 in a single formulation.

118. The method of claim 117, comprising mixing four or more species of immunogenic complexes in a single formulation, wherein the four or more immunogenic complexes are disclosed in any of claims 104-106.

119. The method of claim 117, wherein each species of the immunogenic complexes comprises least one fusion protein is a fusion protein selected from any of claims 79-97.

120. Use of the immunogenic composition of any of claims 98-110, or the vaccine of claim 116 to induce an immune response to a subject.

121. Use of a vaccine composition of claim 116, or a fusion protein of any of claims 79-97 to induce an immune response in a subject.

122. A method to induce an immune response to a subject, comprising administering to the subject a pharmaceutical composition comprising the immunogenic composition of any of claims 89-110, or the vaccine of claim 116.

123. A method to induce an immune response in a subject, comprising administering a vaccine comprising a fusion protein of any of claims 79-97.

124. The method of claim 123, wherein the immune response is an antibody or B cell response.

125. The method of claims 123-124, wherein the immune response is a CD4+ T cell response, including Th1, Tₕ2, or Tₕ17 response, or a CD8+ T cell response, or CD4+/CD8+ T cell response.

126. The method of claims 123-125, wherein the immune response is:
an antibody or B cell response; and
a T cell response.

127. The method of claims 123-126, wherein the immune response is to: at least the first antigenic polysaccharide, or the second antigenic polysaccharides, or both the first and second antigenic polysaccharide, or at least one polypeptide antigen.

128. The method of claims 123-127, wherein the immune response is an antibody or B cell response to at least the first antigenic polysaccharide and/or second antigenic polysaccharide, and a CD4+ T cell response, including Tₕ 1, Tₕ2, or Tₕ 17 response, or a CD8+ T cell response, or CD4+/CD8+ T cell response to at least one polypeptide antigen.

129. The method of claims 123-128, wherein the immune response is an antibody or B cell response to at least the first antigenic polysaccharide and/or the second antigenic polysaccharide, and an antibody or B cell response and a CD4+ T cell response, including Tₕ1, Tₕ2, or Tₕ 17 response, or a CD8+ T cell response, or CD4+/CD8+ T cell response to at least one polypeptide antigen.

130. The method of claims 123-129, wherein the immune response protects the subject from at least one serotype of Shigella.

131. The method of claim 130, wherein the immune response protects the subject from at least one serotype of Shigella, selected from Shigella Flexeri serotypes 2a, 3a, 6 or Shigella Sonnei.

132. The immunogenic composition of any of claims 98-110, wherein herein the immunogenic composition, upon administration to a subject, elicits (i) an immune response to the at least the OSP antigenic polysaccharide and (ii) an immune response to at least one of the polypeptide antigens, e.g., IpaB or SseB, or both,in the subject.

133. The immunogenic composition of any of claims 98-110, wherein the immune response to the OSP antigenic polysaccharide, and/or at least one of the one polypeptide antigens (e.g., IpaB and/or SseB) comprises an antibody or B cell response.

134. The immunogenic composition of any of claims 98-110, wherein the immune response to the OSP antigenic polysaccharide, and/or at least one of the one polypeptide antigens (e.g., IpaB and/or SseB) comprises a T cell response.

135. The immunogenic composition of any of claims 98-110, wherein the immune response to the OSP antigenic polysaccharide, and/or at least one of the one polypeptide antigens (e.g., IpaB and/or SseB) comprises a CD4+ T cell response, including Tₕ1, Tₕ2, or Tₕ17 response, or a CD8+ T cell response, or a CD4+/CD8+ T cell response.

136. The immunogenic composition of any of claims 98-110, wherein the immune response to the OSP antigenic polysaccharide, and/or at least one of the one polypeptide antigens (e.g., IpaB and/or SseB) comprises:
an antibody or B cell response; and
a T cell response.

137. The immunogenic composition of any of claims 98-110, wherein the immunogenic composition, upon administration to a subject, elicits at least (i) an antibody or B cell response to the OSP antigenic polysaccharide, and (ii) an antibody or B cell response to at least one of polypeptide antigen (e.g., IpaB and/or SseB), in the subject.

138. The immunogenic composition of any of claims 98-110, wherein the immunogenic composition, upon administration to a subject, elicits at least (i) an antibody or B cell response to the OSP antigenic polysaccharide, and (ii) an antibody or B cell response and a CD4+ T cell response, including Th1, Tₕ2, or Tₕ17 response, or a CD8+ T cell response, or a CD4+/CD8+ T cell response to at least one of polypeptide antigens (e.g., IpaB and/or SseB) in the subject.

139. The immunogenic composition of any of claims 98-110, wherein the immunogenic composition, upon administration to a subject, elicits at least (i) an antibody or B cell response to the at least the OMP antigenic polysaccharide, and (ii) a CD4+ T cell response, including Tₕ1, Tₕ2, or Tₕ17 response, or a CD8+ T cell response, or a CD4+/CD8+ T cell response to at least one of polypeptide antigen, e.g., IpaB, in the subject.

140. A heterologous nucleic acid sequence encoding a fusion proteincomprising, in any order:
a. a biotin-binding moiety and
b.a IpaB polypeptide,
wherein the fusion protein comprises (i) an amino acid sequence that is at least 80% identical to SEQ ID NO: 3 (Rhavi) and (ii) an amino acid sequence that is at least 80% identical to SEQ ID NO: 5(IpaB).

141. A heterologous nucleic acid sequence encoding a fusion proteincomprising, in any order:
a. a biotin-binding moiety and
b.a SseB polypeptide,
wherein the fusion protein comprises (i) an amino acid sequence that is at least 80% identical to SEQ ID NO: 3 (Rhavi) and (ii) an amino acid sequence that is at least 80% identical to SEQ ID NO: 4 (SseB).

142. The heterologous nucleic acid of claim 140 or 141, wherein the nucleic acid sequence comprises a nucleic acid sequence of SEQ ID NO: 60 or a nucleic acid sequence at least 85% sequence identity to SEQ ID NO: 60, and a nucleic acid sequence of SEQ ID NO: 46 or a nucleic acid sequence at least 85% sequence identity to SEQ ID NO: 46.

143. The heterologous nucleic acid of claim 140, wherein the nucleic acid sequence comprises SEQ ID NO: 45, or a nucleic acid sequence having at least 85% sequence identity to SEQ ID NO: 45.

144. The heterologous nucleic acid of any of claims 140-143, wherein the heterologous nucleic acid further comprises a nucleic acid that encodes at least one linker, or a nucleic acid that encodes an expression tag, or both.

145. The heterologous nucleic acid sequence of claim 144, wherein the nucleic acid sequence encoding at least one linker is located between SEQ ID NO: 60 and SEQ ID NO: 46, or sequences at having least 85% to SEQ ID NO: 60 or 46, thereof.

146. The heterologous nucleic acid sequence of claim 144, wherein the nucleic acid sequence encoding at least one linker encodes a linker having an amino acid sequence selected from any of: GGGSS, GGGGSSS or AAA and any of: SEQ ID NOS: 39-60 and SEQ ID NO: 62, 63, 108, 109 as disclosed in U.S. application Ser. No. 16/568,646.

147. The heterologous nucleic acid sequence of claim 140, wherein the heterologous nucleic acid is selected from one of the following, in the following order:
a. a nucleic acid sequence of SEQ ID NO: 60 or a nucleic acid sequence at least 85% sequence identity to SEQ ID NO: 60, and a nucleic acid sequence of SEQ ID NO: 46 or a nucleic acid sequence at least 85% sequence identity to SEQ ID NO: 46, or
b.a nucleic acid sequence of SEQ ID NO: 46 or a nucleic acid sequence at least 85% sequence identity to SEQ ID NO: 46, and a nucleic acid sequence of SEQ ID NO: 60 or a nucleic acid sequence at least 85% sequence identity to SEQ ID NO: 60.

148. A cell transfected with the nucleic acid sequence of claim 140 or 141.

149. The cell of claim 148, wherein the cell is transfected with the nucleic acid sequence of claim 143.

150. The cell of claim 148, wherein the cell is transfected with the nucleic acid sequence of claim 142 or 147.

151. The cell of any of claims 148-149, wherein the cell is an expression host cell.

152. The cell of claim 151, wherein the expression host cell is selected from the group consisting of: E. coli, an insect cell line or a mammalian cell line.

153. The cell of claim 152, wherein the insect cell line is baculovirus expression system.

154. The cell of claim 152, wherein the mammalian cell line is a human cell line or Chinese Hamster ovary (CHO) cell line.

155. The cell of claim 152, wherein the heterologous nucleic acid sequence is codon-optimized to improve expression in the host cell.

156. The cell of claim 152, wherein the nucleic acid sequence further comprises a nucleic acid sequence encoding one or more of: polyadenylation sequence or termination sequence, a signal sequence.

157. An expression vector comprising the nucleic acid sequence of any of claims 140-147.

158. The expression vector of claim 157, comprising the nucleic acid sequence of claim 143.

159. The expression vector of claim 157, comprising the nucleic acid sequence of claim 142 or 147.

160. The expression vector of any of claims 148-156, comprising the expression vector of claim 157.

161. The expression vector of any of claims 148-156, comprising the expression vector of claim 158.

162. The expression vector any of claims 148-156, comprising the expression vector of claim 142 or 147.

163. The expression vector of any of claims 140-147, wherein the nucleic acid encodes a fusion protein comprising a biotin-binding moiety having an amino acid sequence of at least 80%, or 90%, or 95% sequence identity to SEQ ID NO: 3 that has any one or more of the amino acid modifications: N80, T108, N118, S119A, N138A.

164. The expression vector of any of claims 140-147 or 163, wherein a biotin-binding moiety comprises an amino acid sequence of at least 80%, or 90%, or 95%, 98%, 99% or 100% sequence identity to SEQ ID NO: 55, wherein the biotin binding moiety has at least one or more of the amino acid modifications: N80, T108, N118, S119A, N138A.


SEQUENCE LISTING (66 sequences)

SEQ ID NO: 1 - protein, 335 aa, synthetic construct
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGSSSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 2 - protein, 719 aa, synthetic construct
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGSSHNVSTTTTGFPLAKILASTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSELDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 3 - protein, 136 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 4 - protein, 196 aa, synthetic construct
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 5 - protein, 580 aa, synthetic construct
MHNVSTTTTGFPLAKILASTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSELDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 6 - protein, 765 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQAAAFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 7 - skipped

SEQ ID NO: 8 - protein, 367 aa, synthetic construct
FRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 9 - protein, 252 aa, synthetic construct
TSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQ

SEQ ID NO: 10 - protein, 720 aa, synthetic construct
MHNVSTTTTGFPLAKILASTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSELDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTAGGGSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 11 - protein, 336 aa, synthetic construct
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYSGGGSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 12 - protein, 620 aa, synthetic construct
MTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATN

SEQ ID NO: 13 - protein, 620 aa, synthetic construct
MFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATNTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQ

SEQ ID NO: 14 - skipped

SEQ ID NO: 15 - protein, 755 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATNTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQ

SEQ ID NO: 16 - protein, 755 aa, synthetic construct
MTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATNFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 17 - protein, 755 aa, synthetic construct
MFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATNTSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 18 - protein, 765 aa, synthetic construct
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSFRQPSQTALKDEPTHLVVAKEGSVASSVLLSGTVTAKNEQYVYFDASKGDLDEILVSVGDKVSEGQALVKYSSSEAQAAYDSASRAVARADRHINELNQARNEAASAPAPQLPAPVGGEDATVQSPTPVAGNSVASIDAQLGDARDARADAAAQLSKAQSQLDATTVLSTLEGTVVEVNSNVSKSPTGASQVMVHIVSNENLQVKGELSEYNLANLSVGQEVSFTSKVYPDKKWTGKLSYISDYPKNNGEAASPAAGNNTGSKYPYTIDVTGEVGDLKQGFSVNIEVKSKTKAILVPVSSLVMDDSKNYVWIVDEQQKAKKVEVSLGNADAENQEITSGLTNGAKVISNPTSSLEEGKEVKADEATNAAATSGDNWSKYQSNKSITIGFDSTFVPMGFAQKDGSYAGFDIDLATAVFEKYGITVNWQPIDWDLKEAELTKGTIDLIWNGYSATDERREKVAFSNSYMKNEQVLVTKKSSGITTAKDMTGKTLGAQAGSSGYADFEANPEILKNIVANKEANQYQTFNEALIDLKNDRIDGLLIDRVYANYYLEAEGVLNDYNVFTVGLETEAFAVGARKEDTNLVKKINEAFSSLYKDGKFQEISQKWFGEDVATKEVKEGQ

SEQ ID NO: 19-20 - skipped (2 entries)

SEQ ID NO: 21 - protein, 7 aa, synthetic construct
GGGGSSS

SEQ ID NO: 22 - protein, 5 aa, synthetic construct
  REGION 1..>5: note = The entire sequence of amino acids 1-5 can be repeated one or more times.
GGGGS

SEQ ID NO: 23 - protein, 6 aa, synthetic construct
GGGGGG

SEQ ID NO: 24 - protein, 15 aa, synthetic construct
GGGGSGGGGSGGGGS

SEQ ID NO: 25 - protein, 30 aa, synthetic construct
GGGGSGGGGSGGGGSGGGGSGGGGSGGGGS

SEQ ID NO: 26 - protein, 18 aa, synthetic construct
KESGSVSSEQLAQFRSLD

SEQ ID NO: 27 - protein, 14 aa, synthetic construct
EGKSSGSGSESKST

SEQ ID NO: 28 - skipped

SEQ ID NO: 29 - protein, 8 aa, synthetic construct
GGGGGGGG

SEQ ID NO: 30 - protein, 12 aa, synthetic construct
GSAGSAAGSGEF

SEQ ID NO: 31 - protein, 5 aa, synthetic construct
  REGION 1..>5: note = The entire sequence of amino acids 1-5 can be repeated one or more times.
EAAAK

SEQ ID NO: 32 - protein, 7 aa, synthetic construct
  REGION 2..6: note = The entire sequence of amino acids 2-6 can be repeated one or more times.
AEAAAKA

SEQ ID NO: 33 - protein, 46 aa, synthetic construct
AEAAAKEAAAKEAAAKEAAAKALEAEAAAKEAAAKEAAAKEAAAKA

SEQ ID NO: 34 - protein, 7 aa, synthetic construct
  REGION 2..6: note = The entire sequence of amino acids 2-6 can be repeated one or more times.
  REGION 1..>7: note = The entire sequence of amino acids 1-7 can be repeated one or more times.
AEAAAKA

SEQ ID NO: 35 - protein, 12 aa, synthetic construct
AEAAAKEAAAKA

SEQ ID NO: 36-39 - skipped (4 entries)

SEQ ID NO: 40 - protein, 14 aa, synthetic construct
APAPAPAPAPAPAP

SEQ ID NO: 41 - protein, 21 aa, synthetic construct
GAPGGGGGAAAAAGGGGGGAP

SEQ ID NO: 42 - protein, 39 aa, synthetic construct
GAPGGGGGAAAAAGGGGGGAPGGGGGAAAAAGGGGGGAP

SEQ ID NO: 43 - protein, 57 aa, synthetic construct
GAPGGGGGAAAAAGGGGGGAPGGGGGAAAAAGGGGGGAPGGGGGAAAAAGGGGGGAP

SEQ ID NO: 44 - skipped

SEQ ID NO: 45 - DNA, 2205 bp, synthetic construct
  source 1..2205: mol_type = other DNA
atggcagatctcaattggatatcggccggccactttgatgcatccaatttcaaggacttctctagcattgcatctgcgagcagctcttggcagaatcaaagtgggtctactatgattatccaagtcgattcattcggcaatgtatctggccagtatgttaatcgtgcccaaggcactggttgtcagaatagcccttatccgcttacaggccgcgtaaacgggacatttatcgcgttctctgtcggctggaataacagtaccgagaattgcaacagtgccacaggctggacaggttatgcgcaagtcaacggaaacaacacagaaatcgtcacaagttggaatttggcatacgaaggaggatcaggtccggccatcgaacaaggacaagatacgtttcaatacgtgcccaccacagagaacaagagtcttctgaaggacggaggtggcagttccagtgcacgtttgcacaacgtctctactacgaccactggatttccattggcgaagatcttagctagtacggagcttggtgataatacgatccaggcagccaacgacgcagccaacaaattgttttcgctgacaatcgctgatttgacagctaaccaaaacattaacaccacgaatgctcattcgacatcaaatattttgattcctgagcttaaagctcctaaatccttgaatgcctcgtcccagttgacattattgatcggtaacttaatccagatcttaggtgaaaagtctttaactgcgttgacaaataaaatcacggcgtggaaatcacagcaacaggcgcgccaacaaaaaaatcttgaattttccgataaaatcaacacccttttatctgaaacagaaggtctgactcgtgactatgaaaaacagattaacaagctgaaaaacgcagactctaaaatcaaggatctggagaataagatcaaccagatccaaacgcgcctgtctgagctggatccggagtcacccgaaaagaaaaagttgagccgtgaagagattcaactgacgatcaaaaaggacgcggcggtgaaggatcgtactttgattgagcagaagacgttgagtattcactcgaaattgacagataagtcgatgcagttggagaaggagatcgacagctttagcgcatttagcaatacggcctcggcagaacaattatcgacgcagcaaaaatctttgacgggccttgcctctgttacccaactgatggcgacatttatccagttggtaggcaagaacaatgaagagagtttaaagaacgacctggccctgtttcagtcgttgcaggaatcgcgcaagactgaaatggagcgcaagtcggacgaatacgcggcagaggttcgtaaagccgaagagctgaaccgcgtaatgggttgtgtcggcaagatcttgggagcactgcttactatcgtaagcgtagttgccgcagccttttcaggaggggcatcattggcattagccgccgtgggtttggcgttgatggttactgatgcgattgtgcaagccgcgacgggaaactccttcatggagcaggcccttaaccccatcatgaaagctgttattgagccactgattaagttgctgagcgacgcctttacaaaaatgttagaaggcctgggtgttgactcgaaaaaggcgaaaatgatcgggagcatcttaggcgctatcgcaggggctcttgtccttgtcgccgccgttgttttggtggccaccgtcggcaagcaagcagccgccaaactggccgaaaacattggaaagatcatcggaaaaactttgaccgatttgatcccgaagttcttgaagaatttttcatctcaacttgacgatctgattacgaatgcagttgcccgtttaaataagttcttgggcgcggctggtgatgaggtaattagtaagcaaatcatttctacccacttgaaccaagcggttttattgggtgaatcagtaaactccgctacacaggcgggcggaagcgtcgccagcgcggtctttcaaaattctgcttcgactaaccttgcagacctgacgctgtctaaatatcaggtggagcaattatctaagtatatttcggaggctatcgaaaagttcggacagttgcaagaggtgattgccgatcttctggctagcatgagcaattctcaggctaaccgcacagacgtagctaaagccattttgcagcaaaccaccgcctga

SEQ ID NO: 46 - DNA, 1743 bp, synthetic construct
  source 1..1743: mol_type = other DNA
atgcataatgtaagcaccacaaccactggttttcctcttgccaaaatattggcttccactgagcttggagacaatactatccaagctgcaaatgatgcagctaacaaattattttctcttacaattgctgatcttactgctaaccaaaatattaatacaactaatgcacactcaacttcaaatatattaatccctgaacttaaagcaccaaagtcattaaatgcaagttcccaactaacgcttttaattggaaaccttattcaaatactcggtgaaaaatctttaactgcattaacaaataaaattactgcttggaagtcccagcaacaggcaagacagcaaaaaaacctagaattctccgataaaattaacactcttctatctgaaactgaaggactaaccagagactatgaaaaacaaattaataaactaaaaaacgcagattctaaaataaaagacctagaaaataaaattaaccaaattcaaacaagattatccgaactcgacccagagtcaccagaaaagaaaaaattaagccgggaagaaatacaactcactatcaaaaaagacgcagcagttaaagacaggacattgattgagcagaaaaccctgtcaattcatagcaaacttacagataaatcaatgcaactcgaaaaagaaatagactctttttctgcattttcaaacacagcatctgctgaacagctatcaacccagcagaaatcattaaccggacttgccagtgttactcaattgatggcaacctttattcaactagttggaaaaaataatgaagaatctttaaaaaatgatctggctctattccagtctctccaagaatcaagaaaaactgaaatggagagaaaatctgatgagtatgctgctgaagtacgtaaagcagaagaactcaacagagtaatgggttgtgttgggaaaatacttggggcacttttaactatcgttagtgttgttgcagcagctttttctggaggagcctctctagcactggcagctgttggtttagctcttatggttacggatgctatagtacaagcagcgaccggcaattccttcatggaacaagccctgaatccgatcatgaaagcagtcattgaacccttaatcaaactcctttcagatgcatttacaaaaatgctcgaaggcttgggcgtcgactcgaaaaaagccaaaatgattggctctattctgggggcaatcgcaggcgctcttgtcctagttgcagcagtcgttctcgtagccactgttggtaaacaggcagcagcaaaacttgcagaaaatattggcaaaataataggtaaaaccctcacagaccttataccaaagtttctcaagaatttttcttctcaactggacgatttaatcactaatgctgttgccagattaaataaatttcttggtgcagcgggtgatgaagtaatatccaaacaaattatttccacccatttaaaccaagcagttttattaggagaaagtgttaactctgccacacaagcgggaggaagtgtcgcttctgctgttttccagaacagcgcgtcgacaaatctagcagacctgacattatcgaaatatcaagttgaacaactgtcaaaatatatcagtgaagcaatagaaaaattcggccaattgcaggaagtaattgcagatctattagcctcaatgtccaactctcaggctaatagaactgatgttgcaaaagcaattttgcaacaaactactgcttga

SEQ ID NO: 47 - protein, 6 aa, synthetic construct
GGGGGG

SEQ ID NO: 48 - protein, 8 aa, synthetic construct
GGGGGGGG

SEQ ID NO: 49 - protein, 5 aa, synthetic construct
EAAAK

SEQ ID NO: 50-54 - skipped (5 entries)

SEQ ID NO: 55 - protein, 135 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGNATEIVTSWNLAYEGGSGPAIEQGQDTFQYVPTTENKSLLKD

SEQ ID NO: 56 - protein, 918 aa, synthetic construct
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGSSHNVSTTTTGFPLAKILASTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSELDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTAAAAMSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 57 - protein, 918 aa, synthetic construct
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGSSMSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYSAAAHNVSTTTTGFPLAKILASTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSELDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 58 - protein, 6 aa, synthetic construct
HHHHHH

SEQ ID NO: 59 - protein, 10 aa, synthetic construct
MSYYHHHHHH

SEQ ID NO: 60 - DNA, 405 bp, unidentified
  source 1..405: mol_type = unassigned DNA
tttgatgcgagtaactttaaagatttcagctctattgcgagcgccagtagctcttggcagaatcagagtggcagcaccatgattatccaggtggattctttcggcaacgttagtggccagtatgttaatcgtgcgcagggcacgggttgccagaactctccgtacccgctgaccggccgcgtgaatggcacgtttatcgccttcagcgttggctggaacaattctaccgaaaactgtaatagtgcaaccggctggacgggttatgcgcaggtgaacggtaacaataccgaaattgttacgagctggaatctggcctatgaaggcggttctggcccggcaatcgaacagggtcaggatacctttcagtacgttccgaccacggaaaacaaaagcctgctgaaagat

SEQ ID NO: 61 - DNA, 597 bp, Salmonella enterica
  source 1..597: mol_type = unassigned DNA
  misc_feature 1..597: note = From Salmonella enterica subsp. enterica serovar Typhimurium str. LT2.
agcataatgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcatga

SEQ ID NO: 62 - DNA, 1101 bp, unidentified
  source 1..1101: mol_type = unassigned DNA
tttagacaaccttctcagactgctctaaaagatgagcctactcatcttgttgttgccaaggaaggaagcgtggcctcctctgttttattgtcagggacagtaacagcaaaaaatgaacaatatgtttattttgatgctagtaagggtgatttagatgaaatccttgtttctgtgggcgataaggtcagcgaagggcaggctttagtcaagtacagtagttcagaagcgcaggcggcctatgattcagctagtcgagcagtagctagggcagatcgtcatatcaatgaactcaatcaagcacgaaatgaagccgcttcagctccggctccacagttaccagcgccagtaggaggagaagatgcaacggtgcaaagcccaactccagtggctggaaattctgttgcttctattgacgctcaattgggtgatgcccgtgatgcgcgtgcagatgctgcggcgcaattaagcaaggctcaaagtcaattggatgcaacaactgttctcagtaccctagagggaactgtggtcgaagtcaatagcaatgtttctaaatctccaacaggggcgagtcaagttatggttcatattgtcagcaatgaaaatttacaagtcaagggagaattgtctgagtacaatctagccaacctttctgtaggtcaagaagtaagctttacttctaaagtgtatcctgataaaaaatggactgggaaattaagctatatttctgactatcctaaaaacaatggtgaagcagctagtccagcagccgggaataatacaggttctaaatacccttatactattgatgtgacaggcgaggttggtgatttgaaacaaggtttttctgtcaacattgaggttaaaagcaaaactaaggctattcttgttcctgttagcagtctagtaatggatgatagtaaaaattatgtctggattgtggatgaacaacaaaaggctaaaaaagttgaggtttcattgggaaatgctgacgcagaaaatcaagaaatcacttctggtttaacgaacggtgctaaggtcatcagtaatccaacatcttccttggaagaaggaaaagaggtgaaggctgatgaagcaactaat

SEQ ID NO: 63 - DNA, 756 bp, unidentified
  source 1..756: mol_type = unassigned DNA
actagtggagataattggtcaaagtaccagtctaacaagtctattactattggatttgatagtacttttgttccaatgggatttgctcagaaagatggttcttatgcaggatttgatattgatttagctacagctgtttttgaaaaatacggaatcacggtaaattggcaaccgattgattgggatttgaaagaagctgaattgacaaaaggaacgattgatctgatttggaatggctattccgctacagacgaacgccgtgaaaaggtggctttcagtaactcatatatgaagaatgagcaggtattggttacgaagaaatcatctggtatcacgactgcaaaggatatgactggaaagacattaggagctcaagctggttcatctggttatgcggactttgaagcaaatccagaaattttgaagaatattgtcgctaataaggaagcgaatcaataccaaacctttaatgaagccttgattgatttgaaaaacgatcgaattgatggtctattgattgaccgtgtctatgcaaactattatttagaagcagaaggtgttttaaacgattataatgtctttacagttggactagaaacagaagcttttgcggttggagcccgtaaggaagatacaaacttggttaagaagataaatgaagctttttctagtctttacaaggacggcaagttccaagaaatcagccaaaaatggtttggagaagatgtagcaaccaaagaagtaaaagaaggacag

SEQ ID NO: 64 - DNA, 2295 bp, synthetic construct
  source 1..2295: mol_type = other DNA
atgttcgacgcatccaactttaaagactttagcagcatcgcgtccgcaagctctagctggcagaatcaatctggtagcaccatgattatccaagtggacagctttggtaacgtcagcggtcaatatgttaatcgtgcacagggtacgggttgtcagaattctccgtacccgctgaccggtcgtgttaacggcacgttcatcgctttcagcgtcggttggaacaattctactgaaaattgcaacagcgcgaccggttggacgggctatgcacaagtgaatggcaataacaccgaaatcgtcacgtcctggaatctggcgtatgagggtggcagcggtccggctattgaacagggccaggataccttccaatacgtccctacgaccgagaataagtcccttctgaaagacggcggtggcggttcgagctcgaccagcggcgacaattggtccaaataccagagcaacaagagcatcacgatcggcttcgacagcacttttgtgccgatgggtttcgcgcaaaaagacggtagctacgcgggtttcgatattgacctggcgaccgctgtctttgagaaatacggcattacggttaattggcagccgattgattgggacctgaaagaggccgaactcaccaaaggcaccatcgacctgatctggaatggttactccgcaaccgatgagcgtcgcgaaaaagttgccttcagcaacagctatatgaagaatgaacaagtgttggtaaccaagaaatctagcggcattacgaccgcgaaagacatgaccggtaagacgctgggtgcgcaggccggtagctctggctatgcggatttcgaggcgaatcctgagattctgaaaaacatcgttgcgaataaagaggcgaaccagtaccagacctttaacgaagcactgatcgacctgaaaaacgatcgcattgacggtctgctgatcgatcgtgtgtacgcgaactattatctggaagccgagggcgttctgaacgattataatgtttttaccgtgggtctggagactgaggcattcgcggttggtgcgcgcaaggaagataccaacctggttaaaaagattaatgaggcatttagctcactgtacaaggacggcaagttccaagaaattagccagaagtggttcggtgaagatgttgcgacgaaagaggttaaagagggccaagcggccgcatttcgccaaccgagccagactgcgttgaaagatgagccgacccatctggttgttgcgaaagagggcagcgtggcatcgagcgtgctgctgagcggtacggttactgccaaaaacgaacaatacgtgtacttcgatgctagcaagggtgatctggatgaaattctggtgagcgtgggtgacaaagttagcgaaggccaggcactggtgaagtattcatcctccgaggcacaggcagcgtacgacagcgcaagccgcgcagtggcgcgtgccgaccgtcacattaacgaattgaaccaagcgcgtaacgaggccgcaagcgcgccagcaccgcagctgccggctccggtgggtggcgaagatgcgacggtgcagagcccgaccccggttgcgggtaattcggtcgccagcatcgatgcgcagctgggtgacgcgcgtgatgcccgtgcggatgcggctgctcaactgagcaaggctcagagccaactggacgcgacgacggtgctgagcaccttggagggtaccgttgtcgaagtcaacagcaatgtgagcaagagcccaacgggtgcgagccaggttatggtccacattgtgagcaatgaaaacttacaggtcaagggtgagctgagcgagtataacctggcgaatctgagcgttggtcaagaggtcagctttaccagcaaggtctacccggataagaaatggaccggcaagttgagctacatcagcgactacccgaagaacaatggcgaggcagcctccccggcagccggcaacaataccggctctaagtatccgtacaccatcgacgtaaccggtgaggtcggcgacctgaaacagggttttagcgtgaatatcgaagtgaagtccaagaccaaggcaattttggttccggttagctccctggtgatggacgatagcaagaattatgtgtggattgtcgacgagcaacagaaagcgaaaaaagttgaagtgagcctgggcaatgctgatgccgagaaccaagaaatcacgtctggtctgaccaacggtgcgaaagttattagcaacccgaccagcagcctggaagagggtaaagaggtcaaagccgacgaagctacgaac

SEQ ID NO: 65 - DNA, 2294 bp, synthetic construct
  source 1..2294: mol_type = other DNA
atgttcgacgcatccaactttaaagactttagcagcatcgcgtccgcaagctctagctggcagaatcaatctggtagcaccatgattatccaagtggacagctttggtaacgtcagcggtcaatatgttaatcgtgcacagggtacgggttgtcagaattctccgtacccgctgaccggtcgtgttaacggcacgttcatcgctttcagcgtcggttggaacaattctactgaaaattgcaacagcgcgaccggttggacgggctatgcacaagtgaatggcaataacaccgaaatcgtcacgtcctggaatctggcgtatgagggtggcagcggtccggctattgaacagggccaggataccttccaatacgtccctacgaccgagaataagtcccttctgaaagacggcggtggcggttcgagctcgtttcgccaaccgagccagactgcgttgaaagatgagccgacccatctggttgttgcgaaagagggcagcgtggcatcgagcgtgctgctgagcggtacggttactgccaaaaacgaacaatacgtgtacttcgatgctagcaagggtgatctggatgaaattctggtgagcgtgggtgacaaagttagcgaaggccaggcactggtgaagtattcatcctccgaggcacaggcagcgtacgacagcgcaagccgcgcagtggcgcgtgccgaccgtcacattaacgaattgaaccaagcgcgtaacgaggccgcaagcgcgccagcaccgcagctgccggctccggtgggtggcgaagatgcgacggtgcagagcccgaccccggttgcgggtaattcggtcgccagcatcgatgcgcagctgggtgacgcgcgtgatgcccgtgcggatgcggctgctcaactgagcaaggctcagagccaactggacgcgacgacggtgctgagcaccttggagggtaccgttgtcgaagtcaacagcaatgtgagcaagagcccaacgggtgcgagccaggttatggtccacattgtgagcaatgaaaacttacaggtcaagggtgagctgagcgagtataacctggcgaatctgagcgttggtcaagaggtcagctttaccagcaaggtctacccggataagaaatggaccggcaagttgagctacatcagcgactacccgaagaacaatggcgaggcagcctccccggcagccggcaacaataccggctctaagtatccgtacaccatcgacgtaaccggtgaggtcggcgacctgaaacagggttttagcgtgaatatcgaagtgaagtccaagaccaaggcaattttggttccggttagctccctggtgatggacgatagcaagaattatgtgtggattgtcgacgagcaacagaaagcgaaaaaagttgaagtgagcctgggcaatgctgatgccgagaaccaagaaatcacgtctggtctgaccaacggtgcgaaagttattagcaacccgaccagcagcctggaagagggtaaagaggtcaaagccgacgaagctacgaaccggccgcaaccagcggcgacaattggtccaaataccagagcaacaagagcatcacgatcggcttcgacagcacttttgtgccgatgggtttcgcgcaaaaagacggtagctacgcgggtttcgatattgacctggcgaccgctgtctttgagaaatacggcattacggttaattggcagccgattgattgggacctgaaagaggccgaactcaccaaaggcaccatcgacctgatctggaatggttactccgcaaccgatgagcgtcgcgaaaaagttgccttcagcaacagctatatgaagaatgaacaagtgttggtaaccaagaaatctagcggcattacgaccgcgaaagacatgaccggtaagacgctgggtgcgcaggccggtagctctggctatgcggatttcgaggcgaatcctgagattctgaaaaacatcgttgcgaataaagaggcgaaccagtaccagacctttaacgaagcactgatcgacctgaaaaacgatcgcattgacggtctgctgatcgatcgtgtgtacgcgaactattatctggaagccgagggcgttctgaacgattataatgtttttaccgtgggtctggagactgaggcattcgcggttggtgcgcgcaaggaagataccaacctggttaaaaagattaatgaggcatttagctcactgtacaaggacggcaagttccaagaaattagccagaagtggttcggtgaagatgttgcgacgaaagaggttaaagagggccaa

SEQ ID NO: 66 - DNA, 1002 bp, synthetic construct
  source 1..1002: mol_type = other DNA
tttgatgcgagtaactttaaagatttcagctctattgcgagcgccagtagctcttggcagaatcagagtggcagcaccatgattatccaggtggattctttcggcaacgttagtggccagtatgttaatcgtgcgcagggcacgggttgccagaactctccgtacccgctgaccggccgcgtgaatggcacgtttatcgccttcagcgttggctggaacaattctaccgaaaactgtaatagtgcaaccggctggacgggttatgcgcaggtgaacggtaacaataccgaaattgttacgagctggaatctggcctatgaaggcggttctggcccggcaatcgaacagggtcaggatacctttcagtacgttccgaccacggaaaacaaaagcctgctgaaagatagcataatgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcatga